SEQUENCE LISTING (263 sequences)

SEQ ID NO: 1 - DNA, 530 bp, Homo sapiens
gggcaggtgagtggctgtggggagaggggttgtcgggctgggccgagctgaccctcgtttccccacaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacaggtgaggcacaggcttcagaagtggccgcaagggaagttcatgggtactgcagggcagggctgggatgggaccctggtactg

SEQ ID NO: 2 - DNA, 25 bp, Artificial Sequence
  Description of Artificial Sequence Synthetic oligonucleotide
cctcacctgtcacatgcacagagag

SEQ ID NO: 3 - DNA, 25 bp, Artificial Sequence
  Description of Artificial Sequence Synthetic oligonucleotide
cctgtcacatgcacagagagctggg

SEQ ID NO: 4 - DNA, 25 bp, Artificial Sequence
  Description of Artificial Sequence Synthetic oligonucleotide
atttggtacttcctctctccatccg

SEQ ID NO: 5 - DNA, 25 bp, Artificial Sequence
  Description of Artificial Sequence Synthetic oligonucleotide
tctccatccgaaagaagtatgaacc

SEQ ID NO: 6 - DNA, 25 bp, Artificial Sequence
  Description of Artificial Sequence Synthetic oligonucleotide
tgggatggaagaaagtgcagggcac

SEQ ID NO: 7 - DNA, 25 bp, Artificial Sequence
  Description of Artificial Sequence Synthetic oligonucleotide
cagccagaaatttggatccatagcc

SEQ ID NO: 8 - DNA, 25 bp, Artificial Sequence
  Description of Artificial Sequence Synthetic oligonucleotide
ccctgtggggaaacgagggtcagct

SEQ ID NO: 9 - DNA, 20 bp, Artificial Sequence
  Description of Artificial Sequence Synthetic oligonucleotide
cacatgcacagagagctggg

SEQ ID NO: 10 - DNA, 20 bp, Artificial Sequence
  Description of Artificial Sequence Synthetic oligonucleotide
gagagctggggagatttgta

SEQ ID NO: 11 - DNA, 20 bp, Artificial Sequence
  Description of Artificial Sequence Synthetic oligonucleotide
tctccatccgaaagaagtat

SEQ ID NO: 12 - DNA, 20 bp, Artificial Sequence
  Description of Artificial Sequence Synthetic oligonucleotide
atccgaaagaagtatgaacc

SEQ ID NO: 13 - DNA, 20 bp, Artificial Sequence
  Description of Artificial Sequence Synthetic oligonucleotide
atgctcagggagcagttgtt

SEQ ID NO: 14 - DNA, 20 bp, Artificial Sequence
  Description of Artificial Sequence Synthetic oligonucleotide
gagtctcctcctgtacttct

SEQ ID NO: 15 - DNA, 20 bp, Artificial Sequence
  Description of Artificial Sequence Synthetic oligonucleotide
cgcacaaaccctcctgtacc

SEQ ID NO: 16 - DNA, 25 bp, Artificial Sequence
  Description of Artificial Sequence Synthetic oligonucleotide
cagtaccagggtcccatcccagccc

SEQ ID NO: 17 - DNA, 25 bp, Artificial Sequence
  Description of Artificial Sequence Synthetic oligonucleotide
ccagggtcccatcccagccctgccc

SEQ ID NO: 18 - DNA, 25 bp, Artificial Sequence
  Description of Artificial Sequence Synthetic oligonucleotide
gtcccatcccagccctgccctgcag

SEQ ID NO: 19 - DNA, 25 bp, Artificial Sequence
  Description of Artificial Sequence Synthetic oligonucleotide
atcccagccctgccctgcagtaccc

SEQ ID NO: 20 - DNA, 25 bp, Artificial Sequence
  Description of Artificial Sequence Synthetic oligonucleotide
agccctgccctgcagtacccatgaa

SEQ ID NO: 21 - DNA, 25 bp, Artificial Sequence
  Description of Artificial Sequence Synthetic oligonucleotide
tgccctgcagtacccatgaacttcc

SEQ ID NO: 22 - DNA, 25 bp, Artificial Sequence
  Description of Artificial Sequence Synthetic oligonucleotide
tgcagtacccatgaacttcccttgc

SEQ ID NO: 23 - DNA, 25 bp, Artificial Sequence
  Description of Artificial Sequence Synthetic oligonucleotide
tacccatgaacttcccttgcggcca

SEQ ID NO: 24 - DNA, 25 bp, Artificial Sequence
  Description of Artificial Sequence Synthetic oligonucleotide
atgaacttcccttgcggccacttct

SEQ ID NO: 25 - DNA, 25 bp, Artificial Sequence
  Description of Artificial Sequence Synthetic oligonucleotide
cttcccttgcggccacttctgaagc

SEQ ID NO: 26 - DNA, 25 bp, Artificial Sequence
  Description of Artificial Sequence Synthetic oligonucleotide
cttgcggccacttctgaagcctgtg

SEQ ID NO: 27 - DNA, 25 bp, Artificial Sequence
  Description of Artificial Sequence Synthetic oligonucleotide
ggccacttctgaagcctgtgcctca

SEQ ID NO: 28 - DNA, 25 bp, Artificial Sequence
  Description of Artificial Sequence Synthetic oligonucleotide
cttctgaagcctgtgcctcacctgt

SEQ ID NO: 29 - DNA, 25 bp, Artificial Sequence
  Description of Artificial Sequence Synthetic oligonucleotide
gaagcctgtgcctcacctgtcacat

SEQ ID NO: 30 - DNA, 25 bp, Artificial Sequence
  Description of Artificial Sequence Synthetic oligonucleotide
ctgtgcctcacctgtcacatgcaca

SEQ ID NO: 31 - DNA, 25 bp, Artificial Sequence
  Description of Artificial Sequence Synthetic oligonucleotide
cacatgcacagagagctggggagat

SEQ ID NO: 32 - DNA, 25 bp, Artificial Sequence
  Description of Artificial Sequence Synthetic oligonucleotide
gcacagagagctggggagatttgta

SEQ ID NO: 33 - DNA, 25 bp, Artificial Sequence
  Description of Artificial Sequence Synthetic oligonucleotide
gagagctggggagatttgtaactgt

SEQ ID NO: 34 - DNA, 25 bp, Artificial Sequence
  Description of Artificial Sequence Synthetic oligonucleotide
ctggggagatttgtaactgtatttg

SEQ ID NO: 35 - DNA, 25 bp, Artificial Sequence
  Description of Artificial Sequence Synthetic oligonucleotide
gagatttgtaactgtatttggtact

SEQ ID NO: 36 - DNA, 25 bp, Artificial Sequence
  Description of Artificial Sequence Synthetic oligonucleotide
ttgtaactgtatttggtacttcctc

SEQ ID NO: 37 - DNA, 25 bp, Artificial Sequence
  Description of Artificial Sequence Synthetic oligonucleotide
actgtatttggtacttcctctctcc

SEQ ID NO: 38 - DNA, 25 bp, Artificial Sequence
  Description of Artificial Sequence Synthetic oligonucleotide
gtacttcctctctccatccgaaaga

SEQ ID NO: 39 - DNA, 25 bp, Artificial Sequence
  Description of Artificial Sequence Synthetic oligonucleotide
tcctctctccatccgaaagaagtat

SEQ ID NO: 40 - DNA, 25 bp, Artificial Sequence
  Description of Artificial Sequence Synthetic oligonucleotide
atccgaaagaagtatgaaccattat

SEQ ID NO: 41 - DNA, 25 bp, Artificial Sequence
  Description of Artificial Sequence Synthetic oligonucleotide
aaagaagtatgaaccattatccctc

SEQ ID NO: 42 - DNA, 25 bp, Artificial Sequence
  Description of Artificial Sequence Synthetic oligonucleotide
agtatgaaccattatccctcctcct

SEQ ID NO: 43 - DNA, 25 bp, Artificial Sequence
  Description of Artificial Sequence Synthetic oligonucleotide
gaaccattatccctcctcctggcgt

SEQ ID NO: 44 - DNA, 25 bp, Artificial Sequence
  Description of Artificial Sequence Synthetic oligonucleotide
attatccctcctcctggcgtctacg

SEQ ID NO: 45 - DNA, 25 bp, Artificial Sequence
  Description of Artificial Sequence Synthetic oligonucleotide
ccctcctcctggcgtctacgatgct

SEQ ID NO: 46 - DNA, 25 bp, Artificial Sequence
  Description of Artificial Sequence Synthetic oligonucleotide
ctcctggcgtctacgatgctcaggg

SEQ ID NO: 47 - DNA, 25 bp, Artificial Sequence
  Description of Artificial Sequence Synthetic oligonucleotide
ggcgtctacgatgctcagggagcag

SEQ ID NO: 48 - DNA, 25 bp, Artificial Sequence
  Description of Artificial Sequence Synthetic oligonucleotide
ctacgatgctcagggagcagttgtt

SEQ ID NO: 49 - DNA, 25 bp, Artificial Sequence
  Description of Artificial Sequence Synthetic oligonucleotide
atgctcagggagcagttgttcctac

SEQ ID NO: 50 - DNA, 25 bp, Artificial Sequence
  Description of Artificial Sequence Synthetic oligonucleotide
cagggagcagttgttcctactggga

SEQ ID NO: 51 - DNA, 25 bp, Artificial Sequence
  Description of Artificial Sequence Synthetic oligonucleotide
agcagttgttcctactgggatcccc

SEQ ID NO: 52 - DNA, 25 bp, Artificial Sequence
  Description of Artificial Sequence Synthetic oligonucleotide
ttgttcctactgggatccccaagga

SEQ ID NO: 53 - DNA, 25 bp, Artificial Sequence
  Description of Artificial Sequence Synthetic oligonucleotide
cctactgggatccccaaggaggcgg

SEQ ID NO: 54 - DNA, 25 bp, Artificial Sequence
  Description of Artificial Sequence Synthetic oligonucleotide
tgggatccccaaggaggcggaatct

SEQ ID NO: 55 - DNA, 25 bp, Artificial Sequence
  Description of Artificial Sequence Synthetic oligonucleotide
tccccaaggaggcggaatctgccct

SEQ ID NO: 56 - DNA, 25 bp, Artificial Sequence
  Description of Artificial Sequence Synthetic oligonucleotide
aaggaggcggaatctgccctgagtc

SEQ ID NO: 57 - DNA, 25 bp, Artificial Sequence
  Description of Artificial Sequence Synthetic oligonucleotide
ggcggaatctgccctgagtctcctc

SEQ ID NO: 58 - DNA, 25 bp, Artificial Sequence
  Description of Artificial Sequence Synthetic oligonucleotide
aatctgccctgagtctcctcctgta

SEQ ID NO: 59 - DNA, 25 bp, Artificial Sequence
  Description of Artificial Sequence Synthetic oligonucleotide
gccctgagtctcctcctgtacttct

SEQ ID NO: 60 - DNA, 25 bp, Artificial Sequence
  Description of Artificial Sequence Synthetic oligonucleotide
gagtctcctcctgtacttcttgatc

SEQ ID NO: 61 - DNA, 25 bp, Artificial Sequence
  Description of Artificial Sequence Synthetic oligonucleotide
tcctcctgtacttcttgatctagct

SEQ ID NO: 62 - DNA, 25 bp, Artificial Sequence
  Description of Artificial Sequence Synthetic oligonucleotide
ctgtacttcttgatctagcttgttt

SEQ ID NO: 63 - DNA, 25 bp, Artificial Sequence
  Description of Artificial Sequence Synthetic oligonucleotide
cttcttgatctagcttgtttgtggc

SEQ ID NO: 64 - DNA, 25 bp, Artificial Sequence
  Description of Artificial Sequence Synthetic oligonucleotide
tgatctagcttgtttgtggccactg

SEQ ID NO: 65 - DNA, 25 bp, Artificial Sequence
  Description of Artificial Sequence Synthetic oligonucleotide
tagcttgtttgtggccactggagag

SEQ ID NO: 66 - DNA, 25 bp, Artificial Sequence
  Description of Artificial Sequence Synthetic oligonucleotide
tgtttgtggccactggagagtccct

SEQ ID NO: 67 - DNA, 25 bp, Artificial Sequence
  Description of Artificial Sequence Synthetic oligonucleotide
gtggccactggagagtccctggata

SEQ ID NO: 68 - DNA, 25 bp, Artificial Sequence
  Description of Artificial Sequence Synthetic oligonucleotide
cactggagagtccctggatataatg

SEQ ID NO: 69 - DNA, 25 bp, Artificial Sequence
  Description of Artificial Sequence Synthetic oligonucleotide
gagagtccctggatataatggctcc

SEQ ID NO: 70 - DNA, 25 bp, Artificial Sequence
  Description of Artificial Sequence Synthetic oligonucleotide
tccctggatataatggctccttccc

SEQ ID NO: 71 - DNA, 25 bp, Artificial Sequence
  Description of Artificial Sequence Synthetic oligonucleotide
ggatataatggctccttcccggaac

SEQ ID NO: 72 - DNA, 25 bp, Artificial Sequence
  Description of Artificial Sequence Synthetic oligonucleotide
taatggctccttcccggaaccagta

SEQ ID NO: 73 - DNA, 25 bp, Artificial Sequence
  Description of Artificial Sequence Synthetic oligonucleotide
gctccttcccggaaccagtaaccat

SEQ ID NO: 74 - DNA, 25 bp, Artificial Sequence
  Description of Artificial Sequence Synthetic oligonucleotide
ttcccggaaccagtaaccatgaact

SEQ ID NO: 75 - DNA, 25 bp, Artificial Sequence
  Description of Artificial Sequence Synthetic oligonucleotide
ggaaccagtaaccatgaactgggga

SEQ ID NO: 76 - DNA, 25 bp, Artificial Sequence
  Description of Artificial Sequence Synthetic oligonucleotide
cagtaaccatgaactggggagttct

SEQ ID NO: 77 - DNA, 25 bp, Artificial Sequence
  Description of Artificial Sequence Synthetic oligonucleotide
accatgaactggggagttcttgtcg

SEQ ID NO: 78 - DNA, 25 bp, Artificial Sequence
  Description of Artificial Sequence Synthetic oligonucleotide
gaactggggagttcttgtcgtagta

SEQ ID NO: 79 - DNA, 25 bp, Artificial Sequence
  Description of Artificial Sequence Synthetic oligonucleotide
ggggagttcttgtcgtagtagggta

SEQ ID NO: 80 - DNA, 25 bp, Artificial Sequence
  Description of Artificial Sequence Synthetic oligonucleotide
gttcttgtcgtagtagggtatggga

SEQ ID NO: 81 - DNA, 25 bp, Artificial Sequence
  Description of Artificial Sequence Synthetic oligonucleotide
tgtcgtagtagggtatgggatggaa

SEQ ID NO: 82 - DNA, 25 bp, Artificial Sequence
  Description of Artificial Sequence Synthetic oligonucleotide
tagtagggtatgggatggaagaaag

SEQ ID NO: 83 - DNA, 25 bp, Artificial Sequence
  Description of Artificial Sequence Synthetic oligonucleotide
gggtatgggatggaagaaagtgcag

SEQ ID NO: 84 - DNA, 25 bp, Artificial Sequence
  Description of Artificial Sequence Synthetic oligonucleotide
tggaagaaagtgcagggcacgagga

SEQ ID NO: 85 - DNA, 25 bp, Artificial Sequence
  Description of Artificial Sequence Synthetic oligonucleotide
gaaagtgcagggcacgaggacgcac

SEQ ID NO: 86 - DNA, 25 bp, Artificial Sequence
  Description of Artificial Sequence Synthetic oligonucleotide
tgcagggcacgaggacgcacaaacc

SEQ ID NO: 87 - DNA, 25 bp, Artificial Sequence
  Description of Artificial Sequence Synthetic oligonucleotide
ggcacgaggacgcacaaaccctcct

SEQ ID NO: 88 - DNA, 25 bp, Artificial Sequence
  Description of Artificial Sequence Synthetic oligonucleotide
gaggacgcacaaaccctcctgtacc

SEQ ID NO: 89 - DNA, 25 bp, Artificial Sequence
  Description of Artificial Sequence Synthetic oligonucleotide
cgcacaaaccctcctgtaccgtcac

SEQ ID NO: 90 - DNA, 25 bp, Artificial Sequence
  Description of Artificial Sequence Synthetic oligonucleotide
aaaccctcctgtaccgtcactgact

SEQ ID NO: 91 - DNA, 25 bp, Artificial Sequence
  Description of Artificial Sequence Synthetic oligonucleotide
ctcctgtaccgtcactgactcctgc

SEQ ID NO: 92 - DNA, 25 bp, Artificial Sequence
  Description of Artificial Sequence Synthetic oligonucleotide
gtaccgtcactgactcctgcacttg

SEQ ID NO: 93 - DNA, 25 bp, Artificial Sequence
  Description of Artificial Sequence Synthetic oligonucleotide
gtcactgactcctgcacttgcagcc

SEQ ID NO: 94 - DNA, 25 bp, Artificial Sequence
  Description of Artificial Sequence Synthetic oligonucleotide
tgactcctgcacttgcagccagaaa

SEQ ID NO: 95 - DNA, 25 bp, Artificial Sequence
  Description of Artificial Sequence Synthetic oligonucleotide
cctgcacttgcagccagaaatttgg

SEQ ID NO: 96 - DNA, 25 bp, Artificial Sequence
  Description of Artificial Sequence Synthetic oligonucleotide
acttgcagccagaaatttggatcca

SEQ ID NO: 97 - DNA, 25 bp, Artificial Sequence
  Description of Artificial Sequence Synthetic oligonucleotide
agaaatttggatccatagccagggc

SEQ ID NO: 98 - DNA, 25 bp, Artificial Sequence
  Description of Artificial Sequence Synthetic oligonucleotide
tttggatccatagccagggcccctg

SEQ ID NO: 99 - DNA, 25 bp, Artificial Sequence
  Description of Artificial Sequence Synthetic oligonucleotide
atccatagccagggcccctgtgggg

SEQ ID NO: 100 - DNA, 25 bp, Artificial Sequence
  Description of Artificial Sequence Synthetic oligonucleotide
tagccagggcccctgtggggaaacg

SEQ ID NO: 101 - DNA, 25 bp, Artificial Sequence
  Description of Artificial Sequence Synthetic oligonucleotide
agggcccctgtggggaaacgagggt

SEQ ID NO: 102 - DNA, 25 bp, Artificial Sequence
  Description of Artificial Sequence Synthetic oligonucleotide
tggggaaacgagggtcagctcggcc

SEQ ID NO: 103 - DNA, 25 bp, Artificial Sequence
  Description of Artificial Sequence Synthetic oligonucleotide
aaacgagggtcagctcggcccagcc

SEQ ID NO: 104 - DNA, 25 bp, Artificial Sequence
  Description of Artificial Sequence Synthetic oligonucleotide
agggtcagctcggcccagcccgaca

SEQ ID NO: 105 - DNA, 25 bp, Artificial Sequence
  Description of Artificial Sequence Synthetic oligonucleotide
cagctcggcccagcccgacaacccc

SEQ ID NO: 106 - DNA, 25 bp, Artificial Sequence
  Description of Artificial Sequence Synthetic oligonucleotide
cggcccagcccgacaacccctctcc

SEQ ID NO: 107 - DNA, 25 bp, Artificial Sequence
  Description of Artificial Sequence Synthetic oligonucleotide
cagcccgacaacccctctccccaca

SEQ ID NO: 108 - DNA, 25 bp, Artificial Sequence
  Description of Artificial Sequence Synthetic oligonucleotide
cgacaacccctctccccacagccac

SEQ ID NO: 109 - DNA, 25 bp, Artificial Sequence
  Description of Artificial Sequence Synthetic oligonucleotide
acccctctccccacagccactcacc

SEQ ID NO: 110 - DNA, 25 bp, Artificial Sequence
  Description of Artificial Sequence Synthetic oligonucleotide
tctccccacagccactcacctgccc

SEQ ID NO: 111 - DNA, 20 bp, Artificial Sequence
  Description of Artificial Sequence Synthetic oligonucleotide
cagtaccagggtcccatccc

SEQ ID NO: 112 - DNA, 20 bp, Artificial Sequence
  Description of Artificial Sequence Synthetic oligonucleotide
ccagggtcccatcccagccc

SEQ ID NO: 113 - DNA, 20 bp, Artificial Sequence
  Description of Artificial Sequence Synthetic oligonucleotide
gtcccatcccagccctgccc

SEQ ID NO: 114 - DNA, 20 bp, Artificial Sequence
  Description of Artificial Sequence Synthetic oligonucleotide
atcccagccctgccctgcag

SEQ ID NO: 115 - DNA, 20 bp, Artificial Sequence
  Description of Artificial Sequence Synthetic oligonucleotide
agccctgccctgcagtaccc

SEQ ID NO: 116 - DNA, 20 bp, Artificial Sequence
  Description of Artificial Sequence Synthetic oligonucleotide
tgccctgcagtacccatgaa

SEQ ID NO: 117 - DNA, 20 bp, Artificial Sequence
  Description of Artificial Sequence Synthetic oligonucleotide
tgcagtacccatgaacttcc

SEQ ID NO: 118 - DNA, 20 bp, Artificial Sequence
  Description of Artificial Sequence Synthetic oligonucleotide
tacccatgaacttcccttgc

SEQ ID NO: 119 - DNA, 20 bp, Artificial Sequence
  Description of Artificial Sequence Synthetic oligonucleotide
atgaacttcccttgcggcca

SEQ ID NO: 120 - DNA, 20 bp, Artificial Sequence
  Description of Artificial Sequence Synthetic oligonucleotide
cttcccttgcggccacttct

SEQ ID NO: 121 - DNA, 20 bp, Artificial Sequence
  Description of Artificial Sequence Synthetic oligonucleotide
cttgcggccacttctgaagc

SEQ ID NO: 122 - DNA, 20 bp, Artificial Sequence
  Description of Artificial Sequence Synthetic oligonucleotide
ggccacttctgaagcctgtg

SEQ ID NO: 123 - DNA, 20 bp, Artificial Sequence
  Description of Artificial Sequence Synthetic oligonucleotide
cttctgaagcctgtgcctca

SEQ ID NO: 124 - DNA, 20 bp, Artificial Sequence
  Description of Artificial Sequence Synthetic oligonucleotide
gaagcctgtgcctcacctgt

SEQ ID NO: 125 - DNA, 20 bp, Artificial Sequence
  Description of Artificial Sequence Synthetic oligonucleotide
ctgtgcctcacctgtcacat

SEQ ID NO: 126 - DNA, 20 bp, Artificial Sequence
  Description of Artificial Sequence Synthetic oligonucleotide
cctcacctgtcacatgcaca

SEQ ID NO: 127 - DNA, 20 bp, Artificial Sequence
  Description of Artificial Sequence Synthetic oligonucleotide
cctgtcacatgcacagagag

SEQ ID NO: 128 - DNA, 20 bp, Artificial Sequence
  Description of Artificial Sequence Synthetic oligonucleotide
gcacagagagctggggagat

SEQ ID NO: 129 - DNA, 20 bp, Artificial Sequence
  Description of Artificial Sequence Synthetic oligonucleotide
ctggggagatttgtaactgt

SEQ ID NO: 130 - DNA, 20 bp, Artificial Sequence
  Description of Artificial Sequence Synthetic oligonucleotide
gagatttgtaactgtatttg

SEQ ID NO: 131 - DNA, 20 bp, Artificial Sequence
  Description of Artificial Sequence Synthetic oligonucleotide
ttgtaactgtatttggtact

SEQ ID NO: 132 - DNA, 20 bp, Artificial Sequence
  Description of Artificial Sequence Synthetic oligonucleotide
actgtatttggtacttcctc

SEQ ID NO: 133 - DNA, 20 bp, Artificial Sequence
  Description of Artificial Sequence Synthetic oligonucleotide
atttggtacttcctctctcc

SEQ ID NO: 134 - DNA, 20 bp, Artificial Sequence
  Description of Artificial Sequence Synthetic oligonucleotide
gtacttcctctctccatccg

SEQ ID NO: 135 - DNA, 20 bp, Artificial Sequence
  Description of Artificial Sequence Synthetic oligonucleotide
tcctctctccatccgaaaga

SEQ ID NO: 136 - DNA, 20 bp, Artificial Sequence
  Description of Artificial Sequence Synthetic oligonucleotide
aaagaagtatgaaccattat

SEQ ID NO: 137 - DNA, 20 bp, Artificial Sequence
  Description of Artificial Sequence Synthetic oligonucleotide
agtatgaaccattatccctc

SEQ ID NO: 138 - DNA, 20 bp, Artificial Sequence
  Description of Artificial Sequence Synthetic oligonucleotide
gaaccattatccctcctcct

SEQ ID NO: 139 - DNA, 20 bp, Artificial Sequence
  Description of Artificial Sequence Synthetic oligonucleotide
attatccctcctcctggcgt

SEQ ID NO: 140 - DNA, 20 bp, Artificial Sequence
  Description of Artificial Sequence Synthetic oligonucleotide
ccctcctcctggcgtctacg

SEQ ID NO: 141 - DNA, 20 bp, Artificial Sequence
  Description of Artificial Sequence Synthetic oligonucleotide
ctcctggcgtctacgatgct

SEQ ID NO: 142 - DNA, 20 bp, Artificial Sequence
  Description of Artificial Sequence Synthetic oligonucleotide
ggcgtctacgatgctcaggg

SEQ ID NO: 143 - DNA, 20 bp, Artificial Sequence
  Description of Artificial Sequence Synthetic oligonucleotide
ctacgatgctcagggagcag

SEQ ID NO: 144 - DNA, 20 bp, Artificial Sequence
  Description of Artificial Sequence Synthetic oligonucleotide
cagggagcagttgttcctac

SEQ ID NO: 145 - DNA, 20 bp, Artificial Sequence
  Description of Artificial Sequence Synthetic oligonucleotide
agcagttgttcctactggga

SEQ ID NO: 146 - DNA, 20 bp, Artificial Sequence
  Description of Artificial Sequence Synthetic oligonucleotide
ttgttcctactgggatcccc

SEQ ID NO: 147 - DNA, 20 bp, Artificial Sequence
  Description of Artificial Sequence Synthetic oligonucleotide
cctactgggatccccaagga

SEQ ID NO: 148 - DNA, 20 bp, Artificial Sequence
  Description of Artificial Sequence Synthetic oligonucleotide
tgggatccccaaggaggcgg

SEQ ID NO: 149 - DNA, 20 bp, Artificial Sequence
  Description of Artificial Sequence Synthetic oligonucleotide
tccccaaggaggcggaatct

SEQ ID NO: 150 - DNA, 20 bp, Artificial Sequence
  Description of Artificial Sequence Synthetic oligonucleotide
aaggaggcggaatctgccct

SEQ ID NO: 151 - DNA, 20 bp, Artificial Sequence
  Description of Artificial Sequence Synthetic oligonucleotide
ggcggaatctgccctgagtc

SEQ ID NO: 152 - DNA, 20 bp, Artificial Sequence
  Description of Artificial Sequence Synthetic oligonucleotide
aatctgccctgagtctcctc

SEQ ID NO: 153 - DNA, 20 bp, Artificial Sequence
  Description of Artificial Sequence Synthetic oligonucleotide
gccctgagtctcctcctgta

SEQ ID NO: 154 - DNA, 20 bp, Artificial Sequence
  Description of Artificial Sequence Synthetic oligonucleotide
tcctcctgtacttcttgatc

SEQ ID NO: 155 - DNA, 20 bp, Artificial Sequence
  Description of Artificial Sequence Synthetic oligonucleotide
ctgtacttcttgatctagct

SEQ ID NO: 156 - DNA, 20 bp, Artificial Sequence
  Description of Artificial Sequence Synthetic oligonucleotide
cttcttgatctagcttgttt

SEQ ID NO: 157 - DNA, 20 bp, Artificial Sequence
  Description of Artificial Sequence Synthetic oligonucleotide
tgatctagcttgtttgtggc

SEQ ID NO: 158 - DNA, 20 bp, Artificial Sequence
  Description of Artificial Sequence Synthetic oligonucleotide
tagcttgtttgtggccactg

SEQ ID NO: 159 - DNA, 20 bp, Artificial Sequence
  Description of Artificial Sequence Synthetic oligonucleotide
tgtttgtggccactggagag

SEQ ID NO: 160 - DNA, 20 bp, Artificial Sequence
  Description of Artificial Sequence Synthetic oligonucleotide
gtggccactggagagtccct

SEQ ID NO: 161 - DNA, 20 bp, Artificial Sequence
  Description of Artificial Sequence Synthetic oligonucleotide
cactggagagtccctggata

SEQ ID NO: 162 - DNA, 20 bp, Artificial Sequence
  Description of Artificial Sequence Synthetic oligonucleotide
gagagtccctggatataatg

SEQ ID NO: 163 - DNA, 20 bp, Artificial Sequence
  Description of Artificial Sequence Synthetic oligonucleotide
tccctggatataatggctcc

SEQ ID NO: 164 - DNA, 20 bp, Artificial Sequence
  Description of Artificial Sequence Synthetic oligonucleotide
ggatataatggctccttccc

SEQ ID NO: 165 - DNA, 20 bp, Artificial Sequence
  Description of Artificial Sequence Synthetic oligonucleotide
taatggctccttcccggaac

SEQ ID NO: 166 - DNA, 20 bp, Artificial Sequence
  Description of Artificial Sequence Synthetic oligonucleotide
gctccttcccggaaccagta

SEQ ID NO: 167 - DNA, 20 bp, Artificial Sequence
  Description of Artificial Sequence Synthetic oligonucleotide
ttcccggaaccagtaaccat

SEQ ID NO: 168 - DNA, 20 bp, Artificial Sequence
  Description of Artificial Sequence Synthetic oligonucleotide
ggaaccagtaaccatgaact

SEQ ID NO: 169 - DNA, 20 bp, Artificial Sequence
  Description of Artificial Sequence Synthetic oligonucleotide
cagtaaccatgaactgggga

SEQ ID NO: 170 - DNA, 20 bp, Artificial Sequence
  Description of Artificial Sequence Synthetic oligonucleotide
accatgaactggggagttct

SEQ ID NO: 171 - DNA, 20 bp, Artificial Sequence
  Description of Artificial Sequence Synthetic oligonucleotide
gaactggggagttcttgtcg

SEQ ID NO: 172 - DNA, 20 bp, Artificial Sequence
  Description of Artificial Sequence Synthetic oligonucleotide
ggggagttcttgtcgtagta

SEQ ID NO: 173 - DNA, 20 bp, Artificial Sequence
  Description of Artificial Sequence Synthetic oligonucleotide
gttcttgtcgtagtagggta

SEQ ID NO: 174 - DNA, 20 bp, Artificial Sequence
  Description of Artificial Sequence Synthetic oligonucleotide
tgtcgtagtagggtatggga

SEQ ID NO: 175 - DNA, 20 bp, Artificial Sequence
  Description of Artificial Sequence Synthetic oligonucleotide
tagtagggtatgggatggaa

SEQ ID NO: 176 - DNA, 20 bp, Artificial Sequence
  Description of Artificial Sequence Synthetic oligonucleotide
gggtatgggatggaagaaag

SEQ ID NO: 177 - DNA, 20 bp, Artificial Sequence
  Description of Artificial Sequence Synthetic oligonucleotide
tgggatggaagaaagtgcag

SEQ ID NO: 178 - DNA, 20 bp, Artificial Sequence
  Description of Artificial Sequence Synthetic oligonucleotide
tggaagaaagtgcagggcac

SEQ ID NO: 179 - DNA, 20 bp, Artificial Sequence
  Description of Artificial Sequence Synthetic oligonucleotide
gaaagtgcagggcacgagga

SEQ ID NO: 180 - DNA, 20 bp, Artificial Sequence
  Description of Artificial Sequence Synthetic oligonucleotide
tgcagggcacgaggacgcac

SEQ ID NO: 181 - DNA, 20 bp, Artificial Sequence
  Description of Artificial Sequence Synthetic oligonucleotide
ggcacgaggacgcacaaacc

SEQ ID NO: 182 - DNA, 20 bp, Artificial Sequence
  Description of Artificial Sequence Synthetic oligonucleotide
gaggacgcacaaaccctcct

SEQ ID NO: 183 - DNA, 20 bp, Artificial Sequence
  Description of Artificial Sequence Synthetic oligonucleotide
aaaccctcctgtaccgtcac

SEQ ID NO: 184 - DNA, 20 bp, Artificial Sequence
  Description of Artificial Sequence Synthetic oligonucleotide
ctcctgtaccgtcactgact

SEQ ID NO: 185 - DNA, 20 bp, Artificial Sequence
  Description of Artificial Sequence Synthetic oligonucleotide
gtaccgtcactgactcctgc

SEQ ID NO: 186 - DNA, 20 bp, Artificial Sequence
  Description of Artificial Sequence Synthetic oligonucleotide
gtcactgactcctgcacttg

SEQ ID NO: 187 - DNA, 20 bp, Artificial Sequence
  Description of Artificial Sequence Synthetic oligonucleotide
tgactcctgcacttgcagcc

SEQ ID NO: 188 - DNA, 20 bp, Artificial Sequence
  Description of Artificial Sequence Synthetic oligonucleotide
cctgcacttgcagccagaaa

SEQ ID NO: 189 - DNA, 20 bp, Artificial Sequence
  Description of Artificial Sequence Synthetic oligonucleotide
acttgcagccagaaatttgg

SEQ ID NO: 190 - DNA, 20 bp, Artificial Sequence
  Description of Artificial Sequence Synthetic oligonucleotide
cagccagaaatttggatcca

SEQ ID NO: 191 - DNA, 20 bp, Artificial Sequence
  Description of Artificial Sequence Synthetic oligonucleotide
agaaatttggatccatagcc

SEQ ID NO: 192 - DNA, 20 bp, Artificial Sequence
  Description of Artificial Sequence Synthetic oligonucleotide
tttggatccatagccagggc

SEQ ID NO: 193 - DNA, 20 bp, Artificial Sequence
  Description of Artificial Sequence Synthetic oligonucleotide
atccatagccagggcccctg

SEQ ID NO: 194 - DNA, 20 bp, Artificial Sequence
  Description of Artificial Sequence Synthetic oligonucleotide
tagccagggcccctgtgggg

SEQ ID NO: 195 - DNA, 20 bp, Artificial Sequence
  Description of Artificial Sequence Synthetic oligonucleotide
agggcccctgtggggaaacg

SEQ ID NO: 196 - DNA, 20 bp, Artificial Sequence
  Description of Artificial Sequence Synthetic oligonucleotide
ccctgtggggaaacgagggt

SEQ ID NO: 197 - DNA, 20 bp, Artificial Sequence
  Description of Artificial Sequence Synthetic oligonucleotide
tggggaaacgagggtcagct

SEQ ID NO: 198 - DNA, 20 bp, Artificial Sequence
  Description of Artificial Sequence Synthetic oligonucleotide
aaacgagggtcagctcggcc

SEQ ID NO: 199 - DNA, 20 bp, Artificial Sequence
  Description of Artificial Sequence Synthetic oligonucleotide
agggtcagctcggcccagcc

SEQ ID NO: 200 - DNA, 20 bp, Artificial Sequence
  Description of Artificial Sequence Synthetic oligonucleotide
cagctcggcccagcccgaca

SEQ ID NO: 201 - DNA, 20 bp, Artificial Sequence
  Description of Artificial Sequence Synthetic oligonucleotide
cggcccagcccgacaacccc

SEQ ID NO: 202 - DNA, 20 bp, Artificial Sequence
  Description of Artificial Sequence Synthetic oligonucleotide
cagcccgacaacccctctcc

SEQ ID NO: 203 - DNA, 20 bp, Artificial Sequence
  Description of Artificial Sequence Synthetic oligonucleotide
cgacaacccctctccccaca

SEQ ID NO: 204 - DNA, 20 bp, Artificial Sequence
  Description of Artificial Sequence Synthetic oligonucleotide
acccctctccccacagccac

SEQ ID NO: 205 - DNA, 20 bp, Artificial Sequence
  Description of Artificial Sequence Synthetic oligonucleotide
tctccccacagccactcacc

SEQ ID NO: 206 - DNA, 20 bp, Artificial Sequence
  Description of Artificial Sequence Synthetic oligonucleotide
ccacagccactcacctgccc

SEQ ID NO: 207 - DNA, 17 bp, Artificial Sequence
  Description of Artificial Sequence Synthetic oligonucleotide
cgctgctgctactgctg

SEQ ID NO: 208 - DNA, 20 bp, Artificial Sequence
  Description of Artificial Sequence Synthetic oligonucleotide
ttctagagtgccagggatga

SEQ ID NO: 209 - DNA, 22 bp, Artificial Sequence
  Description of Artificial Sequence Synthetic oligonucleotide
tgtgggcagacttgacccacag

SEQ ID NO: 210 - DNA, 18 bp, Artificial Sequence
  Description of Artificial Sequence Synthetic oligonucleotide
ggatggagagaggaagta

SEQ ID NO: 211 - DNA, 19 bp, Artificial Sequence
  Description of Artificial Sequence Synthetic oligonucleotide
gtgccagggatgaggattt

SEQ ID NO: 212 - DNA, 24 bp, Artificial Sequence
  Description of Artificial Sequence Synthetic oligonucleotide
tgcatgtgacagacttgacccaca

SEQ ID NO: 213 - DNA, 38 bp, Artificial Sequence
  Description of Artificial Sequence Synthetic oligonucleotide
tctccccagctctctgtgcatgtgacaggtgaggcaca

SEQ ID NO: 214 - DNA, 58 bp, Artificial Sequence
  Description of Artificial Sequence Synthetic oligonucleotide
taatggttcatacttctttcggatggagagaggaagtaccaaatacagttacaaatct

SEQ ID NO: 215 - DNA, 43 bp, Artificial Sequence
  Description of Artificial Sequence Synthetic oligonucleotide
cctcgtgccctgcactttcttccatcccataccctactacgac

SEQ ID NO: 216 - DNA, 43 bp, Artificial Sequence
  Description of Artificial Sequence Synthetic oligonucleotide
aggggccctggctatggatccaaatttctggctgcaagtgcag

SEQ ID NO: 217 - DNA, 45 bp, Artificial Sequence
  Description of Artificial Sequence Synthetic oligonucleotide
acagttacaaatctccccagctctctgtgcatgtgacaggtgagg

SEQ ID NO: 218 - DNA, 40 bp, Artificial Sequence
  Description of Artificial Sequence Synthetic oligonucleotide
ggttcatacttctttcggatggagagaggaagtaccaaat

SEQ ID NO: 219 - DNA, 35 bp, Artificial Sequence
  Description of Artificial Sequence Synthetic oligonucleotide
gcaggagtcagtgacggtacaggagggtttgtgcg

SEQ ID NO: 220 - DNA, 34 bp, Artificial Sequence
  Description of Artificial Sequence Synthetic oligonucleotide
ggccgagctgaccctcgtttccccacaggggccc

SEQ ID NO: 221 - DNA, 23 bp, Artificial Sequence
  Description of Artificial Sequence Synthetic oligonucleotide
cctcacctgtcacatgcacagag

SEQ ID NO: 222 - DNA, 23 bp, Artificial Sequence
  Description of Artificial Sequence Synthetic oligonucleotide
tcacctgtcacatgcacagagag

SEQ ID NO: 223 - DNA, 23 bp, Artificial Sequence
  Description of Artificial Sequence Synthetic oligonucleotide
ctcacctgtcacatgcacagaga

SEQ ID NO: 224 - DNA, 21 bp, Artificial Sequence
  Description of Artificial Sequence Synthetic oligonucleotide
cctcacctgtcacatgcacag

SEQ ID NO: 225 - DNA, 21 bp, Artificial Sequence
  Description of Artificial Sequence Synthetic oligonucleotide
acctgtcacatgcacagagag

SEQ ID NO: 226 - DNA, 21 bp, Artificial Sequence
  Description of Artificial Sequence Synthetic oligonucleotide
tcacctgtcacatgcacagag

SEQ ID NO: 227 - DNA, 25 bp, Artificial Sequence
  Description of Artificial Sequence Synthetic oligonucleotide
tcacctgtcacatgcacagagagct

SEQ ID NO: 228 - DNA, 25 bp, Artificial Sequence
  Description of Artificial Sequence Synthetic oligonucleotide
cctgtgcctcacctgtcacatgcac

SEQ ID NO: 229 - DNA, 25 bp, Artificial Sequence
  Description of Artificial Sequence Synthetic oligonucleotide
gtgcctcacctgtcacatgcacaga

SEQ ID NO: 230 - DNA, 25 bp, Artificial Sequence
  Description of Artificial Sequence Synthetic oligonucleotide
tgcctcacctgtcacatgcacagag

SEQ ID NO: 231 - DNA, 25 bp, Artificial Sequence
  Description of Artificial Sequence Synthetic oligonucleotide
ctcacctgtcacatgcacagagagc

SEQ ID NO: 232 - DNA, 25 bp, Artificial Sequence
  Description of Artificial Sequence Synthetic oligonucleotide
cacctgtcacatgcacagagagctg

SEQ ID NO: 233 - DNA, 25 bp, Artificial Sequence
  Description of Artificial Sequence Synthetic oligonucleotide
acctgtcacatgcacagagagctgg

SEQ ID NO: 234 - DNA, 25 bp, Artificial Sequence
  Description of Artificial Sequence Synthetic oligonucleotide
ctgtcacatgcacagagagctgggg

SEQ ID NO: 235 - DNA, 23 bp, Artificial Sequence
  Description of Artificial Sequence Synthetic oligonucleotide
cctgtcacatgcacagagagctg

SEQ ID NO: 236 - DNA, 23 bp, Artificial Sequence
  Description of Artificial Sequence Synthetic oligonucleotide
tgtcacatgcacagagagctggg

SEQ ID NO: 237 - DNA, 23 bp, Artificial Sequence
  Description of Artificial Sequence Synthetic oligonucleotide
ctgtcacatgcacagagagctgg

SEQ ID NO: 238 - DNA, 22 bp, Artificial Sequence
  Description of Artificial Sequence Synthetic oligonucleotide
tgtcacatgcacagagagctgg

SEQ ID NO: 239 - DNA, 21 bp, Artificial Sequence
  Description of Artificial Sequence Synthetic oligonucleotide
tcacatgcacagagagctggg

SEQ ID NO: 240 - DNA, 21 bp, Artificial Sequence
  Description of Artificial Sequence Synthetic oligonucleotide
tgtcacatgcacagagagctg

SEQ ID NO: 241 - DNA, 25 bp, Artificial Sequence
  Description of Artificial Sequence Synthetic oligonucleotide
ctgtatttggtacttcctctctcca

SEQ ID NO: 242 - DNA, 25 bp, Artificial Sequence
  Description of Artificial Sequence Synthetic oligonucleotide
tgtatttggtacttcctctctccat

SEQ ID NO: 243 - DNA, 25 bp, Artificial Sequence
  Description of Artificial Sequence Synthetic oligonucleotide
gtatttggtacttcctctctccatc

SEQ ID NO: 244 - DNA, 25 bp, Artificial Sequence
  Description of Artificial Sequence Synthetic oligonucleotide
tatttggtacttcctctctccatcc

SEQ ID NO: 245 - DNA, 20 bp, Artificial Sequence
  Description of Artificial Sequence Synthetic oligonucleotide
ctccatccgaaagaagtatg

SEQ ID NO: 246 - DNA, 20 bp, Artificial Sequence
  Description of Artificial Sequence Synthetic oligonucleotide
tccatccgaaagaagtatga

SEQ ID NO: 247 - DNA, 20 bp, Artificial Sequence
  Description of Artificial Sequence Synthetic oligonucleotide
ccatccgaaagaagtatgaa

SEQ ID NO: 248 - DNA, 20 bp, Artificial Sequence
  Description of Artificial Sequence Synthetic oligonucleotide
catccgaaagaagtatgaac

SEQ ID NO: 249 - DNA, 20 bp, Artificial Sequence
  Description of Artificial Sequence Synthetic oligonucleotide
tccgaaagaagtatgaacca

SEQ ID NO: 250 - DNA, 20 bp, Artificial Sequence
  Description of Artificial Sequence Synthetic oligonucleotide
ccgaaagaagtatgaaccat

SEQ ID NO: 251 - DNA, 18 bp, Artificial Sequence
  Description of Artificial Sequence Synthetic oligonucleotide
atccgaaagaagtatgaa

SEQ ID NO: 252 - DNA, 18 bp, Artificial Sequence
  Description of Artificial Sequence Synthetic oligonucleotide
ccgaaagaagtatgaacc

SEQ ID NO: 253 - DNA, 18 bp, Artificial Sequence
  Description of Artificial Sequence Synthetic oligonucleotide
tccgaaagaagtatgaac

SEQ ID NO: 254 - DNA, 18 bp, Artificial Sequence
  Description of Artificial Sequence Synthetic oligonucleotide
ccatccgaaagaagtatg

SEQ ID NO: 255 - DNA, 18 bp, Artificial Sequence
  Description of Artificial Sequence Synthetic oligonucleotide
tccatccgaaagaagtat

SEQ ID NO: 256 - DNA, 18 bp, Artificial Sequence
  Description of Artificial Sequence Synthetic oligonucleotide
gaaagaagtatgaaccat

SEQ ID NO: 257 - DNA, 20 bp, Artificial Sequence
  Description of Artificial Sequence Synthetic oligonucleotide
ccttccctgaaggttcctcc

SEQ ID NO: 258 - DNA, 10 bp, Artificial Sequence
  Description of Artificial Sequence Synthetic probe
tctttcggat

SEQ ID NO: 259 - DNA, 10 bp, Artificial Sequence
  Description of Artificial Sequence Synthetic probe
ggttcatact

SEQ ID NO: 260 - DNA, 21 bp, Artificial Sequence
  Description of Artificial Sequence Synthetic oligonucleotide
cctcacctgtcacatgcacag

SEQ ID NO: 261 - DNA, 25 bp, Artificial Sequence
  Description of Artificial Sequence Synthetic oligonucleotide
cctcacctgtcacatgcacagagag

SEQ ID NO: 262 - DNA, 22 bp, Artificial Sequence
  Description of Artificial Sequence Synthetic oligonucleotide
cctcacctgtcacatgcacagt

SEQ ID NO: 263 - DNA, 25 bp, Artificial Sequence
  Description of Artificial Sequence Synthetic oligonucleotide
cctcacctgtcacatgcacagagag

(19) United States
(12) Patent Application Publication
(10) Pub. No.: US 2025/0092392 A1
(43) Pub. Date: Mar. 20, 2025

(54) ANTISENSE OLIGONUCLEOTIDES AND THEIR USE FOR TREATMENT OF NEURODEGENERATIVE DISORDERS

(71) Applicant: Eisai R&D Management Co., Ltd., Tokyo (JP)

(72) Inventors: Vinod VATHIPADIEKAL, Stoneham, MA (US); Branko MITASEV, North Reading, MA (US); Courtney EASLEY-NEAL, Winchester, MA (US); Hyeong Wook CHOI, Andover, MA (US); Francis G. FANG, Andover, MA (US); John WANG, Andover, MA (US); Praveen VEMULA, Arlington, MA (US); Jung Hwa LEE, Andover, MA (US)

(73) Assignee: Eisai R&D Management Co., Ltd., Tokyo (JP)

(21) Appl. No.: 18/557,876

(22) PCT Filed: Apr. 28, 2022
(86) PCT No.: PCT/US2022/026760

§ 371 Date: Oct. 27, 2023

Related U.S. Application Data
(60) Provisional application No. 63/181,023, filed on Apr. 28, 2021.
(60) Provisional application No. 63/320,651, filed on Mar. 16, 2022.
(60) Provisional application No. 63/334,496, filed on Apr. 25, 2022.

(51) Int. Cl. C12N 15/113

(52) U.S. Cl. CPC C12N 15/113; C12N 2310/11; C12N 2310/314; C12N 2310/321; C12N 2310/3233; C12N 2320/33

(57) ABSTRACT

Novel antisense oligonucleotides that induce Exon-2 skipping in the CD33 gene during pre-mRNA splicing, and their use in the treatment of a neurodegenerative disease, such as Alzheimer's disease, are disclosed.

The present application claims the benefit of priority to U.S. Provisional Application No. 63/181,023, filed Apr. 28, 2021; U.S. Provisional Application No. 63/320,651, filed Mar. 16, 2022; U.S. Provisional Application No. 63/334,496, filed Apr. 25, 2022; the entire contents of each are incorporated herein by reference.

Disclosed herein are novel antisense oligonucleotides (“ASOs”) that may induce exon skipping during pre-mRNA splicing, pharmaceutical compositions comprising the same, and methods of using the same.

Neurodegenerative disorders are a group of disorders characterized by the decline of central nervous system and peripheral nervous system structure and function. While neurodegenerative disorders exhibit heterogeneous symptoms, they can share similar features. One neurodegenerative disease, Alzheimer's Disease, is a neurodegenerative disorder characterized by buildup of amyloid beta plaques and neurofibrillary tangles. It is also the leading cause of dementia. Although some cases of rare familial Alzheimer's Disease involve autosomal dominant mutations to the amyloid beta precursor protein, the majority of cases are late-onset Alzheimer's Disease (LOAD), which do not follow Mendelian inheritance patterns. While the mechanics of LOAD are not completely understood, genome-wide association studies have identified genetic risk factors for LOAD. Scientists have shown the ability of these genes to impact the production, aggregation, or clearance of amyloid beta plaques. One such gene is CD33, also known as Siglec-3. Griciuc et al., Alzheimer's Disease Risk Gene CD33 Inhibits Microglial Uptake of Amyloid Beta, 78 NEURON 631 (2013).

CD33 is expressed in myeloid-derived cells, including macrophages such as microglia, and encodes the CD33 protein. Microglia account for approximately 10% of the cells in the brain and represent the first line of immunological defense. Microglia modulate several important activities in the brain, such as homeostasis, cognition, and neurogenesis. Augusto-Oliveira et al., What Do Microglia Really Do in Healthy Adult Brain?, 8 CELLS 1293 (2019). Microglia cells are known to contribute to neurodegeneration by releasing proinflammatory substances in the central nervous system. Wojtera et al., Microglial cells in neurodegenerative disorders, 43 FOLIA NEUROPATHOLOGY 311 (2005).

CD33 is a transmembrane receptor protein that has an extracellular receptor that binds the ligand sialic acid. The intracellular immunoreceptor tyrosine-based inhibition motif recruits phosphatases upon phosphorylation of its tyrosine residues, leading to suppression of immune cell activity such as phagocytosis. CD33 has been found to inhibit microglial uptake of amyloid beta protein, which suggests that therapies targeting CD33 could be potential LOAD treatment options. Griciuc et al., Alzheimer's Disease Risk Gene CD33 Inhibits Microglial Uptake of Amyloid Beta, 78 NEURON 631 (2013).

Two single nucleotide polymorphisms (SNPs) in the promoter region of the CD33 gene are associated with LOAD: rs3826656 and rs3865444. The rs3865444 SNP comes in two forms, rs3865444-C and rs3865444-A. The first form results in normal length CD33 protein. The second form, rs3865444-A, modulates splicing of CD33 pre-mRNA resulting in skipping of Exon-2 and a CD33 protein lacking the sialic acid binding domain. Malik et al., CD33 Alzheimer's Risk-Altering Polymorphism, CD33 Expression, and Exon 2 Splicing, 33 J. NEUROSCIENCE 13320 (2013).

In eukaryotic genes containing coding (exons) and noncoding (intron) sequences, the noncoding introns are excised from the pre-mRNA transcript and the coding exons are spliced together to form mRNA. If an intron is left in the final mRNA transcript or an exon is left out, the mRNA reading frame may be disrupted during translation of the mRNA. This may result in a non-functional polypeptide sequence or a premature stop codon. The splicing process is further complicated by alternative splicing, where the same pre-mRNA sequence can be spliced into different exon combinations to form multiple mRNA sequences.

Splicing of pre-mRNA is an intricate process involving a multi-megadalton ribonucleoprotein complex called the spliceosome. The spliceosome recognizes specific sequences in pre-mRNA to precisely excise introns and ligate exons. The spliceosome catalyzes intron excision in two transesterification reactions using three conserved RNA sequences. These RNA sequences are the 5′ splice site, 3′ splice site, and the branch site. Will & Luhrmann, Spliceosome Structure and Function, 3 COLD SPRING HARB. PERSPECT. BIOL. 1 (2011).

Splicing begins with the 2′ OH group of the branch site binding to the 5′ splice site via a nucleophilic attack, causing cleavage of the 5′ exon at the 5′ splice site and forming a lariat. Then the 3′ OH group of the 5′ exon attacks the 3′ exon at the 3′ splice site, ligating the 5′ and 3′ exons and cleaving the intron lariat. Will & Luhrmann, Spliceosome Structure and Function, 3 COLD SPRING HARB. PERSPECT. BIOL. 1 (2011). Because the splicing process involves spliceosome recognition sites, 5′ and 3′ splice sites, and the branch site, a mutation in any one of these sites can disrupt the splicing process.

ASOs are polynucleotides designed to bind with specificity to a target nucleotide sequence, thereby affecting one or more aspects of gene expression, such as, transcription, splicing, stability, and/or translation. ASOs may be directed to either RNA or DNA. ASOs directed to RNA can bind to target mRNA sequences, effecting mRNA stability or translation at the ribosome.

ASOs that bind to target sequences in pre-mRNA transcripts can affect the splicing process. In some cases, ASOs may be used to induce exon skipping during pre-mRNA splicing. For example, Duchenne Muscular Dystrophy (DMD) is caused by a mutation that alters the reading frame of dystrophin mRNA during translation, resulting in a premature stop codon and truncated dystrophin protein. ASOs may be utilized to correct the reading frame by inducing skipping of an exon during splicing. Removing an exon of the correct number of base pairs results in a shorter mRNA transcript, but the reading frame may be corrected. Because dystrophin RNA consists of 79 exons, skipping one or several exons during splicing still results in a partly functional protein. Echigoya et al., Multiple Exon Skipping in the Duchenne Muscular Dystrophy Hot Spots: Prospects and Challenges, 8 J. PERS. MED. 41 (2018). The FDA approved an exon-skipping drug called Exondys 51 (eteplirsen) for treatment of DMD in 2016. Dowling, Eteplirsen therapy for Duchenne muscular dystrophy: skipping to the front of the line, 12 NATURE REV. NEUROLOGY 675 (2016).

In other cases, ASOs may be used to prevent or reduce exon skipping during pre-mRNA splicing. As an example, the ASO drug nusinersen (Spinraza®) reduces Exon-7 skipping during splicing of the SMN2 gene to treat spinal muscular atrophy. Son & Yokota, Recent Advances and Clinical Applications of Exon Inclusion for Spinal Muscular Atrophy, in EXON SKIPPING & INCLUSION THERAPIES, 57-68 (2018). The rs3865444-A variant that induces Exon-2 skipping of CD33 conveys protection against LOAD. Malik et al., CD33 Alzheimer's Risk-Altering Polymorphism, CD33 Expression, and Exon 2 Splicing, 33 J. NEUROSCIENCE 13320 (2013). There remains a need, however, for ASOs that successfully induce Exon-2 skipping during pre-mRNA splicing of CD33 and for their use in treating neurodegenerative diseases.

Disclosed herein are ASOs, methods of using such ASOs to induce exon skipping during pre-mRNA splicing, pharmaceutical compositions that comprise such ASOs, and methods of using such compositions to treat neurodegenerative disease.

In some embodiments, disclosed herein is an antisense oligonucleotide of 16-30, such as 18-30, nucleotides in length, which is complementary to a portion of SEQ ID NO:1. In some embodiments, the antisense oligonucleotide is complementary to a portion of: SEQ ID NO:213; SEQ ID NO:214; SEQ ID NO:215; SEQ ID NO:216; SEQ ID NO:217; SEQ ID NO:218; SEQ ID NO:219; and/or SEQ ID NO:220. In some embodiments, the antisense oligonucleotide has a CD33 Exon-2 skipping efficiency of 35% or greater. In some embodiments, the antisense oligonucleotide has a CD33 Exon-2 skipping efficiency of 30% or greater. In some embodiments, the Exon-2 skipping efficiency of the antisense oligonucleotide is 30% or greater according to a Standard Exon-Skipping Efficiency Assay for ASOs. In some embodiments, the antisense oligonucleotide has a CD33 Exon-2 skipping efficiency of 30% or greater according to a Standard Exon-Skipping Efficiency Assay for PMO ASOs when the antisense oligonucleotide comprises phosphorodiamidate morpholino oligomers or according to a Standard Exon-Skipping Efficiency Assay for MOE ASOs when the antisense oligonucleotide comprises methoxyethyl ribose oligomers.

In some embodiments, the antisense oligonucleotide comprises all or a portion of:

a.
PMO-002
(SEQ ID NO: 2)
(5′-CCTCACCTGTCACATGCACAGAGAG-3′);
b.
PMO-003
(SEQ ID NO: 3)
(5′-CCTGTCACATGCACAGAGAGCTGGG-3′);
c.
PMO-036
(SEQ ID NO: 36)
(5′-TTGTAACTGTATTTGGTACTTCCTC-3′);
d.
PMO-037
(SEQ ID NO: 37)
(5′-ACTGTATTTGGTACTTCCTCTCTCC-3′);
e.
PMO-004
(SEQ ID NO: 4)
(5′-ATTTGGTACTTCCTCTCTCCATCCG-3′);
f.
PMO-038
(SEQ ID NO: 38)
(5′-GTACTTCCTCTCTCCATCCGAAAGA-3′);
g.
PMO-039
(SEQ ID NO: 39)
(5′-TCCTCTCTCCATCCGAAAGAAGTAT-3′);
h.
PMO-005
(SEQ ID NO: 5)
(5′-TCTCCATCCGAAAGAAGTATGAACC-3′);
i.
PMO-082
(SEQ ID NO: 82)
(5′-TAGTAGGGTATGGGATGGAAGAAAG-3′);
j.
PMO-083
(SEQ ID NO: 83)
(5′-GGGTATGGGATGGAAGAAAGTGCAG-3′);
k.
PMO-006
(SEQ ID NO: 6)
(5′-TGGGATGGAAGAAAGTGCAGGGCAC-3′);
l.
PMO-096
(SEQ ID NO: 96)
(5′-ACTTGCAGCCAGAAATTTGGATCCA-3′);
m.
PMO-007
(SEQ ID NO: 7)
(5′-CAGCCAGAAATTTGGATCCATAGCC-3′);
n.
PMO-097
(SEQ ID NO: 97)
(5′-AGAAATTTGGATCCATAGCCAGGGC-3′);
o.
PMO-008
(SEQ ID NO: 8)
(5′-CCCTGTGGGGAAACGAGGGTCAGCT-3′);
p.
MOE-009
(SEQ ID NO: 9)
(5′-CACATGCACAGAGAGCTGGG-3′);
q.
MOE-128
(SEQ ID NO: 128)
(5′-GCACAGAGAGCTGGGGAGAT-3′);
r.
MOE-010
(SEQ ID NO: 10)
(5′-GAGAGCTGGGGAGATTTGTA-3′);
s.
MOE-132
(SEQ ID NO: 132)
(5′-ACTGTATTTGGTACTTCCTC-3′);
t.
MOE-135
(SEQ ID NO: 135)
(5′-TCCTCTCTCCATCCGAAAGA-3′);
u.
MOE-011
(SEQ ID NO: 11)
(5′-TCTCCATCCGAAAGAAGTAT-3′);
v.
MOE-012
(SEQ ID NO: 12)
(5′-ATCCGAAAGAAGTATGAACC-3′);
w.
MOE-136
(SEQ ID NO: 136);
(5′-AAAGAAGTATGAACCATTAT-3′);
x.
MOE-013
(SEQ ID NO: 13)
(5′-ATGCTCAGGGAGCAGTTGTT-3′);
y.
MOE-014
(SEQ ID NO: 14)
(5′-GAGTCTCCTCCTGTACTTCT-3′);
z.
MOE-015
(SEQ ID NO: 15)
(5′-CGCACAAACCCTCCTGTACC-3′);
aa.
MOE-183
(SEQ ID NO: 183)
(5′-AAACCCTCCTGTACCGTCAC-3′);
bb.
MOE-184
(SEQ ID NO: 184)
(5′-CTCCTGTACCGTCACTGACT-3′);
cc.
MOE-190
(SEQ ID NO: 190)
(5′-CAGCCAGAAATTTGGATCCA-3′);
dd.
MOE-196
(SEQ ID NO: 196)
(5′-CCCTGTGGGGAAACGAGGGT-3′);
or
ee.
MOE-197
(SEQ ID NO: 197)
(5′-TGGGGAAACGAGGGTCAGCT-3′).

In some embodiments, the antisense oligonucleotide comprises all or a portion of:

a.
PMO-221
(SEQ ID NO: 221)
(5′- CCTCACCTGTCACATGCACAGAG-3′);
b.
PMO-222
(SEQ ID NO: 222)
(5′- TCACCTGTCACATGCACAGAGAG-3′);
c.
PMO-223
(SEQ ID NO: 223)
(5′- CTCACCTGTCACATGCACAGAGA-3′);
d.
PMO-224
(SEQ ID NO: 224)
(5′- CCTCACCTGTCACATGCACAG-3′);
e.
PMO-225
(SEQ ID NO: 225)
(5′- ACCTGTCACATGCACAGAGAG-3′);
f.
PMO-226
(SEQ ID NO: 226)
(5′- TCACCTGTCACATGCACAGAG-3′);
g.
PMO-227
(SEQ ID NO: 227)
(5′- TCACCTGTCACATGCACAGAGAGCT-3′);
h.
PMO-228
(SEQ ID NO: 228)
(5′- CCTGTGCCTCACCTGTCACATGCAC-3′);
i.
PMO-229
(SEQ ID NO: 229)
(5′- GTGCCTCACCTGTCACATGCACAGA-3′);
j.
PMO-230
(SEQ ID NO: 230)
(5′- TGCCTCACCTGTCACATGCACAGAG-3′);
k.
PMO-231
(SEQ ID NO: 231)
(5′- CTCACCTGTCACATGCACAGAGAGC-3′);
l.
PMO-232
(SEQ ID NO: 232)
(5′- CACCTGTCACATGCACAGAGAGCTG-3′);
m.
PMO-233
(SEQ ID NO: 233)
(5′- ACCTGTCACATGCACAGAGAGCTGG-3′);
n.
PMO-234
(SEQ ID NO: 234)
(5′- CTGTCACATGCACAGAGAGCTGGGG-3′);
o.
PMO-235
(SEQ ID NO: 235)
(5′- CCTGTCACATGCACAGAGAGCTG-3′);
p.
PMO-236
(SEQ ID NO: 236)
(5′- TGTCACATGCACAGAGAGCTGGG-3′);
q.
PMO-237
(SEQ ID NO: 237)
(5′- CTGTCACATGCACAGAGAGCTGG-3′);
r.
PMO-238
(SEQ ID NO: 238)
(5′- TGTCACATGCACAGAGAGCTGG-3′);
s.
PMO-239
(SEQ ID NO: 239)
(5′- TCACATGCACAGAGAGCTGGG-3′);
t.
PMO-240
(SEQ ID NO: 240)
(5′- TGTCACATGCACAGAGAGCTG-3′);
u.
PMO-241
(SEQ ID NO: 241)
(5′- CTGTATTTGGTACTTCCTCTCTCCA-3′);
v.
PMO-242
(SEQ ID NO: 242)
(5′- TGTATTTGGTACTTCCTCTCTCCAT-3′);
w.
PMO-243
(SEQ ID NO: 243)
(5′- GTATTTGGTACTTCCTCTCTCCATC-3′);
x.
PMO-244
(SEQ ID NO: 244)
(5′-TATTTGGTACTTCCTCTCTCCATCC-3′);
y.
PMO-324
(SEQ ID NO: 224)
(5′- CCTCACCTGTCACATGCACAG-3′);
Stereopattern: RRRRRRRRRRRRRRRRRRRR
z.
PMO-424
(SEQ ID NO: 224)
(5′- CCTCACCTGTCACATGCACAG-3′);
Stereopattern: SSSSSSSSSSSSSSSSSSSS
aa.
PMO-402
(SEQ ID NO: 002)
(5′- CCTCACCTGTCACATGCACAGAGAG-3′);
Stereopattern: RRRRRRRRRRRRRRRRRRRRRRRR;
or
bb.
PMO-502
(SEQ ID NO: 002)
(5′- CCTCACCTGTCACATGCACAGAGAG-3′);
Stereopattern: SSSSSSSSSSSSSSSSSSSSSSSS.

In some embodiments, the antisense oligonucleotide comprises all or a portion of:

a.
MOE-245
(SEQ ID NO: 245)
(5′-CTCCATCCGAAAGAAGTATG-3′);
b.
MOE-246
(SEQ ID NO: 246)
(5′-TCCATCCGAAAGAAGTATGA-3′);
c.
MOE-247
(SEQ ID NO: 247)
(5′-CCATCCGAAAGAAGTATGAA-3′);
d.
MOE-248
(SEQ ID NO: 248)
(5′-CATCCGAAAGAAGTATGAAC-3′);
e.
MOE-249
(SEQ ID NO: 249)
(5′-TCCGAAAGAAGTATGAACCA-3′);
f.
MOE-250
(SEQ ID NO: 250)
(5′-CCGAAAGAAGTATGAACCAT-3′);
g.
MOE-251
(SEQ ID NO: 251)
(5′-ATCCGAAAGAAGTATGAA-3′);
h.
MOE-252
(SEQ ID NO: 252)
(5′-CCGAAAGAAGTATGAACC-3′);
i.
MOE-253
(SEQ ID NO: 253)
(5′-TCCGAAAGAAGTATGAAC-3′);
j.
MOE-254
(SEQ ID NO: 254)
(5′-CCATCCGAAAGAAGTATG-3′);
k.
MOE-255
(SEQ ID NO: 255)
(5′-TCCATCCGAAAGAAGTAT-3′);
l.
MOE-256
(SEQ ID NO: 256)
(5′- GAAAGAAGTATGAACCAT-3′);
m.
MOE-257
(SEQ ID NO: 012)
(5′- ATC-CGAAAGAAGTATGA-ACC-3′);
n.
MOE-258
(SEQ ID NO: 012)
(5′- ATCC-GAAAGAAGTATG-AACC-3′);
o.
MOE-259
(SEQ ID NO: 012)
(5′- ATCCG-AAAGAAGTAT-GAACC-3′);
p.
MOE-260
(SEQ ID NO: 012)
(5′- ATCCG-AAAGAAGTA-TGAACC-3′);
q.
MOE-261
(SEQ ID NO: 012)
(5′- ATCC-GAAAGA-AGTATG-AACC-3′);
r.
MOE-262
(SEQ ID NO: 012)
(5′- ATCC-gAAAGAAGTATG-aACC-3′);
s.
MOE-263
(SEQ ID NO: 012)
(5′- ATCC-gAAAGAAGTATG-aACC-3′);
t.
MOE-264
(SEQ ID NO: 012)
(5′- ATCC-gAAAGAaGTATG-aACC-3′);
u.
MOE-265
(SEQ ID NO: 252)
(5′-CCGA-aAGAAGTATGAACC-3′);
v.
MOE-266
(SEQ ID NO: 252)
(5′-CCGA-aAGAAGTATG-aACC-3′);
w.
MOE-267
(SEQ ID NO: 252)
(5′-CCGA-aAGAAGtATG-aACC-3′);
x.
MOE-268
(SEQ ID NO: 252)
(5′-CCG-AAAGAAGTATGA-ACC-3′);
y.
MOE-269
(SEQ ID NO: 252)
(5′-CCGA-AAGAAGTATG-AACC-3′);
z.
MOE-270
(SEQ ID NO: 252)
(5′-CCGAA-AGAA-GTATG-AACC-3′);
aa.
MOE-271
(SEQ ID NO: 252)
(5′-CCGAA-AGAAGTAT-GAACC-3′);
bb.
MOE-272
(SEQ ID NO: 252)
(5′-CCG-A-AAGAAGTATGAACC-3′);
cc.
MOE-273
(SEQ ID NO: 252)
(5′-CCG-AA-AGAAGTATGAACC-3′);
dd.
MOE-274
(SEQ ID NO: 252)
(5′-CCGAAAGAAGTATG-A-ACC-3′);
ee.
MOE-275
(SEQ ID NO: 012)
(5′-mAmTfCfCfGfAfAfAfGfAfAfGfTfAfTfGfAfAmCmC-3′);
ff.
MOE-276
(SEQ ID NO: 012)
(5′- fAfTfCfCfGmAmAmAmGmAmAmGmTmAfTfGfAfAfCfC-3′);
gg.
MOE-277
(SEQ ID NO: 012)
(5′- ATCCGAAAGAAGTATGAACC-3′);
Stereopattern: SSSSSSSSSSSSSSSSSSS;
hh.
MOE-278
(SEQ ID NO: 012)
(5′- ATCCGAAAGAAGTATGAACC-3′);
Stereopattern: RRRRRRRRRRRRRRRRRRR;
ii.
MOE-279
(SEQ ID NO: 012)
(5′- ATCCGAAAGAAGTATGAACC-3′);
Stereopattern: SSSRSSSRSSSRSSSRSSS;
jj.
MOE-280
(SEQ ID NO: 012)
(5′- ATCCGAAAGAAGTATGAACC-3′);
Stereopattern: SSSRSSRSSRSSRSSRSSS;
kk.
MOE-281
(SEQ ID NO: 012)
(5′- ATCCGAAAGAAGTATGAACC-3′);
Stereopattern: SSSRSRSRSRSRSRSRSSS;
ll.
MOE-282
(SEQ ID NO: 012)
(5′- ATCCGAAAGAAGTATGAACC-3′);
Stereopattern: SSSSSSRRRRRRRSSSSSS;
mm.
MOE-283
(SEQ ID NO: 012)
(5′- ATCCGAAAGAAGTATGAACC-3′);
Stereopattern: SSSRRSRRSRRSRRSRSSS;
nn.
MOE-284
(SEQ ID NO: 012)
(5′-ATCCGAAAGAAGTATGAACC-3′);
Stereopattern: SSSRRSRRRSRRRSRRSSS;
oo.
MOE-285
(SEQ ID NO: 012)
(5′-ATCCGAAAGAAGTATGAACC-3′);
Stereopattern: SSSSSRRRRRRRRRSSSSS;
pp.
MOE-286
(SEQ ID NO: 012)
(5′-ATCCGAAAGAAGTATGAACC-3′);
Stereopattern: SSSRRRRRRRRRRRRRSSS;
qq.
MOE-287
(SEQ ID NO: 012)
(5′-ATCCGAAAGAAGTATGAACC-3′);
Stereopattern: SSRSSSSSSSSRSRSSSSS;
rr.
MOE-288
(SEQ ID NO: 252)
(5′-CCGAAAGAAGTATGAACC-3′);
Stereopattern: SSSSSSSSSSSSSSSSS;
ss.
MOE-289
(SEQ ID NO: 252)
(5′-CCGAAAGAAGTATGAACC-3′);
Stereopattern: RRRRRRRRRRRRRRRRR;
tt.
MOE-290
(SEQ ID NO: 252
(5′-CCGAAAGAAGTATGAACC-3′);
Stereopattern: SSSRRRRRRRRRRRSSS;
uu.
MOE-291
(SEQ ID NO: 252)
(5′-CCGAAAGAAGTATGAACC-3′);
Stereopattern: RRRRRRRRSSSSSSSSS;
vv.
MOE-292
(SEQ ID NO: 252)
(5′-CCGAAAGAAGTATGAACC-3′);
Stereopattern: SSSSSSSSSRRRRRRRR;
ww.
MOE-293
(SEQ ID NO: 252)
(5′-CCGAAAGAAGTATGAACC-3′);
Stereopattern: SSSRSSRSSSRSSRSSS;
xx.
MOE-294
(SEQ ID NO: 252)
(5′-CCGAAAGAAGTATGAACC-3′);
Stereopattern: SSSRSRSRSRSRSRSSS;
yy.
MOE-295
(SEQ ID NO: 252)
(5′-CCGAAAGAAGTATGAACC-3′);
Stereopattern: SRSSSRSSSRSSSRSSS;
zz.
MOE-296
(SEQ ID NO: 252)
(5′-CCGAAAGAAGTATGAACC-3′);
Stereopattern: SSSOSSRSSSRSSOSSS;
aaa.
MOE-297
(SEQ ID NO: 252)
(5′-CCGAAAGAAGTATGAACC-3′);
Stereopattern: SSSOSRSRSRSSSOSSS;
bbb.
MOE-298
(SEQ ID NO: 252)
(5′-CCGAAAGAAGTATGAACC-3′);
Stereopattern: SOSSSRSSSRSSSOSSS;
ccc.
MOE-299
(SEQ ID NO: 12)
(5′-ATCCGAAAGAAGTATGAACC-3′);
Stereopattern: SSSOSSSRSSSRSSSOSSS;
ddd.
MOE-300
(SEQ ID NO: 252)
(5′-CCGAAAGAAGTATGAACC-3′);
Stereopattern: RRRORRROSSSSSSSSS;
eee.
MOE-301
(SEQ ID NO: 252)
(5′-CCGAAAGAAGTATGAACC-3′);
Stereopattern: SRRORRROSSSSSSSSS;
fff.
MOE-303
(SEQ ID NO: 252)
(5′-CCGAAAGAAGTATGAACC-3′);
Stereopattern: SSOOOSSSSSSSSSSSS;
ggg.
MOE-304
(SEQ ID NO: 252)
(5′-CCGAAAGAAGTATGAACC-3′);
Stereopattern: OOOOOSSSSSSSSSSSS;
hhh.
MOE-305
(SEQ ID NO: 252)
(5′-CCGAAAGAAGTATGAACC-3′);
Stereopattern: SSOSSSOSSOSSSOSSS;
iii.
MOE-306
(SEQ ID NO: 252)
(5′-CCGAAAGAAGTATGAACC-3′);
Stereopattern: SOSSSSOSSSSSSOSSS;
jjj.
MOE-307
(SEQ ID NO: 252)
(5′-CCGAAAGAAGTATGAACC-3′);
Stereopattern: SSOSSSSSSSSSSOSSS;
kkk.
MOE-308
(SEQ ID NO: 12)
(5′-ATCCGAAAGAAGTATGAACC-3′);
Stereopattern: SSSOSSSSSSSSSSSOSSS;
lll.
MOE-309
(SEQ ID NO: 12)
(5′-ATCCGAAAGAAGTATGAACC-3′);
Stereopattern: SSSOSSSSOSSOSSSOSSS;
mmm.
MOE-310
(SEQ ID NO: 252)
(5′-CCGAAAGAAGTATGAACC-3′)
Stereopattern: SSORRRRRSSSSSOSSS;
or
nnn.
MOE-311
(SEQ ID NO: 252)
(5′-CCGAAAGAAGTATGAACC-3′).
Stereopattern: RRRRROSSSSSSSOSSS.

In some embodiments, the antisense oligonucleotide comprises modified sugar moieties. In some embodiments, the modified sugar moieties comprise 2′-O-methoxyethyl ribose (2′-O-MOE). In some embodiments, the modified sugar moieties comprise phosphorodiamidate morpholino oligomers (PMOs). In some embodiments, the antisense oligonucleotide comprises non-natural internucleotide linkages. In some embodiments, the non-natural internucleotide linkages are stereopure. In some embodiments, the non-natural internucleotide linkages are all Sp. In some embodiments, the non-natural internucleotide linkages are all Rp. In some embodiments, the non-natural internucleotide linkages are independently selected from Sp and Rp, i.e., each internucleotide linkage is independently selected to be Sp or Rp. In some embodiments, the non-natural internucleotide linkages are stereorandom. In some embodiments, the antisense oligonucleotide comprises modified nucleobases.

Also provided herein is a composition comprising an antisense oligonucleotide and optionally a pharmaceutically acceptable carrier or excipient.

In some embodiments, the present disclosure provides a method of inducing Exon-2 skipping in the CD33 gene during pre-mRNA splicing, comprising introducing a nucleic acid molecule into a cell, wherein the nucleic acid molecule is an antisense oligonucleotide complementary to a portion of SEQ ID NO:1, wherein the oligonucleotide hybridizes to a target region of the CD33 gene, wherein the oligonucleotide induces Exon-2 skipping during pre-mRNA splicing of the CD33 gene. In some embodiments, the antisense oligonucleotide is complementary to a portion of: SEQ ID NO:213; SEQ ID NO:214; SEQ ID NO:215; SEQ ID NO:216; SEQ ID NO:217; SEQ ID NO:218; SEQ ID NO:219; and/or SEQ ID NO:220. In some embodiments, the Exon-2 skipping efficiency of the antisense oligonucleotide is 30% or greater. In some embodiments, the Exon-2 skipping efficiency of the antisense oligonucleotide is 30% or greater according to a Standard Exon-Skipping Efficiency Assay for ASOs. In some embodiments, the Standard Exon-Skipping Efficiency Assay is a Standard Exon-Skipping Efficiency Assay for PMO ASOs when the antisense oligonucleotide comprises phosphorodiamidate morpholino oligomers or a Standard Exon-Skipping Efficiency Assay for MOE ASOs when the antisense oligonucleotide comprises methoxyethyl ribose oligomers.

In some embodiments, the present disclosure provides a method of inducing Exon-2 skipping in the CD33 gene mentioned above, wherein the antisense oligonucleotide comprises all or a portion of:

a. PMO-002
(SEQ ID NO: 2)
(5′-CCTCACCTGTCACATGCACAGAGAG-3′);
b. PMO-003
(SEQ ID NO: 3)
(5′-CCTGTCACATGCACAGAGAGCTGGG-3′);
c. PMO-036
(SEQ ID NO: 36)
(5′-TTGTAACTGTATTTGGTACTTCCTC-3′);
d. PMO-037
(SEQ ID NO: 37)
(5′-ACTGTATTTGGTACTTCCTCTCTCC-3′);
e. PMO-004
(SEQ ID NO: 4)
(5′-ATTTGGTACTTCCTCTCTCCATCCG-3′);
f. PMO-038
(SEQ ID NO: 38)
(5′-GTACTTCCTCTCTCCATCCGAAAGA-3′);
g. PMO-039
(SEQ ID NO: 39)
(5′-TCCTCTCTCCATCCGAAAGAAGTAT-3′);
h. PMO-005
(SEQ ID NO: 5)
(5′-TCTCCATCCGAAAGAAGTATGAACC-3′);
i. PMO-082
(SEQ ID NO: 82)
(5′-TAGTAGGGTATGGGATGGAAGAAAG-3′);
j. PMO-083
(SEQ ID NO: 83)
(5′-GGGTATGGGATGGAAGAAAGTGCAG-3′);
k. PMO-006
(SEQ ID NO: 6)
(5′-TGGGATGGAAGAAAGTGCAGGGCAC-3′);
l. PMO-096
(SEQ ID NO: 96)
(5′-ACTTGCAGCCAGAAATTTGGATCCA-3′);
m. PMO-007
(SEQ ID NO: 7)
(5′-CAGCCAGAAATTTGGATCCATAGCC-3′);
n. PMO-097
(SEQ ID NO: 97)
(5′-AGAAATTTGGATCCATAGCCAGGGC-3′);
o. PMO-008
(SEQ ID NO: 8)
(5′-CCCTGTGGGGAAACGAGGGTCAGCT-3′);
p. MOE-009
(SEQ ID NO: 9)
(5′-CACATGCACAGAGAGCTGGG-3′);
q. MOE-128
(SEQ ID NO: 128)
(5′-GCACAGAGAGCTGGGGAGAT-3′);
r. MOE-010
(SEQ ID NO: 10)
(5′-GAGAGCTGGGGAGATTTGTA-3′);
s. MOE-132
(SEQ ID NO: 132)
(5′-ACTGTATTTGGTACTTCCTC-3′);
t. MOE-135
(SEQ ID NO: 135)
(5′-TCCTCTCTCCATCCGAAAGA-3′);
u. MOE-011
(SEQ ID NO: 11)
(5′-TCTCCATCCGAAAGAAGTAT-3′);
v. MOE-012
(SEQ ID NO: 12)
(5′-ATCCGAAAGAAGTATGAACC-3′);
w. MOE-136
(SEQ ID NO: 136)
(5′-AAAGAAGTATGAACCATTAT-3′);
x. MOE-013
(SEQ ID NO: 13)
(5′-ATGCTCAGGGAGCAGTTGTT-3′);
y. MOE-014
(SEQ ID NO: 14)
(5′-GAGTCTCCTCCTGTACTTCT-3′);
z. MOE-015
(SEQ ID NO: 15)
(5′-CGCACAAACCCTCCTGTACC-3′);
aa. MOE-183
(SEQ ID NO: 183)
(5′-AAACCCTCCTGTACCGTCAC-3′);
bb. MOE-184
(SEQ ID NO: 184)
(5′-CTCCTGTACCGTCACTGACT-3′);
cc. MOE-190
(SEQ ID NO: 190)
(5′-CAGCCAGAAATTTGGATCCA-3′);
dd. MOE-196
(SEQ ID NO: 196)
(5′-CCCTGTGGGGAAACGAGGGT-3′);
or
ee. MOE-197
(SEQ ID NO: 197)
(5′-TGGGGAAACGAGGGTCAGCT-3′).

In some embodiments, the present disclosure provides a method of inducing Exon-2 skipping in the CD33 gene mentioned above, wherein the antisense oligonucleotide comprises all or a portion of:

a. PMO-221
(SEQ ID NO: 221)
(5′-CCTCACCTGTCACATGCACAGAG-3′);
b. PMO-222
(SEQ ID NO: 222)
(5′-TCACCTGTCACATGCACAGAGAG-3′);
c. PMO-223
(SEQ ID NO: 223)
(5′-CTCACCTGTCACATGCACAGAGA-3′);
d. PMO-224
(SEQ ID NO: 224)
(5′-CCTCACCTGTCACATGCACAG-3′);
e. PMO-225
(SEQ ID NO: 225)
(5′-ACCTGTCACATGCACAGAGAG-3′);
f. PMO-226
(SEQ ID NO: 226)
(5′-TCACCTGTCACATGCACAGAG-3′);
g. PMO-227
(SEQ ID NO: 227)
(5′-TCACCTGTCACATGCACAGAGAGCT-3′);
h. PMO-228
(SEQ ID NO: 228)
(5′-CCTGTGCCTCACCTGTCACATGCAC-3′);
i. PMO-229
(SEQ ID NO: 229)
(5′-GTGCCTCACCTGTCACATGCACAGA-3′);
j. PMO-230
(SEQ ID NO: 230)
(5′-TGCCTCACCTGTCACATGCACAGAG-3′);
k. PMO-231
(SEQ ID NO: 231)
(5′-CTCACCTGTCACATGCACAGAGAGC-3′);
l. PMO-232
(SEQ ID NO: 232)
(5′-CACCTGTCACATGCACAGAGAGCTG-3′);
m. PMO-233
(SEQ ID NO: 233)
(5′-ACCTGTCACATGCACAGAGAGCTGG-3′);
n. PMO-234
(SEQ ID NO: 234)
(5′-CTGTCACATGCACAGAGAGCTGGGG-3′);
o. PMO-235
(SEQ ID NO: 235)
(5′-CCTGTCACATGCACAGAGAGCTG-3′);
p. PMO-236
(SEQ ID NO: 236)
(5′-TGTCACATGCACAGAGAGCTGGG-3′);
q. PMO-237
(SEQ ID NO: 237)
(5′-CTGTCACATGCACAGAGAGCTGG-3′);
r. PMO-238
(SEQ ID NO: 238)
(5′-TGTCACATGCACAGAGAGCTGG-3′);
s. PMO-239
(SEQ ID NO: 239)
(5′-TCACATGCACAGAGAGCTGGG-3′);
t. PMO-240
(SEQ ID NO: 240)
(5′-TGTCACATGCACAGAGAGCTG-3′);
u. PMO-241
(SEQ ID NO: 241)
(5′-CTGTATTTGGTACTTCCTCTCTCCA-3′);
v. PMO-242
(SEQ ID NO: 242)
(5′-TGTATTTGGTACTTCCTCTCTCCAT-3′);
w. PMO-243
(SEQ ID NO: 243)
(5′-GTATTTGGTACTTCCTCTCTCCATC-3′);
x. PMO-244
(SEQ ID NO: 244)
(5′-TATTTGGTACTTCCTCTCTCCATCC-3′);
y. PMO-324
(SEQ ID NO: 224)
(5′-CCTCACCTGTCACATGCACAG-3′);
Stereopattern:
RRRRRRRRRRRRRRRRRRRR
z. PMO-424
(SEQ ID NO: 224)
(5′-CCTCACCTGTCACATGCACAG-3′);
Stereopattern:
SSSSSSSSSSSSSSSSSSSS
aa. PMO-402
(SEQ ID NO: 002)
(5′-CCTCACCTGTCACATGCACAGAGAG-3′);
Stereopattern:
RRRRRRRRRRRRRRRRRRRRRRRR;
or
bb. PMO-502
(SEQ ID NO: 002)
(5′-CCTCACCTGTCACATGCACAGAGAG-3′);
Stereopattern:
SSSSSSSSSSSSSSSSSSSSSSSS.

In some embodiments, the present disclosure provides a method of inducing Exon-2 skipping in the CD33 gene mentioned above, wherein the antisense oligonucleotide comprises all or a portion of:

a. MOE-245
(SEQ ID NO: 245)
(5′-CTCCATCCGAAAGAAGTATG-3′);
b. MOE-246
(SEQ ID NO: 246)
(5′-TCCATCCGAAAGAAGTATGA-3′);
c. MOE-247
(SEQ ID NO: 247)
(5′-CCATCCGAAAGAAGTATGAA-3′);
d. MOE-248
(SEQ ID NO: 248)
(5′-CATCCGAAAGAAGTATGAAC-3′);
e. MOE-249
(SEQ ID NO: 249)
(5′-TCCGAAAGAAGTATGAACCA-3′);
f. MOE-250
(SEQ ID NO: 250)
(5′-CCGAAAGAAGTATGAACCAT-3′);
g. MOE-251
(SEQ ID NO: 251)
(5′-ATCCGAAAGAAGTATGAA-3′);
h. MOE-252
(SEQ ID NO: 252)
(5′-CCGAAAGAAGTATGAACC-3′);
i. MOE-253
(SEQ ID NO: 253)
(5′-TCCGAAAGAAGTATGAAC-3′);
j. MOE-254
(SEQ ID NO: 254)
(5′-CCATCCGAAAGAAGTATG-3′);
k. MOE-255
(SEQ ID NO: 255)
(5′-TCCATCCGAAAGAAGTAT-3′);
l. MOE-256
(SEQ ID NO: 256)
(5′-GAAAGAAGTATGAACCAT-3′);
m. MOE-257
(SEQ ID NO: 012)
(5′-ATC-CGAAAGAAGTATGA-ACC-3′);
n. MOE-258
(SEQ ID NO: 012)
(5′-ATCC-GAAAGAAGTATG-AACC-3′);
o. MOE-259
(SEQ ID NO: 012)
(5′-ATCCG-AAAGAAGTAT-GAACC-3′);
p. MOE-260
(SEQ ID NO: 012)
(5′-ATCCG-AAAGAAGTA-TGAACC-3′);
q. MOE-261
(SEQ ID NO: 012)
(5′-ATCC-GAAAGA-AGTATG-AACC-3′);
r. MOE-262
(SEQ ID NO: 012)
(5′-ATCC-gAAAGAAGTATG-aACC-3′);
s. MOE-263
(SEQ ID NO: 012)
(5′-ATCC-gAAAGAAGTATG-aACC-3′);
t. MOE-264
(SEQ ID NO: 012)
(5′-ATCC-gAAAGAaGTATG-aACC-3′);
u. MOE-265
(SEQ ID NO: 252)
(5′-CCGA-aAGAAGTATGAACC-3′);
v. MOE-266
(SEQ ID NO: 252)
(5′-CCGA-aAGAAGTATG-aACC-3′);
w. MOE-267
(SEQ ID NO: 252)
(5′-CCGA-aAGAAGtATG-aACC-3′);
x. MOE-268
(SEQ ID NO: 252)
(5′-CCG-AAAGAAGTATGA-ACC-3′);
y. MOE-269
(SEQ ID NO: 252)
(5′-CCGA-AAGAAGTATG-AACC-3′);
z. MOE-270
(SEQ ID NO: 252)
(5′-CCGAA-AGAA-GTATG-AACC-3′);
aa. MOE-271
(SEQ ID NO: 252)
(5′-CCGAA-AGAAGTAT-GAACC-3′);
bb MOE-272
(SEQ ID NO: 252)
(5′-CCG-A-AAGAAGTATGAACC-3′);
cc. MOE-273
(SEQ ID NO: 252)
(5′-CCG-AA-AGAAGTATGAACC-3′);
dd. MOE-274
(SEQ ID NO: 252)
(5′-CCGAAAGAAGTATG-A-ACC-3′);
ee. MOE-275
(SEQ ID NO: 012)
(5′-mAmTfCfCfGfAfAfAfGfAfAfGfTfAfTfGfAfAmCmC-3′);
ff. MOE-276
(SEQ ID NO: 012)
(5′-fAfTfCfCfGmAmAmAmGmAmAmGmTmAfTfGfAfAfCfC-3′);
gg. MOE-277
(SEQ ID NO: 012)
(5′-ATCCGAAAGAAGTATGAACC-3′);
Stereopattern:
SSSSSSSSSSSSSSSSSSS;
hh. MOE-278
(SEQ ID NO: 012)
(5′-ATCCGAAAGAAGTATGAACC-3′);
Stereopattern:
RRRRRRRRRRRRRRRRRRR;
ii. MOE-279
(SEQ ID NO: 012)
(5′-ATCCGAAAGAAGTATGAACC-3′);
Stereopattern:
SSSRSSSRSSSRSSSRSSS;
jj. MOE-280
(SEQ ID NO: 012)
(5′-ATCCGAAAGAAGTATGAACC-3′);
Stereopattern:
SSSRSSRSSRSSRSSRSSS;
kk. MOE-281
(SEQ ID NO: 012)
(5′-ATCCGAAAGAAGTATGAACC-3′);
Stereopattern:
SSSRSRSRSRSRSRSRSSS;
ll. MOE-282
(SEQ ID NO: 012)
(5′-ATCCGAAAGAAGTATGAACC-3′);
Stereopattern:
SSSSSSRRRRRRRSSSSSS;
mm. MOE-283
(SEQ ID NO: 012)
(5′-ATCCGAAAGAAGTATGAACC-3′);
Stereopattern:
SSSRRSRRSRRSRRSRSSS;
nn. MOE-284
(SEQ ID NO: 012)
(5′-ATCCGAAAGAAGTATGAACC-3′);
Stereopattern:
SSSRRSRRRSRRRSRRSSS;
oo. MOE-285
(SEQ ID NO: 012)
(5′-ATCCGAAAGAAGTATGAACC-3′);
Stereopattern:
SSSSSRRRRRRRRRSSSSS;
pp. MOE-286
(SEQ ID NO: 012)
(5′-ATCCGAAAGAAGTATGAACC-3′);
Stereopattern:
SSSRRRRRRRRRRRRRSS;
qq. MOE-287
(SEQ ID NO: 012)
(5′-ATCCGAAAGAAGTATGAACC-3′);
Stereopattern:
SSRSSSSSSSSRSRSSSSS;
rr. MOE-288
(SEQ ID NO: 252)
(5′-CCGAAAGAAGTATGAACC-3′);
Stereopattern:
SSSSSSSSSSSSSSSSS;
ss. MOE-289
(SEQ ID NO: 252)
(5′-CCGAAAGAAGTATGAACC-3′);
Stereopattern:
RRRRRRRRRRRRRRRRR;
tt. MOE-290
(SEQ ID NO: 252
(5′-CCGAAAGAAGTATGAACC-3′);
Stereopattern:
SSSRRRRRRRRRRRSSS;
uu. MOE-291
(SEQ ID NO: 252)
(5′-CCGAAAGAAGTATGAACC-3′);
Stereopattern:
RRRRRRRRSSSSSSSSS;
vv. MOE-292
(SEQ ID NO: 252)
(5′-CCGAAAGAAGTATGAACC-3′);
Stereopattern:
SSSSSSSSSRRRRRRRR;
ww. MOE-293
(SEQ ID NO: 252)
(5′-CCGAAAGAAGTATGAACC-3′);
Stereopattern:
SSSRSSRSSSRSSRSSS;
xx. MOE-294
(SEQ ID NO: 252)
(5′-CCGAAAGAAGTATGAACC-3′);
Stereopattern:
SSSRSRSRSRSRSRSSS;
yy. MOE-295
(SEQ ID NO: 252)
(5′-CCGAAAGAAGTATGAACC-3′);
Stereopattern:
SRSSSRSSSRSSSRSSS;
zz. MOE-296
(SEQ ID NO: 252)
(5′-CCGAAAGAAGTATGAACC-3′);
Stereopattern:
SSSOSSRSSSRSSOSSS;
aaa. MOE-297
(SEQ ID NO: 252)
(5′-CCGAAAGAAGTATGAACC-3′);
Stereopattern:
SSSOSRSRSRSSSOSSS;
bbb. MOE-298
(SEQ ID NO: 252)
(5′-CCGAAAGAAGTATGAACC-3′);
Stereopattern:
SOSSSRSSSRSSSOSSS;
ccc. MOE-299
(SEQ ID NO: 12)
(5′-ATCCGAAAGAAGTATGAACC-3′);
Stereopattern:
SSSOSSSRSSSRSSSOSSS;
ddd. MOE-300
(SEQ ID NO: 252)
(5′-CCGAAAGAAGTATGAACC-3′);
Stereopattern:
RRRORRROSSSSSSSSS;
eee. MOE-301
(SEQ ID NO: 252)
(5′-CCGAAAGAAGTATGAACC-3′);
Stereopattern:
SRRORRROSSSSSSSSS;
fff. MOE-303
(SEQ ID NO: 252)
(5′-CCGAAAGAAGTATGAACC-3′);
Stereopattern:
SSOOOSSSSSSSSSSSS;
ggg. MOE-304
(SEQ ID NO: 252)
(5′-CCGAAAGAAGTATGAACC-3′);
Stereopattern:
OOOOOSSSSSSSSSSSS;
hhh. MOE-305
(SEQ ID NO: 252)
(5′-CCGAAAGAAGTATGAACC-3′);
Stereopattern:
SSOSSSOSSOSSSOSSS;
iii. MOE-306
(SEQ ID NO: 252)
(5′-CCGAAAGAAGTATGAACC-3′);
Stereopattern:
SOSSSSOSSSSSSOSSS;
jjj. MOE-307
(SEQ ID NO: 252)
(5′-CCGAAAGAAGTATGAACC-3′);
Stereopattern:
SSOSSSSSSSSSSOSSS;
kkk. MOE-308
(SEQ ID NO: 12)
(5′-ATCCGAAAGAAGTATGAACC-3′);
Stereopattern:
ssSOSSSSSSSSSSSOSSS;
lll. MOE-309
(SEQ ID NO: 12)
(5′-ATCCGAAAGAAGTATGAACC-3′);
Stereopattern:
SSSOSSSSOSSOSSSOSSS;
mmm. MOE-310
(SEQ ID NO: 252)
(5′-CCGAAAGAAGTATGAACC-3′);
Stereopattern:
SSORRRRRSSSSSOSSS;
or
nnn. MOE-311
(SEQ ID NO: 252)
(5′-CCGAAAGAAGTATGAACC-3′).
Stereopattern:
RRRRROSSSSSSSOSSS.

In some embodiments, the present disclosure provides a method of inducing Exon-2 skipping in the CD33 gene mentioned above, wherein the cell is an animal cell. In some embodiments, the cell is a human cell.

In some embodiments, the present disclosure provides a method of treating a subject having a neurodegenerative disease comprising administering a therapeutically effective amount of an antisense oligonucleotide of 16-30 nucleotides in length, wherein the antisense oligonucleotide is complementary to a portion of SEQ ID NO:1, and wherein the antisense oligonucleotide has a CD33 Exon-2 skipping efficiency of 30% or greater according to a Standard Exon-Skipping Efficiency Assay for the antisense oligonucleotide.

In some embodiments, the present disclosure provides a method of treating a subject having a neurodegenerative disease mentioned above, wherein the antisense oligonucleotide comprises all or a portion of:

a. PMO-002
(SEQ ID NO: 2)
(5′-CCTCACCTGTCACATGCACAGAGAG-3′);
b. PMO-003
(SEQ ID NO: 3)
(5′-CCTGTCACATGCACAGAGAGCTGGG-3′);
c. PMO-036
(SEQ ID NO: 36)
(5′-TTGTAACTGTATTTGGTACTTCCTC-3′);
d. PMO-037
(SEQ ID NO: 37)
(5′-ACTGTATTTGGTACTTCCTCTCTCC-3′);
e. PMO-004
(SEQ ID NO: 4)
(5′-ATTTGGTACTTCCTCTCTCCATCCG-3′);
f. PMO-038
(SEQ ID NO: 38)
(5′-GTACTTCCTCTCTCCATCCGAAAGA-3′);
g. PMO-039
(SEQ ID NO: 39)
(5′-TCCTCTCTCCATCCGAAAGAAGTAT-3′);
h. PMO-005
(SEQ ID NO: 5)
(5′-TCTCCATCCGAAAGAAGTATGAACC-3′);
i. PMO-082
(SEQ ID NO: 82)
(5′-TAGTAGGGTATGGGATGGAAGAAAG-3′);
j. PMO-083
(SEQ ID NO: 83)
(5′-GGGTATGGGATGGAAGAAAGTGCAG-3′);
k. PMO-006
(SEQ ID NO: 6)
(5′-TGGGATGGAAGAAAGTGCAGGGCAC-3′);
l. PMO-096
(SEQ ID NO: 96)
(5′-ACTTGCAGCCAGAAATTTGGATCCA-3′);
m. PMO-007
(SEQ ID NO: 7)
(5′-CAGCCAGAAATTTGGATCCATAGCC-3′);
n. PMO-097
(SEQ ID NO: 97)
(5′-AGAAATTTGGATCCATAGCCAGGGC-3′);
o. PMO-008
(SEQ ID NO: 8)
(5′-CCCTGTGGGGAAACGAGGGTCAGCT-3′);
p. MOE-009
(SEQ ID NO: 9)
(5′-CACATGCACAGAGAGCTGGG-3′);
q. MOE-128
(SEQ ID NO: 128)
(5′-GCACAGAGAGCTGGGGAGAT-3′);
r. MOE-010
(SEQ ID NO: 10)
(5′-GAGAGCTGGGGAGATTTGTA-3′);
s. MOE-132
(SEQ ID NO: 132)
(5′-ACTGTATTTGGTACTTCCTC-3′);
t. MOE-135
(SEQ ID NO: 135)
(5′-TCCTCTCTCCATCCGAAAGA-3′);
u. MOE-011
(SEQ ID NO: 11)
(5′-TCTCCATCCGAAAGAAGTAT-3′);
v. MOE-012
(SEQ ID NO: 12)
(5′-ATCCGAAAGAAGTATGAACC-3′);
w. MOE-136
(SEQ ID NO: 136)
(5′-AAAGAAGTATGAACCATTAT-3′);
x. MOE-013
(SEQ ID NO: 13)
(5′-ATGCTCAGGGAGCAGTTGTT-3′);
y. MOE-014
(SEQ ID NO: 14)
(5′-GAGTCTCCTCCTGTACTTCT-3′);
z. MOE-015
(SEQ ID NO: 15)
(5′-CGCACAAACCCTCCTGTACC-3′);
aa. MOE-183
(SEQ ID NO: 183)
(5′-AAACCCTCCTGTACCGTCAC-3′);
bb. MOE-184
(SEQ ID NO: 184)
(5′-CTCCTGTACCGTCACTGACT-3′);
cc. MOE-190
(SEQ ID NO: 190)
(5′-CAGCCAGAAATTTGGATCCA-3′);
dd. MOE-196
(SEQ ID NO: 196)
(5′-CCCTGTGGGGAAACGAGGGT-3′);
or
ee. MOE-197
(SEQ ID NO: 197)
(5′-TGGGGAAACGAGGGTCAGCT-3′).

In some embodiments, the present disclosure provides a method of treating a subject having a neurodegenerative disease mentioned above, wherein the antisense oligonucleotide comprises all or a portion of:

a. PMO-221
(SEQ ID NO: 221)
(5′-CCTCACCTGTCACATGCACAGAG-3′);
b. PMO-222
(SEQ ID NO: 222)
(5′-TCACCTGTCACATGCACAGAGAG-3′);
c. PMO-223
(SEQ ID NO: 223)
(5′-CTCACCTGTCACATGCACAGAGA-3′);
d. PMO-224
(SEQ ID NO: 224)
(5′-CCTCACCTGTCACATGCACAG-3′);
e. PMO-225
(SEQ ID NO: 225)
(5′-ACCTGTCACATGCACAGAGAG-3′);
f. PMO-226
(SEQ ID NO: 226)
(5′-TCACCTGTCACATGCACAGAG-3′);
g. PMO-227
(SEQ ID NO: 227)
(5′-TCACCTGTCACATGCACAGAGAGCT-3′);
h. PMO-228
(SEQ ID NO: 228)
(5′-CCTGTGCCTCACCTGTCACATGCAC-3′);
i. PMO-229
(SEQ ID NO: 229)
(5′-GTGCCTCACCTGTCACATGCACAGA-3′);
j. PMO-230
(SEQ ID NO: 230)
(5′-TGCCTCACCTGTCACATGCACAGAG-3′);
k. PMO-231
(SEQ ID NO: 231)
(5′-CTCACCTGTCACATGCACAGAGAGC-3′);
l. PMO-232
(SEQ ID NO: 232)
(5′-CACCTGTCACATGCACAGAGAGCTG-3′);
m. PMO-233
(SEQ ID NO: 233)
(5′-ACCTGTCACATGCACAGAGAGCTGG-3′);
n. PMO-234
(SEQ ID NO: 234)
(5′-CTGTCACATGCACAGAGAGCTGGGG-3′);
o. PMO-235
(SEQ ID NO: 235)
(5′-CCTGTCACATGCACAGAGAGCTG-3′);
p. PMO-236
(SEQ ID NO: 236)
(5′-TGTCACATGCACAGAGAGCTGGG-3′);
q. PMO-237
(SEQ ID NO: 237)
(5′-CTGTCACATGCACAGAGAGCTGG-3′);
r. PMO-238
(SEQ ID NO: 238)
(5′-TGTCACATGCACAGAGAGCTGG-3′);
s. PMO-239
(SEQ ID NO: 239)
(5′-TCACATGCACAGAGAGCTGGG-3′);
t. PMO-240
(SEQ ID NO: 240)
(5′-TGTCACATGCACAGAGAGCTG-3′);
u. PMO-241
(SEQ ID NO: 241)
(5′-CTGTATTTGGTACTTCCTCTCTCCA-3′);
v. PMO-242
(SEQ ID NO: 242)
(5′-TGTATTTGGTACTTCCTCTCTCCAT-3′);
w. PMO-243
(SEQ ID NO: 243)
(5′-GTATTTGGTACTTCCTCTCTCCATC-3′);
x. PMO-244
(SEQ ID NO: 244)
(5′-TATTTGGTACTTCCTCTCTCCATCC-3′);
y. PMO-324
(SEQ ID NO: 224)
(5′-CCTCACCTGTCACATGCACAG-3′);
Stereopattern:
RRRRRRRRRRRRRRRRRRRR
z. PMO-424
(SEQ ID NO: 224)
(5′-CCTCACCTGTCACATGCACAG-3′);
Stereopattern:
SSSSSSSSSSSSSSSSSSSS
aa. PMO-402
(SEQ ID NO: 002)
(5′-CCTCACCTGTCACATGCACAGAGAG-3′);
Stereopattern:
RRRRRRRRRRRRRRRRRRRRRRRR;
or
bb. PMO-502
(SEQ ID NO: 002)
(5′-CCTCACCTGTCACATGCACAGAGAG-3′);
Stereopattern:
SSSSSSSSSSSSSSSSSSSSSSSS.

In some embodiments, the present disclosure provides a method of treating a subject having a neurodegenerative disease mentioned above, wherein the antisense oligonucleotide comprises all or a portion of:

a. MOE-245
(SEQ ID NO: 245)
(5′-CTCCATCCGAAAGAAGTATG-3′);
b. MOE-246
(SEQ ID NO: 246)
(5′-TCCATCCGAAAGAAGTATGA-3′);
c. MOE-247
(SEQ ID NO: 247)
(5′-CCATCCGAAAGAAGTATGAA-3′);
d. MOE-248
(SEQ ID NO: 248)
(5′-CATCCGAAAGAAGTATGAAC-3′);
e. MOE-249
(SEQ ID NO: 249)
(5′-TCCGAAAGAAGTATGAACCA-3′);
f. MOE-250
(SEQ ID NO: 250)
(5′-CCGAAAGAAGTATGAACCAT-3′);
g. MOE-251
(SEQ ID NO: 251)
(5′-ATCCGAAAGAAGTATGAA-3′);
h. MOE-252
(SEQ ID NO: 252)
(5′-CCGAAAGAAGTATGAACC-3′);
i. MOE-253
(SEQ ID NO: 253)
(5′-TCCGAAAGAAGTATGAAC-3′);
j. MOE-254
(SEQ ID NO: 254)
(5′-CCATCCGAAAGAAGTATG-3′);
k. MOE-255
(SEQ ID NO: 255)
(5′-TCCATCCGAAAGAAGTAT-3′);
l. MOE-256
(SEQ ID NO: 256)
(5′-GAAAGAAGTATGAACCAT-3′);
m. MOE-257
(SEQ ID NO: 012)
(5′-ATC-CGAAAGAAGTATGA-ACC-3′);
n. MOE-258
(SEQ ID NO: 012)
(5′-ATCC-GAAAGAAGTATG-AACC-3′);
o. MOE-259
(SEQ ID NO: 012)
(5′-ATCCG-AAAGAAGTAT-GAACC-3′);
p. MOE-260
(SEQ ID NO: 012)
(5′-ATCCG-AAAGAAGTA-TGAACC-3′);
q. MOE-261
(SEQ ID NO: 012)
(5′-ATCC-GAAAGA-AGTATG-AACC-3′);
r. MOE-262
(SEQ ID NO: 012)
(5′-ATCC-gAAAGAAGTATG-aACC-3′);
s. MOE-263
(SEQ ID NO: 012)
(5′-ATCC-gAAAGAAGTATG-aACC-3′);
t. MOE-264
(SEQ ID NO: 012)
(5′-ATCC-gAAAGAaGTATG-aACC-3′);
u. MOE-265
(SEQ ID NO: 252)
(5′-CCGA-aAGAAGTATGAACC-3′);
v. MOE-266
(SEQ ID NO: 252)
(5′-CCGA-aAGAAGTATG-aACC-3′);
w. MOE-267
(SEQ ID NO: 252)
(5′-CCGA-aAGAAGtATG-aACC-3′);
x. MOE-268
(SEQ ID NO: 252)
(5′-CCG-AAAGAAGTATGA-ACC-3′);
y. MOE-269
(SEQ ID NO: 252)
(5′-CCGA-AAGAAGTATG-AACC-3′);
z. MOE-270
(SEQ ID NO: 252)
(5′-CCGAA-AGAA-GTATG-AACC-3′);
aa. MOE-271
(SEQ ID NO: 252)
(5′-CCGAA-AGAAGTAT-GAACC-3′);
bb MOE-272
(SEQ ID NO: 252)
(5′-CCG-A-AAGAAGTATGAACC-3′);
cc. MOE-273
(SEQ ID NO: 252)
(5′-CCG-AA-AGAAGTATGAACC-3′);
dd. MOE-274
(SEQ ID NO: 252)
(5′-CCGAAAGAAGTATG-A-ACC-3′);
ee. MOE-275
(SEQ ID NO: 012)
(5′-mAmTfCfCfGfAfAfAfGfAfAfGfTfAfTfGfAfAmCmC-3′);
ff. MOE-276
(SEQ ID NO: 012)
(5′-fAfTfCfCfGmAmAmAmGmAmAmGmTmAfTfGfAfAfCfC-3′);
gg. MOE-277
(SEQ ID NO: 012)
(5′-ATCCGAAAGAAGTATGAACC-3′);
Stereopattern:
SSSSSSSSSSSSSSSSSSS;
hh. MOE-278
(SEQ ID NO: 012)
(5′-ATCCGAAAGAAGTATGAACC-3′);
Stereopattern:
RRRRRRRRRRRRRRRRRRR;
ii. MOE-279
(SEQ ID NO: 012)
(5′-ATCCGAAAGAAGTATGAACC-3′);
Stereopattern:
SSSRSSSRSSSRSSSRSSS;
jj. MOE-280
(SEQ ID NO: 012)
(5′-ATCCGAAAGAAGTATGAACC-3′);
Stereopattern:
SSSRSSRSSRSSRSSRSSS;
kk. MOE-281
(SEQ ID NO: 012)
(5′-ATCCGAAAGAAGTATGAACC-3′);
Stereopattern:
SSSRSRSRSRSRSRSRSSS;
ll. MOE-282
(SEQ ID NO: 012)
(5′-ATCCGAAAGAAGTATGAACC-3′);
Stereopattern:
SSSSSSRRRRRRRSSSSSS;
mm. MOE-283
(SEQ ID NO: 012)
(5′-ATCCGAAAGAAGTATGAACC-3′);
Stereopattern:
SSSRRSRRSRRSRRSRSSS;
nn. MOE-284
(SEQ ID NO: 012)
(5′-ATCCGAAAGAAGTATGAACC-3′);
Stereopattern:
SSSRRSRRRSRRRSRRSSS;
oo. MOE-285
(SEQ ID NO: 012)
(5′-ATCCGAAAGAAGTATGAACC-3′);
Stereopattern:
SSSSSRRRRRRRRRSSSSS;
pp. MOE-286
(SEQ ID NO: 012)
(5′-ATCCGAAAGAAGTATGAACC-3′);
Stereopattern:
SSSRRRRRRRRRRRRRSS;
qq. MOE-287
(SEQ ID NO: 012)
(5′-ATCCGAAAGAAGTATGAACC-3′);
Stereopattern:
SSRSSSSSSSSRSRSSSSS;
rr. MOE-288
(SEQ ID NO: 252)
(5′-CCGAAAGAAGTATGAACC-3′);
Stereopattern:
SSSSSSSSSSSSSSSSS;
ss. MOE-289
(SEQ ID NO: 252)
(5′-CCGAAAGAAGTATGAACC-3′);
Stereopattern:
RRRRRRRRRRRRRRRRR;
tt. MOE-290
(SEQ ID NO: 252
(5′-CCGAAAGAAGTATGAACC-3′);
Stereopattern:
SSSRRRRRRRRRRRSSS;
uu. MOE-291
(SEQ ID NO: 252)
(5′-CCGAAAGAAGTATGAACC-3′);
Stereopattern:
RRRRRRRRSSSSSSSSS;
vv. MOE-292
(SEQ ID NO: 252)
(5′-CCGAAAGAAGTATGAACC-3′);
Stereopattern:
SSSSSSSSSRRRRRRRR;
ww. MOE-293
(SEQ ID NO: 252)
(5′-CCGAAAGAAGTATGAACC-3′);
Stereopattern:
SSSRSSRSSSRSSRSSS;
xx. MOE-294
(SEQ ID NO: 252)
(5′-CCGAAAGAAGTATGAACC-3′);
Stereopattern:
SSSRSRSRSRSRSRSSS;
yy. MOE-295
(SEQ ID NO: 252)
(5′-CCGAAAGAAGTATGAACC-3′);
Stereopattern:
SRSSSRSSSRSSSRSSS;
zz. MOE-296
(SEQ ID NO: 252)
(5′-CCGAAAGAAGTATGAACC-3′);
Stereopattern:
SSSOSSRSSSRSSOSSS;
aaa. MOE-297
(SEQ ID NO: 252)
(5′-CCGAAAGAAGTATGAACC-3′);
Stereopattern:
SSSOSRSRSRSSSOSSS;
bbb. MOE-298
(SEQ ID NO: 252)
(5′-CCGAAAGAAGTATGAACC-3′);
Stereopattern:
SOSSSRSSSRSSSOSSS;
ccc. MOE-299
(SEQ ID NO: 12)
(5′-ATCCGAAAGAAGTATGAACC-3′);
Stereopattern:
SSSOSSSRSSSRSSSOSSS;
ddd. MOE-300
(SEQ ID NO: 252)
(5′-CCGAAAGAAGTATGAACC-3′);
Stereopattern:
RRRORRROSSSSSSSSS;
eee. MOE-301
(SEQ ID NO: 252)
(5′-CCGAAAGAAGTATGAACC-3′);
Stereopattern:
SRRORRROSSSSSSSSS;
fff. MOE-303
(SEQ ID NO: 252)
(5′-CCGAAAGAAGTATGAACC-3′);
Stereopattern:
SSOOOSSSSSSSSSSSS;
ggg. MOE-304
(SEQ ID NO: 252)
(5′-CCGAAAGAAGTATGAACC-3′);
Stereopattern:
OOOOOSSSSSSSSSSSS;
hhh. MOE-305
(SEQ ID NO: 252)
(5′-CCGAAAGAAGTATGAACC-3′);
Stereopattern:
SSOSSSOSSOSSSOSSS;
iii. MOE-306
(SEQ ID NO: 252)
(5′-CCGAAAGAAGTATGAACC-3′);
Stereopattern:
SOSSSSOSSSSSSOSSS;
jjj. MOE-307
(SEQ ID NO: 252)
(5′-CCGAAAGAAGTATGAACC-3′);
Stereopattern:
SSOSSSSSSSSSSOSSS;
kkk. MOE-308
(SEQ ID NO: 12)
(5′-ATCCGAAAGAAGTATGAACC-3′);
Stereopattern:
ssSOSSSSSSSSSSSOSSS;
lll. MOE-309
(SEQ ID NO: 12)
(5′-ATCCGAAAGAAGTATGAACC-3′);
Stereopattern:
SSSOSSSSOSSOSSSOSSS;
mmm. MOE-310
(SEQ ID NO: 252)
(5′-CCGAAAGAAGTATGAACC-3′);
Stereopattern:
SSORRRRRSSSSSOSSS;
or
nnn. MOE-311
(SEQ ID NO: 252)
(5′-CCGAAAGAAGTATGAACC-3′).
Stereopattern:
RRRRROSSSSSSSOSSS.

In some embodiments, the present disclosure provides a method of treating a subject having a neurodegenerative disease mentioned above, wherein the neurodegenerative disease is Alzheimer's Disease.

In some embodiments, the present disclosure provides an antisense oligonucleotide mentioned above for use in a method of inducing Exon-2 skipping in the CD33 gene during pre-mRNA splicing, comprising introducing a nucleic acid molecule into a cell, wherein the nucleic acid molecule is an antisense oligonucleotide that is complementary to a portion of SEQ ID NO:1, that hybridizes to a target region of the CD33 gene, and that induces Exon-2 skipping during pre-mRNA splicing of the CD33 gene. In some embodiments, the antisense oligonucleotide is complementary to a portion of: SEQ ID NO:213; SEQ ID NO:214; SEQ ID NO:215; SEQ ID NO:216; SEQ ID NO:217; SEQ ID NO:218; SEQ ID NO:219; and/or SEQ ID NO:220. In some embodiments, the Exon-2 skipping efficiency of the antisense oligonucleotide is 30% or greater. In some embodiments, the Exon-2 skipping efficiency of the antisense oligonucleotide is 30% or greater according to a Standard Exon-Skipping Efficiency Assay for ASOs. In some embodiments, the Standard Exon-Skipping Efficiency Assay is a Standard Exon-Skipping Efficiency Assay for PMO ASOs when the antisense oligonucleotide comprises phosphorodiamidate morpholino oligomers or a Standard Exon-Skipping Efficiency Assay for MOE ASOs when the antisense oligonucleotide comprises methoxyethyl ribose oligomers.

In some embodiments, the present disclosure provides an antisense oligonucleotide mentioned above for use in a method of inducing Exon-2 skipping in the CD33 gene mentioned above, wherein the antisense oligonucleotide comprises all or a portion of:

a. PMO-002
(SEQ ID NO: 2)
(5′-CCTCACCTGTCACATGCACAGAGAG-3′);
b. PMO-003
(SEQ ID NO: 3)
(5′-CCTGTCACATGCACAGAGAGCTGGG-3′);
c. PMO-036
(SEQ ID NO: 36)
(5′-TTGTAACTGTATTTGGTACTTCCTC-3′);
d. PMO-037
(SEQ ID NO: 37)
(5′-ACTGTATTTGGTACTTCCTCTCTCC-3′);
e. PMO-004
(SEQ ID NO: 4)
(5′-ATTTGGTACTTCCTCTCTCCATCCG-3′);
f. PMO-038
(SEQ ID NO: 38)
(5′-GTACTTCCTCTCTCCATCCGAAAGA-3′);
g. PMO-039
(SEQ ID NO: 39)
(5′-TCCTCTCTCCATCCGAAAGAAGTAT-3′);
h. PMO-005
(SEQ ID NO: 5)
(5′-TCTCCATCCGAAAGAAGTATGAACC-3′);
i. PMO-082
(SEQ ID NO: 82)
(5′-TAGTAGGGTATGGGATGGAAGAAAG-3′);
j. PMO-083
(SEQ ID NO: 83)
(5′-GGGTATGGGATGGAAGAAAGTGCAG-3′);
k. PMO-006
(SEQ ID NO: 6)
(5′-TGGGATGGAAGAAAGTGCAGGGCAC-3′);
l. PMO-096
(SEQ ID NO: 96)
(5′-ACTTGCAGCCAGAAATTTGGATCCA-3′);
m. PMO-007
(SEQ ID NO: 7)
(5′-CAGCCAGAAATTTGGATCCATAGCC-3′);
n. PMO-097
(SEQ ID NO: 97)
(5′-AGAAATTTGGATCCATAGCCAGGGC-3′);
o. PMO-008
(SEQ ID NO: 8)
(5′-CCCTGTGGGGAAACGAGGGTCAGCT-3′);
p. MOE-009
(SEQ ID NO: 9)
(5′-CACATGCACAGAGAGCTGGG-3′);
q. MOE-128
(SEQ ID NO: 128)
(5′-GCACAGAGAGCTGGGGAGAT-3′);
r. MOE-010
(SEQ ID NO: 10)
(5′-GAGAGCTGGGGAGATTTGTA-3′);
s. MOE-132
(SEQ ID NO: 132)
(5′-ACTGTATTTGGTACTTCCTC-3′);
t. MOE-135
(SEQ ID NO: 135)
(5′-TCCTCTCTCCATCCGAAAGA-3′);
u. MOE-011
(SEQ ID NO: 11)
(5′-TCTCCATCCGAAAGAAGTAT-3′);
v. MOE-012
(SEQ ID NO: 12)
(5′-ATCCGAAAGAAGTATGAACC-3′);
w. MOE-136
(SEQ ID NO: 136)
(5′-AAAGAAGTATGAACCATTAT-3′);
x. MOE-013
(SEQ ID NO: 13)
(5′-ATGCTCAGGGAGCAGTTGTT-3′);
y. MOE-014
(SEQ ID NO: 14)
(5′-GAGTCTCCTCCTGTACTTCT-3′);
z. MOE-015
(SEQ ID NO: 15)
(5′-CGCACAAACCCTCCTGTACC-3′);
aa. MOE-183
(SEQ ID NO: 183)
(5′-AAACCCTCCTGTACCGTCAC-3′);
bb. MOE-184
(SEQ ID NO: 184)
(5′-CTCCTGTACCGTCACTGACT-3′);
cc. MOE-190
(SEQ ID NO: 190)
(5′-CAGCCAGAAATTTGGATCCA-3′);
dd. MOE-196
(SEQ ID NO: 196)
(5′-CCCTGTGGGGAAACGAGGGT-3′);
or
ee. MOE-197
(SEQ ID NO: 197)
(5′-TGGGGAAACGAGGGTCAGCT-3′).

In some embodiments, the present disclosure provides an antisense oligonucleotide mentioned above for use in a method of inducing Exon-2 skipping in the CD33 gene mentioned above, wherein the antisense oligonucleotide comprises all or a portion of:

a. PMO-221
(SEQ ID NO: 221)
(5′-CCTCACCTGTCACATGCACAGAG-3′);
b. PMO-222
(SEQ ID NO: 222)
(5′-TCACCTGTCACATGCACAGAGAG-3′);
c. PMO-223
(SEQ ID NO: 223)
(5′-CTCACCTGTCACATGCACAGAGA-3′);
d. PMO-224
(SEQ ID NO: 224)
(5′-CCTCACCTGTCACATGCACAG-3′);
e. PMO-225
(SEQ ID NO: 225)
(5′-ACCTGTCACATGCACAGAGAG-3′);
f. PMO-226
(SEQ ID NO: 226)
(5′-TCACCTGTCACATGCACAGAG-3′);
g. PMO-227
(SEQ ID NO: 227)
(5′-TCACCTGTCACATGCACAGAGAGCT-3′);
h. PMO-228
(SEQ ID NO: 228)
(5′-CCTGTGCCTCACCTGTCACATGCAC-3′);
i. PMO-229
(SEQ ID NO: 229)
(5′-GTGCCTCACCTGTCACATGCACAGA-3′);
j. PMO-230
(SEQ ID NO: 230)
(5′-TGCCTCACCTGTCACATGCACAGAG-3′);
k. PMO-231
(SEQ ID NO: 231)
(5′-CTCACCTGTCACATGCACAGAGAGC-3′);
l. PMO-232
(SEQ ID NO: 232)
(5′-CACCTGTCACATGCACAGAGAGCTG-3′);
m. PMO-233
(SEQ ID NO: 233)
(5′-ACCTGTCACATGCACAGAGAGCTGG-3′);
n. PMO-234
(SEQ ID NO: 234)
(5′-CTGTCACATGCACAGAGAGCTGGGG-3′);
o. PMO-235
(SEQ ID NO: 235)
(5′-CCTGTCACATGCACAGAGAGCTG-3′);
p. PMO-236
(SEQ ID NO: 236)
(5′-TGTCACATGCACAGAGAGCTGGG-3′);
q. PMO-237
(SEQ ID NO: 237)
(5′-CTGTCACATGCACAGAGAGCTGG-3′);
r. PMO-238
(SEQ ID NO: 238)
(5′-TGTCACATGCACAGAGAGCTGG-3′);
s. PMO-239
(SEQ ID NO: 239)
(5′-TCACATGCACAGAGAGCTGGG-3′);
t. PMO-240
(SEQ ID NO: 240)
(5′-TGTCACATGCACAGAGAGCTG-3′);
u. PMO-241
(SEQ ID NO: 241)
(5′-CTGTATTTGGTACTTCCTCTCTCCA-3′);
v. PMO-242
(SEQ ID NO: 242)
(5′-TGTATTTGGTACTTCCTCTCTCCAT-3′);
w. PMO-243
(SEQ ID NO: 243)
(5′-GTATTTGGTACTTCCTCTCTCCATC-3′);
x. PMO-244
(SEQ ID NO: 244)
(5′-TATTTGGTACTTCCTCTCTCCATCC-3′);
y. PMO-324
(SEQ ID NO: 224)
(5′-CCTCACCTGTCACATGCACAG-3′);
Stereopattern:
RRRRRRRRRRRRRRRRRRRR
z. PMO-424
(SEQ ID NO: 224)
(5′-CCTCACCTGTCACATGCACAG-3′);
Stereopattern:
SSSSSSSSSSSSSSSSSSSS
aa. PMO-402
(SEQ ID NO: 002)
(5′-CCTCACCTGTCACATGCACAGAGAG-3′);
Stereopattern:
RRRRRRRRRRRRRRRRRRRRRRRR;
or
bb. PMO-502
(SEQ ID NO: 002)
(5′-CCTCACCTGTCACATGCACAGAGAG-3′);
Stereopattern:
SSSSSSSSSSSSSSSSSSSSSSSS.

In some embodiments, the present disclosure provides an antisense oligonucleotide mentioned above for use in a method of inducing Exon-2 skipping in the CD33 gene mentioned above, wherein the antisense oligonucleotide comprises all or a portion of:

a. MOE-245
(SEQ ID NO: 245)
(5′-CTCCATCCGAAAGAAGTATG-3′);
b. MOE-246
(SEQ ID NO: 246)
(5′-TCCATCCGAAAGAAGTATGA-3′);
c. MOE-247
(SEQ ID NO: 247)
(5′-CCATCCGAAAGAAGTATGAA-3′);
d. MOE-248
(SEQ ID NO: 248)
(5′-CATCCGAAAGAAGTATGAAC-3′);
e. MOE-249
(SEQ ID NO: 249)
(5′-TCCGAAAGAAGTATGAACCA-3′);
f. MOE-250
(SEQ ID NO: 250)
(5′-CCGAAAGAAGTATGAACCAT-3′);
g. MOE-251
(SEQ ID NO: 251)
(5′-ATCCGAAAGAAGTATGAA-3′);
h. MOE-252
(SEQ ID NO: 252)
(5′-CCGAAAGAAGTATGAACC-3′);
i. MOE-253
(SEQ ID NO: 253)
(5′-TCCGAAAGAAGTATGAAC-3′);
j. MOE-254
(SEQ ID NO: 254)
(5′-CCATCCGAAAGAAGTATG-3′);
k. MOE-255
(SEQ ID NO: 255)
(5′-TCCATCCGAAAGAAGTAT-3′);
l. MOE-256
(SEQ ID NO: 256)
(5′-GAAAGAAGTATGAACCAT-3′);
m. MOE-257
(SEQ ID NO: 012)
(5′-ATC-CGAAAGAAGTATGA-ACC-3′);
n. MOE-258
(SEQ ID NO: 012)
(5′-ATCC-GAAAGAAGTATG-AACC-3′);
o. MOE-259
(SEQ ID NO: 012)
(5′-ATCCG-AAAGAAGTAT-GAACC-3′);
p. MOE-260
(SEQ ID NO: 012)
(5′-ATCCG-AAAGAAGTA-TGAACC-3′);
q. MOE-261
(SEQ ID NO: 012)
(5′-ATCC-GAAAGA-AGTATG-AACC-3′);
r. MOE-262
(SEQ ID NO: 012)
(5′-ATCC-gAAAGAAGTATG-aACC-3′);
s. MOE-263
(SEQ ID NO: 012)
(5′-ATCC-gAAAGAAGTATG-aACC-3′);
t. MOE-264
(SEQ ID NO: 012)
(5′-ATCC-gAAAGAaGTATG-aACC-3′);
u. MOE-265
(SEQ ID NO: 252)
(5′-CCGA-aAGAAGTATGAACC-3′);
v. MOE-266
(SEQ ID NO: 252)
(5′-CCGA-aAGAAGTATG-aACC-3′);
w. MOE-267
(SEQ ID NO: 252)
(5′-CCGA-aAGAAGtATG-aACC-3′);
x. MOE-268
(SEQ ID NO: 252)
(5′-CCG-AAAGAAGTATGA-ACC-3′);
y. MOE-269
(SEQ ID NO: 252)
(5′-CCGA-AAGAAGTATG-AACC-3′);
z. MOE-270
(SEQ ID NO: 252)
(5′-CCGAA-AGAA-GTATG-AACC-3′);
aa. MOE-271
(SEQ ID NO: 252)
(5′-CCGAA-AGAAGTAT-GAACC-3′);
bb MOE-272
(SEQ ID NO: 252)
(5′-CCG-A-AAGAAGTATGAACC-3′);
cc. MOE-273
(SEQ ID NO: 252)
(5′-CCG-AA-AGAAGTATGAACC-3′);
dd. MOE-274
(SEQ ID NO: 252)
(5′-CCGAAAGAAGTATG-A-ACC-3′);
ee. MOE-275
(SEQ ID NO: 012)
(5′-mAmTfCfCfGfAfAfAfGfAfAfGfTfAfTfGfAfAmCmC-3′);
ff. MOE-276
(SEQ ID NO: 012)
(5′-fAfTfCfCfGmAmAmAmGmAmAmGmTmAfTfGfAfAfCfC-3′);
gg. MOE-277
(SEQ ID NO: 012)
(5′-ATCCGAAAGAAGTATGAACC-3′);
Stereopattern:
SSSSSSSSSSSSSSSSSSS;
hh. MOE-278
(SEQ ID NO: 012)
(5′-ATCCGAAAGAAGTATGAACC-3′);
Stereopattern:
RRRRRRRRRRRRRRRRRRR;
ii. MOE-279
(SEQ ID NO: 012)
(5′-ATCCGAAAGAAGTATGAACC-3′);
Stereopattern:
SSSRSSSRSSSRSSSRSSS;
jj. MOE-280
(SEQ ID NO: 012)
(5′-ATCCGAAAGAAGTATGAACC-3′);
Stereopattern:
SSSRSSRSSRSSRSSRSSS;
kk. MOE-281
(SEQ ID NO: 012)
(5′-ATCCGAAAGAAGTATGAACC-3′);
Stereopattern:
SSSRSRSRSRSRSRSRSSS;
ll. MOE-282
(SEQ ID NO: 012)
(5′-ATCCGAAAGAAGTATGAACC-3′);
Stereopattern:
SSSSSSRRRRRRRSSSSSS;
mm. MOE-283
(SEQ ID NO: 012)
(5′-ATCCGAAAGAAGTATGAACC-3′);
Stereopattern:
SSSRRSRRSRRSRRSRSSS;
nn. MOE-284
(SEQ ID NO: 012)
(5′-ATCCGAAAGAAGTATGAACC-3′);
Stereopattern:
SSSRRSRRRSRRRSRRSSS;
oo. MOE-285
(SEQ ID NO: 012)
(5′-ATCCGAAAGAAGTATGAACC-3′);
Stereopattern:
SSSSSRRRRRRRRRSSSSS;
pp. MOE-286
(SEQ ID NO: 012)
(5′-ATCCGAAAGAAGTATGAACC-3′);
Stereopattern:
SSSRRRRRRRRRRRRRSS;
qq. MOE-287
(SEQ ID NO: 012)
(5′-ATCCGAAAGAAGTATGAACC-3′);
Stereopattern:
SSRSSSSSSSSRSRSSSSS;
rr. MOE-288
(SEQ ID NO: 252)
(5′-CCGAAAGAAGTATGAACC-3′);
Stereopattern:
SSSSSSSSSSSSSSSSS;
ss. MOE-289
(SEQ ID NO: 252)
(5′-CCGAAAGAAGTATGAACC-3′);
Stereopattern:
RRRRRRRRRRRRRRRRR;
tt. MOE-290
(SEQ ID NO: 252
(5′-CCGAAAGAAGTATGAACC-3′);
Stereopattern:
SSSRRRRRRRRRRRSSS;
uu. MOE-291
(SEQ ID NO: 252)
(5′-CCGAAAGAAGTATGAACC-3′);
Stereopattern:
RRRRRRRRSSSSSSSSS;
vv. MOE-292
(SEQ ID NO: 252)
(5′-CCGAAAGAAGTATGAACC-3′);
Stereopattern:
SSSSSSSSSRRRRRRRR;
ww. MOE-293
(SEQ ID NO: 252)
(5′-CCGAAAGAAGTATGAACC-3′);
Stereopattern:
SSSRSSRSSSRSSRSSS;
xx. MOE-294
(SEQ ID NO: 252)
(5′-CCGAAAGAAGTATGAACC-3′);
Stereopattern:
SSSRSRSRSRSRSRSSS;
yy. MOE-295
(SEQ ID NO: 252)
(5′-CCGAAAGAAGTATGAACC-3′);
Stereopattern:
SRSSSRSSSRSSSRSSS;
zz. MOE-296
(SEQ ID NO: 252)
(5′-CCGAAAGAAGTATGAACC-3′);
Stereopattern:
SSSOSSRSSSRSSOSSS;
aaa. MOE-297
(SEQ ID NO: 252)
(5′-CCGAAAGAAGTATGAACC-3′);
Stereopattern:
SSSOSRSRSRSSSOSSS;
bbb. MOE-298
(SEQ ID NO: 252)
(5′-CCGAAAGAAGTATGAACC-3′);
Stereopattern:
SOSSSRSSSRSSSOSSS;
ccc. MOE-299
(SEQ ID NO: 12)
(5′-ATCCGAAAGAAGTATGAACC-3′);
Stereopattern:
SSSOSSSRSSSRSSSOSSS;
ddd. MOE-300
(SEQ ID NO: 252)
(5′-CCGAAAGAAGTATGAACC-3′);
Stereopattern:
RRRORRROSSSSSSSSS;
eee. MOE-301
(SEQ ID NO: 252)
(5′-CCGAAAGAAGTATGAACC-3′);
Stereopattern:
SRRORRROSSSSSSSSS;
fff. MOE-303
(SEQ ID NO: 252)
(5′-CCGAAAGAAGTATGAACC-3′);
Stereopattern:
SSOOOSSSSSSSSSSSS;
ggg. MOE-304
(SEQ ID NO: 252)
(5′-CCGAAAGAAGTATGAACC-3′);
Stereopattern:
OOOOOSSSSSSSSSSSS;
hhh. MOE-305
(SEQ ID NO: 252)
(5′-CCGAAAGAAGTATGAACC-3′);
Stereopattern:
SSOSSSOSSOSSSOSSS;
iii. MOE-306
(SEQ ID NO: 252)
(5′-CCGAAAGAAGTATGAACC-3′);
Stereopattern:
SOSSSSOSSSSSSOSSS;
jjj. MOE-307
(SEQ ID NO: 252)
(5′-CCGAAAGAAGTATGAACC-3′);
Stereopattern:
SSOSSSSSSSSSSOSSS;
kkk. MOE-308
(SEQ ID NO: 12)
(5′-ATCCGAAAGAAGTATGAACC-3′);
Stereopattern:
ssSOSSSSSSSSSSSOSSS;
lll. MOE-309
(SEQ ID NO: 12)
(5′-ATCCGAAAGAAGTATGAACC-3′);
Stereopattern:
SSSOSSSSOSSOSSSOSSS;
mmm. MOE-310
(SEQ ID NO: 252)
(5′-CCGAAAGAAGTATGAACC-3′);
Stereopattern:
SSORRRRRSSSSSOSSS;
or
nnn. MOE-311
(SEQ ID NO: 252)
(5′-CCGAAAGAAGTATGAACC-3′).
Stereopattern:
RRRRROSSSSSSSOSSS.

In some embodiments, the cell is an animal cell. In some embodiments, the animal cell is a human cell.

In some embodiments, the method of inducing Exon-2 skipping is performed in vitro. In some embodiments, the method of inducing Exon-2 skipping is performed in vivo.

In some embodiments, the present disclosure provides an antisense oligonucleotide mentioned above for use in a method of treating a subject having a neurodegenerative disease comprising administering a therapeutically effective amount of an antisense oligonucleotide of 16-30 nucleotides in length, wherein the antisense oligonucleotide is complementary to a portion of SEQ ID NO:1, and wherein the antisense oligonucleotide has a CD33 Exon-2 skipping efficiency of 30% or greater according to a Standard Exon-Skipping Efficiency Assay for the antisense oligonucleotide.

In some embodiments, the present disclosure provides an antisense oligonucleotide mentioned above for use in a method of treating a subject having a neurodegenerative disease mentioned above, wherein the antisense oligonucleotide comprises all or a portion of:

a. PMO-002
(SEQ ID NO: 2)
(5′-CCTCACCTGTCACATGCACAGAGAG-3′);
b. PMO-003
(SEQ ID NO: 3)
(5′-CCTGTCACATGCACAGAGAGCTGGG-3′);
c. PMO-036
(SEQ ID NO: 36)
(5′-TTGTAACTGTATTTGGTACTTCCTC-3′);
d. PMO-037
(SEQ ID NO: 37)
(5′-ACTGTATTTGGTACTTCCTCTCTCC-3′);
e. PMO-004
(SEQ ID NO: 4)
(5′-ATTTGGTACTTCCTCTCTCCATCCG-3′);
f. PMO-038
(SEQ ID NO: 38)
(5′-GTACTTCCTCTCTCCATCCGAAAGA-3′);
g. PMO-039
(SEQ ID NO: 39)
(5′-TCCTCTCTCCATCCGAAAGAAGTAT-3′);
h. PMO-005
(SEQ ID NO: 5)
(5′-TCTCCATCCGAAAGAAGTATGAACC-3′);
i. PMO-082
(SEQ ID NO: 82)
(5′-TAGTAGGGTATGGGATGGAAGAAAG-3′);
j. PMO-083
(SEQ ID NO: 83)
(5′-GGGTATGGGATGGAAGAAAGTGCAG-3′);
k. PMO-006
(SEQ ID NO: 6)
(5′-TGGGATGGAAGAAAGTGCAGGGCAC-3′);
l. PMO-096
(SEQ ID NO: 96)
(5′-ACTTGCAGCCAGAAATTTGGATCCA-3′);
m. PMO-007
(SEQ ID NO: 7)
(5′-CAGCCAGAAATTTGGATCCATAGCC-3′);
n. PMO-097
(SEQ ID NO: 97)
(5′-AGAAATTTGGATCCATAGCCAGGGC-3′);
o. PMO-008
(SEQ ID NO: 8)
(5′-CCCTGTGGGGAAACGAGGGTCAGCT-3′);
p. MOE-009
(SEQ ID NO: 9)
(5′-CACATGCACAGAGAGCTGGG-3′);
q. MOE-128
(SEQ ID NO: 128)
(5′-GCACAGAGAGCTGGGGAGAT-3′);
r. MOE-010
(SEQ ID NO: 10)
(5′-GAGAGCTGGGGAGATTTGTA-3′);
s. MOE-132
(SEQ ID NO: 132)
(5′-ACTGTATTTGGTACTTCCTC-3′);
t. MOE-135
(SEQ ID NO: 135)
(5′-TCCTCTCTCCATCCGAAAGA-3′);
u. MOE-011
(SEQ ID NO: 11)
(5′-TCTCCATCCGAAAGAAGTAT-3′);
v. MOE-012
(SEQ ID NO: 12)
(5′-ATCCGAAAGAAGTATGAACC-3′);
w. MOE-136
(SEQ ID NO: 136)
(5′-AAAGAAGTATGAACCATTAT-3′);
x. MOE-013
(SEQ ID NO: 13)
(5′-ATGCTCAGGGAGCAGTTGTT-3′);
y. MOE-014
(SEQ ID NO: 14)
(5′-GAGTCTCCTCCTGTACTTCT-3′);
z. MOE-015
(SEQ ID NO: 15)
(5′-CGCACAAACCCTCCTGTACC-3′);
aa. MOE-183
(SEQ ID NO: 183)
(5′-AAACCCTCCTGTACCGTCAC-3′);
bb. MOE-184
(SEQ ID NO: 184)
(5′-CTCCTGTACCGTCACTGACT-3′);
cc. MOE-190
(SEQ ID NO: 190)
(5′-CAGCCAGAAATTTGGATCCA-3′);
dd. MOE-196
(SEQ ID NO: 196)
(5′-CCCTGTGGGGAAACGAGGGT-3′);
or
ee. MOE-197
(SEQ ID NO: 197)
(5′-TGGGGAAACGAGGGTCAGCT-3′).

In some embodiments, the present disclosure provides an antisense oligonucleotide mentioned above for use in a method of treating a subject having a neurodegenerative disease mentioned above, wherein the antisense oligonucleotide comprises all or a portion of:

a. PMO-221
(SEQ ID NO: 221)
(5′-CCTCACCTGTCACATGCACAGAG-3′);
b. PMO-222
(SEQ ID NO: 222)
(5′-TCACCTGTCACATGCACAGAGAG-3′);
c. PMO-223
(SEQ ID NO: 223)
(5′-CTCACCTGTCACATGCACAGAGA-3′);
d. PMO-224
(SEQ ID NO: 224)
(5′-CCTCACCTGTCACATGCACAG-3′);
e. PMO-225
(SEQ ID NO: 225)
(5′-ACCTGTCACATGCACAGAGAG-3′);
f. PMO-226
(SEQ ID NO: 226)
(5′-TCACCTGTCACATGCACAGAG-3′);
g. PMO-227
(SEQ ID NO: 227)
(5′-TCACCTGTCACATGCACAGAGAGCT-3′);
h. PMO-228
(SEQ ID NO: 228)
(5′-CCTGTGCCTCACCTGTCACATGCAC-3′);
i. PMO-229
(SEQ ID NO: 229)
(5′-GTGCCTCACCTGTCACATGCACAGA-3′);
j. PMO-230
(SEQ ID NO: 230)
(5′-TGCCTCACCTGTCACATGCACAGAG-3′);
k. PMO-231
(SEQ ID NO: 231)
(5′-CTCACCTGTCACATGCACAGAGAGC-3′);
l. PMO-232
(SEQ ID NO: 232)
(5′-CACCTGTCACATGCACAGAGAGCTG-3′);
m. PMO-233
(SEQ ID NO: 233)
(5′-ACCTGTCACATGCACAGAGAGCTGG-3′);
n. PMO-234
(SEQ ID NO: 234)
(5′-CTGTCACATGCACAGAGAGCTGGGG-3′);
o. PMO-235
(SEQ ID NO: 235)
(5′-CCTGTCACATGCACAGAGAGCTG-3′);
p. PMO-236
(SEQ ID NO: 236)
(5′-TGTCACATGCACAGAGAGCTGGG-3′);
q. PMO-237
(SEQ ID NO: 237)
(5′-CTGTCACATGCACAGAGAGCTGG-3′);
r. PMO-238
(SEQ ID NO: 238)
(5′-TGTCACATGCACAGAGAGCTGG-3′);
s. PMO-239
(SEQ ID NO: 239)
(5′-TCACATGCACAGAGAGCTGGG-3′);
t. PMO-240
(SEQ ID NO: 240)
(5′-TGTCACATGCACAGAGAGCTG-3′);
u. PMO-241
(SEQ ID NO: 241)
(5′-CTGTATTTGGTACTTCCTCTCTCCA-3′);
v. PMO-242
(SEQ ID NO: 242)
(5′-TGTATTTGGTACTTCCTCTCTCCAT-3′);
w. PMO-243
(SEQ ID NO: 243)
(5′-GTATTTGGTACTTCCTCTCTCCATC-3′);
x. PMO-244
(SEQ ID NO: 244)
(5′-TATTTGGTACTTCCTCTCTCCATCC-3′);
y. PMO-324
(SEQ ID NO: 224)
(5′-CCTCACCTGTCACATGCACAG-3′);
Stereopattern:
RRRRRRRRRRRRRRRRRRRR
z. PMO-424
(SEQ ID NO: 224)
(5′-CCTCACCTGTCACATGCACAG-3′);
Stereopattern:
SSSSSSSSSSSSSSSSSSSS
aa. PMO-402
(SEQ ID NO: 002)
(5′-CCTCACCTGTCACATGCACAGAGAG-3′);
Stereopattern:
RRRRRRRRRRRRRRRRRRRRRRRR;
or
bb. PMO-502
(SEQ ID NO: 002)
(5′-CCTCACCTGTCACATGCACAGAGAG-3′);
Stereopattern:
SSSSSSSSSSSSSSSSSSSSSSSS.

In some embodiments, the present disclosure provides an antisense oligonucleotide mentioned above for use in a method of treating a subject having a neurodegenerative disease mentioned above, wherein the antisense oligonucleotide comprises all or a portion of:

a. MOE-245
(SEQ ID NO: 245)
(5′-CTCCATCCGAAAGAAGTATG-3′);
b. MOE-246
(SEQ ID NO: 246)
(5′-TCCATCCGAAAGAAGTATGA-3′);
c. MOE-247
(SEQ ID NO: 247)
(5′-CCATCCGAAAGAAGTATGAA-3′);
d. MOE-248
(SEQ ID NO: 248)
(5′-CATCCGAAAGAAGTATGAAC-3′);
e. MOE-249
(SEQ ID NO: 249)
(5′-TCCGAAAGAAGTATGAACCA-3′);
f. MOE-250
(SEQ ID NO: 250)
(5′-CCGAAAGAAGTATGAACCAT-3′);
g. MOE-251
(SEQ ID NO: 251)
(5′-ATCCGAAAGAAGTATGAA-3′);
h. MOE-252
(SEQ ID NO: 252)
(5′-CCGAAAGAAGTATGAACC-3′);
i. MOE-253
(SEQ ID NO: 253)
(5′-TCCGAAAGAAGTATGAAC-3′);
j. MOE-254
(SEQ ID NO: 254)
(5′-CCATCCGAAAGAAGTATG-3′);
k. MOE-255
(SEQ ID NO: 255)
(5′-TCCATCCGAAAGAAGTAT-3′);
l. MOE-256
(SEQ ID NO: 256)
(5′-GAAAGAAGTATGAACCAT-3′);
m. MOE-257
(SEQ ID NO: 012)
(5′-ATC-CGAAAGAAGTATGA-ACC-3′);
n. MOE-258
(SEQ ID NO: 012)
(5′-ATCC-GAAAGAAGTATG-AACC-3′);
o. MOE-259
(SEQ ID NO: 012)
(5′-ATCCG-AAAGAAGTAT-GAACC-3′);
p. MOE-260
(SEQ ID NO: 012)
(5′-ATCCG-AAAGAAGTA-TGAACC-3′);
q. MOE-261
(SEQ ID NO: 012)
(5′-ATCC-GAAAGA-AGTATG-AACC-3′);
r. MOE-262
(SEQ ID NO: 012)
(5′-ATCC-gAAAGAAGTATG-aACC-3′);
s. MOE-263
(SEQ ID NO: 012)
(5′-ATCC-gAAAGAAGTATG-aACC-3′);
t. MOE-264
(SEQ ID NO: 012)
(5′-ATCC-gAAAGAaGTATG-aACC-3′);
u. MOE-265
(SEQ ID NO: 252)
(5′-CCGA-aAGAAGTATGAACC-3′);
v. MOE-266
(SEQ ID NO: 252)
(5′-CCGA-aAGAAGTATG-aACC-3′);
w. MOE-267
(SEQ ID NO: 252)
(5′-CCGA-aAGAAGtATG-aACC-3′);
x. MOE-268
(SEQ ID NO: 252)
(5′-CCG-AAAGAAGTATGA-ACC-3′);
y. MOE-269
(SEQ ID NO: 252)
(5′-CCGA-AAGAAGTATG-AACC-3′);
z. MOE-270
(SEQ ID NO: 252)
(5′-CCGAA-AGAA-GTATG-AACC-3′);
aa. MOE-271
(SEQ ID NO: 252)
(5′-CCGAA-AGAAGTAT-GAACC-3′);
bb MOE-272
(SEQ ID NO: 252)
(5′-CCG-A-AAGAAGTATGAACC-3′);
cc. MOE-273
(SEQ ID NO: 252)
(5′-CCG-AA-AGAAGTATGAACC-3′);
dd. MOE-274
(SEQ ID NO: 252)
(5′-CCGAAAGAAGTATG-A-ACC-3′);
ee. MOE-275
(SEQ ID NO: 012)
(5′-mAmTfCfCfGfAfAfAfGfAfAfGfTfAfTfGfAfAmCmC-3′);
ff. MOE-276
(SEQ ID NO: 012)
(5′-fAfTfCfCfGmAmAmAmGmAmAmGmTmAfTfGfAfAfCfC-3′);
gg. MOE-277
(SEQ ID NO: 012)
(5′-ATCCGAAAGAAGTATGAACC-3′);
Stereopattern:
SSSSSSSSSSSSSSSSSSS;
hh. MOE-278
(SEQ ID NO: 012)
(5′-ATCCGAAAGAAGTATGAACC-3′);
Stereopattern:
RRRRRRRRRRRRRRRRRRR;
ii. MOE-279
(SEQ ID NO: 012)
(5′-ATCCGAAAGAAGTATGAACC-3′);
Stereopattern:
SSSRSSSRSSSRSSSRSSS;
jj. MOE-280
(SEQ ID NO: 012)
(5′-ATCCGAAAGAAGTATGAACC-3′);
Stereopattern:
SSSRSSRSSRSSRSSRSSS;
kk. MOE-281
(SEQ ID NO: 012)
(5′-ATCCGAAAGAAGTATGAACC-3′);
Stereopattern:
SSSRSRSRSRSRSRSRSSS;
ll. MOE-282
(SEQ ID NO: 012)
(5′-ATCCGAAAGAAGTATGAACC-3′);
Stereopattern:
SSSSSSRRRRRRRSSSSSS;
mm. MOE-283
(SEQ ID NO: 012)
(5′-ATCCGAAAGAAGTATGAACC-3′);
Stereopattern:
SSSRRSRRSRRSRRSRSSS;
nn. MOE-284
(SEQ ID NO: 012)
(5′-ATCCGAAAGAAGTATGAACC-3′);
Stereopattern:
SSSRRSRRRSRRRSRRSSS;
oo. MOE-285
(SEQ ID NO: 012)
(5′-ATCCGAAAGAAGTATGAACC-3′);
Stereopattern:
SSSSSRRRRRRRRRSSSSS;
pp. MOE-286
(SEQ ID NO: 012)
(5′-ATCCGAAAGAAGTATGAACC-3′);
Stereopattern:
SSSRRRRRRRRRRRRRSS;
qq. MOE-287
(SEQ ID NO: 012)
(5′-ATCCGAAAGAAGTATGAACC-3′);
Stereopattern:
SSRSSSSSSSSRSRSSSSS;
rr. MOE-288
(SEQ ID NO: 252)
(5′-CCGAAAGAAGTATGAACC-3′);
Stereopattern:
SSSSSSSSSSSSSSSSS;
ss. MOE-289
(SEQ ID NO: 252)
(5′-CCGAAAGAAGTATGAACC-3′);
Stereopattern:
RRRRRRRRRRRRRRRRR;
tt. MOE-290
(SEQ ID NO: 252
(5′-CCGAAAGAAGTATGAACC-3′);
Stereopattern:
SSSRRRRRRRRRRRSSS;
uu. MOE-291
(SEQ ID NO: 252)
(5′-CCGAAAGAAGTATGAACC-3′);
Stereopattern:
RRRRRRRRSSSSSSSSS;
vv. MOE-292
(SEQ ID NO: 252)
(5′-CCGAAAGAAGTATGAACC-3′);
Stereopattern:
SSSSSSSSSRRRRRRRR;
ww. MOE-293
(SEQ ID NO: 252)
(5′-CCGAAAGAAGTATGAACC-3′);
Stereopattern:
SSSRSSRSSSRSSRSSS;
xx. MOE-294
(SEQ ID NO: 252)
(5′-CCGAAAGAAGTATGAACC-3′);
Stereopattern:
SSSRSRSRSRSRSRSSS;
yy. MOE-295
(SEQ ID NO: 252)
(5′-CCGAAAGAAGTATGAACC-3′);
Stereopattern:
SRSSSRSSSRSSSRSSS;
zz. MOE-296
(SEQ ID NO: 252)
(5′-CCGAAAGAAGTATGAACC-3′);
Stereopattern:
SSSOSSRSSSRSSOSSS;
aaa. MOE-297
(SEQ ID NO: 252)
(5′-CCGAAAGAAGTATGAACC-3′);
Stereopattern:
SSSOSRSRSRSSSOSSS;
bbb. MOE-298
(SEQ ID NO: 252)
(5′-CCGAAAGAAGTATGAACC-3′);
Stereopattern:
SOSSSRSSSRSSSOSSS;
ccc. MOE-299
(SEQ ID NO: 12)
(5′-ATCCGAAAGAAGTATGAACC-3′);
Stereopattern:
SSSOSSSRSSSRSSSOSSS;
ddd. MOE-300
(SEQ ID NO: 252)
(5′-CCGAAAGAAGTATGAACC-3′);
Stereopattern:
RRRORRROSSSSSSSSS;
eee. MOE-301
(SEQ ID NO: 252)
(5′-CCGAAAGAAGTATGAACC-3′);
Stereopattern:
SRRORRROSSSSSSSSS;
fff. MOE-303
(SEQ ID NO: 252)
(5′-CCGAAAGAAGTATGAACC-3′);
Stereopattern:
SSOOOSSSSSSSSSSSS;
ggg. MOE-304
(SEQ ID NO: 252)
(5′-CCGAAAGAAGTATGAACC-3′);
Stereopattern:
OOOOOSSSSSSSSSSSS;
hhh. MOE-305
(SEQ ID NO: 252)
(5′-CCGAAAGAAGTATGAACC-3′);
Stereopattern:
SSOSSSOSSOSSSOSSS;
iii. MOE-306
(SEQ ID NO: 252)
(5′-CCGAAAGAAGTATGAACC-3′);
Stereopattern:
SOSSSSOSSSSSSOSSS;
jjj. MOE-307
(SEQ ID NO: 252)
(5′-CCGAAAGAAGTATGAACC-3′);
Stereopattern:
SSOSSSSSSSSSSOSSS;
kkk. MOE-308
(SEQ ID NO: 12)
(5′-ATCCGAAAGAAGTATGAACC-3′);
Stereopattern:
ssSOSSSSSSSSSSSOSSS;
lll. MOE-309
(SEQ ID NO: 12)
(5′-ATCCGAAAGAAGTATGAACC-3′);
Stereopattern:
SSSOSSSSOSSOSSSOSSS;
mmm. MOE-310
(SEQ ID NO: 252)
(5′-CCGAAAGAAGTATGAACC-3′);
Stereopattern:
SSORRRRRSSSSSOSSS;
or
nnn. MOE-311
(SEQ ID NO: 252)
(5′-CCGAAAGAAGTATGAACC-3′).
Stereopattern:
RRRRROSSSSSSSOSSS.

In some embodiments, the present disclosure provides a method of treating a subject having a neurodegenerative disease mentioned above, wherein the neurodegenerative disease is Alzheimer's Disease.

### BRIEF DESCRIPTION OF THE FIGURES

This application file contains figures in color. Copies of this patent application publication with color drawing(s) will be provided by the Office upon request and payment of the necessary fee.

FIG. 1 shows the levels of CD33 mRNA in plasma and cerebral spinal fluid in patients relative to the rs3865444 SNP. C=rs3865444-C, A=rs3865444-A.

FIG. 2 shows various cognitive results in patients with the rs3865444-A allele vs. patients with the rs201074739 indel frameshift allele.

FIG. 3 shows various physiological results in patients with the rs3865444-A allele vs. patients with the rs201074739 indel allele.

FIG. 4 shows the levels of CD33 mRNA in plasma and cerebral spinal fluid in patients relative to the rs201074739 indel.

FIG. 5 shows the exon skipping efficiencies of several PMO sequences at different concentrations.

FIG. 6 shows the exon skipping efficiencies of several MOE sequences at different concentrations.

FIG. 7 shows the fold change (in vivo ability to increase Exon-2-skipped CD33 mRNA) of two ASOs relative to control (PBS) in mouse hippocampus at two dose levels. D2-CD33=Exon-2-skipped CD33 mRNA, PMO-002=SEQ ID NO:2, MOE-012=SEQ ID NO:12.

FIG. 8 shows the fold change (in vivo ability to increase Exon-2-skipped CD33 mRNA) of two ASOs relative to control (PBS) in mouse cortex at two dose levels. D2-CD33=Exon-2-skipped CD33 mRNA, PMO-002=SEQ ID NO:2, MOE-012=SEQ ID NO:12.

FIG. 9 shows the percent Exon-2 skipping in CD33 mRNA in mouse cortex and hippocampus for PMO-221, PMO-224, PMO-232, PMO-233, PMO-237, PMO-238, PMO-002, and PMO-003. D2-CD33=Exon-2-skipped CD33 mRNA.

FIG. 10 shows the fold change (in vivo ability to increase Exon-2-skipped CD33 mRNA) of (i) PMO-224 relative to control (PBS) in mouse cortex and hippocampus at three dose levels and (ii) PMO-002 relative to control (PBS) in mouse cortex and hippocampus at one dose level. D2-CD33=Exon-2-skipped CD33 mRNA.

FIG. 11 shows HPLC chromatogram and HRMS trace of PMO-424.

FIG. 12 shows HPLC chromatogram and HRMS trace of PMO-324.

FIG. 13 shows Tm of PMO-324, PMO-424, and PMO-224.

FIG. 14 shows HPLC chromatogram and HRMS trace of PMO-502.

FIG. 15 shows HPLC chromatogram and HRMS trace of PMO-402.

FIG. 16 shows Tm of PMO-402, PMO-502, and PMO-002.

FIG. 17 shows chromatogram of PMO-424 with N3′-trityl group (resin cleaved).

FIG. 18 shows the fold change (in vivo ability to increase Exon-2-skipped CD33 mRNA) of PMO-324 and PMO-424 relative to control (PBS) in mouse cortex and hippocampus at two dose levels. D2-CD33=Exon-2-skipped CD33 mRNA.

FIG. 19 shows the fold change (in vivo ability to increase Exon-2-skipped CD33 mRNA) of PMO-402 and PMO-502 relative to control (PBS) in mouse cortex and hippocampus at two dose levels. D2-CD33=Exon-2-skipped CD33 mRNA.

FIG. 20 shows the melting temperature of MOE-012, MOE-277, and MOE-278.

FIG. 21 shows the HPLC elution profile of stereopure ASOs MOE-288 to MOE-292 and stereorandom ASO MOE-252.

FIG. 22 shows the in vivo activity of ASOs MOE-012 and MOE-246 to MOE-256 with 100 μg dosing.

FIG. 23 shows the in vivo activity of ASOs MOE-012 and MOE-257 to MOE-261 with 100 μg dosing.

FIG. 24 shows the in vivo activity of ASOs MOE-262 to MOE-267 and MOE-252 with 30 μg dosing.

FIG. 25 shows the in vivo activity of ASOs MOE-277 and MOE-279 to MOE-284 with 30 μg dosing.

FIG. 26 shows the in vivo activity of ASOs MOE-252, MOE-288, MOE-291, and MOE-292 with 30 μg and 100 μg dosing, and MOE-289 and MOE-290 with 30 μg dosing.

FIG. 27 shows the in vivo activity of ASOs MOE-293 to MOE-299 with 30 μg and 100 μg dosing.

FIG. 28 shows the in vivo activity of ASOs MOE-300, MOE-301 and MOE-303 to MOE-311 with 100 μg dosing

FIG. 29 shows the in vivo activity of MOE-279 with 10 μg, 30 μg, 60 μg, and 100 μg dosing.

FIG. 30 shows the duration of the skipping effect with a single 100 μg ICV dose of MOE-277 (up to 150 days).

FIG. 31 shows the brain concentration of MOE-277 after a single 100 μg ICV dose (up to 150 days).

### DEFINITIONS

The term “oligonucleotide” is used herein to refer to a nucleotide sequence comprising at least ten DNA or RNA nucleotides.

The term “antisense oligonucleotide,” abbreviated as “ASO,” is used herein to refer to a nucleotide sequence comprising an antisense sequence that is sufficiently complementary to a target nucleotide sequence in order to form a stable double stranded hybrid with the target nucleotide sequence. In some embodiments, the target nucleotide sequence is an RNA nucleotide sequence. Unless otherwise specified, ASOs represented herein are displayed in the 5′ to 3′ orientation.

The term “nucleobase” is used herein to refer to a base that is a component of a nucleoside. Example nucleobases include adenine, guanine, thymine, cytosine, and uracil.

The term “nucleoside” is used herein to refer to a nucleobase covalently linked to a sugar. Examples of naturally occurring and non-natural nucleosides are described below.

The term “nucleotide” is used herein to refer to a nucleoside covalently linked to a phosphate group. Examples of naturally occurring nucleotides include adenosine, thymidine, uridine, cytidine, 5-methylcytidine, and guanosine. Description and examples of non-natural nucleotides are described below.

Within the ASO structure, the phosphate groups are commonly referred to as forming the “internucleotide linkages” of the ASO. The naturally occurring internucleotide linkage of RNA and DNA is a 3′ to 5′ phosphodiester linkage. A “phosphoramidate” group comprises phosphorus having three attached oxygen atoms and one attached nitrogen atom, while a “phosphorodiamidate” group comprises phosphorus having two attached oxygen atoms and two attached nitrogen atoms. A “phosphorotriamidate” group (or a phosphoric acid triamide group) comprises phosphorus having one attached oxygen atom and three attached nitrogen atoms. In the uncharged or the cationic internucleotide linkages of the morpholino-based ASOs described herein, one nitrogen is always pendant to the linkage chain. The second nitrogen, in a phosphorodiamidate linkage, is typically the ring nitrogen in a morpholino ring structure.

The term “non-natural” is used herein to refer to molecules that contain man-made modifications relative to their naturally occurring counterparts. In some embodiments, “non-natural” may refer to one or more nucleotide subunits having at least one modification selected from (i) a modified internucleotide linkage, e.g., an internucleotide linkage other than the standard phosphodiester linkage found in naturally-occurring oligonucleotides, (ii) modified sugar moieties, e.g., moieties other than ribose or deoxyribose moieties found in naturally occurring oligonucleotides, (iii) modified nucleobases, e.g., bases other than those found in naturally occurring oligonucleotides, or (iv) a any combination of the foregoing. In some embodiments, the ASO is chosen from ASOs that do not have a phosphorus atom in the internucleotide linkage (backbone). In some embodiments, the ASO has a phosphorodiamidate or phosphorothioate modified internucleotide linkage (backbone).

The term “morpholino” is used herein to refer to a nucleotide that contains a morpholinyl ring instead of a ribose.

The term “morpholino-based ASO” is used herein to refer to an ASO with at least one nucleotide containing a morpholinyl ring instead of a ribose.

The term “stereo-controlled” is used herein to describe when a nucleotide and/or an oligonucleotide is designed or selected to have a particular stereochemistry. In some embodiments, the nucleobase portion of a nucleotide or oligonucleotide, including any and all non-natural modifications, is stereo-controlled. In some embodiments, the nucleoside portion of a nucleotide or oligonucleotide, including any and all non-natural modifications, is stereo-controlled. In some embodiments, the internucleotide linkage portion of a nucleotide or oligonucleotide, including any and all non-natural modifications, is stereo-controlled. In some embodiments, a nucleotide may comprise one or a combination of these stereo-controlled portions. In some embodiments, an oligonucleotide may comprise a combination of nucleotides that comprise a combination of stereo-controlled nucleotides. In some embodiments, an oligonucleotide may comprise a combination of nucleotides that are stereo-controlled and not stereo-controlled. In some embodiments, the proportion of stereo-controlled nucleotides ranges from 10%-100%, such as 15%-100%, 20%-100%, 30%-100%, 40%-100%, 50%-100%, 50%-90%, 50%-95%, 60%-100%, 60%-90%, 60%-95%, 70%-100%, 70%-90%, 70%-95%, 80-100%, 80%-90%, 80%-95%, 90-100%, 90%-95%, 90%-96%, 90%-97%, 90%-98%, 90%-99%, 95%-98%, 95%-99%, 95-100%, 50%-90%, or 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%, of nucleotides.

When applied to nucleotides, the term “stereopure” is used herein to describe when at least 90% of nucleotides in an oligonucleotide are stereo-controlled. In some embodiments, the proportion of stereo-controlled nucleotides in a stereopure ASO ranges from 90-100%, 95-100%, 90%-95%, 90%-96%, 90%-97%, 90%-98%, 90%-99%, 95%-98%, 95%-99%, or 90%, 91%, 92%, 93%, 94%, 95%, 96%, 97%, 98%, 99%, or 100%, or at least 90%, 91%, 92%, 93%, 94%, 95%, 96%, 97%, 98%, or 99%, of nucleotides. In some embodiments, all or a portion of nucleotides within an oligonucleotide are stereo-controlled so that they are stereopure in the same way, i.e., all or a portion of the nucleotides are stereo-controlled, and they are designed or selected to have the same stereochemistry. In some embodiments, all or a portion of nucleotides within an oligonucleotide are stereo-controlled so that they are not stereopure in the same way, i.e., all or a portion of the nucleotides are stereo-controlled, but they are designed or selected to have different stereochemistry. When applied to the internucleotide linkage portion of an oligonucleotide, the term “stereopure” is used to describe when at least 90% of the internucleotide linkages are stereo-controlled. In some embodiments, the proportion of stereo-controlled internucleotide linkages in a stereopure ASO ranges from 90-100%, 95-100%, 90%-95%, 90%-96%, 90%-97%, 90%-98%, 90%-99%, 95%-98%, 95%-99%, or 90%, 91%, 92%, 93%, 94%, 95%, 96%, 97%, 98%, 99%, or 100%, or at least 90%, 91%, 92%, 93%, 94%, 95%, 96%, 97%, 98%, or 99%, of internucleotide linkages. In some embodiments, all or a portion of internucleotide linkages within an oligonucleotide are stereo-controlled so that they are stereopure in the same way, i.e., all or a portion of the internucleotide linkages are stereo-controlled, and they are designed or selected to have the same stereochemistry. In some embodiments, all or a portion of internucleotide linkages within an oligonucleotide are stereo-controlled so that they are not stereopure in the same way, i.e., all or a portion of the internucleotide linkages are stereo-controlled, but they are designed or selected to have different stereochemistry. In some embodiments, the internucleotide linkages are phosphorodiamidate linkages. In some embodiments, the internucleotide linkages are phosphorothioate linkages.

Stereochemistry for (Rp, Sp) and phosphate (PO) internucleotide linkages is illustrated as the following

Sp [symbol] Rp [symbol] PO [symbol]
Stereochemistry for Rp, Sp, and PO internucleotide linkages is also illustrated as follows: S=Sp, R=Rp, O=phosphate.

For example, the stereochemistry of the internucleotide linkages of MOE-298 can be shown using either of the following illustrations:

(SEQ ID NO: 252)
5′- [symbol] [symbol] [symbol] -3′,
or
(5′-CCGAAAGAAGTATGAACC-3′);
Stereopattern:
SOSSSRSSSRSSSOSSS.

When applied to nucleotides, the term “stereorandom” is used herein to describe when the nucleotides in an oligonucleotide are not stereo-controlled. When applied to internucleotide linkages, the term “stereorandom” is used herein to describe when the internucleotide linkages in an oligonucleotide are not stereo-controlled. In some embodiments, the internucleotide linkages are phosphorodiamidate linkages. In some embodiments, the internucleotide linkages are phosphorothioate linkages.

The term “complementary” is used herein to describe when the corresponding positions of at least two nucleotide sequences are occupied by nucleotides which can hydrogen bond with each other.

The term “hybridize” is used herein to describe the binding of two complementary nucleotide sequences, forming one double stranded molecule. When a sufficient number of corresponding nucleotides in two sequences can hydrogen bond with each other, i.e., they are sufficiently complementary, they may form a stable hybrid. It is understood in the art that 100% complementarity is not necessary for an ASO to hybridize with a target sequence.

The term “sufficient complementarity” is used herein to indicate a level of complementarity sufficient to permit an ASO to bind to its target sequence and form a stable hybrid. In some embodiments, the complementarity of the ASO and the target sequence is at least 99%, or 98%, or 97%, or 96%, or 95%, or 94%, or 93%, or 92%, or 91%, or 90%, or 89%, or 88%, or 87%, or 86%, or 85%, or 84%, or 83%, or 82%, or 81%, or 80%, or 79%, or 78%, or 77%, or 76%, or 75%, or 74%, or 73%, or 72%, or 71%, or 70%.

The term “sequence similarity” is used herein to express the similarity of two ASOs. Sequence similarity is expressed as a percentage of nucleotides shared between two ASOs. It is understood that identical sequences have 100% sequence similarity.

The terms “target region” and “target sequence” are used interchangeably herein to designate a nucleotide sequence to which an ASO will hybridize under physiological conditions. It is not necessary for the ASO and the target region to be 100% complementary, so long as there is sufficient complementarity for the ASO to hybridize to the target sequence and form a stable hybrid. The ASO may hybridize to all or a portion of the target sequence.

The terms “treat,” “treating,” or “treatment” are used herein to refer to ameliorating a disease or disorder (i.e., slowing or arresting or reducing the development of the disease or at least one of the clinical symptoms thereof). The terms also refer to alleviating or ameliorating at least one physical parameter including those which may not be discernible by the patient. The terms also refer to modulating the disease or disorder, either physically (e.g., through stabilization of a discernible symptom), physiologically, (e.g., through stabilization of a physical parameter), or both.

The terms “prevent,” “preventing,” or “prevention” are used herein to refer to inhibiting or delaying the onset of a disease or disorder.

The term “therapeutically effective amount” is used herein to refer to the amount of a therapeutic agent or composition effective in prevention or treatment of a disorder or disease. In some embodiments, this includes an amount of a therapeutic agent or composition effective in the prevention or treatment of a neurodegenerative disease.

The term “pharmaceutically acceptable” is used herein to refer to a molecular entity or composition that is pharmaceutically useful and not biologically or otherwise undesirable.

The term “carrier” is used herein to refer to a diluent, adjuvant, excipient, or vehicle with which the compound is administered.

The term “excipient” as used herein refers to any ingredient in a pharmaceutical composition other than the active ingredient.

As used herein, “skipping efficiency” of an oligonucleotide is calculated using the following formula:

$$Skipping \% = \frac{( Skipped Value )}{( Skipped ) * ( Un - skipped )} \times 100$$

and is represented on a scale of 0 to 100, wherein 100 represents 100% skipping of CD33 Exon-2. “Skipping efficiency” of an oligonucleotide as used herein is experimentally determined using one of three Standard Exon-Skipping Efficiency Assays depending on the type of antisense oligonucleotide. For antisense oligonucleotides comprising phosphorodiamidate morpholino oligomers, the Standard Exon-Skipping Efficiency Assay for PMO ASOs defined below is used; for antisense oligonucleotides comprising methoxyethyl ribose oligomers, the Standard Exon-Skipping Efficiency Assay for MOE ASOs defined below is used; and for antisense oligonucleotides that do not comprise phosphorodiamidate morpholino or methoxyethyl ribose oligomers, the Standard Exon-Skipping Efficiency Assay for non-PMOs and non-MOEs described below is used.

The Standard Exon-Skipping Efficiency Assay for PMO ASOs includes using U-188 MG cells that were cultured and maintained using appropriate media suggested in the vendor protocols (Dulbecco's Modified Eagle's Medium containing 10% fetal bovine serum). The Assay is performed in 96 well plate format, seeding about 20,000 cells per well and treating with the PMO ASO at a concentration of 0.5 μM using the Endo-Porter protocol. Cells are incubated at 37° C. in a cell culture incubator for 48 hours before isolating the total RNA. Total RNA is isolated and converted to cDNA per vendor protocol, then Taqman gene expression assays are used to quantify Exon-2 skipped CD33 (Forward primer: CGCTGCTGCTACTGCTG (SEQ ID NO:207); Reverse Primer: TTCTAGAGTGCCAGGGATGA (SEQ ID NO:208); and probe: TGTGGGCAGACTTGACCCACAG (SEQ ID NO:209)) and un-skipped CD33 (Forward primer: GGATGGAGAGAGGAAGTA (SEQ ID NO:210); Reverse Primer: GTGCCAGGGATGAGGATTT (SEQ ID NO:211); and probe: TGCATGTGACAGACTTGACCCACA (SEQ ID NO:212)) mRNA transcripts. Human house-keeping genes such as HPRT1 (Assay ID: Hs02800695_m1; ThermoFisher Scientific) or GAPDH1 (Hs99999905_m1; ThermoFisher Scientific) expressions are used to normalize the target transcript expressions.

The Standard Exon-Skipping Efficiency Assay for MOE ASOs includes using U-188 MG cells that are cultured and maintained using appropriate media suggested in the vendor protocols (Dulbecco's Modified Eagle's Medium containing 10% fetal bovine serum). The Assay is performed in 96 well plate format, seeding about 20,000 cells per well and treating with the MOE ASO at a concentration of 10 nM using the Lipofectamine protocol. Cells are incubated at 37° C. in a cell culture incubator for 48 hours before isolating the total RNA. Total RNA is isolated and converted to cDNA per vendor protocol, then Taqman gene expression assays are used to quantify Exon-2 skipped CD33 (Forward primer: CGCTGCTGCTACTGCTG (SEQ ID NO:207); Reverse Primer: TTCTAGAGTGCCAGGGATGA (SEQ ID NO:208); and probe: TGTGGGCAGACTTGACCCACAG (SEQ ID NO:209)) and un-skipped CD33 (Forward primer: GGATGGAGAGAGGAAGTA (SEQ ID NO:210); Reverse Primer: GTGCCAGGGATGAGGATTT (SEQ ID NO:211); and probe: TGCATGTGACAGACTTGACCCACA (SEQ ID NO:212)) mRNA transcripts. Human house-keeping genes such as HPRT1 (Assay ID: Hs02800695_m1; ThermoFisher Scientific) or GAPDH1(Hs99999905_m1; ThermoFisher Scientific) expressions are used to normalize the target transcript expressions.

For ASOs that are neither PMOs or MOEs, the Standard Exon-Skipping Efficiency Assay for non-PMOs and non-MOEs includes using U-188 MG cells that are cultured and maintained using appropriate media suggested in the vendor protocols (Dulbecco's Modified Eagle's Medium containing 10% fetal bovine serum). The Assay is performed in 96 well plate format, seeding about 20,000 cells per well and treating with the ASO at a concentration of 10 nM using the Lipofectamine protocol. Cells are incubated at 37° C. in a cell culture incubator for 48 hours before isolating the total RNA. Total RNA is isolated and converted to cDNA per vendor protocol, then Taqman gene expression assays are used to quantify Exon-2 skipped CD33 (Forward primer: CGCTGCTGCTACTGCTG (SEQ ID NO:207); Reverse Primer: TTCTAGAGTGCCAGGGATGA (SEQ ID NO:208); and probe: TGTGGGCAGACTTGACCCACAG (SEQ ID NO:209)) and un-skipped CD33 (Forward primer: GGATGGAGAGAGGAAGTA (SEQ ID NO:210); Reverse Primer: GTGCCAGGGATGAGGATTT (SEQ ID NO:211); and probe: TGCATGTGACAGACTTGACCCACA (SEQ ID NO:212)) mRNA transcripts. Human house-keeping genes such as HPRT1 (Assay ID: Hs02800695_m1; ThermoFisher Scientific) or GAPDH1(Hs99999905_m1; ThermoFisher Scientific) expressions are used to normalize the target transcript expressions.

As an alternative to the Lipofectamine protocol, free uptake (without transfection reagents) may be used for the Standard Exon-Skipping Efficiency Assay.

In some embodiments, the antisense oligonucleotide has a CD33 Exon-2 skipping efficiency of 25% to 99%, such as 25%, 30%, 35%, 40%, 45%, 50%, 55%, 60%, 65%, 70%, 75%, 80%, 85%, 90%, 95%, or 99%. In some embodiments, the antisense oligonucleotide has a CD33 Exon-2 skipping efficiency of 50% to 99%. In some embodiments, the antisense oligonucleotide has a CD33 Exon-2 skipping efficiency of at least 30%.

Unless otherwise defined, all other scientific and technical terms have the same meaning as commonly understood to one of ordinary skill in the art. Such scientific and technical terms are explained in the literature, for example: J. Sambrook, E. F. Fritsch, and T Maniatis, Molecular Cloning: A Laboratory Manual, Second Edition, Books 1-3, Cold Spring Harbor Laboratory Press (1989); Martin, Remington's Pharmaceutical Sciences, 18th Edition, Mack Publishing Co (1990); Glover, DNA Cloning: A Practical Approach, Volumes I and II, MRL Press, Ltd. (1985); and Ausubel, F et al., Current Protocols in Molecular Biology, Greene Publishing Associates/Wiley Intersciences (2002).

Disclosed herein are novel ASOs. In some embodiments, the ASOs are directed to a target sequence in the CD33 pre-mRNA. In some embodiments, the ASOs are directed to all or a portion of a 16- to 30-nucleotide target sequence in the CD33 pre-mRNA, represented in SEQ ID NO:1 (5′-GGGCAGGTGA GTGGCTGTGG GGAGAGGGGT TGTCGGGCTG GGCCGAGCTG ACCCTCGTTT CCCCACAGGG GCCCTGGCTA TGGATCCAAA TTTCTGGCTG CAAGTGCAGG AGTCAGTGAC GGTACAGGAG GGTTTGTGCG TCCTCGTGCC CTGCACTTTC TTCCATCCCA TACCCTACTA CGACAAGAAC TCCCCAGTTC ATGGTTACTG GTTCCGGGAA GGAGCCATTA TATCCAGGGA CTCTCCAGTG GCCACAAACA AGCTAGATCA AGAAGTACAG GAGGAGACTC AGGGCAGATT CCGCCTCCTT GGGGATCCCA GTAGGAACAA CTGCTCCCTG AGCATCGTAG ACGCCAGGAG GAGGGATAAT GGTTCATACT TCTTTCGGAT GGAGAGAGGA AGTACCAAAT ACAGTTACAA ATCTCCCCAG CTCTCTGTGC ATGTGACAGG TGAGGCACAG GCTTCAGAAG TGGCCGCAAG GGAAGTTCAT GGGTACTGCA GGGCAGGGCT GGGATGGGAC CCTGGTACTG-3′). SEQ ID NO:1 includes Exon-2 and portions of the bordering introns of the CD33 gene. This 16- to 30-nucleotide target sequence is involved in Exon-2 skipping that also occurs when CD33 mRNA includes the rs3865444-A SNP. When this Exon-2 skipping occurs, pre-mRNA containing the SNP is spliced so that Exon-2 is not included in the final transcript.

In some embodiments, the ASOs are 16-30 nucleotides long. In some embodiments, the nucleotides are 20-30 nucleotides long. In some embodiments, the ASOs are 25-30 nucleotides long. In some embodiments, the ASOs are 21-30 nucleotides long. In some embodiments, the ASOs are 21-25 nucleotides long. In some embodiments, the ASOs are 18-21 nucleotides long. In some embodiments, the ASOs are 18-25 nucleotides long. In some embodiments, the ASOs are 16, 17, 18, 19, 20, 21, 22, 23, 24, 25, 26, 27, 28, 29, or 30 nucleotides long.

In some embodiments, the antisense oligonucleotide comprises 16-30, such as 18-30, nucleotides. In some embodiments, the antisense oligonucleotide consists of 16-30, such as 18-30, nucleotides.

Also disclosed herein are novel ASOs complementary to all or a portion of a 10- to 16-nucleotide target sequence in the CD33 pre-mRNA, represented in SEQ ID NO:1, which includes Exon-2 and portions of the bordering introns of the CD33 gene. In some embodiments, the ASOs are 10-14 nucleotides long. In some embodiments, the ASOs are 10, 11, 12, 13, 14, 15, or 16 nucleotides long.

In some embodiments, the ASOs are directed to the 16- to 30-nt target sequence, are sufficiently complimentary to the target sequence to form a stable hybrid, and are 16-30 nucleotides in length. In some embodiments, these ASOs are sufficiently complimentary to all or a portion of the 25-nt target sequence.

In some embodiments, the ASOs have one of the specific sequences disclosed in Table 3 or 4. In some embodiments, the ASOs may share sequence similarity with one of the ASOs disclosed in Table 3 or 4. In some embodiments, the ASO shares at least 99%, or 98%, or 97%, or 96%, or 95%, or 94%, or 93%, or 92%, or 91%, or 90%, or 89%, or 88%, or 87%, or 86%, or 85%, or 84%, or 83%, or 82%, or 81%, or 80%, or 79%, or 78%, or 77%, or 76%, or 75%, or 74%, or 73%, or 72%, or 71%, or 70% sequence similarity with one of the ASOs disclosed in Table 3 or 4.

In some embodiments, at least some nucleobases of the ASOs will have thymine instead of uracil or will have uracil instead of thymine. In some embodiments, at least some nucleosides of the ASOs will have deoxyribose replaced with ribose, or will have ribose replaced with deoxyribose.

In some embodiments, the ASOs comprise at least one chemically modified nucleotide. In some embodiments, the at least one chemical modification of the nucleotide is chosen from chemical modification of at least one nucleobase, chemical modification of at least one sugar moiety, chemical modification of at least one phosphate, and any combination of these modifications. In some embodiments, the at least one chemical modification improves the ability of the nucleotide to resist nuclease degradation.

Non-limiting examples of chemical modifications useful in this disclosure include chemical modifications of an ASO's phosphate backbone and chemically modified (i.e., non-natural) internucleoside linkage(s). In some embodiments, the ASO is chosen from ASOs having a chemically modified phosphate backbone. In some embodiments, the ASO is chosen from ASOs that do not have a phosphorus atom in the backbone. In some embodiments, the ASO has a phosphorodiamidate or phosphorothioate modified backbone. In some embodiments, the modified backbone is stereo-controlled.

Additional non-limiting examples of chemical modifications useful in this disclosure include chemical modifications of at least one sugar moiety in an ASO. In some embodiments, the ASO comprises at least one chemically modified sugar moiety. In some embodiments, the chemically modified sugar moiety is chosen from sugar moieties substituted in at least one position on the sugar moiety in the ASO. In some embodiments, the ASO is chosen from ASOs that are substituted in at least one position on the sugar chosen from the 2′, 3′ and 5′ positions. In some embodiments, the at least one substituent on the ASOs' sugar moieties is chosen from hydroxyl; fluoro; and substituted or unsubstituted, linear or branched C₁-C₁₀ alkyl groups, substituted or unsubstituted, linear or branched C₂-C₁₀ alkenyl groups, substituted or unsubstituted, linear or branched C₂-C₁₀ alkynyl groups, substituted or unsubstituted, linear or branched C₇-C₁₇ alkaryl groups, substituted or unsubstituted, linear or branched C₃-C₁₀ allyl groups, and substituted or unsubstituted, linear or branched C₇-C₁₇ aralkyl groups, each of which groups may optionally further comprise at least one heteroatom. In some embodiments, the sugar moiety comprises at least one substituent chosen from methoxy, aminopropoxy, methoxyethoxy, dimethylaminoethoxy, and dimethylaminoethoxyethoxy. In some embodiments, the sugar moiety is chosen from pyranoses, derivatives of pyranoses, deoxypyranoses, derivatives of deoxypyranoses, riboses, derivatives of riboses, deoxyriboses, and derivatives of deoxyribose. In some embodiments, the substituted sugar moiety is chosen from methoxyethyl substitute sugar moieties, including 2′-O-methoxyethyl. In some embodiments, the sugar moiety is stereo-controlled.

In some embodiments, the sugar moiety is modified in a manner that creates a bicyclic sugar moiety. In some embodiments, the bicyclic sugar moiety is formed from a bridge modification between the 4′ and 2′ furanose ring atoms. In some embodiments, the bridge modification comprises at least one group that forms a bridge between the 4′ and 2′ furanose ring atoms. In some embodiments, at least one nucleotide in a given ASO has a bridge modification.

In some embodiments, the sugar moiety comprises fewer than 5 ring atoms, such as 4 ring atoms. In some embodiments, the sugar moiety comprises more than 5 ring atoms, such as 6 ring atoms. In some embodiments, the sugar moiety is modified to include a morpholino. Morpholino-based ASOs refer to an ASO comprising morpholino subunits supporting a nucleobase and, instead of a ribose, containing a morpholinyl ring. Non-limiting examples of internucleotide linkages for such morpholino-based ASOs include, for example, phosphoramidate or phosphorodiamidate internucleotide linkages joining the morpholinyl ring nitrogen of one morpholino subunit to the 4′ exocyclic carbon of an adjacent morpholino subunit. Each morpholino subunit comprises a purine or pyrimidine nucleobase, which may bind by base-specific hydrogen bonding to a nucleobase in an oligonucleotide. In some embodiments, the morpholino-based ASO may include at least one further modification.

In some embodiments, both the sugar moiety and the internucleoside linkage between the nucleobase and the sugar moiety of at least one nucleotide unit in the ASO are replaced with non-natural groups. In some embodiments, the nucleobase units are maintained for hybridization with an appropriate nucleic acid target compound. In some embodiments, the ASO is chosen from peptide nucleic acids (PNAs). In some embodiments, the sugar-backbone of at least one oligonucleotide in the PNA is replaced with an amide-containing backbone, for example, an aminoethylglycine backbone. In some embodiments, the nucleobases are retained and are bound directly or indirectly to aza nitrogen atoms of the amide portion of the backbone.

In some embodiments, the ASOs may further comprise at least one nucleobase (often referred to as “base”) modification or substitutions, for example, 5-substituted pyrimidines, 6-azapyrimidines, and N-2, N-6, and 0-6 substituted purines, including 2-aminopropyladenine, 5-propynyluracil, and 5-propynylcytosine. Certain nucleobases are particularly useful for increasing the binding affinity of the oligomeric compounds of the disclosure. For example, 5-methylcytosine substitutions have been shown to increase nucleic acid duplex stability by 0.6-1.2° C. In some embodiments, the modified nucleobase is stereo-controlled.

It is not necessary for all positions in a given ASO to be uniformly modified, and in fact, more than one of the aforementioned modifications may be incorporated in a single nucleoside within an ASO. ASOs may contain at least one region wherein the ASO is modified to confer upon them increased resistance to nuclease degradation, increased cellular uptake, and/or an additional region for increased binding affinity for the target nucleic acid.

Due to potential three-dimensional variation of the sugar moieties, nucleobases, and internucleotide linkages, some nucleotides may share the same molecular formula but have a different spatial arrangement, i.e., some nucleotides may be stereoisomers. In some embodiments, the stereochemistry of nucleotides within a given ASO are not controlled so as to make the ASO stereorandom. In some embodiments, the nucleotides within a given ASO are stereo-controlled. In some embodiments, the nucleotides within a given ASO are stereo-controlled so as to make the ASO stereopure. In some embodiments a given ASO is a combination of stereo-controlled and stereorandom nucleotides.

In some ASOs, it is possible for some modifications to the sugar moieties, nucleobases, internucleotide linkages and/or stereo-controlled nucleotides to be arranged in regions that create a particular motif for the ASO. In some embodiments, the ASO comprises at least two regions. In some embodiments, the ASO comprises three regions: one region near the 5′ end of the ASO, one region near the 3′ end of the ASO, and a gap region between the two other regions. This type of arrangement is known as a gapmer motif. The length of each motif can be equal to other motifs within the ASO, or the length of each motif can be independent of the length of other motifs within the ASO. In some embodiments, one or more sugar moieties in an ASO are modified so that a block of sugar moieties in one region of the ASO are different from a block of sugar moieties in a different region of the ASO. In some embodiments, an ASO comprises modified sugar moieties arranged in a gapmer motif. In some embodiments, one or more nucleobases in an ASO are modified so that a block of nucleobases in one region of the ASO are different from a block of nucleobases in a different region of the ASO. In some embodiments, an ASO comprises modified nucleobases arranged in a gapmer motif. In some embodiments, one or more internucleotide linkages in an ASO are modified so that a block of internucleotide linkages in one region of the ASO are different from a block of internucleotide linkages in a different region of the ASO. In some embodiments, a given ASO comprises modified internucleotide linkages arranged in a gapmer motif. In some embodiments, one or more stereo-controlled nucleotides in an ASO are modified so that a block of stereo-controlled nucleotides in one region of the ASO are different from a block of stereo-controlled nucleotides in a different region of the ASO. In some embodiments, an ASO comprises stereo-controlled nucleotides arranged in a gapmer motif. In some embodiments, an ASO has more than one motif. In some embodiments, an ASO has more than one motif independent of each other.

### Manufacturing Antisense Oligonucleotides

The antisense molecules used in accordance with this disclosure may be made through well-known techniques of solid phase synthesis. Equipment for such synthesis is available from several sources including, for example, Applied Biosystems (Foster City, Calif.). One method for synthesizing oligonucleotides on a modified solid support is described in U.S. Pat. No. 4,458,066.

Any other methods for such synthesis known in the art may additionally or alternatively be employed. It is well known to use similar techniques to prepare oligonucleotides, such as phosphorothioates and alkylated derivatives. In one such automated embodiment, diethyl-phosphoramidites are used as starting materials and may be synthesized as described by Beaucage, et al., Tetrahedron Letters, 22:1859-1862 (1981).

In some embodiments, the ASOs are synthesized in a way so that all nucleotides of the ASO are stereopure.

In some embodiments, the ASOs are synthesized in vitro and do not include antisense compositions of biological origin. In some embodiments, the ASOs may also be mixed, encapsulated, conjugated or otherwise associated with other molecules, molecule structures, or mixtures of compounds, as for example, liposomes, lipids, receptor targeted molecules for assisting in uptake, distribution and/or absorption. Further information about synthesis of certain ASOs according to some embodiments is included in the Examples below.

### Methods of Inducing Exon-2 Skipping During Pre-mRNA Splicing

In some embodiments, the ASOs are used to induce Exon-2 skipping during processing of CD33 pre-mRNA. In some embodiments, at least one ASO disclosed herein is used to induce Exon-2 skipping in CD33 pre-mRNA during pre-mRNA splicing. In some embodiments, the at least one ASO is introduced into a cell, wherein the at least one ASO comprises all or a portion of SEQ ID NO:1, wherein the ASO hybridizes to a target region of the CD33 gene, and wherein the ASO induces Exon-2 skipping during pre-mRNA splicing of the CD33 gene. In some embodiments, the ASO administered to induce Exon-2 skipping during pre-mRNA splicing comprises one of SEQ ID NOS:2-15, 36-39, 82, 83, 96, 97, 128, 132, 135, 136, 183, 184, 190, 196, or 197.

In some embodiments, the at least one ASO is administered by itself, as a so-called “naked” ASO. In some embodiments, the at least one naked ASO is synthesized in vitro. In some embodiments, the at least one naked ASO is introduced into a cell to directly hybridize to a target region of the CD33 gene to induce Exon-2 skipping during pre-mRNA splicing.

Certain methods of introducing “naked” ASOs or expression vectors encoding ASOs into a cell are well known in the art. In some embodiments, an ASO or expression vector encoding an ASO can be introduced by transfection using known transfection agents. In some embodiments, the use of an excipient or transfection agent aids in delivery of the ASO or expression vector encoding the ASO as defined herein to a cell and/or into a cell. In some embodiments, excipients or transfection agents are capable of forming complexes, nanoparticles, micelles, vesicles, and/or liposomes that deliver each ASO or expression vector encoding each ASO as defined herein, complexed or trapped in a vesicle or liposome through a cell membrane. Many of these excipients are known in the art. Suitable excipients or transfection agents include LipofectAMINE™ 2000 (Invitrogen), Endo-Porter peptide, polyethylenimine (PEI; ExGen500 (MBI Fermentas)), or derivatives thereof, or similar cationic polymers, including polypropyleneimine or polyethylenimine copolymers (PECs) and derivatives, synthetic amphiphils (SAINT-18), Lipofectin™, DOTAP and/or viral capsid proteins that are capable of self-assembly into particles that can deliver each ASO as defined herein to a cell. Such excipients have been shown to efficiently deliver an oligonucleotide such as ASOs to a wide variety of cultured cells. Their high transfection potential is combined with an excepted low to moderate toxicity in terms of overall cell survival. The ease of structural modification can be used to allow further modifications and the analysis of their further (in vivo) nucleic acid transfer characteristics and toxicity.

In some embodiments, the ASO is administered in the form of an expression vector, wherein the expression vector encodes an RNA transcript comprising the sequence of the ASO. When placed under conditions conducive to expression of the encoded ASO, the expression vector can express the encoded ASO, which can hybridize to a target region of the CD33 gene to induce Exon-2 skipping during pre-mRNA splicing. The expression vector can be a viral or non-viral vector. In some embodiments, there is provided a plasmid-based expression vector comprising an expression cassette or a transcription cassette that drives expression or transcription of an ASO for redirecting splicing.

In some embodiments, a cell can be provided with an ASO for redirecting splicing by plasmid-derived ASO expression or viral expression provided by cytolomegalovirus-, adenovirus-, or adeno-associated virus-based vectors. In some embodiments, expression may be driven by an RNA polymerase II promoter (Pol II) such as a U7 RNA promoter or an RNA polymerase III (Pol Ill) promoter, such as a U6 RNA promoter. In some embodiments, the delivery vehicle is an expression vector. In some embodiments, plasmids and artificial chromosomes are usable for targeted homologous recombination and integration in the human genome of cells may be applied for delivery of an ASO for redirecting splicing.

### Therapeutic Methods

Disclosed herein are methods of treating a subject having a neurodegenerative disease comprising administering at least one ASO disclosed herein. In some embodiments, the methods comprise administering a therapeutically effective amount of at least one ASO disclosed herein. In some embodiments, the methods comprise administering a therapeutically effective amount of at least one ASO that hybridizes to all or a portion of SEQ ID NO:1. In some embodiments, the methods comprise administering a therapeutically effective amount of at least one ASO comprising one of SEQ ID NOS:2-10. In some embodiments, the neurodegenerative disease is characterized by a mutation in the CD33 gene. In some embodiments, the neurodegenerative disease is characterized by an aberrant microglial phenotype. In some embodiments, the neurodegenerative disease is Alzheimer's Disease, microfibromialgia, or multiple sclerosis.

In some embodiments, the ASO administered to a subject having a neurodegenerative disease may be administered in a pharmaceutical composition. In some embodiments, the amount of ASO administered in a pharmaceutical composition may be dependent on the subject being treated, the subject's weight, the manner of administration, and the judgment of the prescribing physician. For example, in some embodiments, a dosing schedule may involve the daily or semi-daily administration of the pharmaceutical composition at a perceived dosage of about 1 μg to about 1000 mg. In some embodiments, intermittent administration, such as on a monthly or yearly basis, of a dose of the pharmaceutical composition may be employed. In accordance with standard dosing regimens, in some embodiments, physicians will readily determine optimum dosages and will be able to readily modify administration to achieve such dosages.

A therapeutically effective amount of a compound or composition disclosed herein can be measured by the therapeutic effectiveness of the compound. In some embodiments, the dosages, however, may be varied depending upon the requirements of the patient, the severity of the condition being treated, and the compound being used. In some embodiments, the therapeutically effective amount of a disclosed compound is sufficient to establish a maximal plasma concentration. In some embodiments, preliminary doses as, for example, determined according to animal tests, and the scaling of dosages for human administration is performed according to art-accepted practices.

In some embodiments, toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. In some embodiments, compositions that exhibit large therapeutic indices are desirable.

In some embodiments, data obtained from the cell culture assays or animal studies can be used in formulating a range of dosage for use in humans. In some embodiments, therapeutically effective dosages achieved in one animal model may be converted for use in another animal, including humans, using conversion factors known in the art (see, e.g., Freireich et al., Cancer Chemother. Reports 50(4):219-244 (1966).

The ASOs herein may be administered in a pharmaceutical composition comprising therapeutically effective amounts of an ASO together with pharmaceutically acceptable excipients, diluents, preservatives, solubilizers, emulsifiers, adjuvants and/or carriers. In some embodiments, such compositions include diluents of various buffer content (e.g., Tris-HCl, acetate, phosphate), pH, and ionic strength, and additives such as detergents and solubilizing agents (e.g., Tween 80, Polysorbate 80), anti-oxidants (e.g., ascorbic acid, sodium metabisulfite), preservatives (e.g., Thimersol, benzyl alcohol), and bulking substances (e.g., lactose, mannitol). In some embodiments, the material may be incorporated into particulate preparations of polymeric compounds such as polylactic acid, polyglycolic acid, etc. or into liposomes. In some embodiments, Hyaluronic acid may also be used. Such compositions may influence the physical state, stability, rate of in vivo release, and/or rate of in vivo clearance of the present ASOs and derivatives. In some embodiments, the compositions may be prepared in liquid form, or may be in dried powder, such as lyophilized form.

### Administration

In some embodiments, a pharmaceutical composition comprising an ASO and a pharmaceutically acceptable carrier or excipient may be prepared for administration according to techniques well known in the pharmaceutical industry. In some embodiments, such techniques include combining the ASO with the carrier and/or excipient(s) into association in a unit dosage form.

In some embodiments, compositions suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. In some embodiments, such formulations may be prepared by any suitable method which includes the step of bringing into association at least one embodiment of the present disclosure as the active compound and at least one carrier or excipient (which may constitute one or more accessory ingredients). In some embodiments, the at least one carrier is acceptable in the sense of being compatible with the other ingredients of the formulation and is not deleterious to the recipient. In some embodiments, the carrier may be a solid or a liquid, or both, and may be formulated with at least one compound described herein as the active compound in a unit-dose formulation, for example, a tablet, which may contain from about 0.05% to about 95% by weight of the at least one active compound. In some embodiments, other pharmacologically active substances may also be present including other compounds. In some embodiments, the formulations of the present disclosure may be prepared by any of the well-known techniques of pharmacy consisting essentially of admixing the components.

For solid compositions, in some embodiments, conventional nontoxic solid carriers include, for example, pharmaceutical grades of mannitol, lactose, starch, magnesium stearate, sodium saccharin, talc, cellulose, glucose, sucrose, magnesium carbonate, and the like. In some embodiments, liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, in some embodiments, suitable formulations may be prepared by uniformly and intimately admixing the at least one active compound of the present disclosure with a liquid or finely divided solid carrier, or both, and then, if desired, shaping the product. For example, in some embodiments, a tablet may be prepared by compressing or molding a powder or granules of at least one embodiment of the present disclosure, which may be optionally combined with one or more accessory ingredients. In some embodiments, compressed tablets may be prepared by compressing, in a suitable machine, at least one embodiment of the present disclosure in a free-flowing form, such as a powder or granules, which may be optionally mixed with a binder, lubricant, inert diluent and/or surface active/dispersing agent(s). In some embodiments, molded tablets may be made by molding, in a suitable machine, where the powdered form of at least one embodiment of the present disclosure is moistened with an inert liquid diluent.

In some embodiments, formulations suitable for buccal (sub-lingual) administration include lozenges comprising at least one embodiment of the present disclosure in a flavored base, for example, sucrose and acacia or tragacanth, and pastilles comprising the at least one compound in an inert base such as gelatin and glycerin or sucrose and acacia.

In some embodiments, formulations suitable for parenteral administration comprise sterile aqueous preparations of at least one embodiment of the present disclosure, which are approximately isotonic with the blood of the intended recipient. In some embodiments, these preparations are administered intravenously, although administration may also be affected by subcutaneous, intramuscular, intraperitoneal, intracerebroventricular, or intradermal injection. In some embodiments, these preparations are administered via osmotic pump. In some embodiments, such preparations may conveniently be prepared by admixing at least one embodiment described herein with water and rendering the resulting solution sterile and isotonic with the blood. In some embodiments, injectable compositions according to the present disclosure may contain from about 0.1 to about 5% w/w of the active compound.

In some embodiments, formulations suitable for rectal administration are presented as unit-dose suppositories. In some embodiments, these may be prepared by admixing at least one embodiment as described herein with one or more conventional solid carriers, for example, cocoa butter, and then shaping the resulting mixture.

In some embodiments, formulations suitable for topical application to the skin may take the form of an ointment, cream, lotion, paste, gel, spray, aerosol, or oil. In some embodiments, carriers and excipients which may be used include Vaseline, lanoline, polyethylene glycols, alcohols, and combinations of two or more thereof. In some embodiments, the ASO is generally present at a concentration of from about 0.1% to about 15% w/w of the composition, for example, from about 0.5 to about 2%.

### EXAMPLES

The following Examples serve to more fully describe the invention. They are meant for illustrative purposes and are not meant to limit the invention in any way.

### ABBREVIATIONS

ASO: antisense oligonucleotide
DNA: deoxyribonucleic acid
cDNA: complementary deoxyribonucleic acid
RNA: ribonucleic acid
mRNA: messenger ribonucleic acid
PMO: phosphorodiamidate morpholino oligomer
MOE: methoxyethyl
LOAD: late onset Alzheimer's Disease
SNP: single nucleotide polymorphism
PNA: peptide nucleic acid
DOTAP: 1,2 dioleoyl 3 trimethylammoniopropane
PEI: polyethylenimine
PEC: polyethylenimine copolymers
HRMS: high resolution mass spectrometry
MW: molecular weight
SP: stereopure
UPLC: ultra performance liquid chromatography
MS: mass spectrometry
MTBE: Methyl tert-butyl ether
DCM: dichloromethane
TFA: trifluoroacetic acid
RT: room temperature
H: hour
Min: minute
EtOAc: ethyl acetate
HPRT1: hypoxanthine phosphoribosyltransferase 1
GAPDH1: glyceraldehyde 3 phosphate dehydrogenase 1
NTC: non-targeting control
WP: well plate
Bz—benzoyl
CE—2-cyanoethyl
Trt—trityl
iPr—isopropyl
Sar —Sarcosine
ESI-TOF-MS—electrospray ionization—time-of-flight mass spectrometry

### Example 1: Reducing or Interfering with Full Length CD33

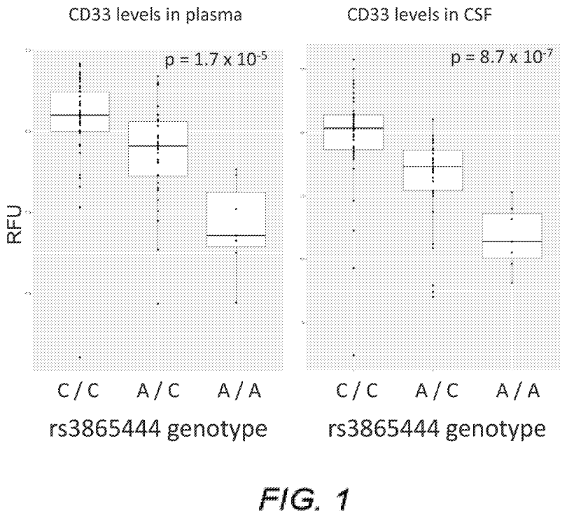

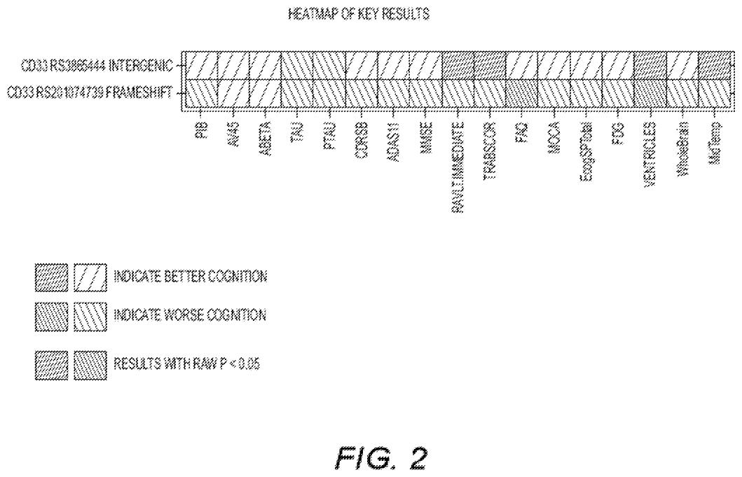

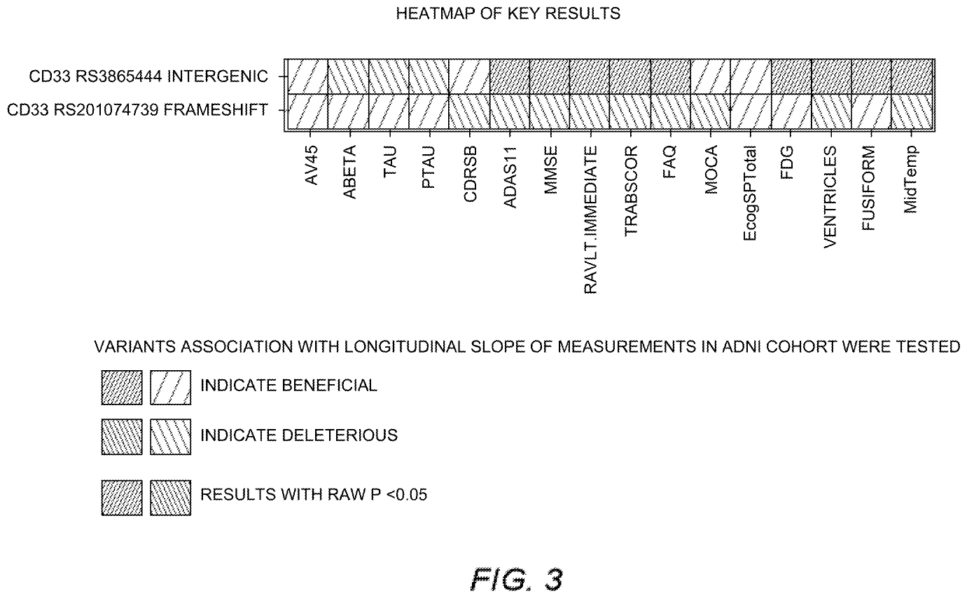

SNP rs3865444 was reported to be associated with an increased skipping of Exon-2 of CD33 and with reduced levels of full length CD33 on the surface of monocytes. The allele was found to be associated with decreased levels of full length CD33 in human cerebrospinal fluid (CSF) and plasma when measured using Somascan technology (FIG. 1). In a study by the Alzheimer's Disease Neuroimaging Initiative (ADNI), the allele was found to be associated with decreased ventricle volume and increased midtemporal volume, which are both consistent with protection against Alzheimer's Disease (FIG. 2). Moreover, in the longitudinal analysis, the allele was associated with improved slope for Alzheimer's Disease Assessment Scale (ADAS) 11, mini-mental state examination (MMSE), Rey Auditory Verbal Learning Test (RAVLT) immediate, Trial Making Test-B (TRABSCOR), Functional Activities Questionnaire (FAQ), ¹⁸F-fluorodeoxyglucose-positron emission tomography (FDG PET), ventricle volume, fusiform gyrus, and midtemporal volume (FIG. 3), indicating protection against the disease.

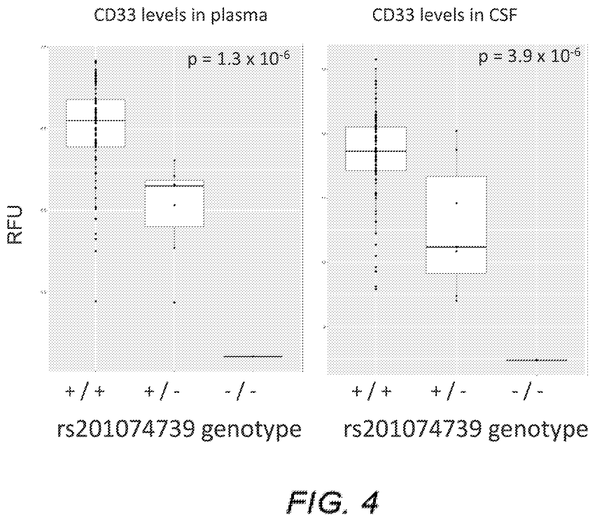

On the other hand, rs201074739 is a 4-base pair deletion in exon3 of the CD33 gene. This causes a frameshift in the open reading frame and a premature translation termination. The indel was associated with decreased levels of full length CD33 in human CSF and plasma when measured using SomaScan technology (FIG. 4). However, this indel has not been associated with a reduced risk of the disease so far. Moreover, it was associated with increased ventricle volume and a worse functional activities questionnaire (FAQ) score, suggesting a deleterious effect (FIG. 2).

Accordingly, successfully inducing Exon-2 skipping of CD33 may have therapeutic benefits.

### Example 2: General ASO Formulas

PMO oligonucleotides were designed for screening. The designed oligonucleotides listed in Tables 1 and 3 below were made by GeneTools LLC (www.gene-tools.com). Table 1 lists the top PMO oligonucleotides with their deconvoluted MS data. Table 3 includes the top PMO oligonucleotides in Table 1, as well as other PMO oligonucleotides. All PMO oligonucleotides listed in Tables 1 and 3 below contain a phosphorodiamidate-attached sarcosine linker (Sar) at the 5′ end. All PMO oligonucleotides in Tables 1 and 3 below were synthesized with unmodified cytosine PMO nucleotide. All PMO oligonucleotides listed in Tables 1 and 3 below have stereorandom internucleotide linkages, and thus are called stereorandom PMO oligonucleotides. The general formula of the PMO oligonucleotides listed in Tables 1 and 3 below is:

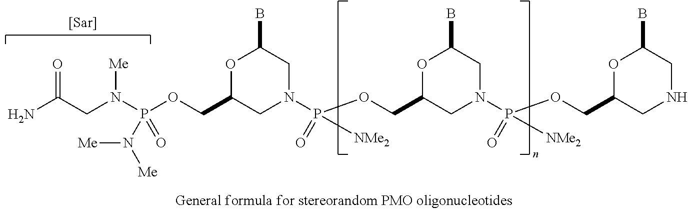

TABLE 1

| | | | MW | |
|---|---|---|---|---|
| | | | (with | |
| | | SEQ ID | Sarcosine | MS |
| ASO # | Seq. | NO | linker) | observed |
| PMO-002 | 5′ -CCTCACCTGTCACATGCACAGAGAG- | 2 | 8398.06 | 8403.0 |
| | 3′ | | | |
| PMO-003 | 5′ -CCTGTCACATGCACAGAGAGCTGGG- | 3 | 8494.11 | 8498.4 |
| | 3′ | | | |
| PMO-004 | 5′ -ATTTGGTACTTCCTCTCTCCATCCG-3′ | 4 | 8311.96 | 8316.4 |
| PMO-005 | 5′ -TCTCCATCCGAAAGAAGTATGAACC- | 5 | 8421.09 | 8423.7 |
| | 3′ | | | |
| PMO-006 | 5′ - | 6 | 8647.24 | 8649.7 |
| | TGGGATGGAAGAAAGTGCAGGGCAC-3′ | | | |
| PMO-007 | 5′ -CAGCCAGAAATTTGGATCCATAGCC- | 7 | 8437.09 | 8444.7 |
| | 3′ | | | |
| PMO-008 | 5′ - | 8 | 8550.14 | 8552.5 |
| | CCCTGTGGGGAAACGAGGGTCAGCT-3′ | | | |

MOE oligonucleotides were designed for screening. The designed oligonucleotides listed in Tables 2 and 4 below were made by either Integrated DNA Technologies (www.idtdna.com) or GeneDesign (Ajinomoto Bio Pharma, https://ajibio-pharma.com/). Table 2 lists the top MOE sequences with their deconvoluted MS data. All MOE oligonucleotide listed in Tables 2 and 4 below contain a hydroxyl at the 5′ end. All MOE oligonucleotides listed in Tables 2 and 4 below contain 2′-O-MOE-modified ribonucleotides with phosphorothioate backbone except when noted. All MOE oligonucleotides listed in Tables 2 and 4 below were synthesized with 5-methylcytosine 2′-O-MOE ribonucleotide. All MOE oligonucleotides listed in Tables 2 and 4 below have stereorandom internucleotide linkages, and thus are called stereorandom MOE oligonucleotides. The general formula of the MOE oligonucleotides listed in Tables 2 and 4 depicted as free form is:

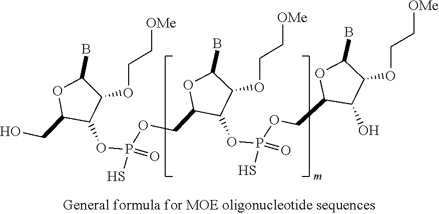

TABLE 2

| | | SEQ. | MW as | MS |
|---|---|---|---|---|
| | | ID | free | |
| ASO # | Seq. (5′ to 3′) | NO: | form | observed |
| MOE-009 | CACATGCACAGAGAGCTGGG | 9 | 8031.7 | 8033.80 |
| MOE-010 | GAGAGCTGGGGAGATTTGTA | 10 | 8077.1 | 8077.31 |
| MOE-011 | TCTCCATCCGAAAGAAGTAT | 11 | 7940.9 | 7942.85 |
| MOE-012 | ATCCGAAAGAAGTATGAACC | 12 | 7985.2 | 7986.62 |
| MOE-013 | ATGCTCAGGGAGCAGTTGTT | 13 | 8016.1 | 8016.17 |
| MOE-014 | GAGTCTCCTCCTGTACTTCT | 14 | 7894.4 | 7895.30 |
| MOE-015 | CGCACAAACCCTCCTGTACC | 15 | 7893.0 | 7893.84 |

### Example 3: Synthesis of Pmo-302 (Stereopure Internucleotide Linkages (Sp))

Synthesis of Stereopure PMO-302 Oligonucleotide with Unfunctionalized 5′-OH (CCTCACCTGTCACATGCACAGAGAG (SEQ ID NO: 2)).

Monomers used in the synthesis of PMO-302 are as follows: (reported in WO2017024264A2):

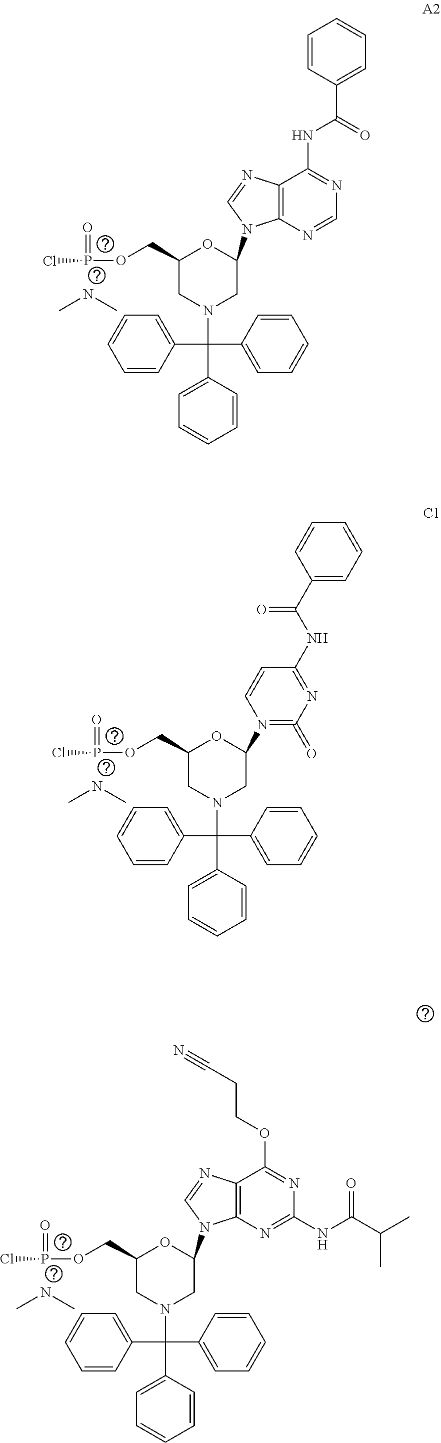

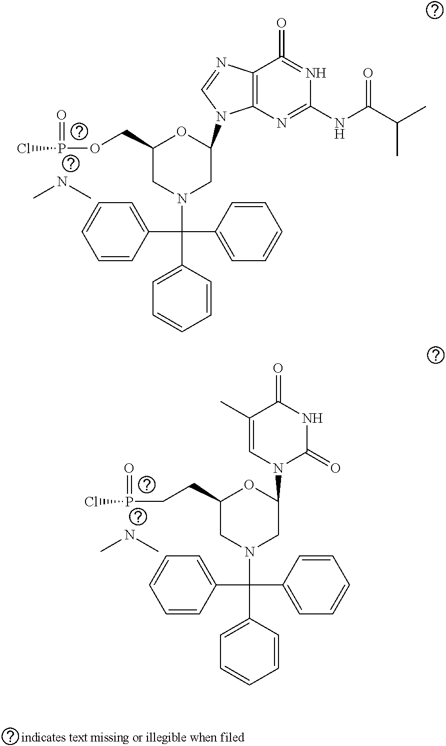

Synthesis of PMO-302 with 5′-OH and Stereopure Internucleotide Linkages: 2-Mer Synthesis:

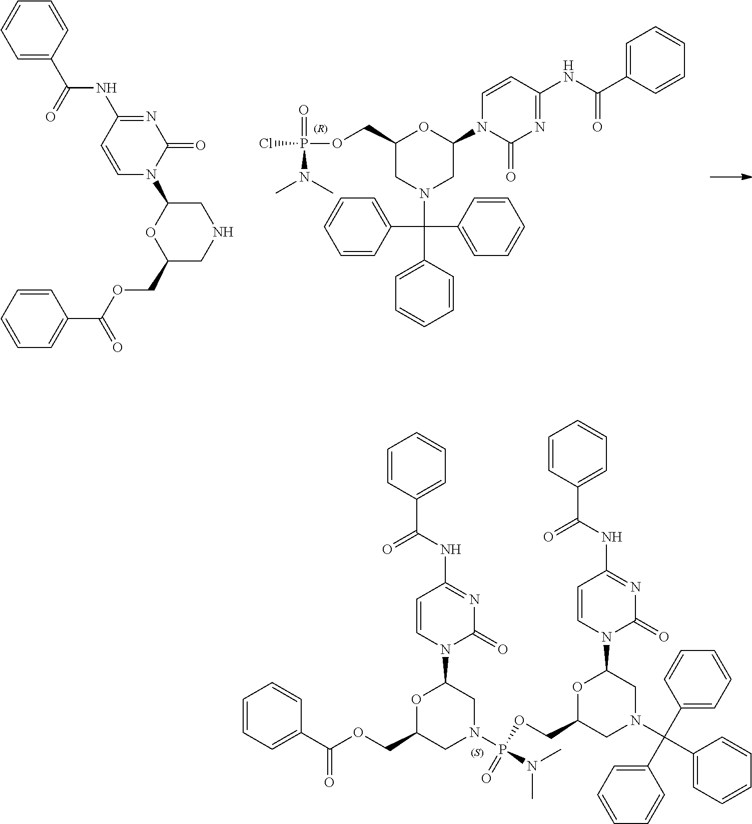

Unless otherwise noted all liquid ingredients were added via an appropriate size syringe. All reactions were conducted under N₂ atmosphere. Filtrations and workup were done open to the air. Filtrations were conducted on a glass sintered funnel.

A flask with the amine ((2S,6R)-6-(4-benzamido-2-oxopyrimidin-1(2H)-yl)morpholin-2-yl)methyl benzoate (130 mg) was equipped with a stir bar and rubber septum. The atmosphere was exchanged with nitrogen and sparged. After 5 minutes, added 1,3-dimethyl-2-imidazolidinone (2.2 mL) followed by 1,2,2,6,6-pentamethylpiperidine (164 μL) via syringe at rt and the mixture was allowed to form a solution. Solid ((2S,6R)-6-(4-benzamido-2-oxopyrimidin-1(2H)-yl)-4-tritylmorpholin-2-yl)methyl (R)-dimethyl phosphoramidochloridate (219 mg) was added in one portion and the flask was sealed with the rubber septum. Stirring was continued for 3 h and the reaction monitored by UPLC MS. Upon completion, while stirring, MTBE (11.7 mL) was added over 1 minutes. Precipitate was formed towards the end of the addition. n-Heptane (10 mL) was added. The oily mixture was allowed to settle for 10 minutes. While the heavy oil was settled, the cloudy supernatant was transferred by decantation to a 30 mL vial and centrifuged. This formed an additional oily residue on the bottom. The solvent was removed by decantation and the two oily residues were combined by dissolving into 1 mL of DCM and purified by flash chromatography with 0-5% MeOH in DCM. The fractions containing desired product were dried under vacuum to obtain ((2S,6R)-6-(4-benzamido-2-oxopyrimidin-1(2H)-yl)-4-((S)-(((2S,6R)-6-(4-benzamido-2-oxopyrimidin-1(2H)-yl)-4-tritylmorpholin-2-yl)methoxy)(dimethylamino)phosphoryl)morpholin-2-yl)methyl benzoate (270 mg) as a white foam. MS (ESI) m/z: [M+H]⁺ Calcd for C₆₀H₅₈N₉O₁₀P: 1096.40; Found: 1096.53. Deprotection of 2-mer:

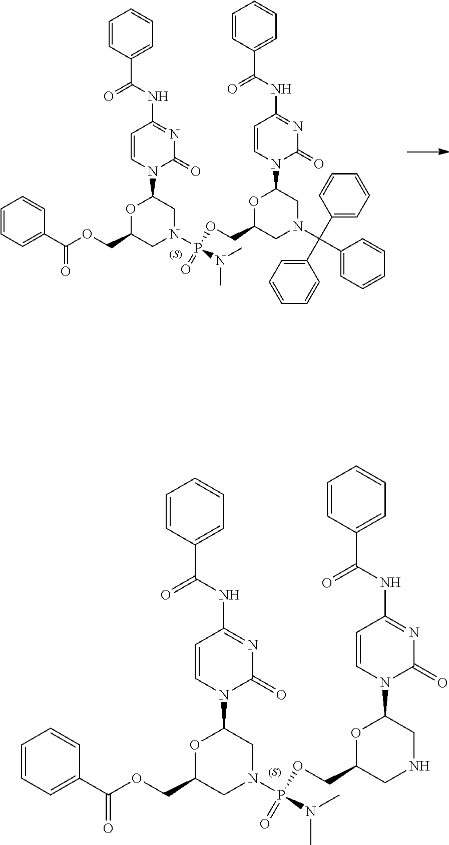

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-tritylmorpholin-2 yl)methoxy)(dimethylamino)phosphoryl)morpholin-2-yl)methyl benzoate (350 mg) was added DCM (3.5 mL). Added ethanol (186 μL) via syringe at rt. Added TFA (160 μL) at rt dropwise over 30 seconds. Stirred for 30 min and monitored by UPLC-MS. Upon completion, added MTBE (14 mL) over 1 minute with a syringe. The suspension was stirred for 10 min and then sonicated. Filtered over a sintered filter funnel and rinsed with MTBE 10 mL (2×5 mL). The solids were dried, transferred to a new flask and then dissolved by addition of DCM (3.5 mL). 1,2,2,6,6-pentamethylpiperidine (292 μL) was added via syringe. After 10 min at rt added MTBE (15.8 mL) over 1 minute. White solids were formed. After 10 minutes the slurry was sonicated, filtered and rinsed with MTBE (e.g. 2×10 mL). Dried for 20 minutes under air flow and 1 hour under vacuum to obtain ((2S,6R)-6-(4-benzamido-2-oxopyrimidin-1(2H)-yl)-4-((S)-(((2S,6R)-6-(4-benzamido-2-oxopyrimidin-1(2H)-yl)morpholin-2-yl)methoxy)(dimethylamino)phosphoryl)morpholin-2-yl)methyl benzoate (243 mg). MS (ESI) m/z: [M+H]⁺ Calcd for C₄₁H₄₄N₉₀O₁₀P: 854.29; Found: 854.65.

### 3-Mer Synthesis:

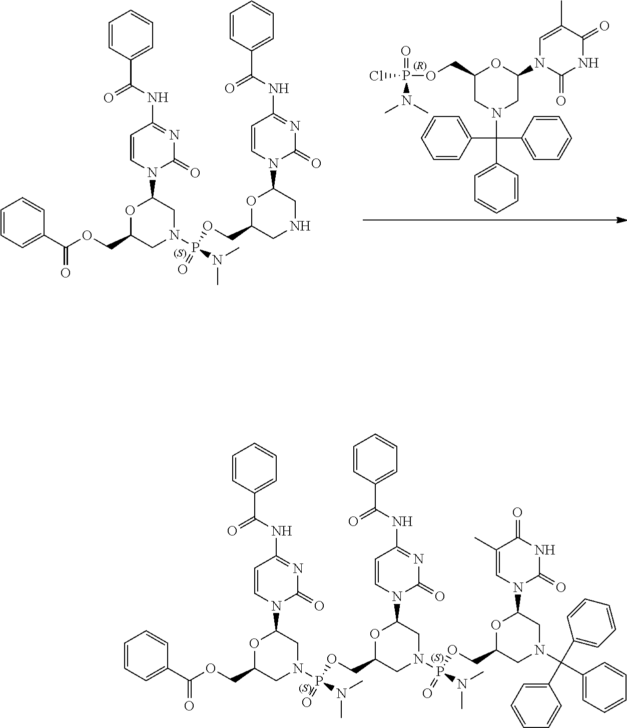

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)morpholin-2-yl)methoxy)(dimethylamino)phosphoryl)morpholin-2-yl)methyl benzoate (215 mg) were added 1,3-Dimethyl-2-imidazolidinone (2 mL) and 1,2,2,6,6-pentamethylpiperidine (138 μL) under nitrogen. After 2-min added ((2S,6R)-6-(5-methyl-2,4-dioxo-3,4-dihydropyrimidin-1(2H)-yl)-4-tritylmorpholin-2-yl)methyl-(R)-dimethylphosphoramidochloridate (169 mg) and the reaction was stirred for 3 h at rt. Upon completion, added ethyl acetate (2.6 mL) and then MTBE (14 mL). The resulting white precipitate was filtered, rinsed with MTBE (2×5 mL) and dried under vacuum to obtain ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(dimethylamino)(((2S,6R)-6-(5-methyl-2,4-dioxo-3,4-dihydropyrimidin-1(2H)-yl)-4-tritylmorpholin-2-yl)methoxy)phosphoryl)morpholin-2-yl)methoxy)(dimethylamino)phosphoryl)morpholin-2-yl)methyl benzoate (350 mg). MS (ESI) m/z: [M+H]⁺ Calcd for C₇₂H₇₇N₁₃O₁₅P₂: 1426.51; Found: 1427.74. Deprotection of 3-mer:

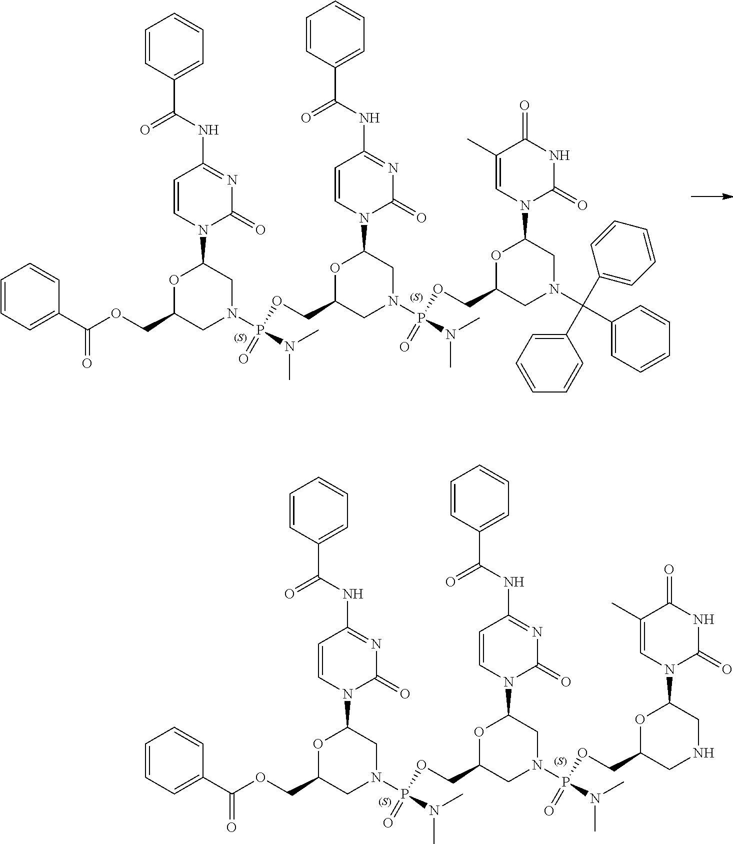

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(dimethylamino)(((2S,6R)-6-(5-methyl-2,4-dioxo-3,4-dihydropyrimidin-1(2H)-yl)-4-tritylmorpholin-2-yl)methoxy)phosphoryl)morpholin-2-yl)methoxy)(dimethylamino)phosphoryl)morpholin-2-yl)methyl benzoate (350 mg) and DCM (4.2 mL). Added ethanol (143 μL) and then slowly at rt added TFA (95 μL). The reaction mixture was stirred for 2 hours at rt. Upon completion, added MTBE (15 mL). The solids were filtered and rinsed with MTBE (10 mL). The solids were dried and then transferred to a flask and dissolved by addition of DCM (2.7 mL). Added 1,2,2,6,6-pentamethylpiperidine (224 μL) and rt and the reaction mixture was stirred at rt for 10 minutes. Added MTBE (15 mL) and the resulting slurry was stirred for 10 minutes and sonicated. Filtered and rinsed with MTBE (10 mL). Obtained trimer as free base (297 mg). MS (ESI) m/z: [M+H]⁺ Calcd for C₅₃H₆₃N₁₃O₁₅P₂: 1184.40; Found: 1185.

### 4-Mer Synthesis:

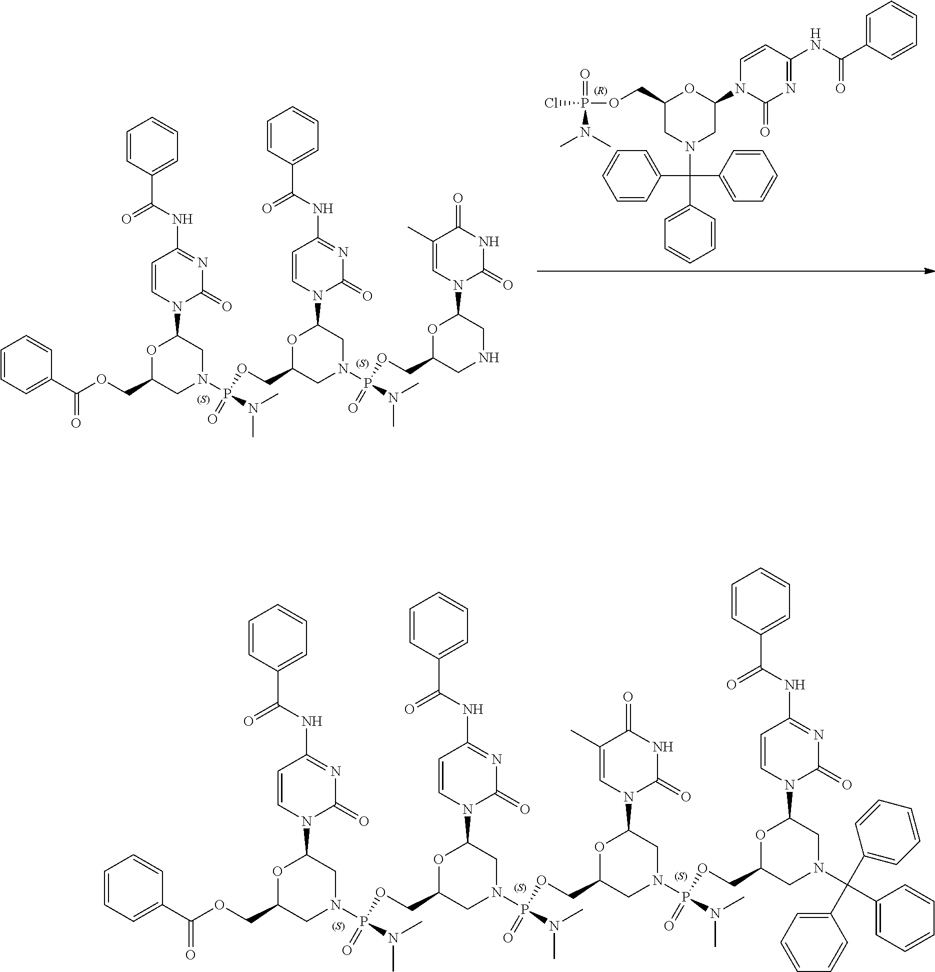

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(dimethylamino)(((2S,6R)-6-(5-methyl-2,4-dioxo-3,4-dihydropyrimidin-1(2H)-yl)morpholin-2-yl)methoxy)phosphoryl)morpholin-2-yl)methoxy)(dimethylamino)phosphoryl)morpholin-2-yl)methyl benzoate (292 mg) and the flask was purged with nitrogen. Added 1,3-Dimethyl-2-imidazolidinone (2.9 mL) and then 1,2,2,6,6-pentamethylpiperidine (135 μL). ((2S,6R)-6-(4-Benzamido-2-oxopyrimidin-1(2H)-yl)-4-tritylmorpholin-2-yl)methyl (R)-dimethylphosphoramidochloridate (207 mg) was added in one portion and the reaction was stirred at rt for at least 1h monitoring for completion by HPLC-MS. Ethyl acetate (2.9 mL) was charged followed by MTBE (14 mL). The slurry was stirred for 15 minutes and filtered, washed with MTBE 2×5 mL. The resulting solids were dried under vacuum for 10 minutes and then collected to a new flask, dried under vacuum to afford 430 mg of 4-mer. MS (ESI) m/z: [M+H]⁺ Calcd for C₆₀H₉₉N₁₈O₂₀P₃: 1846.65; Found: 1847.

Deprotection of 4-mer:

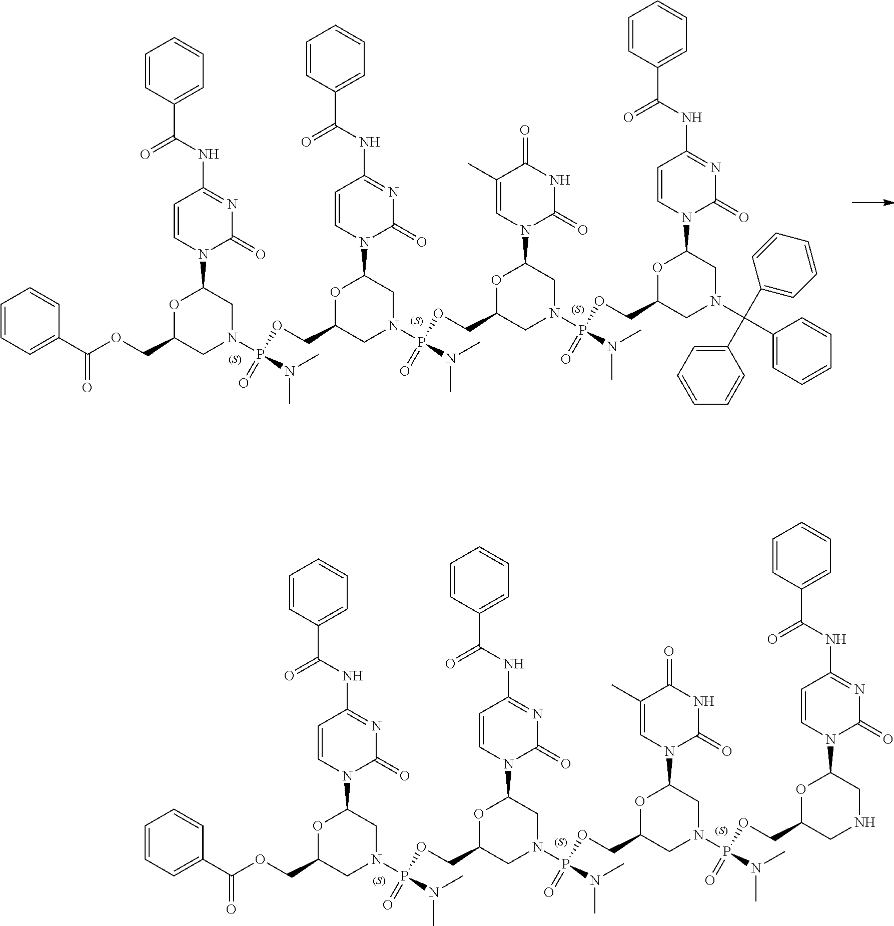

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-tritylmorpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (430 mg). Added DCM (4.3 mL) and ethanol (272 μL). After a solution was formed, added TFA (135 μL). The reaction mixture was stirred for 2.5h when it was deemed completed by HPLC analysis. Added ethyl acetate (3.0 mL) and MTBE (10.8 mL) over 1 minute. Solid precipitate were formed during MTBE addition. Upon completed MTBE addition, the solids were stirred for 10 minutes and sonicated three times. Filtered and rinsed with MTBE 2×5 mL. The solids were dried and then dissolved in DCM (4.3 mL) and treated with 1,2,2,6,6-pentamethylpiperidine (319 μL). After 5 min, the desired product was precipitated by addition of ethyl acetate (3.0 mL) and MTBE (10.8 mL) over 1 minute. The solids were filtered, rinsed with MTBE and dried under vacuum overnight to afford ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (330 mg). MS (ESI) m/z: [M+H]⁺ Calcd for C₇₁H₈₅N₁₈O₂₀P₃: 1603.54; Found: 1605.

5-mer synthesis:

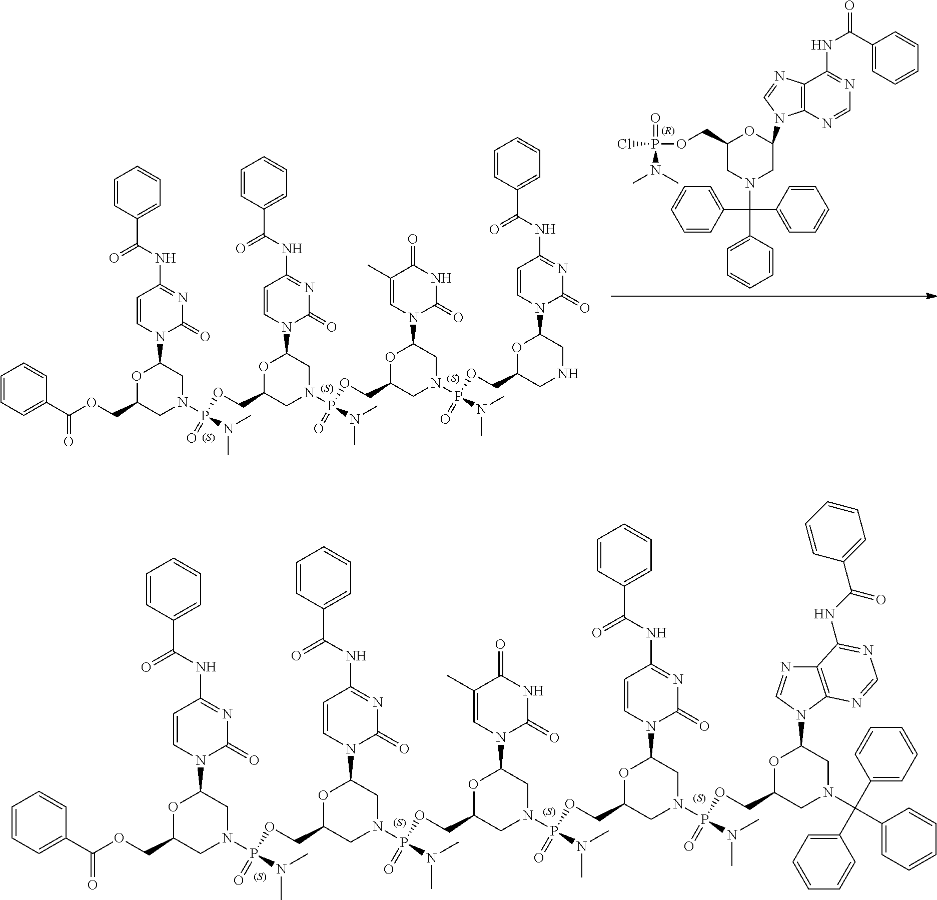

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (330 mg) was added 1,3-Dimethyl-2-imidazolidinone (3.3 mL) and 1,2,2,6,6-pentamethylpiperidine (113 μL). After the residue fully dissolved, added ((2S,6R)-6-(6-benzamido-9H-purin-9-yl)-4-tritylmorpholin-2-yl)methyl (R)-dimethylphosphoramidochloridate (178 mg) at rt. The reaction mixture was stirred at rt for 5 h and then added ethyl acetate (6.6 mL) and MTBE (13.2 mL). The white precipitate was filtered and dried. The solid was dissolved in DCM 2 mL and purified by automated silica gel chromatography on a 25 g cartridge with 0-20% MeOH in DCM. Afforded 345 mg of desired product 5-mer. MS (ESI) m/z: Calcd for C₁₀H₁₂₁N₂₅O₂₄P₄: [(M+2H)/2]+1145.4; Found: 1145.6.

Deprotection of 5-mer:

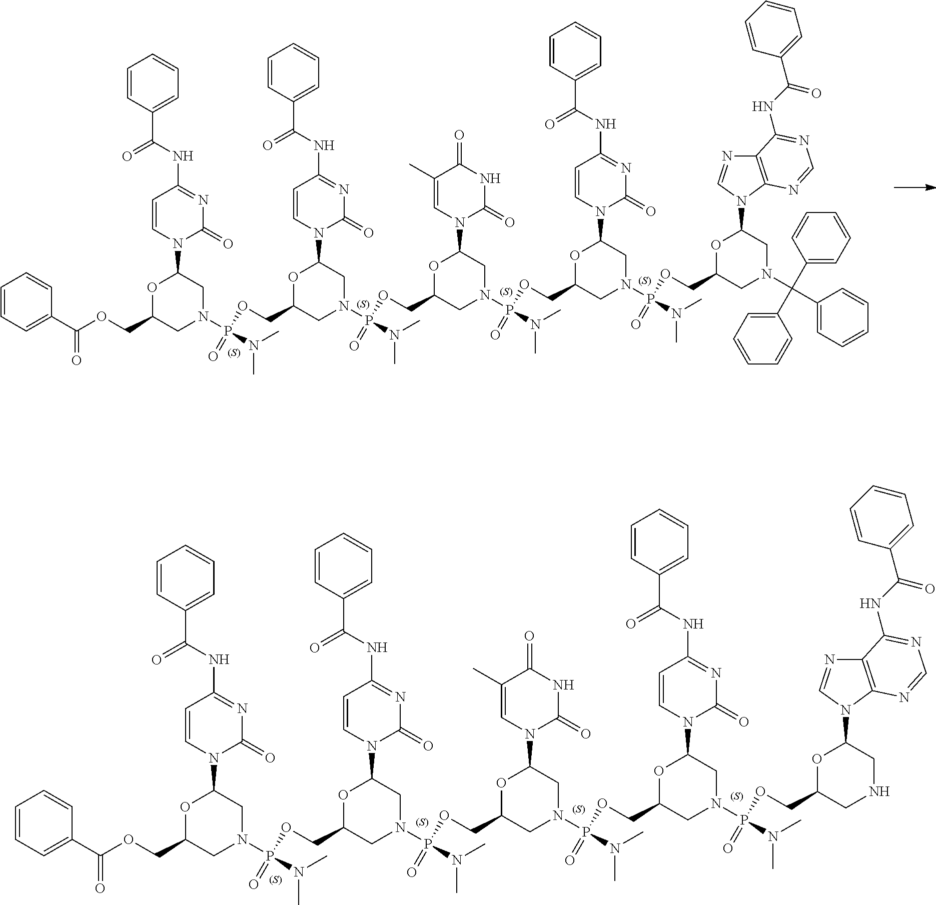

To a flask was added ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(6-benzamido-9H-purin-9-yl)-4-tritylmorpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (328 mg). Added DCM (3.1 mL) and then ethanol (167 μL). Added TFA (66.2 μL) at rt and stirred at rt for 3h. Added additional 3 drops (ca 15 μL) of TFA. The reaction was monitored by HPLC-MS and upon completion (disappearance of starting material peak), added ethyl acetate (11.8 mL) followed by stirring for 5 min. Filtered and rinsed with EtOAc (2 mL) and MTBE (5 mL). Additional solids were formed in the mother liquor which were also harvested by second filtration. The combined solids were placed into a reaction flask. Added DCM (2.3 mL) and 1,2,2,6,6-pentamethylpiperidine (209 μL). Stirred for 15 min then added EtOAc (2.6 mL) and MTBE (10.5 mL). The resulting solids were filtered and rinsed by MTBE 2×3 mL then dried under vacuum, collected to afford 280 mg deprotected 5-mer. MS (ESI) m/z: Calcd for C₉₀H₁₀₇N₂₅O₂₄P₄: [(M+2H)/2]⁺ 1023.8; Found: 1024.12.

6-mer synthesis:

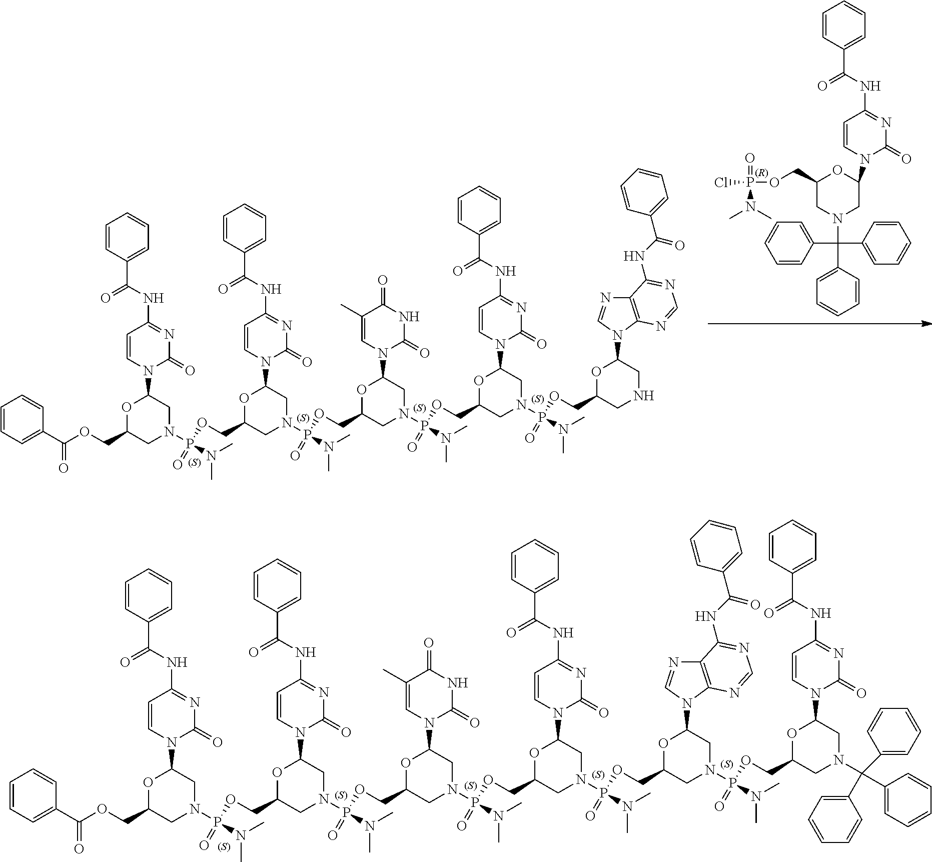

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (265 mg) was added 1,3-Dimethyl-2-imidazolidinone (2.7 mL). Added 1,2,2,6,6-pentamethylpiperidine (71.0 μL). Added ((2S,6R)-6-(4-benzamido-2-oxopyrimidin-1(2H)-yl)-4-tritylmorpholin-2-yl)methyl (R)-dimethylphosphoramidochloridate (108 mg) at rt as solid. After 3 hours at rt, upon completion as judged by HPLC analysis, added EtOAc (5.3 mL) and MTBE (10.6 mL) over 2-3 min each. Filtered and rinsed with MTBE 2×3 mL. After drying with air flow for 2-3 min the solids turned to sticky mass. The solids transferred to same flask with 10 mL DCM and concentrated under vacuum. Isolated ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-tritylmorpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (354 mg). MS (ESI) m/z: Calcd for C₁₂₇H₁₄₃N₃₀O₂₉P₅: [M+2H/2]+1354.97; Found: 1354.73.

Deprotection of 6-mer:

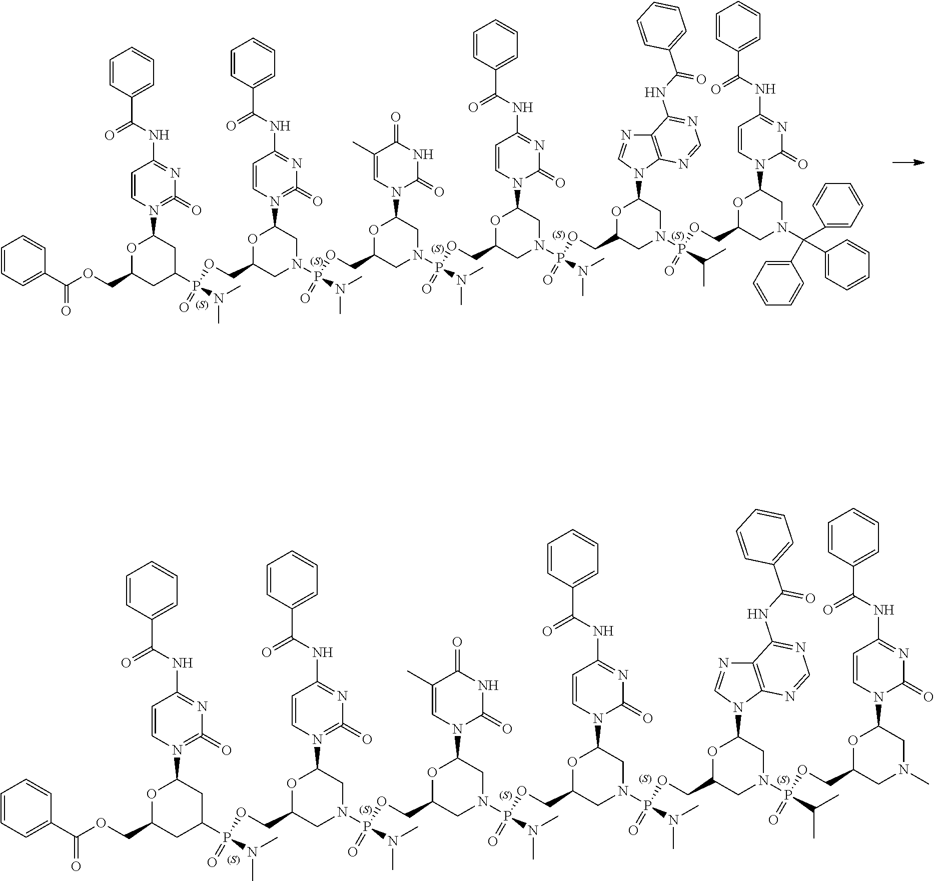

To a flask with the dried evaporated solids from previous step (6-mer) was added DCM (3.2 mL) and ethanol (155 μL). After the solids dissolved completely, added TFA (71.7 μL). The mixture was stirred for 2 h and the reaction was not completed according to HPLC analysis. Added additional 50 μL TFA and continued to stir for additional 6h. Added EtOAc (2.9 mL) then MTBE (11 mL). The resulting solids were filtered and rinsed with 4:1 MTBE/EtOAc (12 mL). Isolated ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S, 6R)-6-(4-benzamido-2-oxopyrimidin-1(2H)-yl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (295 mg). MS (ESI) m/z: Calcd for C₁₀₈H₁₂₉N₃₀O₂₉P₅: [(M+2H)/2]+1233.92; Found: 1233.68.

Synthesis of 7-mer:

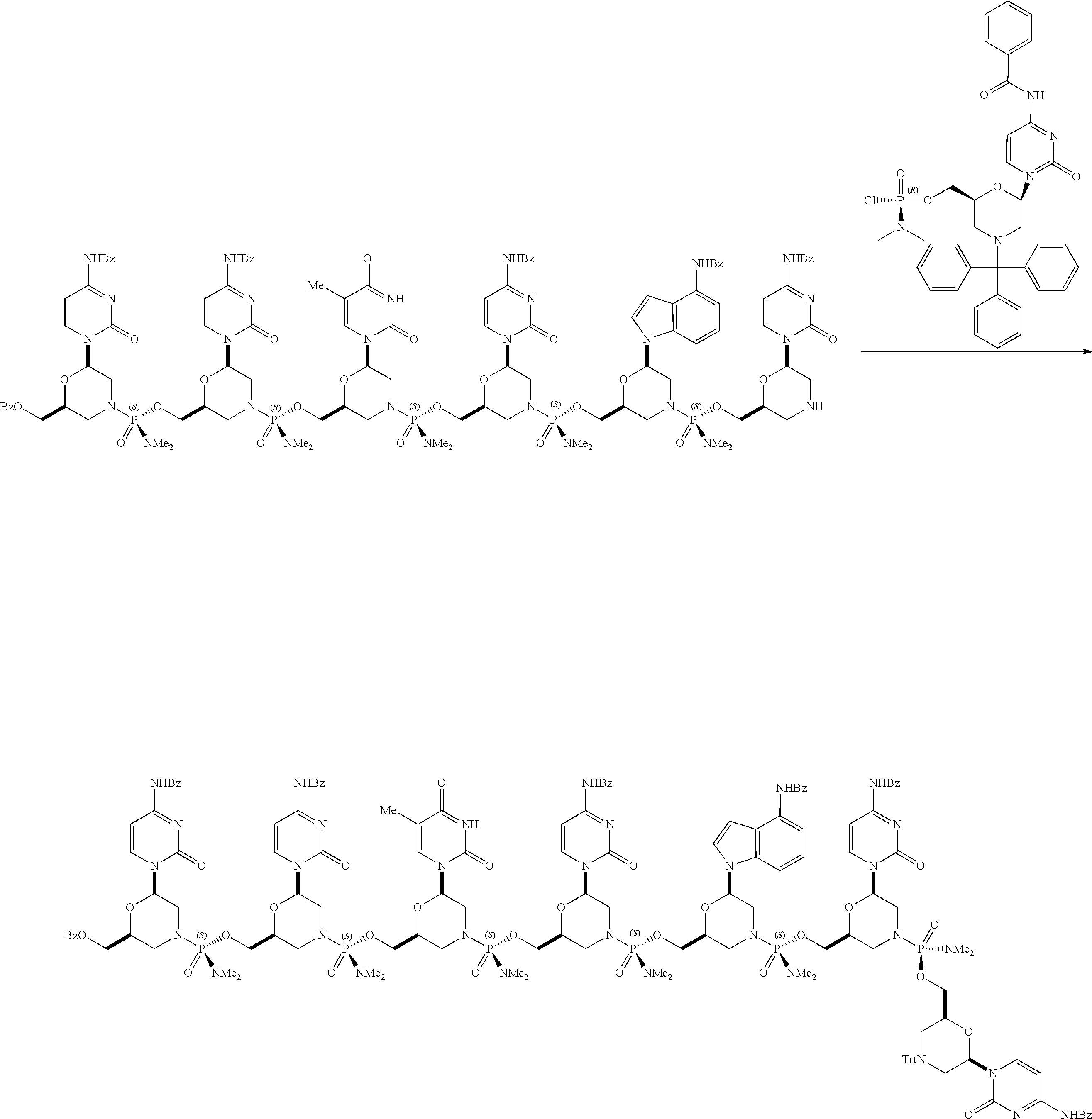

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (295 mg) was added 1,3-Dimethyl-2-imidazolidinone (2.9 mL) and then 1,2,2,6,6-pentamethylpiperidine (65.6 μL) at rt. ((2S,6R)-6-(4-benzamido-2-oxopyrimidin-1(2H)-yl)-4-tritylmorpholin-2-yl)methyl (R)-dimethylphosphoramidochloridate was added at rt as a solid (100 mg). The reaction was stirred for 3 h at rt. Upon completion by HPLC analysis, added EtOAc (5.9 mL) and MTBE (11.8 mL) over 2-3 min each. The solids were filtered and rinsed with MTBE 2×5 mL. After drying with air flow for 2-3 min the solids were transferred to a flask and dried under vacuum for 1h to afford 7-mer (430 mg). MS (ESI) m/z: Calcd for C₁₄₅H₁₆₅N₃₅O₃₄P₆: [(M+2H)/2]+1564.85; Found: 1564.77. Deprotection of 7-mer:

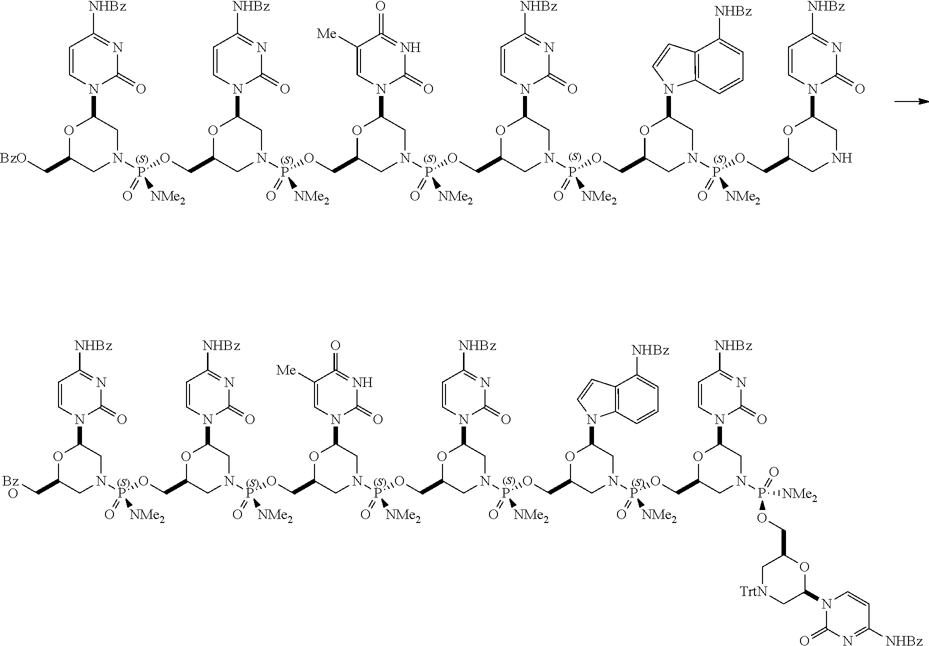

To a flask with ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(4-benzamido-2-oxopyrimidin-1(2H)-yl)-4-tritylmorpholin-2-yl)methoxy)(dimethylamino)phosphoryl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (374 mg) was added DCM (3.0 mL) and ethanol (140 μL). Added TFA (138 μL) dropwise at rt over 30 sec. After 30 minutes, added EtOAc (7.5 mL) and added MTBE (7.5 mL). Filtered and rinsed with MTBE 2×3 mL. The solids were dried in the filter funnel under air flow and then transferred to a flask. Dissolved in DCM (3.9 mL) and EtOH (140 μL), and added 1,2,2,6,6-pentamethylpiperidine (109 μL). After 10 minutes, the solution was treated with added EtOAc (7.5 mL) and MTBE (7.5 mL). The resulting solids were filtered and rinsed with MTBE 2×3 mL. The solids were dried in funnel and then transferred to a flask, dried under vacuum to obtain total ((2S,6R)-6-(4-benzamido-2-oxopyrimidin-1(2H)-yl)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-4-((S)-(((2S,6R)-6-(4-benzamido-2-oxopyrimidin-1(2H)-yl)-4-((S)-(((2S,6R)-6-(4-benzamido-2-oxopyrimidin-1(2H)-yl)morpholin-2-yl)methoxy)(dimethylamino)phosphoryl)morpholin-2-yl)methoxy)(dimethylamino)phosphoryl)-6-(6-benzamido-9H-purin-9-yl)morpholin-2-yl)methoxy)(dimethylamino)phosphoryl)morpholin-2-yl)methoxy)(dimethylamino)phosphoryl)-6-(5-methyl-2,4-dioxo-3,4-dihydropyrimidin-1(2H)-yl)morpholin-2-yl)methoxy)(dimethylamino)phosphoryl)morpholin-2-yl)methoxy)(dimethylamino)phosphoryl)morpholin-2-yl)methyl benzoate (345 mg). MS (ESI) m/z: Calcd for C₁₂₆H₁₅₁N₃₅O₃₄P₆: [(M+2H)/2]+1443.48; Found: 1444.

From 8-mer to 25-mer, general procedures were used for coupling, deprotection and free basing:

General procedure A for coupling: To a flask with dried PMO oligonucleotide (free base PMO oligonucleotide) (1 wt, 1 equiv.) was added 1,3-dimethyl-2-imidazolidinone (6-10 volumes compared to free base PMO oligonucleotide) and then 1,2,2,6,6-pentamethylpiperidine (3-5 equiv.). The mixture was stirred and sonicated until all solids dissolved. Activated monomer (R)-dimethylphosphoramidochloridate (1.3-2.5 equiv.) was added as a solid in a single portion under N₂ atmosphere. The reaction mixture was stirred for minimum of 3h (18-24h at stages 15-25mer) and monitored for completion by UPLC MS (>99.5% target by UV or no detectable starting material mass). Additional (R)-dimethylphosphoramidochloridate may be added if target conversion criteria is not reached. Upon completion, the reaction mixture was charged with EtOAc (10-40 vols) and MTBE (10-40 volumes as compared to free base PMO oligonucleotide) to form a white precipitate. The solids were, filtered on a sintered funnel, rinsed with EtOAc/MTBE 1:1, dried under vacuum and collected to afford “trityl-protected PMO oligonucleotide” for the next step. General yield 90-100%.

### General Procedure B for Trityl Deprotection and Free Basing:

Trityl deblock solution was prepared as follows: To a flask were added DCM (8 mL), 2,2,2-trifluoroethanol (2 mL), 4-cyanopyridine (100 mg), ethanol (100 μL) and trifluoroacetic acid (105 mg) in that order. The solution was mixed until all components are dissolved and then used in deprotection as is.

Step 1—trityl deprotection: To a flask with “trityl-protected PMO oligonucleotide” (1 wt, 1 equiv.) was added trityl deblock solution (8 volumes compared to trityl-protected PMO oligonucleotide mass). The reaction mixture was stirred for 5-30 minutes and monitored by UPLC MS. Upon completion (>99.5% target), added EtOAc (10-40 vols) and MTBE (10-40 volumes) to form a white precipitate. The solids were filtered on a sintered funnel, rinsed with EtOAc/MTBE 1:1, dried under vacuum and collected to afford “TFA salt PMO oligonucleotide” for the next step.

Step 2—free basing: To a flask with “TFA salt PMO oligonucleotide” (1 wt, 1 equiv.) was added DCM (7-10 vols compared to TFA salt PMO oligonucleotide mass) and EtOH (0.3-0.5 vol). The solution was treated with 1,2,2,6,6-pentamethylpiperidine (5 equiv.). The reaction mixture was stirred for 5-10 minutes and then treated with EtOAc (10-40 vols) and MTBE (10-40 volumes) to form a white precipitate. The solids were rinsed with EtOAc/MTBE 1:1, dried under vacuum and collected for the next step.

Coupling to 8-mer:

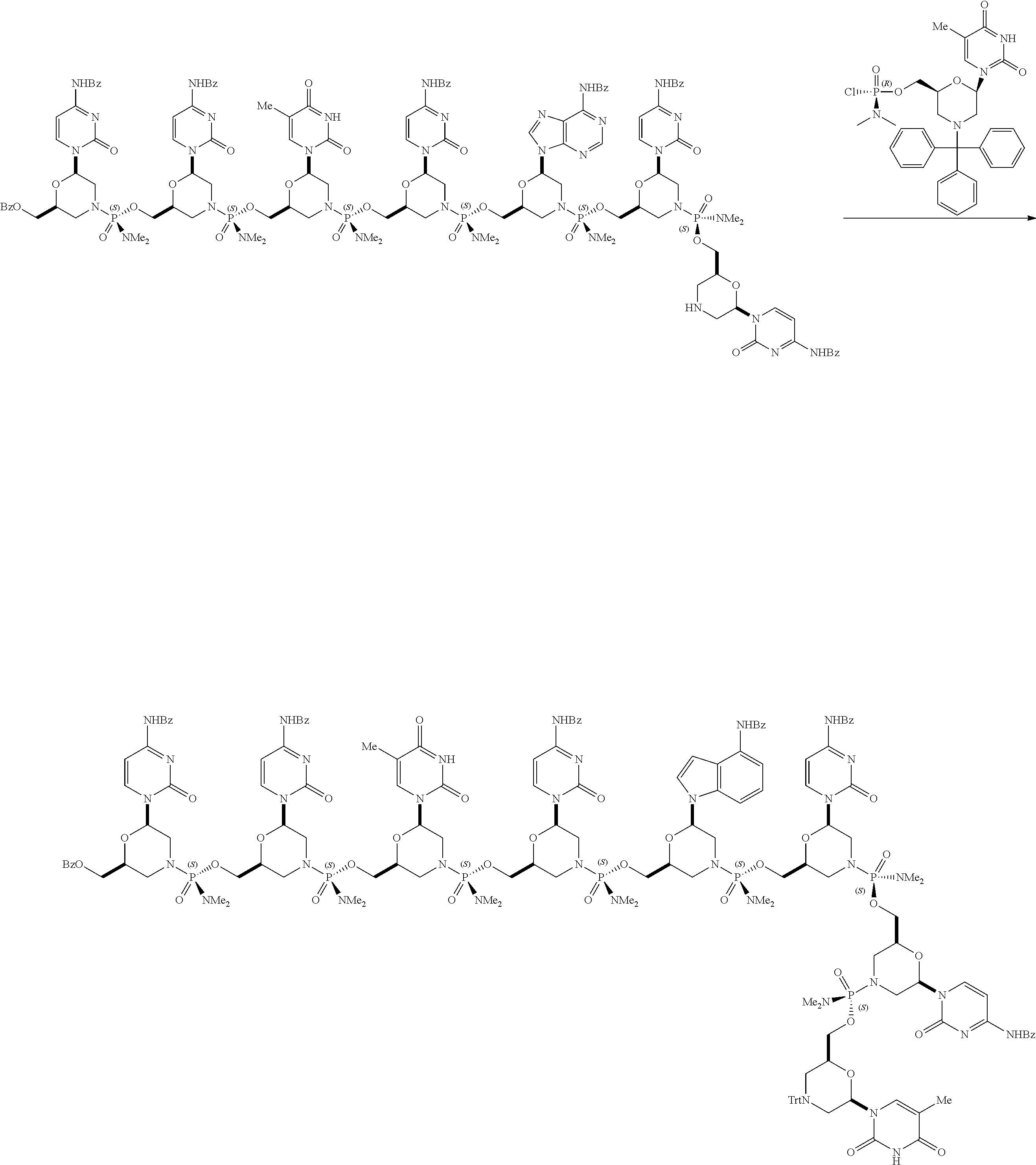

Using General Procedure A: reaction of 7-mer (340 mg) with ((2S,6R)-6-(5-methyl-2,4-dioxo-3,4-dihydropyrimidin-1(2H)-yl)-4-tritylmorpholin-2-yl)methyl (R)-dimethylphosphoramidochloridate (86 mg) afforded 8-mer (403 mg). MS (ESI) m/z: Calcd for C₁₅₇H₁₈₄N₃₉O₃₉P₇: [M+2H/2]+1730.09; Found: 1730.

Deprotection of 8-mer:

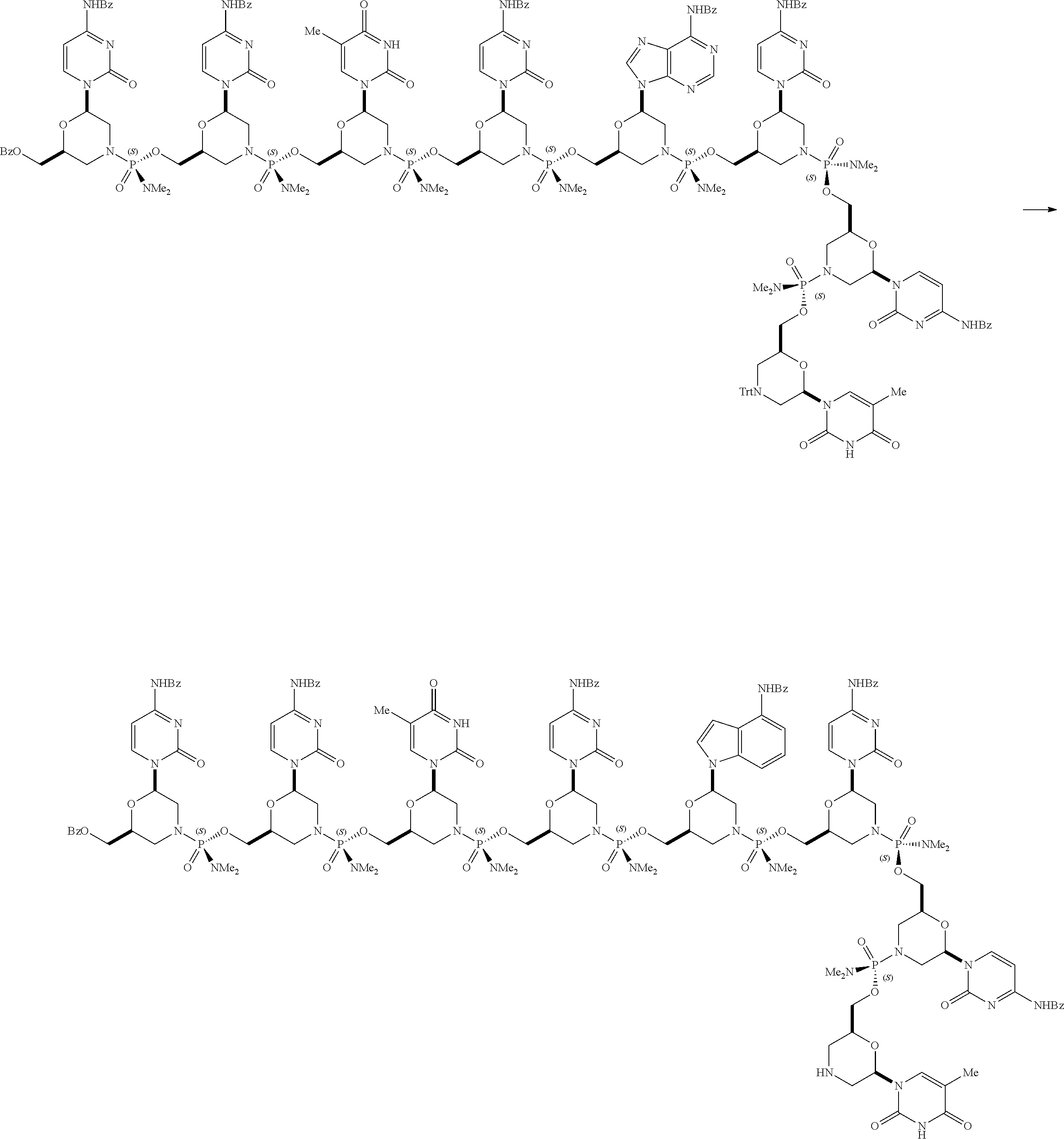

Using General Procedure B: reaction of trityl protected 8-mer (380 mg) afforded free base 8-mer (353 mg). MS (ESI) m/z: Calcd for C₁₃₈H₁₇₀N₃₉O₃₉P₇: [M+2H/2]+1608.53; Found: 1609.

Coupling to 9-mer:

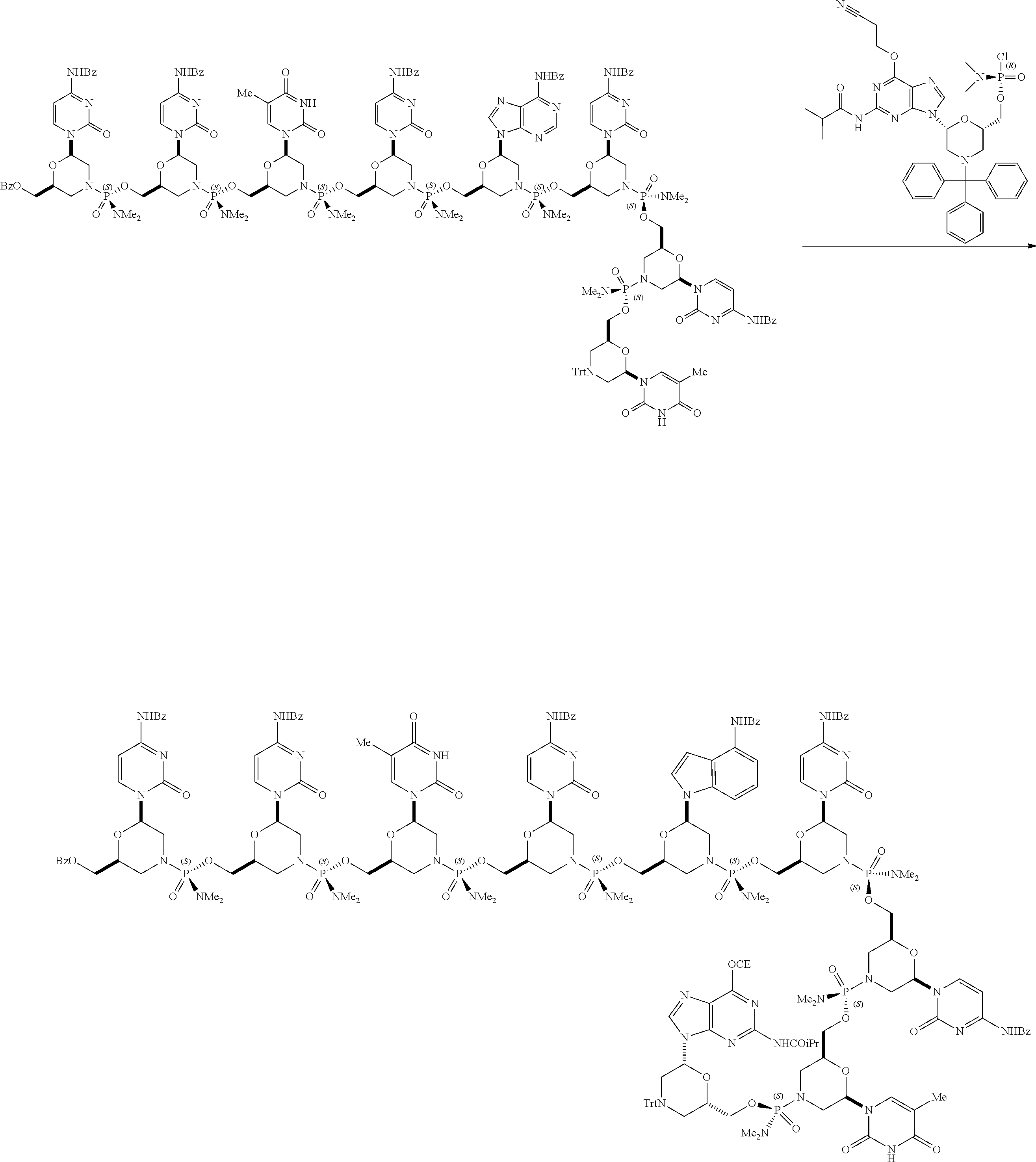

Using General Procedure A: reaction of 8-mer (370 mg) with ((2S,6R)-6-(6-(2-cyanoethoxy)-2-isobutyramido-9H-purin-9-yl)-4-tritylmorpholin-2-yl)methyl (R)-dimethylphosphoramidochloridate (105 mg) afforded 9-mer (453 mg).

Deprotection of 9-mer:

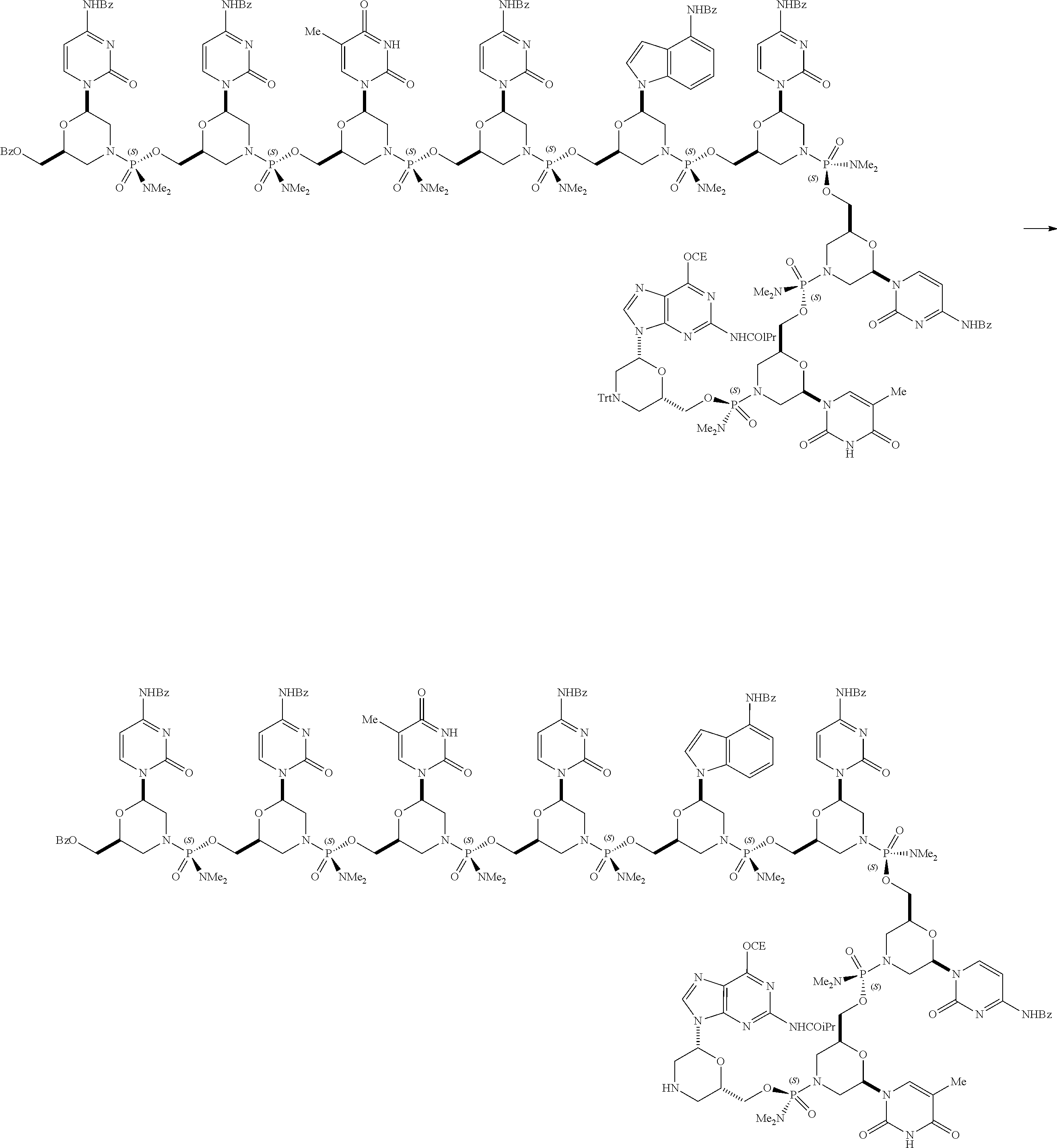

Using General Procedure B: reaction of trityl protected 9-mer (453 mg) afforded free base 9-mer (411 mg).

Coupling to 10-mer:

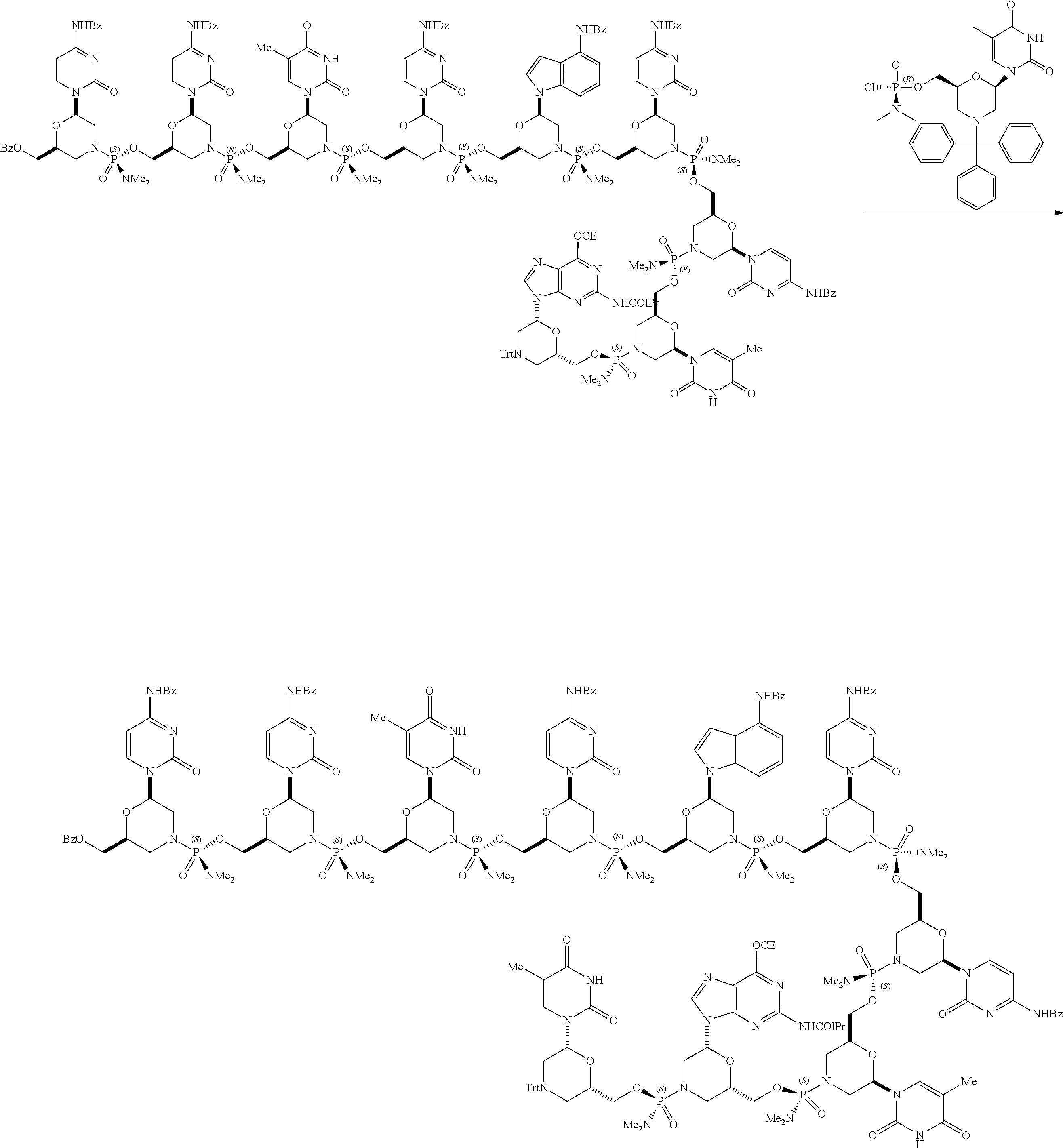

Using General Procedure A: reaction of 9-mer (405 mg) with ((2S,6R)-6-(5-methyl-2,4-dioxo-3,4-dihydropyrimidin-1(2H)-yl)-4-tritylmorpholin-2-yl)methyl (R)-dimethyl phosphoramidochloridate (80 mg) afforded 10-mer (469 mg). MS (ESI) m/z: Calcd for C₁₈₈H₂₃₀N51049P₉: [M+3H/3]+1422.8; Found: 1423.3.

Deprotection of 10-mer:

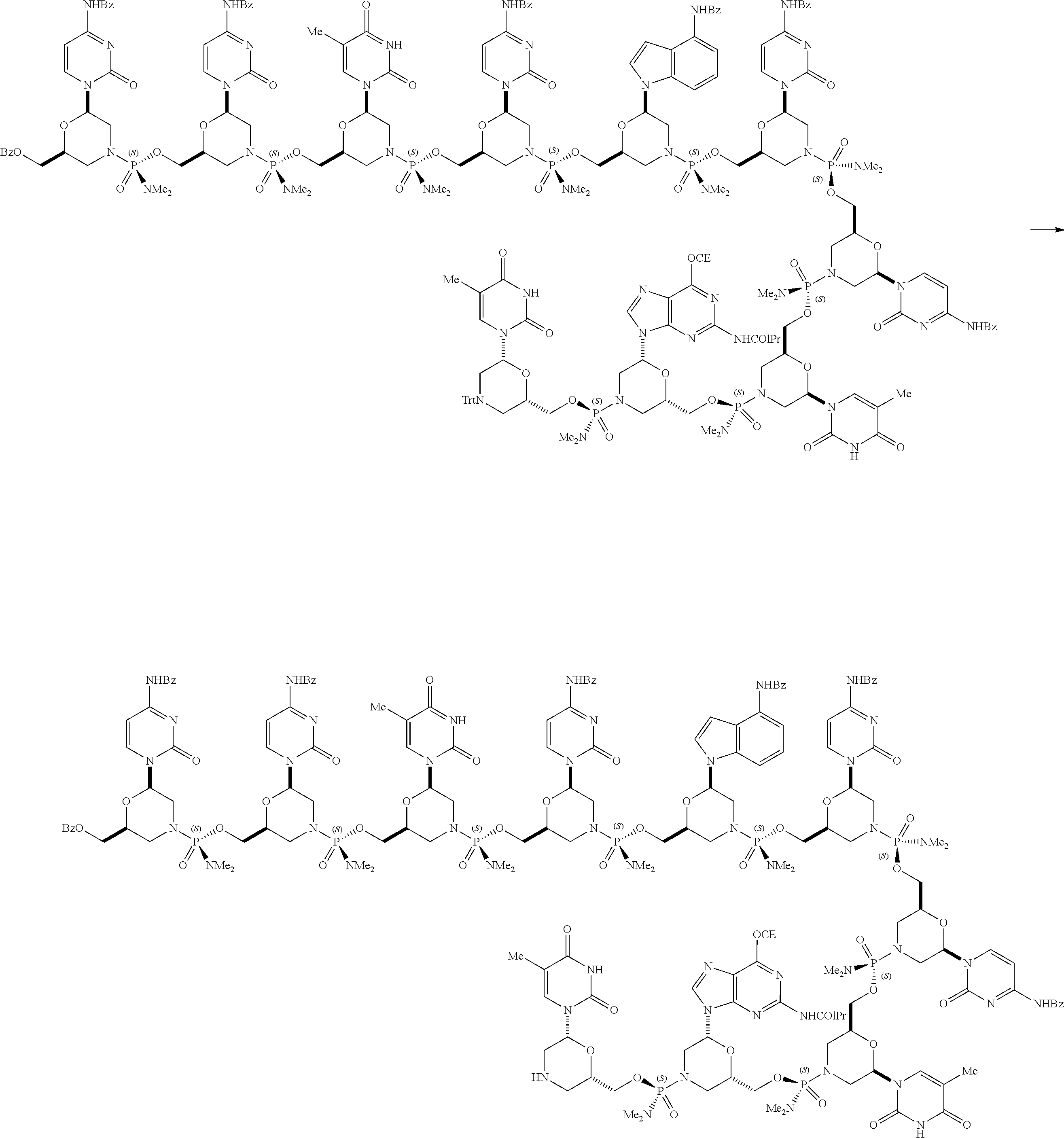

Using General Procedure B: reaction of trityl protected 10-mer (450 mg) afforded free base 10-mer (435 mg).

Coupling to 11-mer:

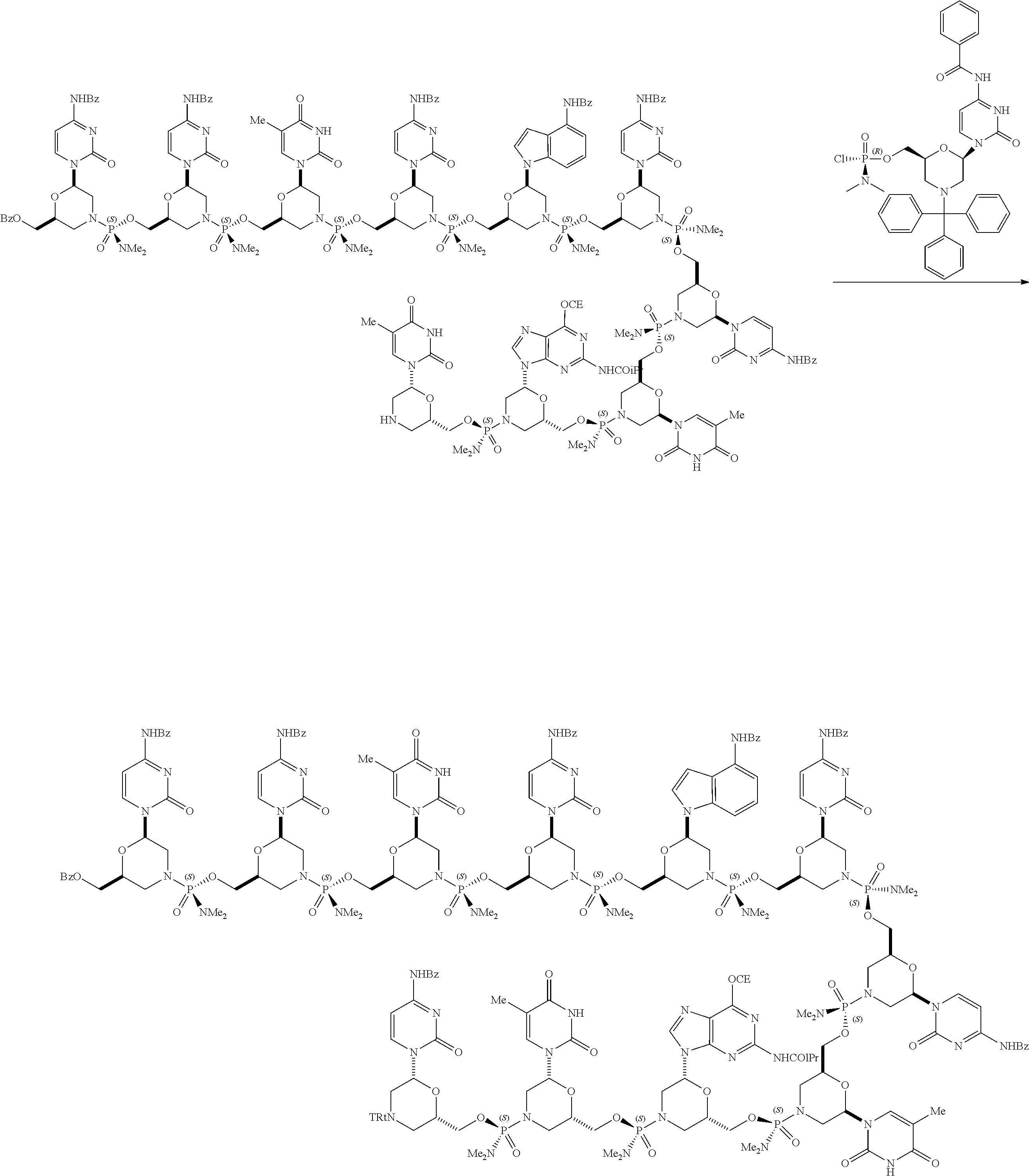

Using General Procedure A: reaction of 10-mer (435 mg) with ((2S,6R)-6-(4-benzamido-2-oxopyrimidin-1(2H)-yl)-4-tritylmorpholin-2-yl)methyl (R)-dimethylphosphoramidochloridate (98 mg) afforded 11-mer (512 mg).

Deprotection of 11-mer:

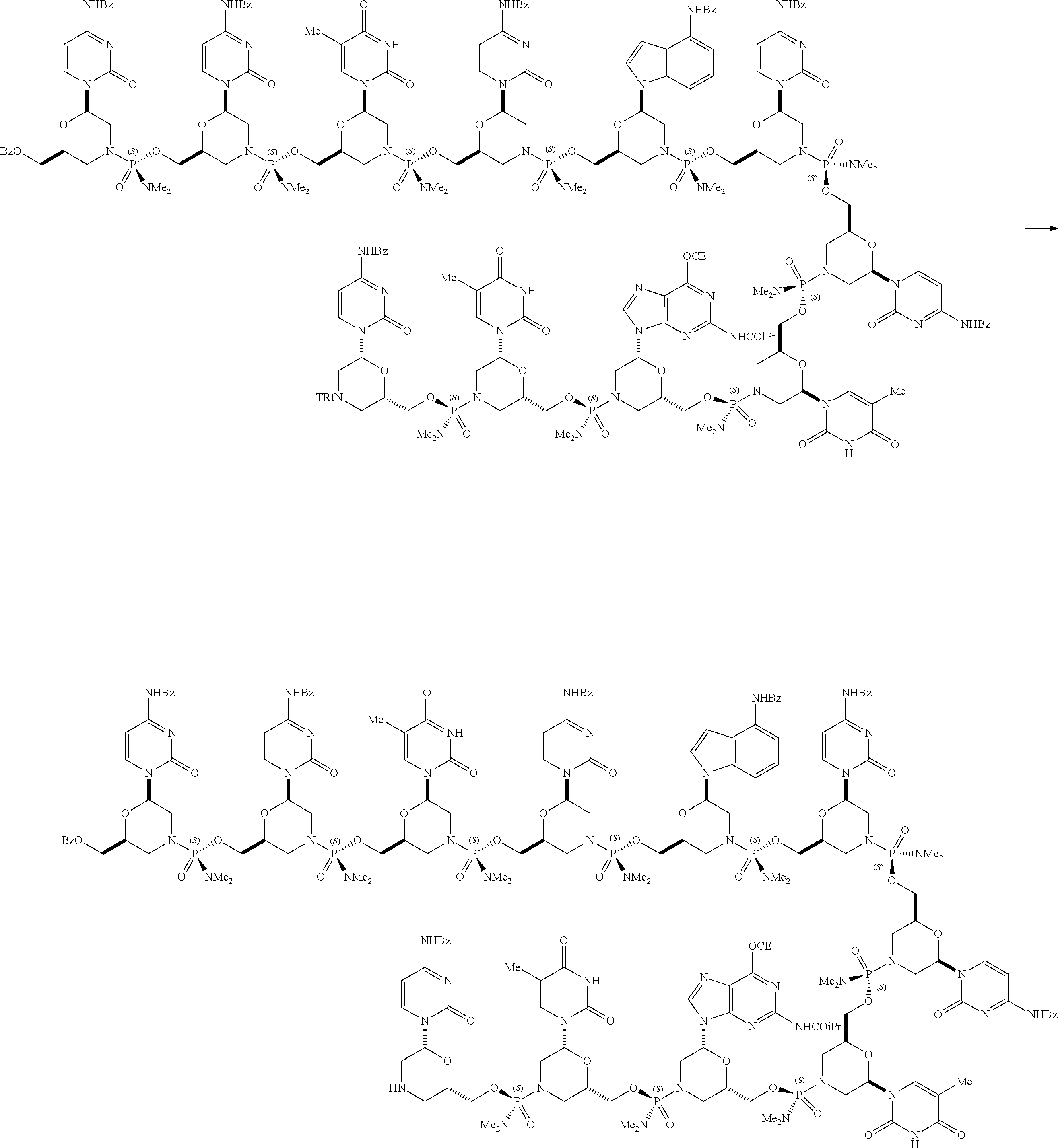

Using General Procedure B: reaction of trityl protected 11-mer (500 mg) afforded free base 11-mer (481 mg).

Coupling to 12-mer:

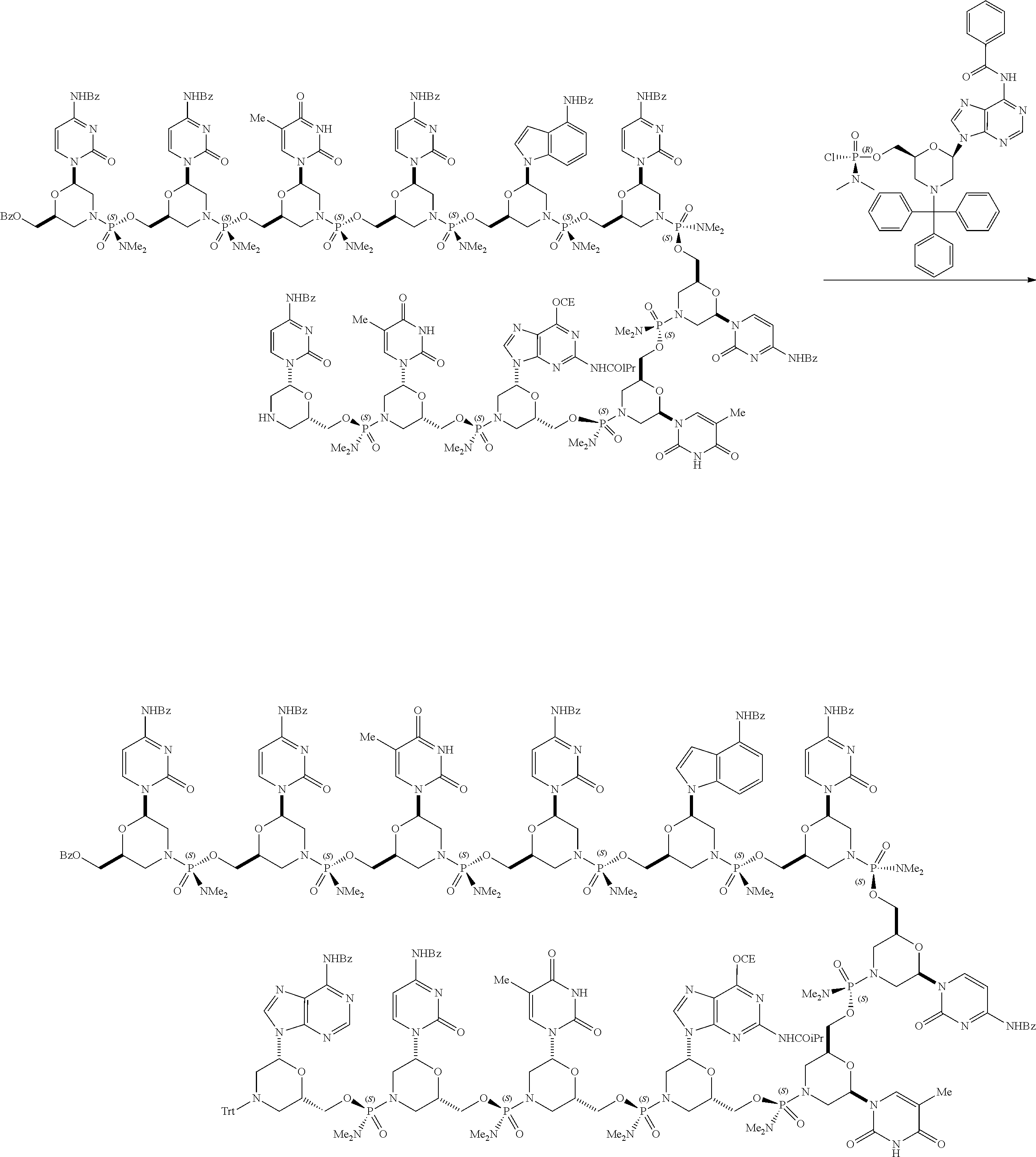

Using General Procedure A: reaction of 11-mer (481 mg) with ((2S,6R)-6-(6-benzamido-9H-purin-9-yl)-4-tritylmorpholin-2-yl)methyl (R)-dimethylphosphoramidochloridate (102 mg) afforded 12-mer (525 mg).

Deprotection of 12-mer:

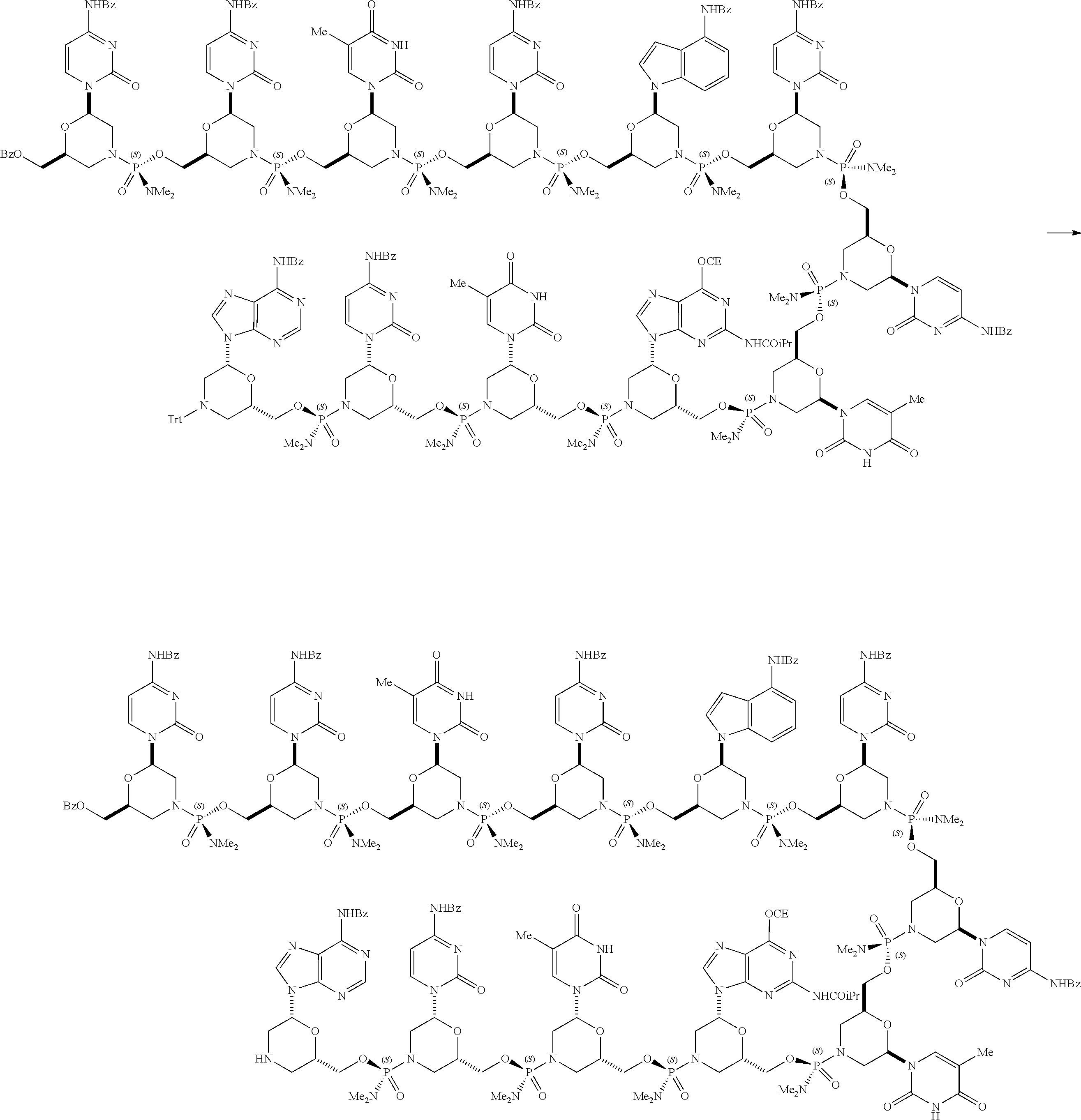

Using General Procedure B: reaction of trityl protected 12-mer (525 mg) afforded free base 12-mer (490 mg).

Coupling to 13-mer:

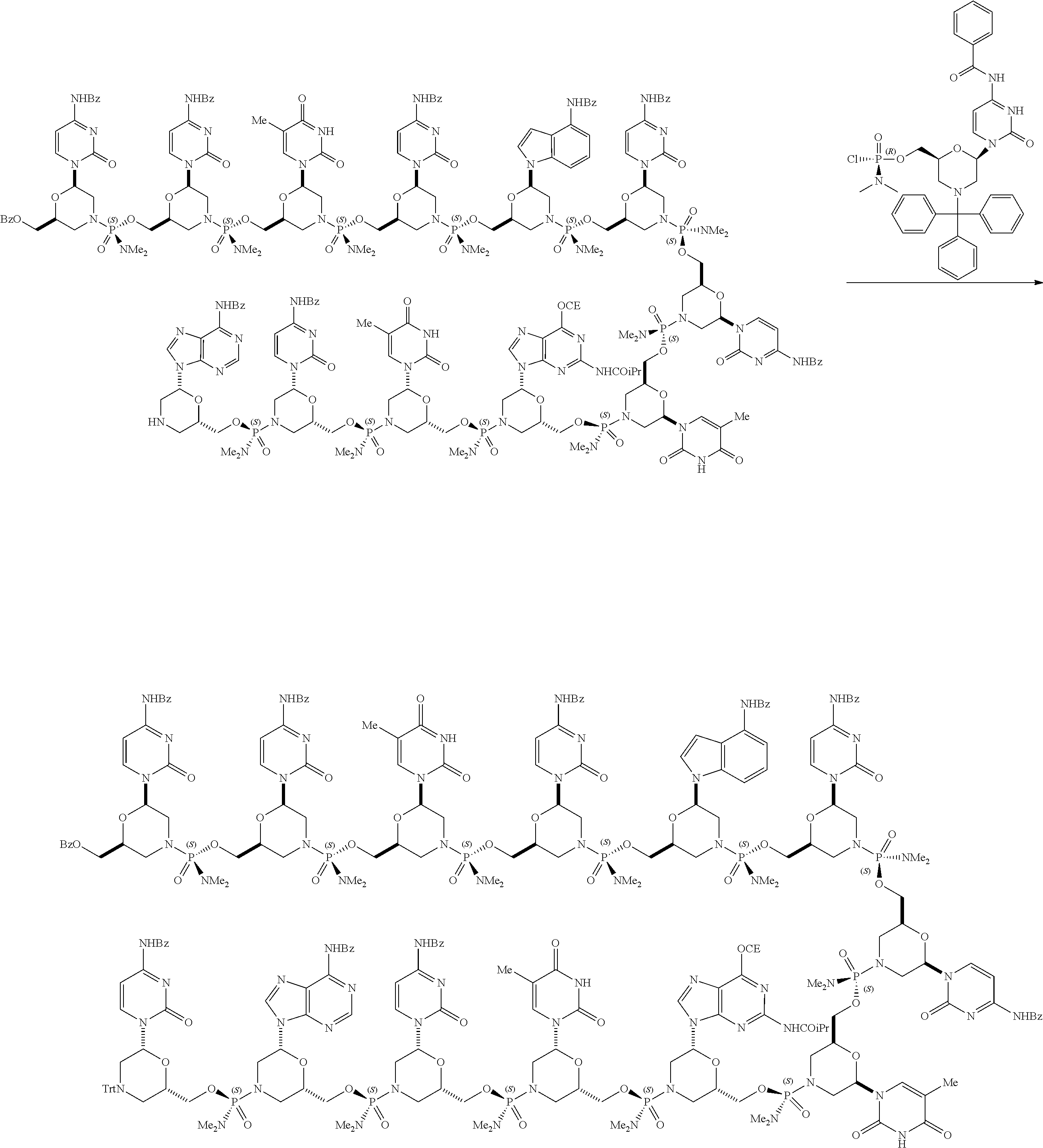

Using General Procedure A: reaction of 12-mer (484 mg) with ((2S,6R)-6-(4-benzamido-2-oxopyrimidin-1(2H)-yl)-4-tritylmorpholin-2-yl)methyl (R)-dimethylphosphoramidochloridate (86 mg) afforded 13-mer (550 mg).

Deprotection of 13-mer:

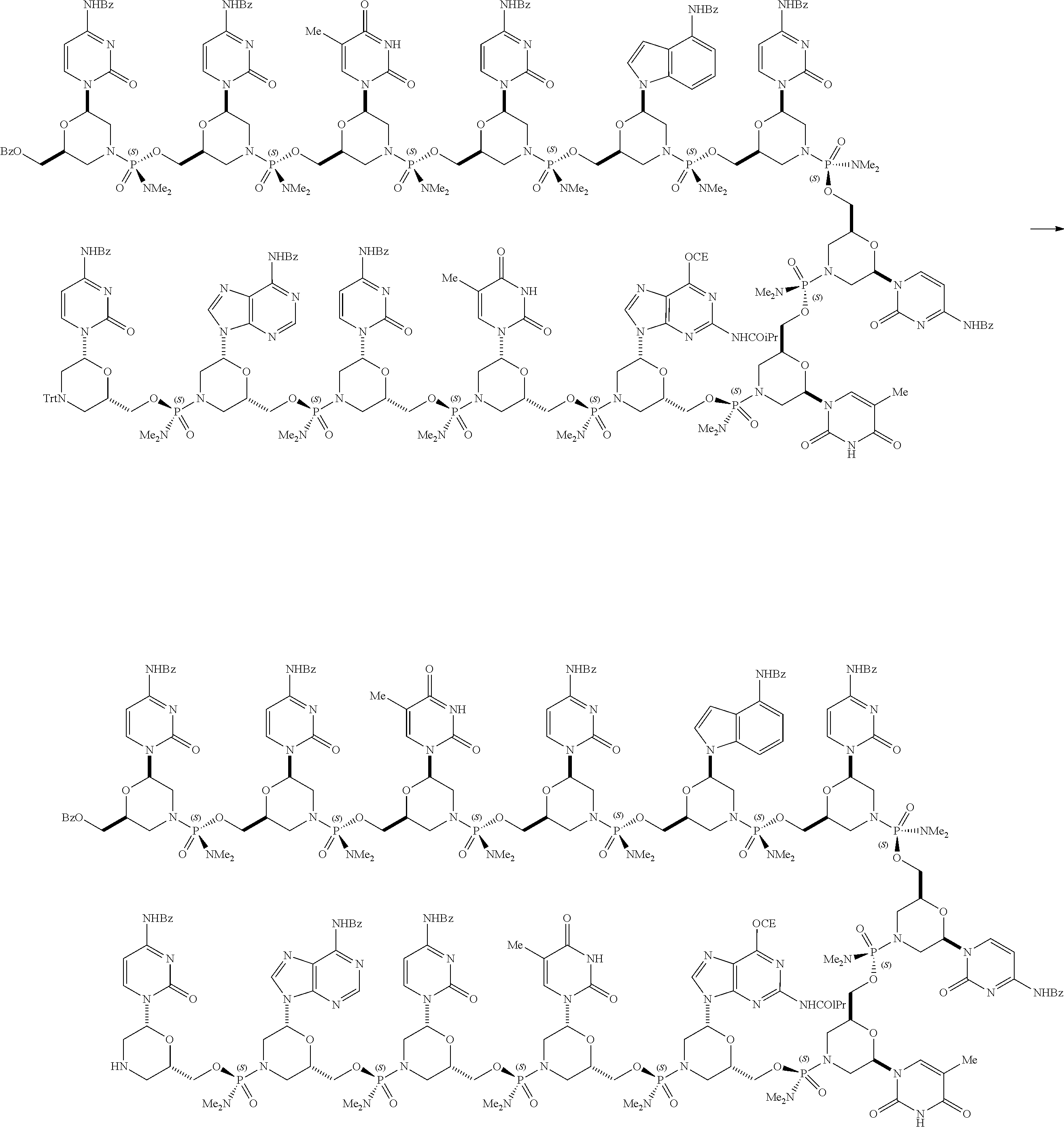

Using General Procedure B: reaction of trityl protected 13-mer (550 mg) afforded free base 13-mer (550 mg).

Coupling to 14-mer:

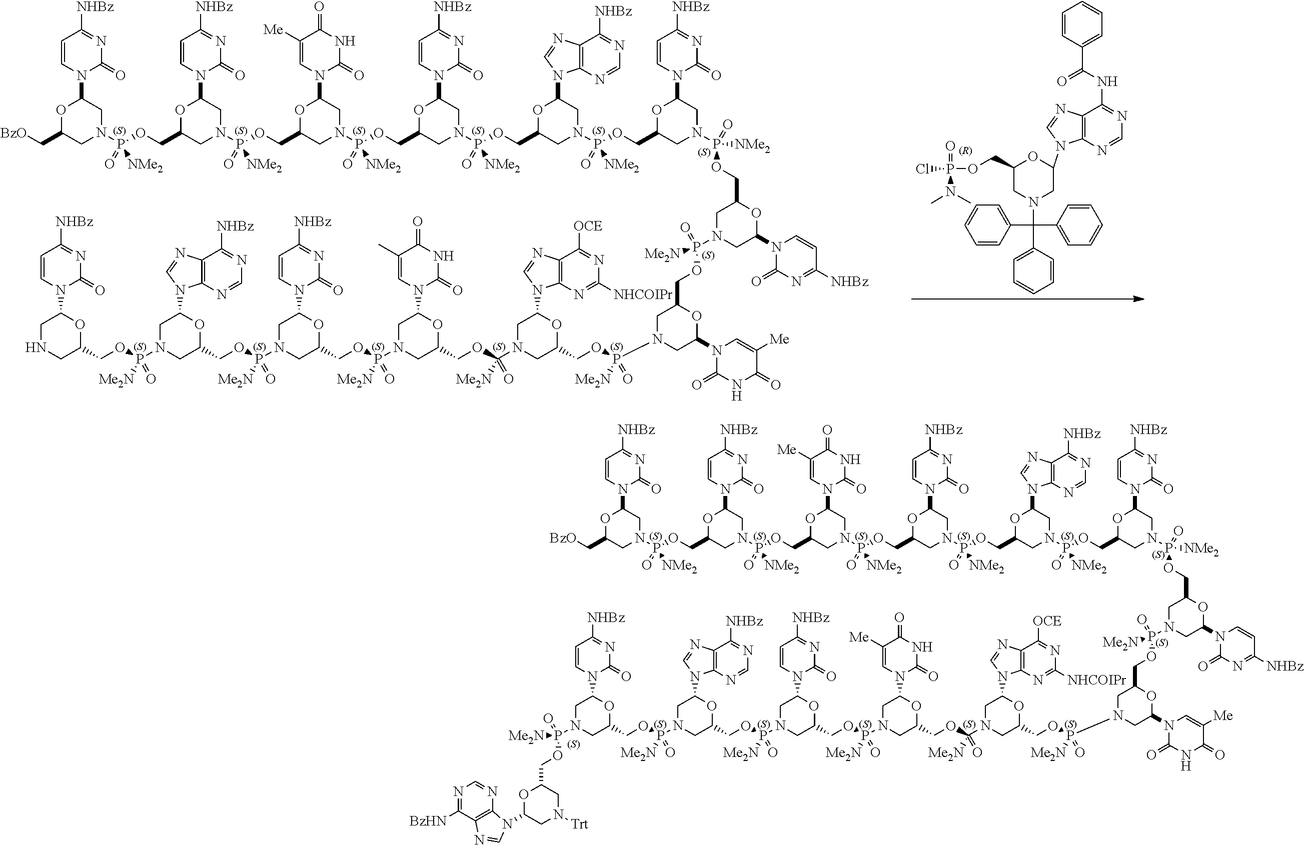

Using General Procedure A: reaction of 13-mer (550 mg) with ((2S,6R)-6-(6-benzamido-9H-purin-9-yl)-4-tritylmorpholin-2-yl)methyl (R)-dimethylphosphoramidochloridate (94 mg) afforded 14-mer (621 mg).

Deprotection of 14-mer:

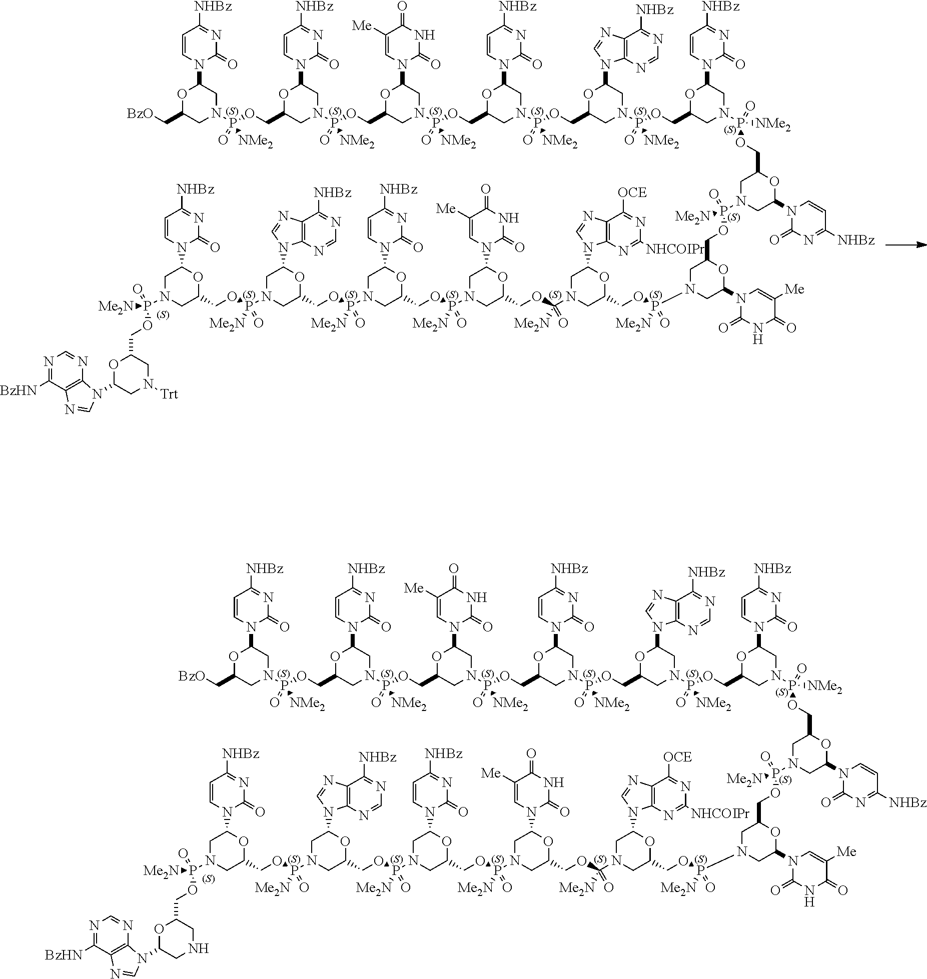

Using General Procedure B: reaction of trityl protected 14-mer (621 mg) afforded free base 14-mer (596 mg).

Coupling to 15-mer:

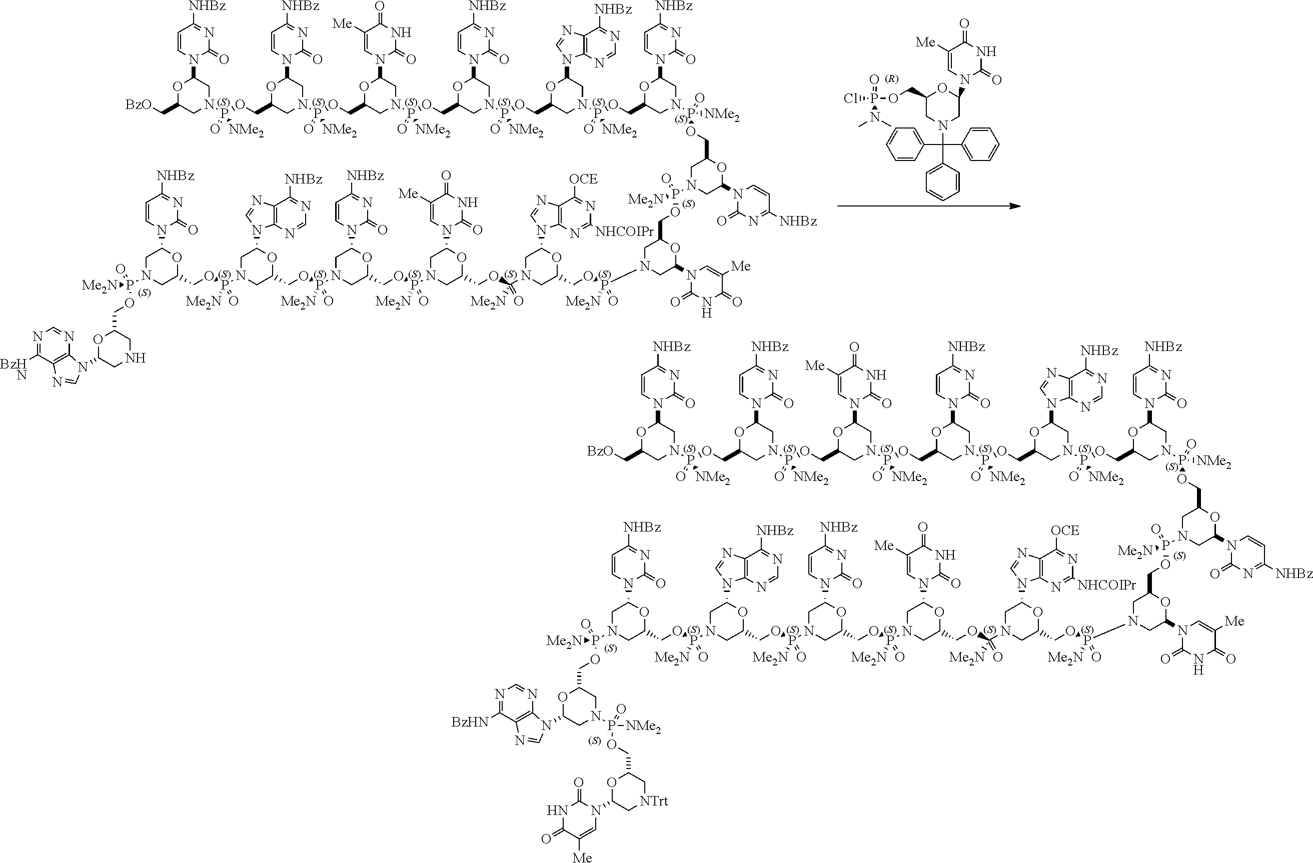

Using General Procedure A: reaction of 14-mer (596 mg) with ((2S,6R)-6-(5-methyl-2,4-dioxo-3,4-dihydropyrimidin-1(2H)-yl)-4-tritylmorpholin-2-yl)methyl (R)-dimethyl phosphoramidochloridate (84 mg) afforded 15-mer (655 mg). MS (ESI) m/z: Calcd for C₂₇₄H₃₃₇N₇₉O₇₂P₁₄: [M+4H/4]+1581.28; Found: 1582.

Deprotection of 15-mer:

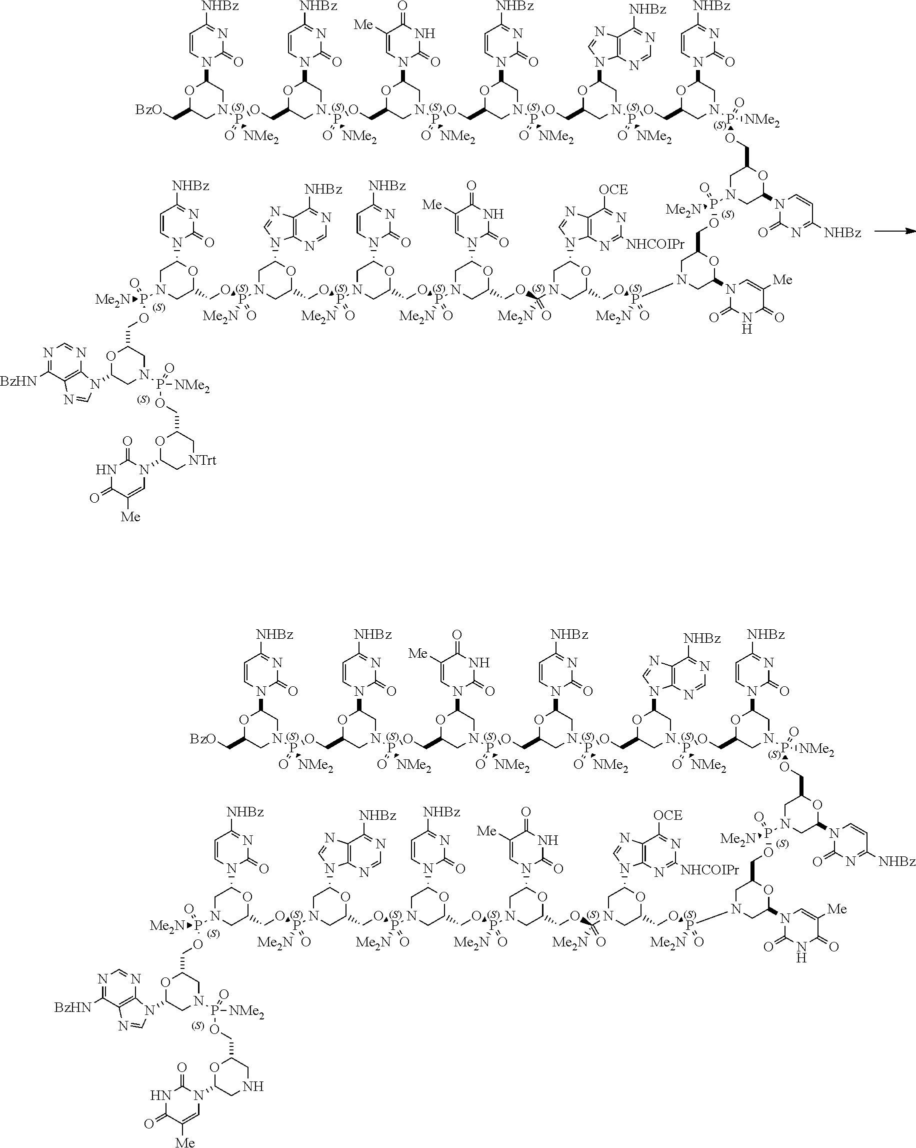

Using General Procedure B: reaction of trityl protected 15-mer (650 mg) afforded free base 15-mer (613 mg). MS (ESI) m/z: Calcd for C₂₅₅H₃₂₃N₇₉O₇₂P₁₄: [M+4H/4]+1520.76; Found: 1521.

Coupling to 16-mer:

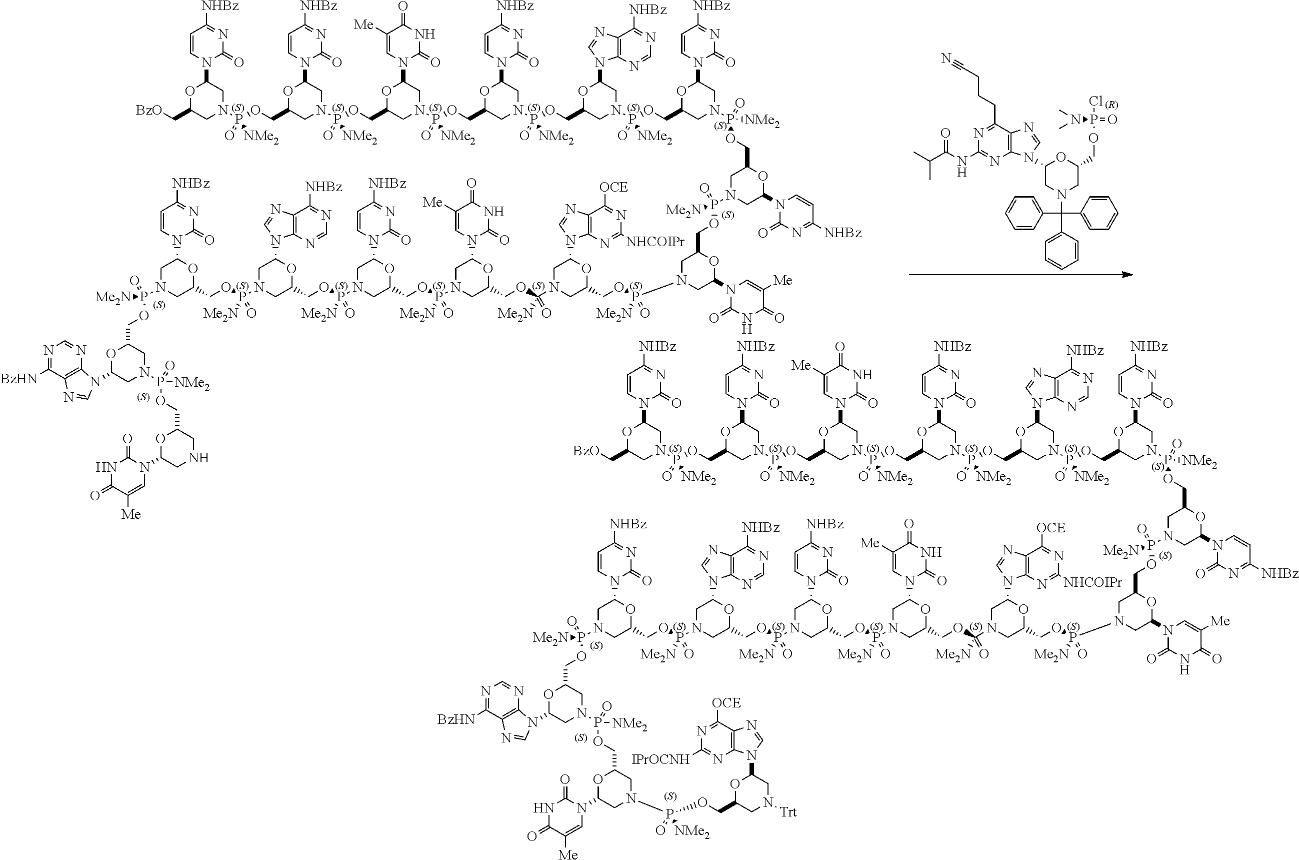

Using General Procedure A: reaction of 15-mer (613 mg) with ((2S,6R)-6-(6-(2-cyanoethoxy)-2-isobutyramido-9H-purin-9-yl)-4-tritylmorpholin-2-yl)methyl (R)-dimethyl phosphoramidochloridate (103 mg) afforded 16-mer (680 mg).

Deprotection of 16-mer:

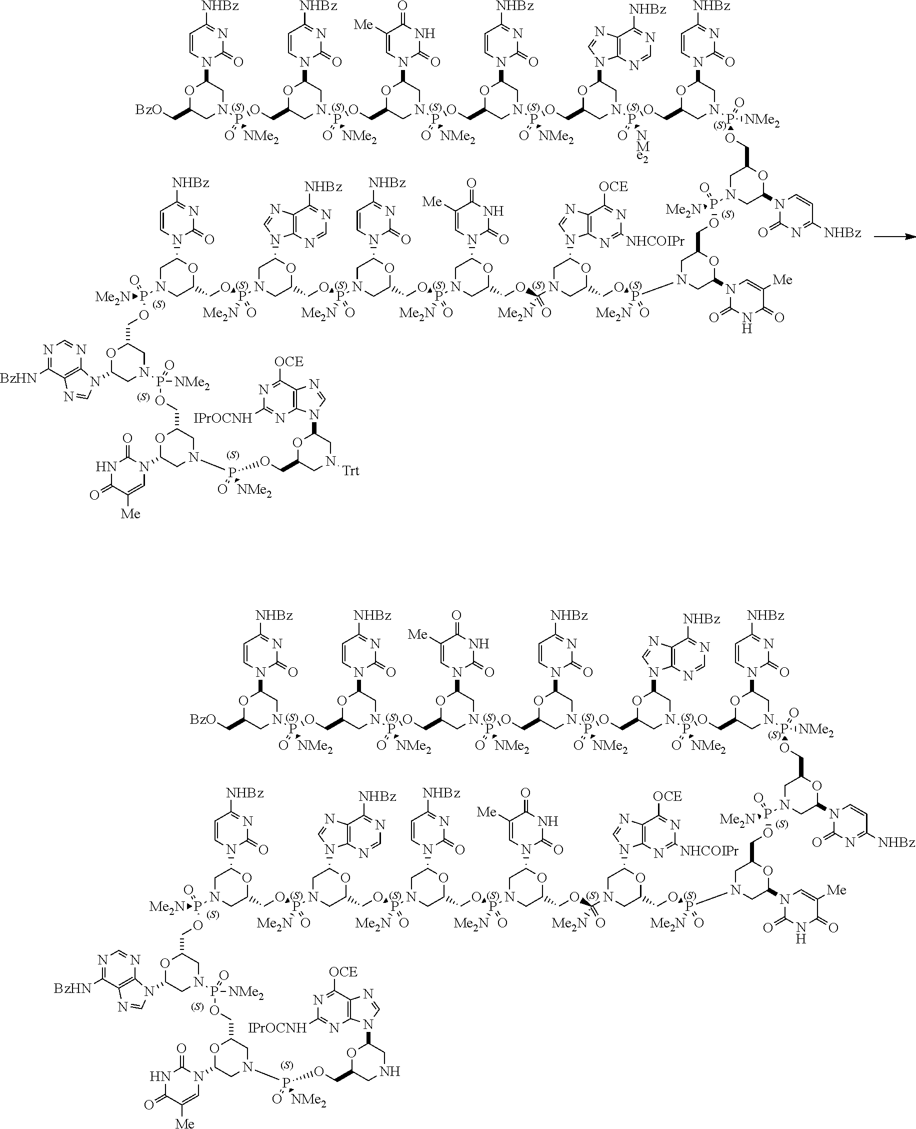

Using General Procedure B: reaction of trityl protected 16-mer (680 mg) afforded free base 16-mer (623 mg).

Coupling to 17-mer:

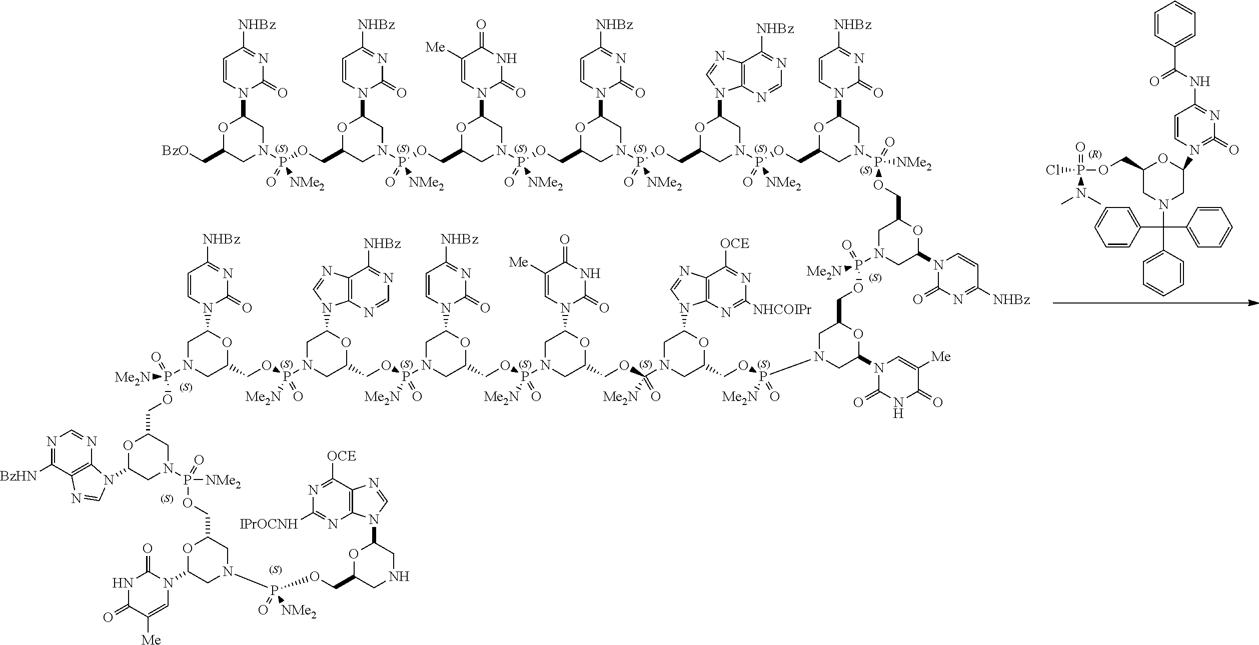

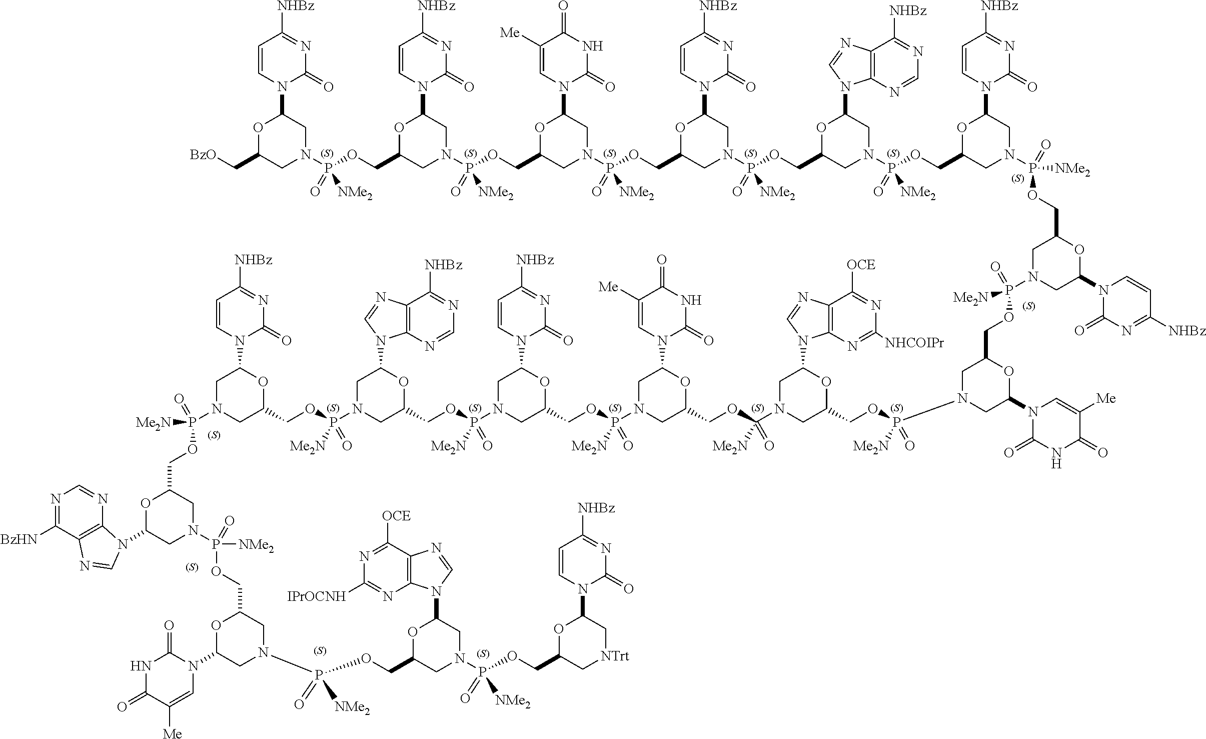

Using General Procedure A: reaction of 16-mer (623 mg) with ((2S,6R)-6-(4-benzamido-2-oxopyrimidin-1(2H)-yl)-4-tritylmorpholin-2-yl)methyl (R)-dimethylphosphoramidochloridate (93 mg) afforded 17-mer (690 mg).

Deprotection of 17-mer:

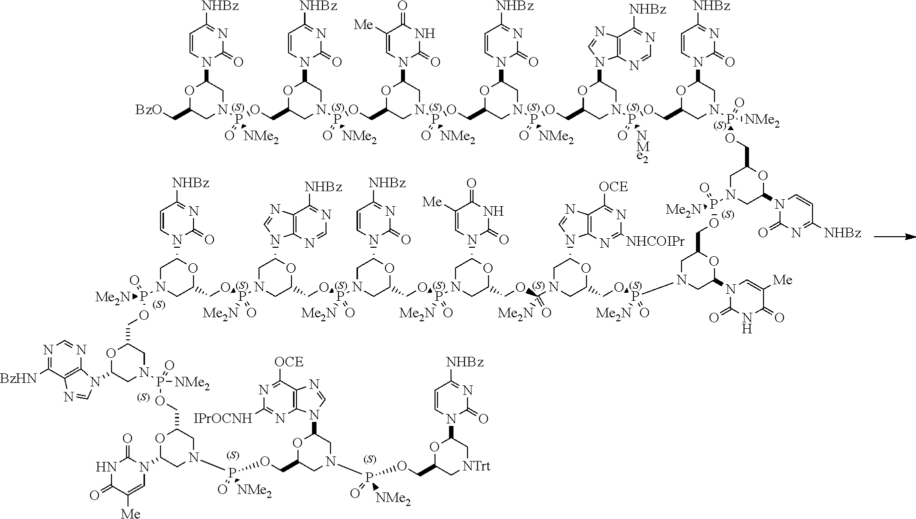

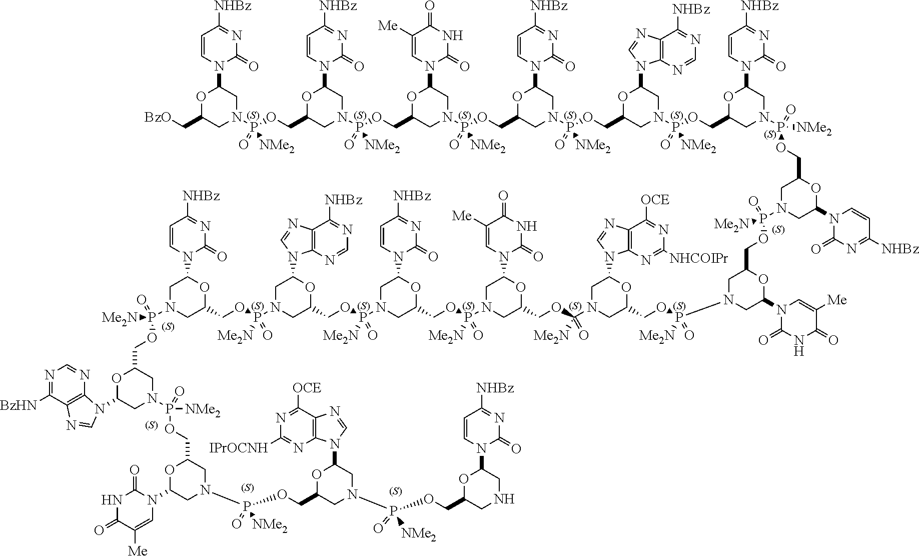

Using General Procedure B: reaction of trityl protected 17-mer (690 mg) afforded free base 17-mer (670 mg).

Coupling to 18-mer:

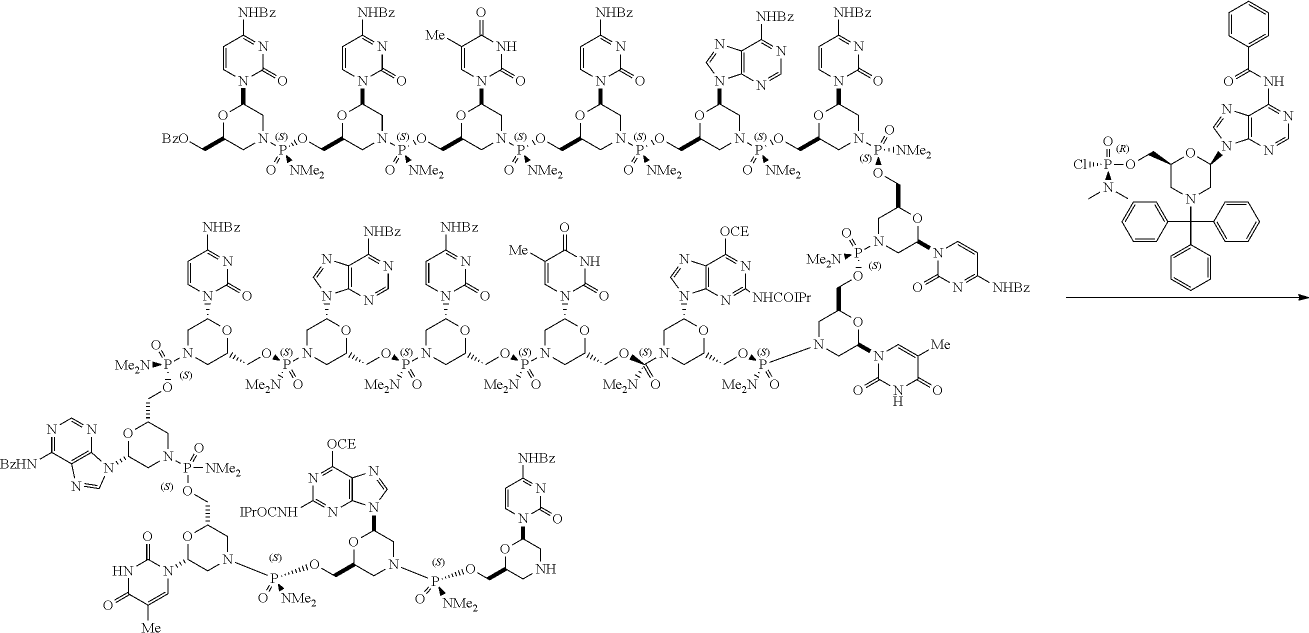

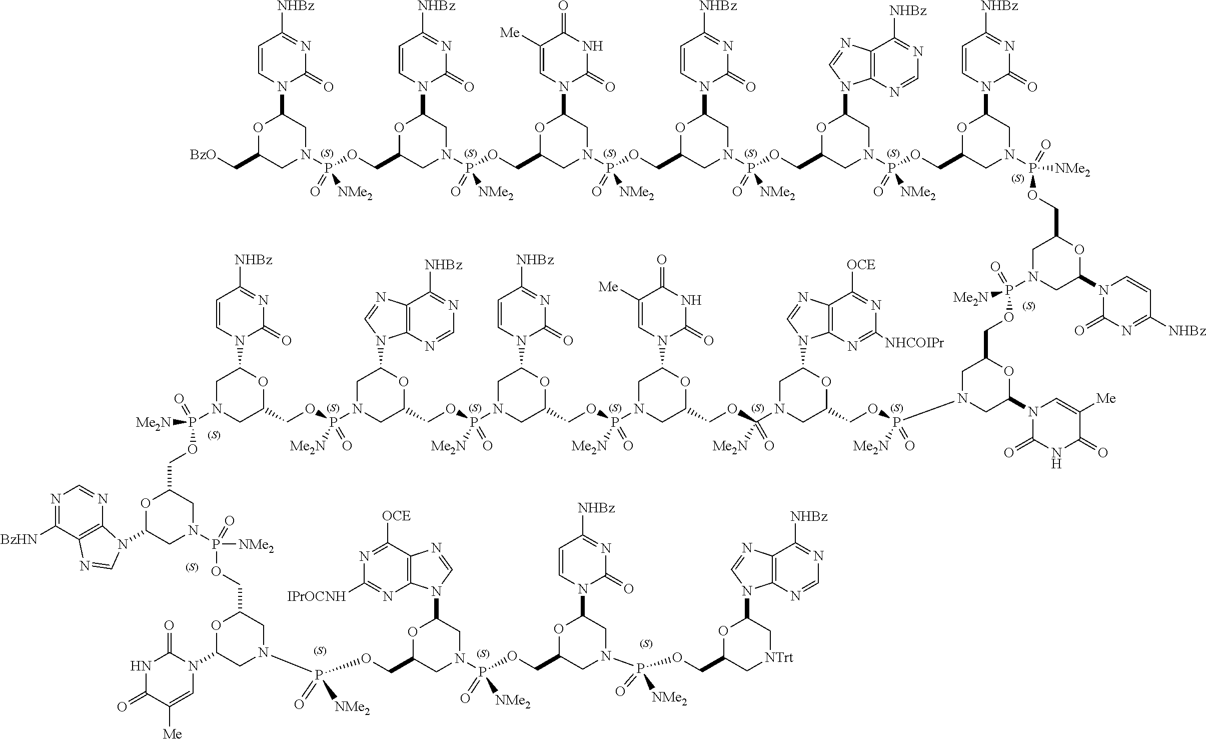

Using General Procedure A: reaction of 17-mer (673 mg) with ((2S,6R)-6-(6-benzamido-9H-purin-9-yl)-4-tritylmorpholin-2-yl)methyl (R)-dimethylphosphoramidochloridate (98 mg) afforded 18-mer (740 mg).

Deprotection of 18-mer:

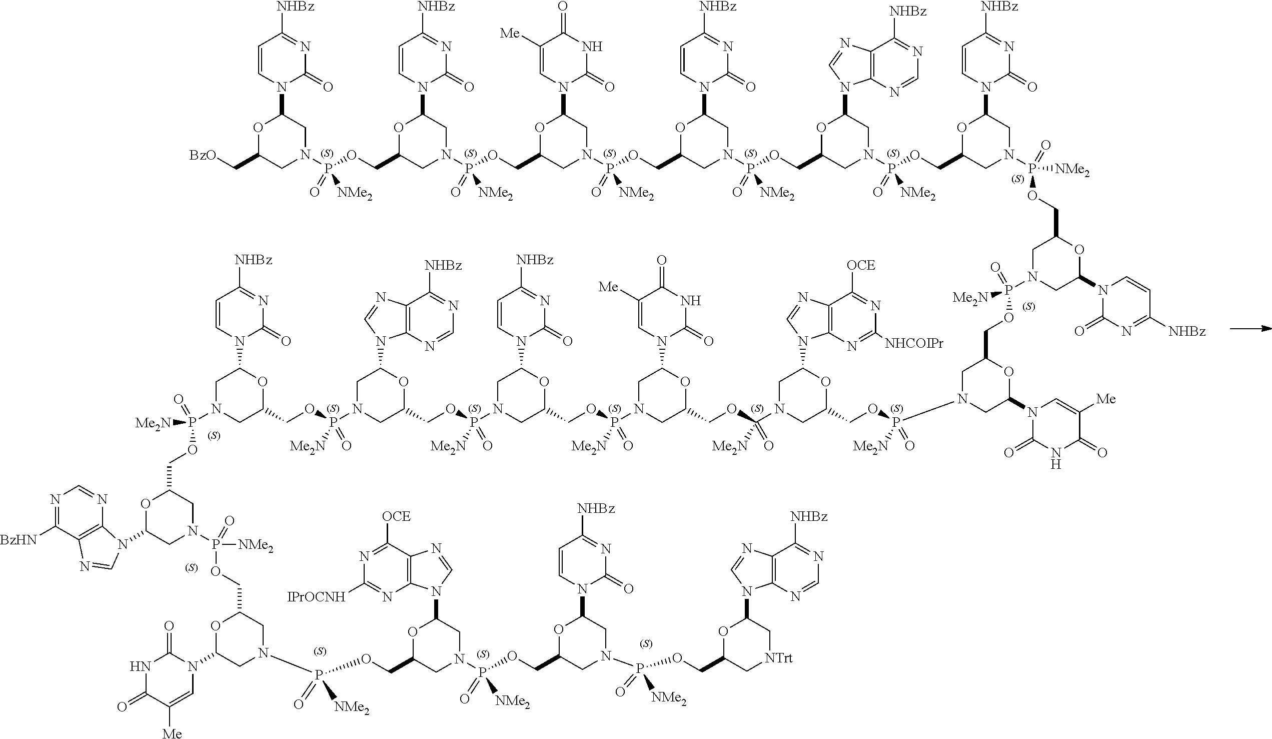

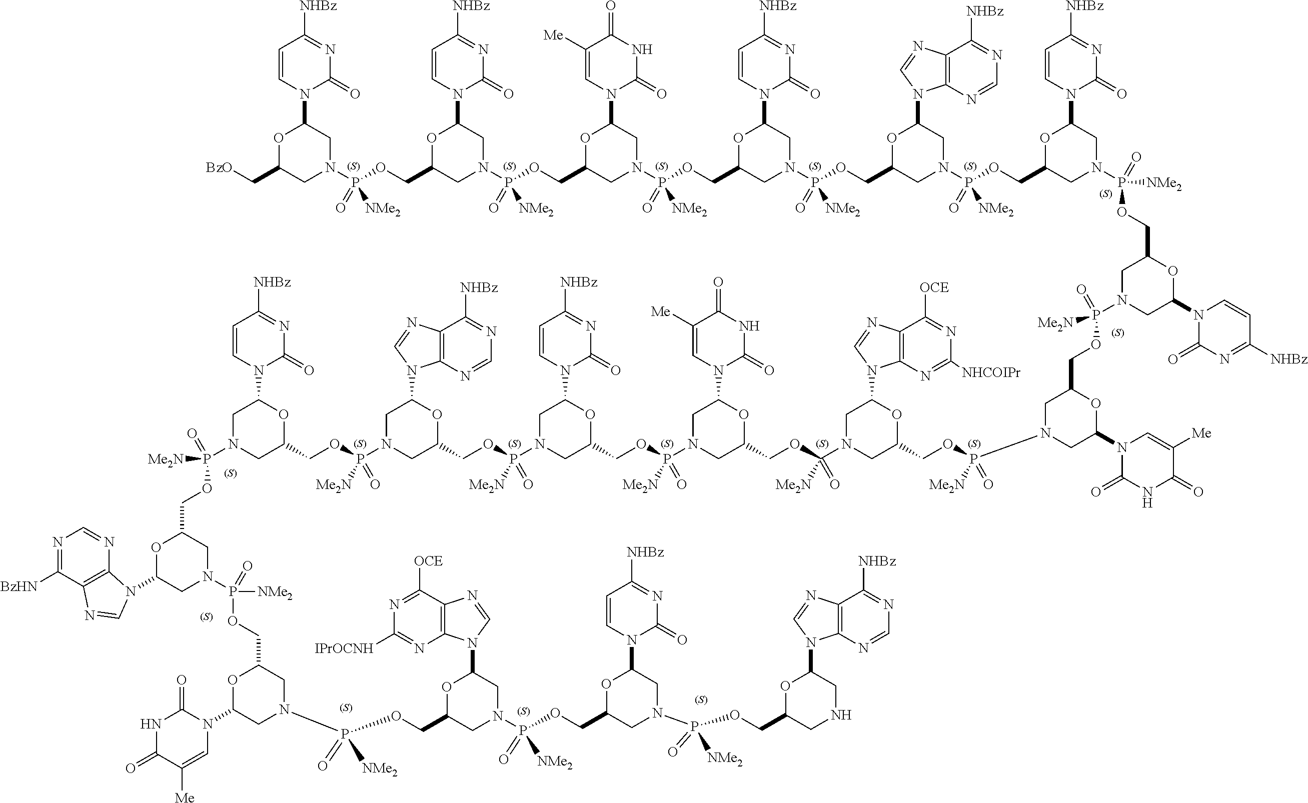

Using General Procedure B: reaction of trityl protected 18-mer (739 mg) afforded free base 18-mer (675 mg).

Coupling to 19-mer:

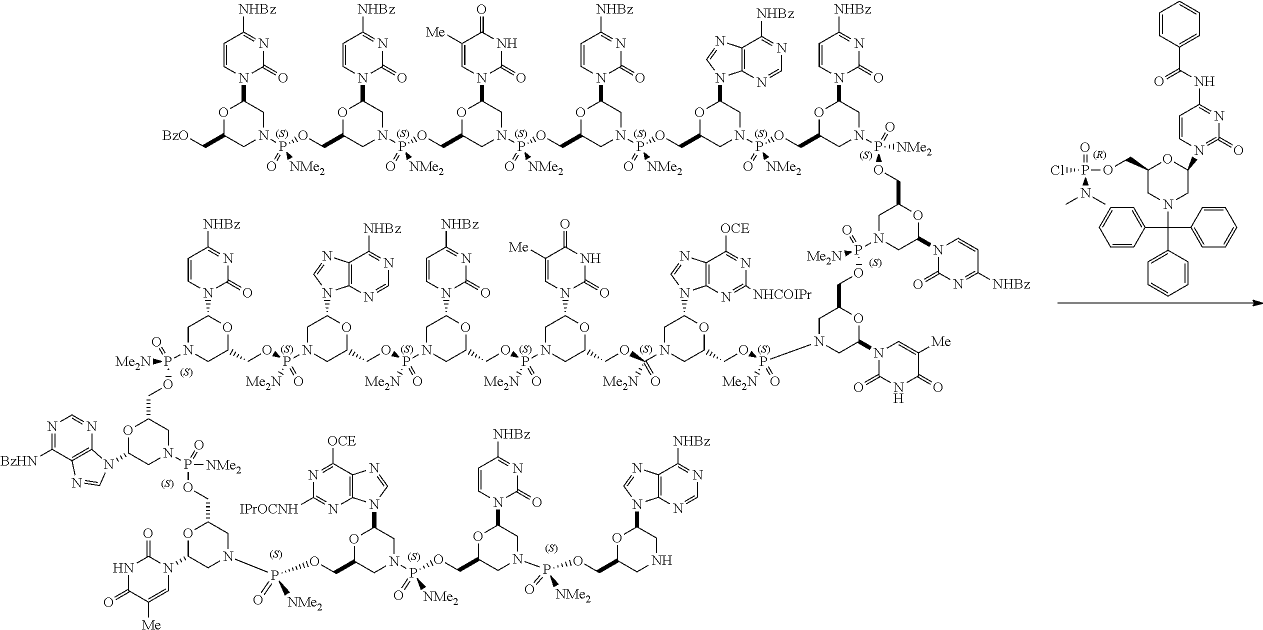

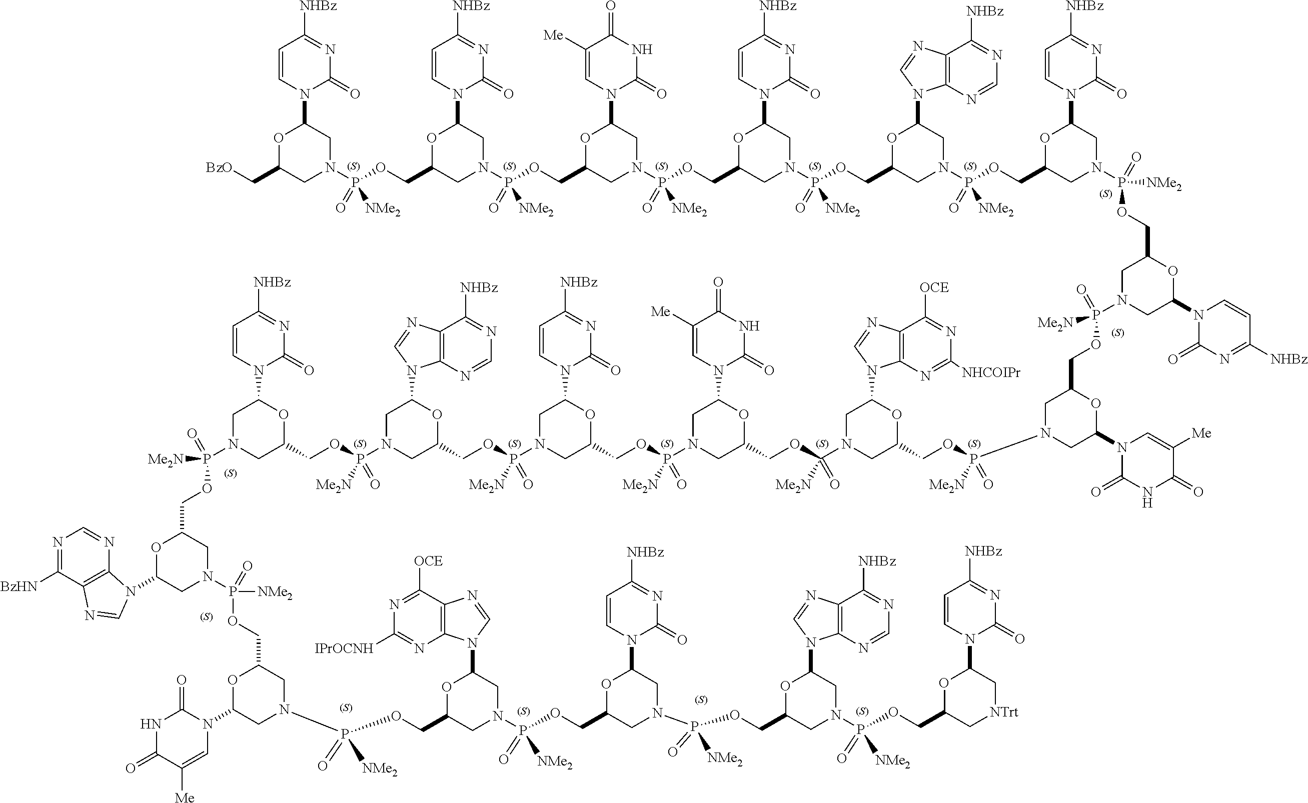

Using General Procedure A: reaction of 18-mer (675 mg) with ((2S,6R)-6-(4-benzamido-2-oxopyrimidin-1(2H)-yl)-4-tritylmorpholin-2-yl)methyl (R)-dimethylphosphoramidochloridate (127 mg) afforded 19-mer (735 mg).

Deprotection of 19-mer:

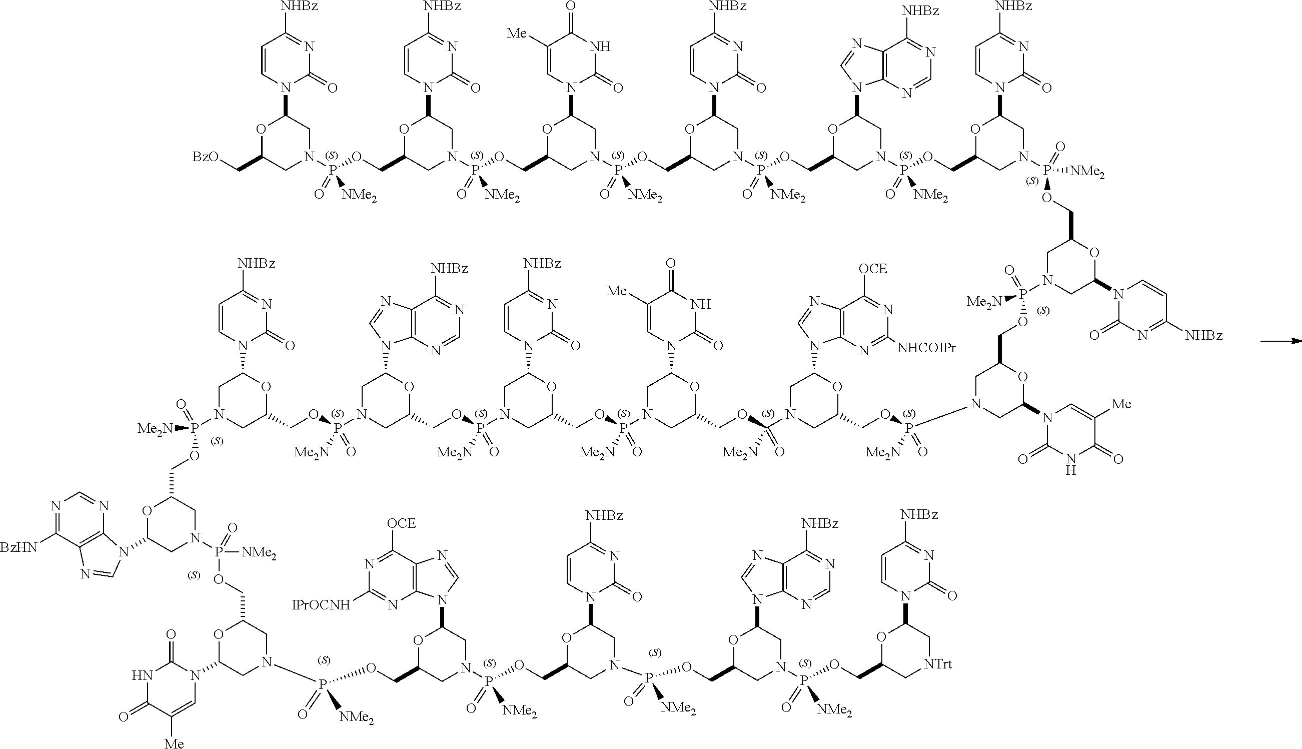

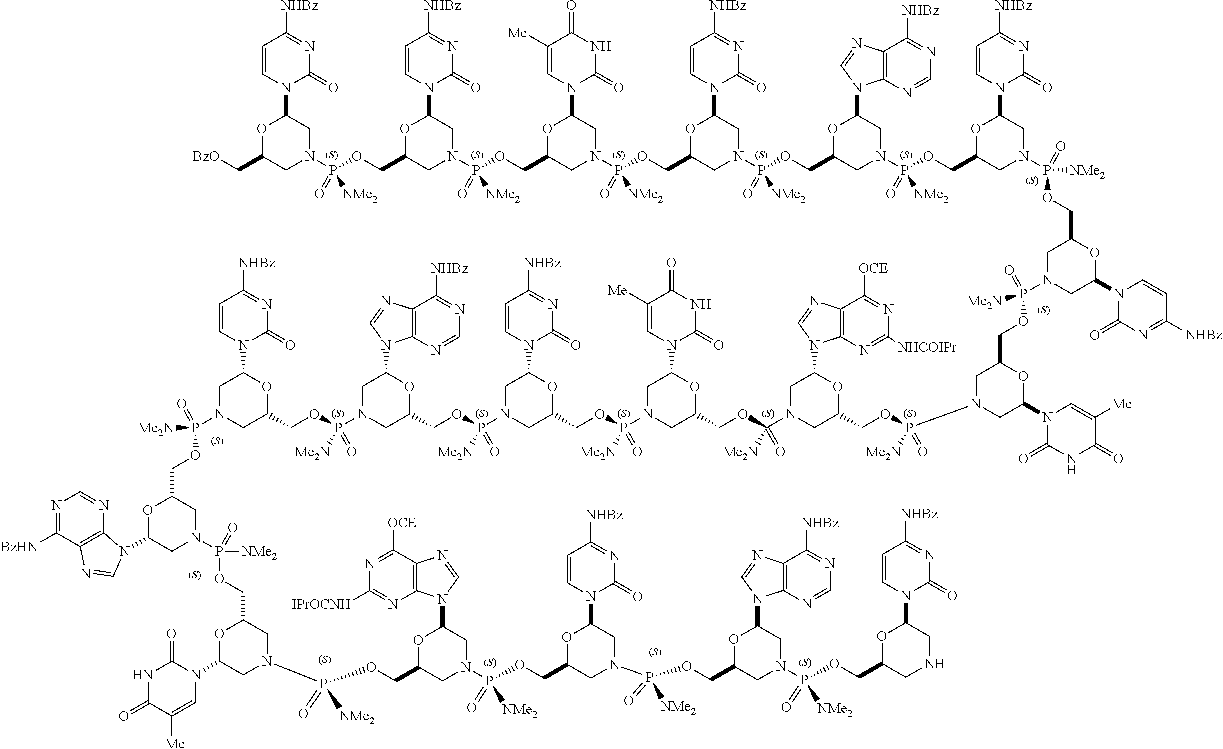

Using General Procedure B: reaction of trityl protected 19-mer (735 mg) afforded free base 19-mer (732 mg).

Coupling to 20-mer:

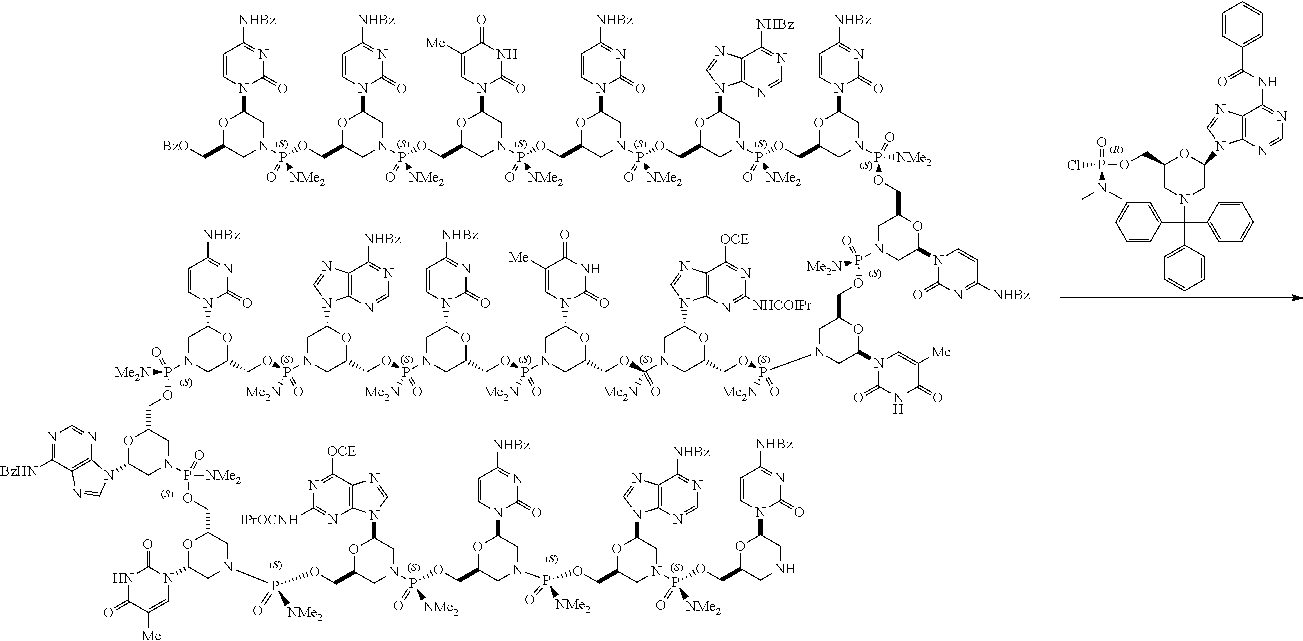

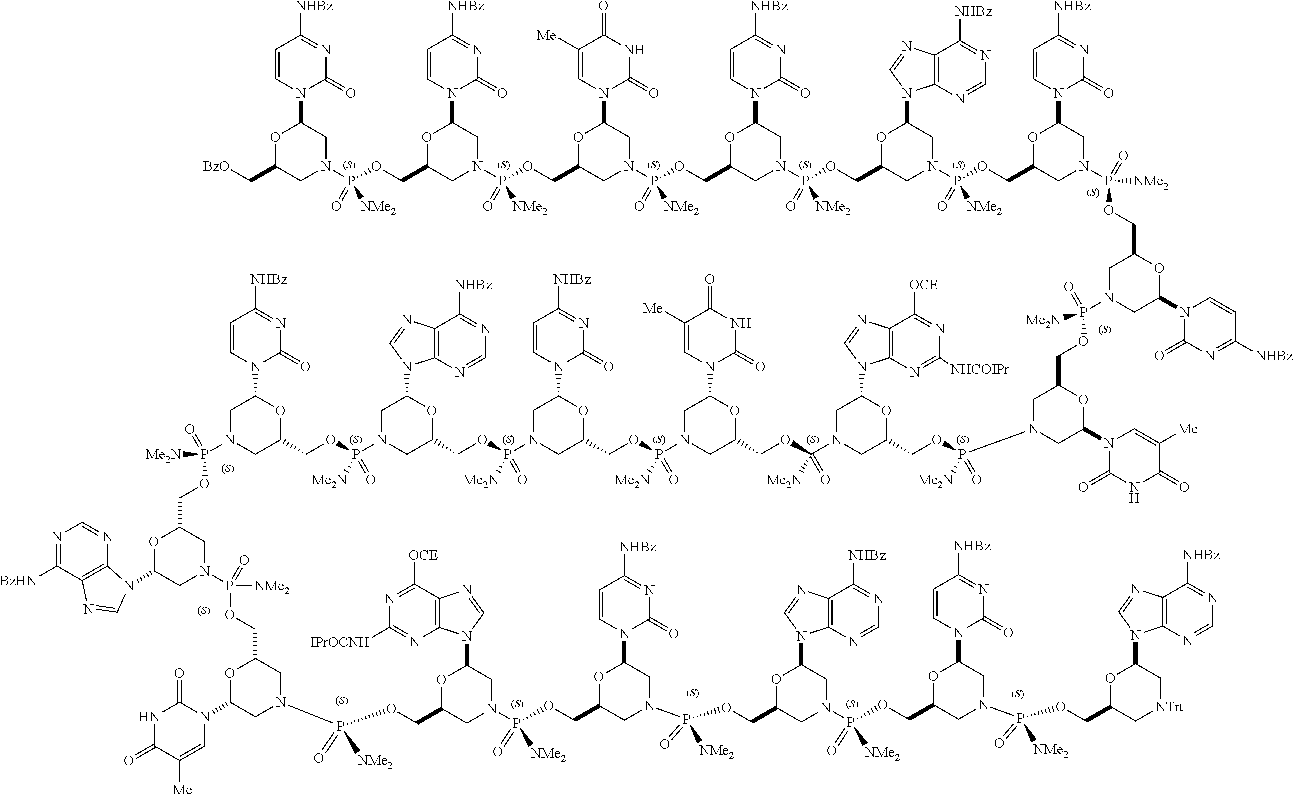

Using General Procedure A: reaction of 19-mer (732 mg) with ((2S,6R)-6-(6-benzamido-9H-purin-9-yl)-4-tritylmorpholin-2-yl)methyl (R)-dimethylphosphoramidochloridate (135 mg) afforded 20-mer (790 mg). MS (ESI) m/z: Calcd for C₃₆₇H₄₅₂N₁₁₁O₉₅P₁₉: [M+5H/5]+1706; Found: 1707.

Deprotection of 20-mer:

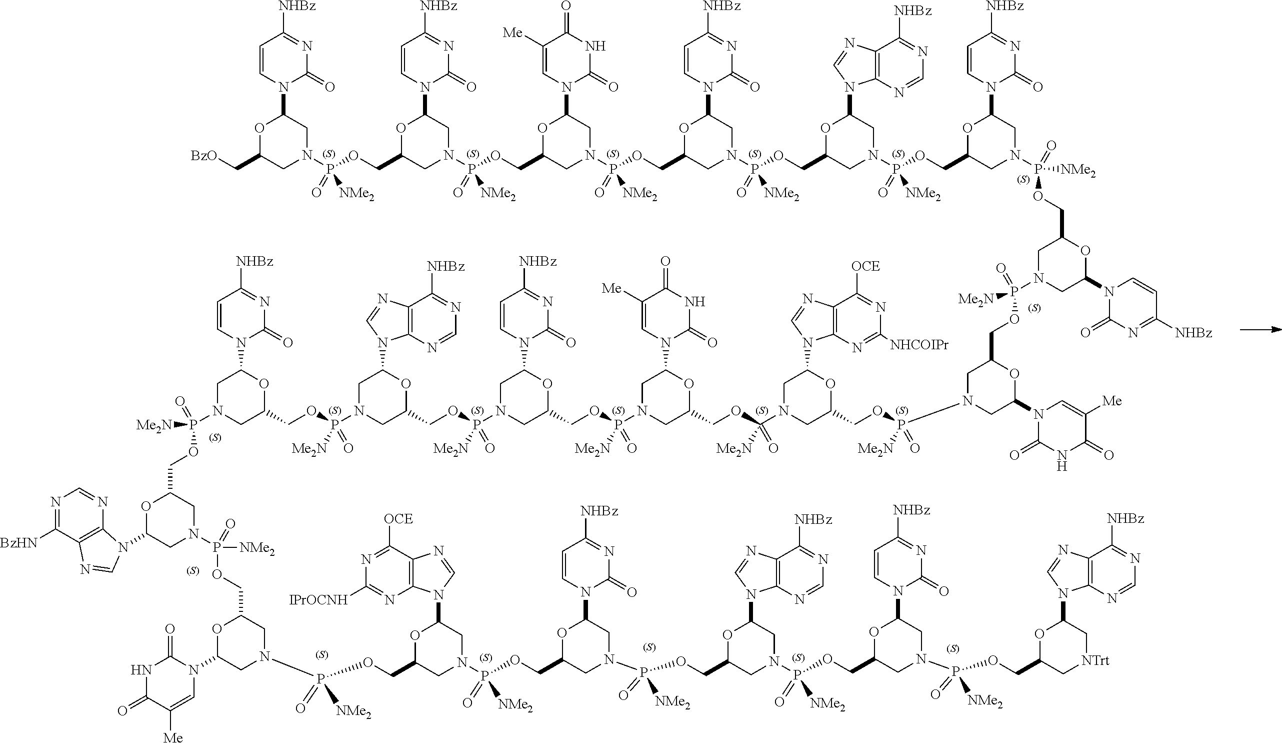

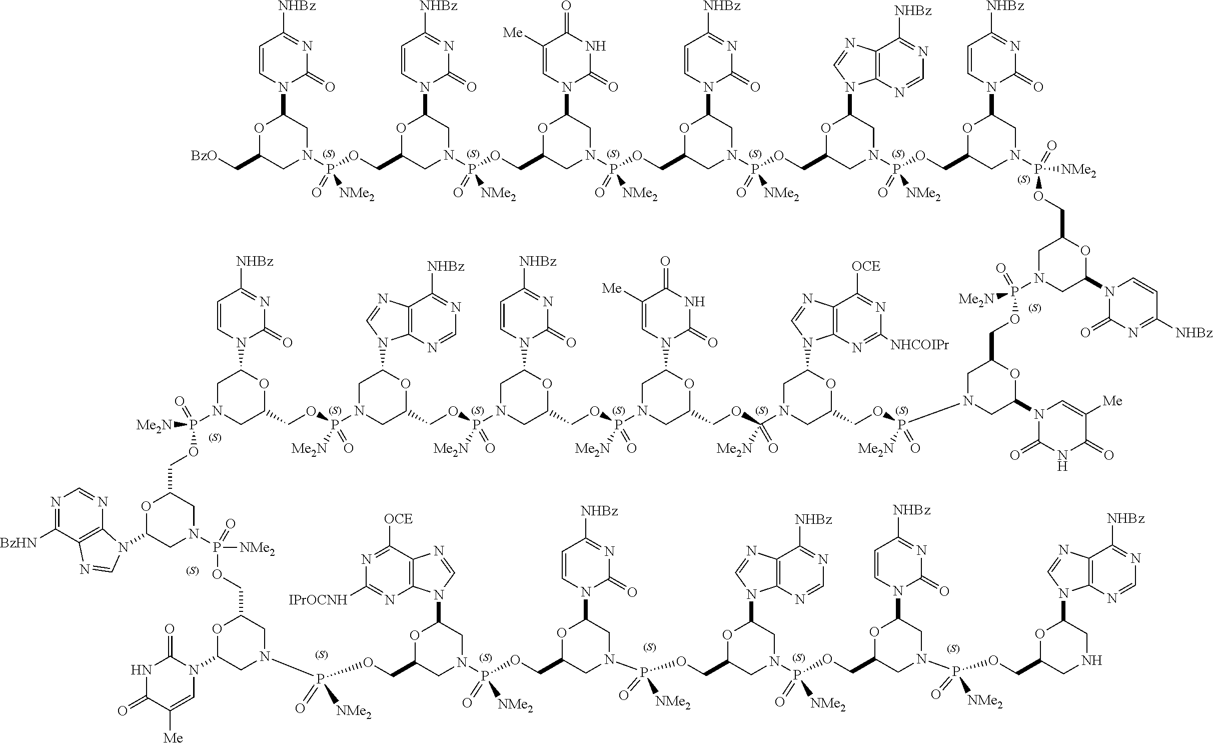

Using General Procedure B: reaction of trityl protected 20-mer (790 mg) afforded free base 20-mer (743 mg). MS (ESI) m/z: Calcd for C₃₄₈H₄₃₈N₁₁₁O₉₅P₁₉: [M+5H/5]+1657.6; Found: 1658.

Coupling to 21-mer:

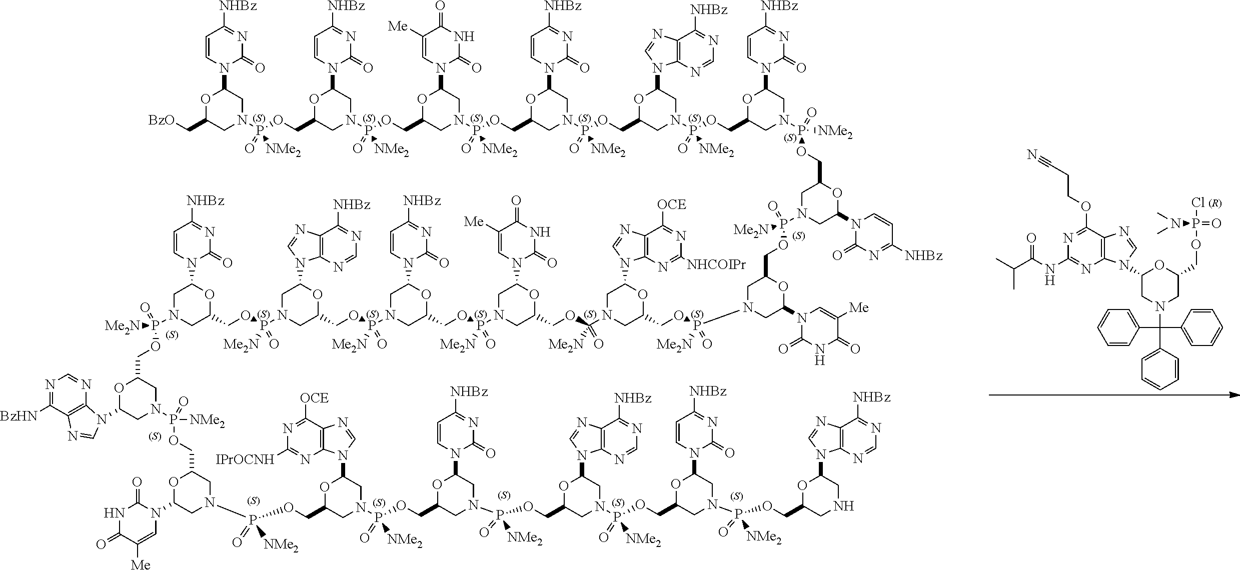

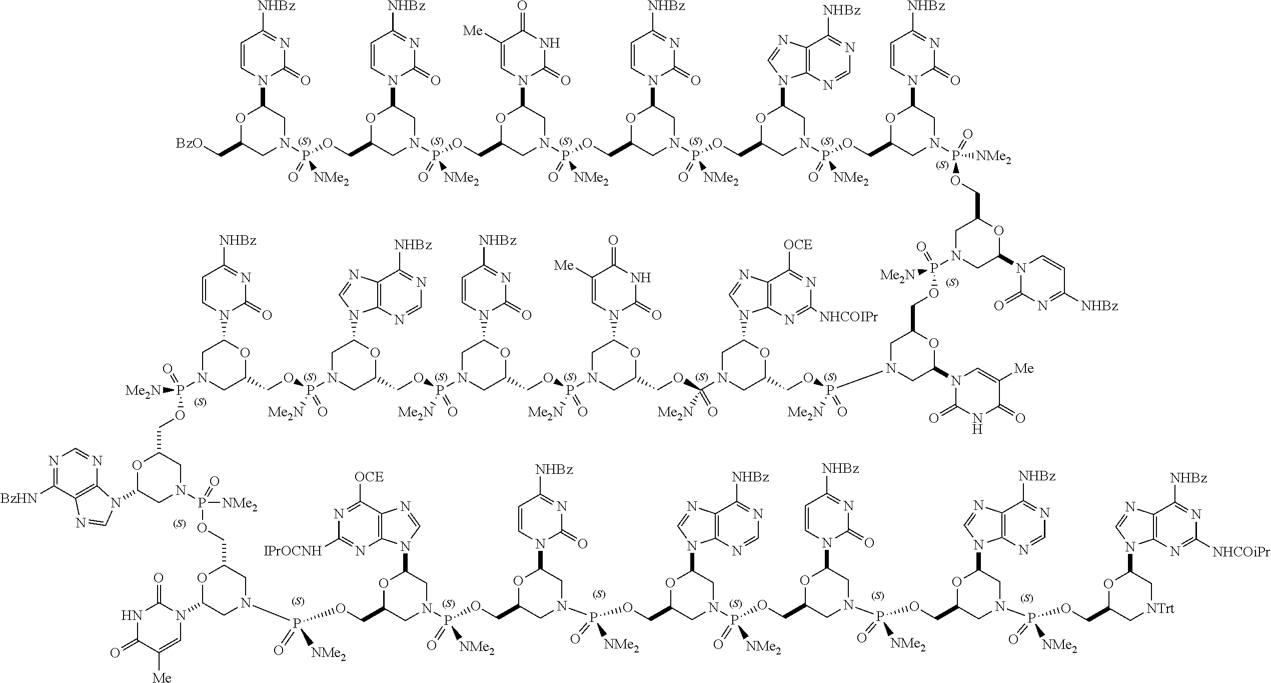

Using General Procedure A: reaction of 20-mer (743 mg) with ((2S,6R)-6-(6-(2-cyanoethoxy)-2-isobutyramido-9H-purin-9-yl)-4-tritylmorpholin-2-yl)methyl (R)-dimethyl phosphoramidochloridate (129 mg) afforded 21-mer (795 mg).

Deprotection of 21-mer:

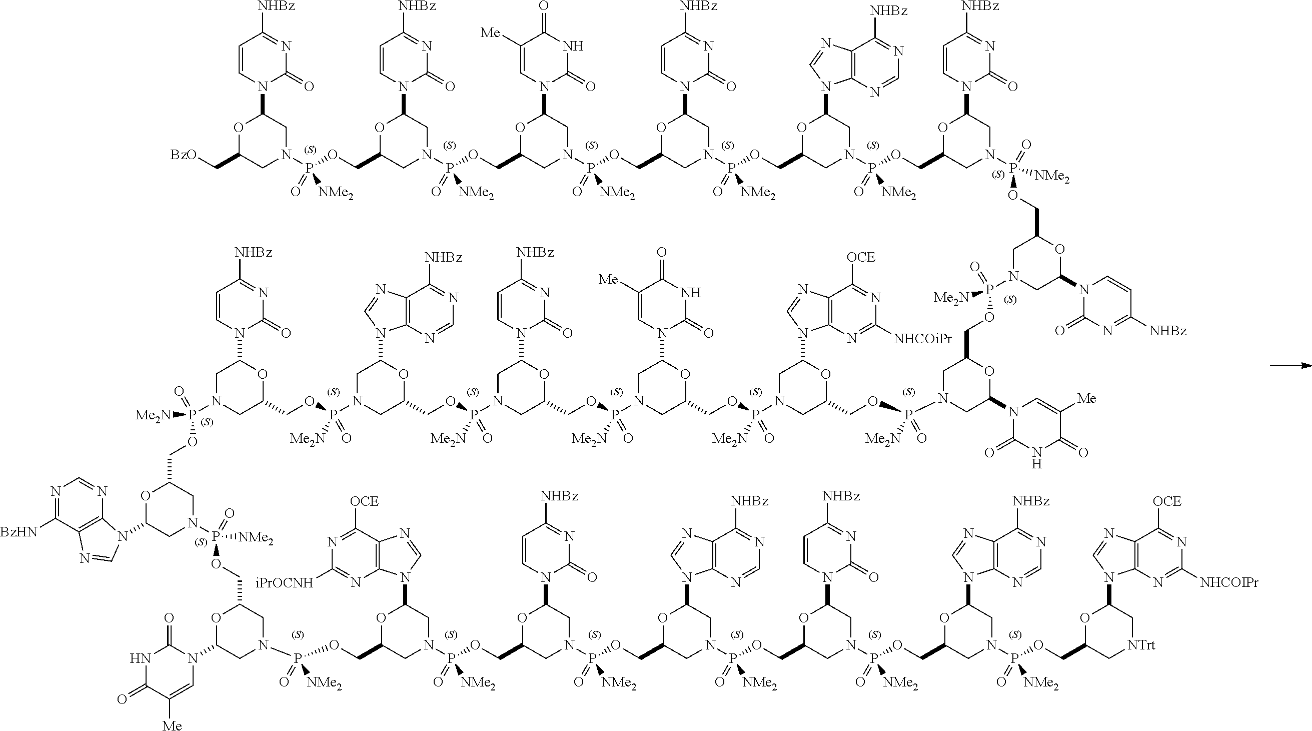

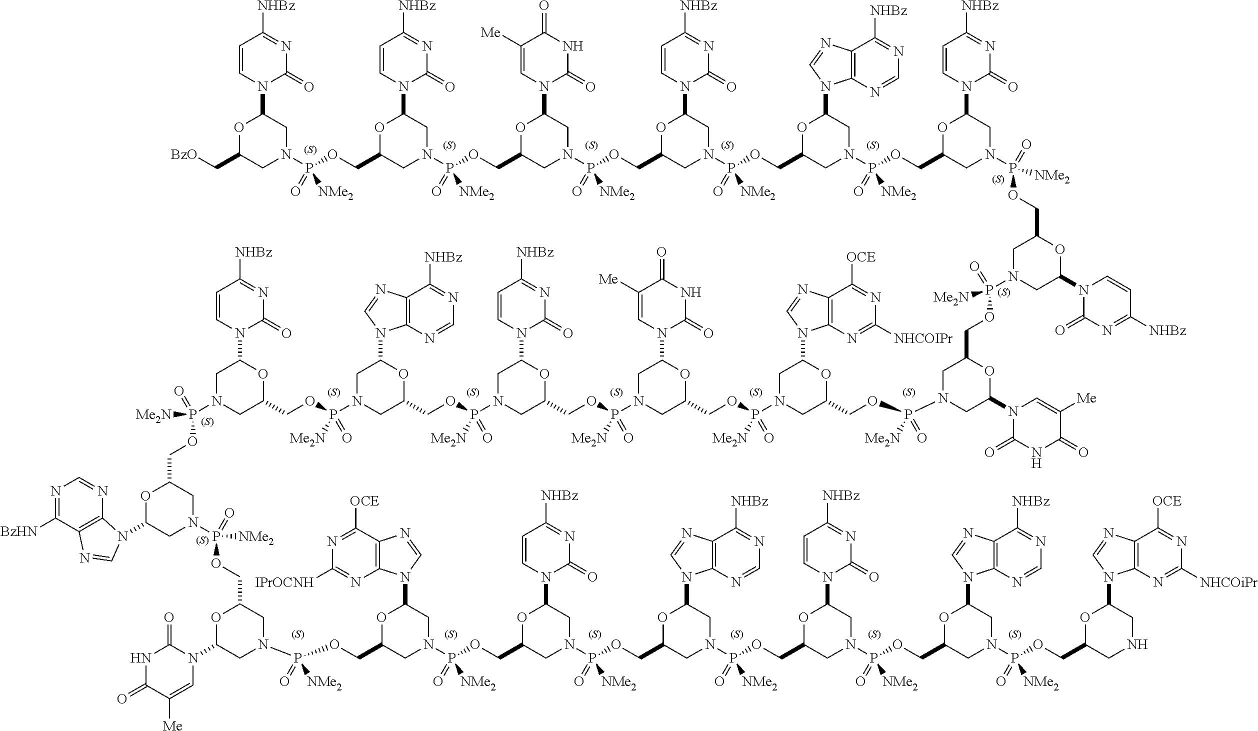

Using General Procedure B: reaction of trityl protected 21-mer (800 mg) afforded free base 21-mer (756 mg).

Coupling to 22-mer:

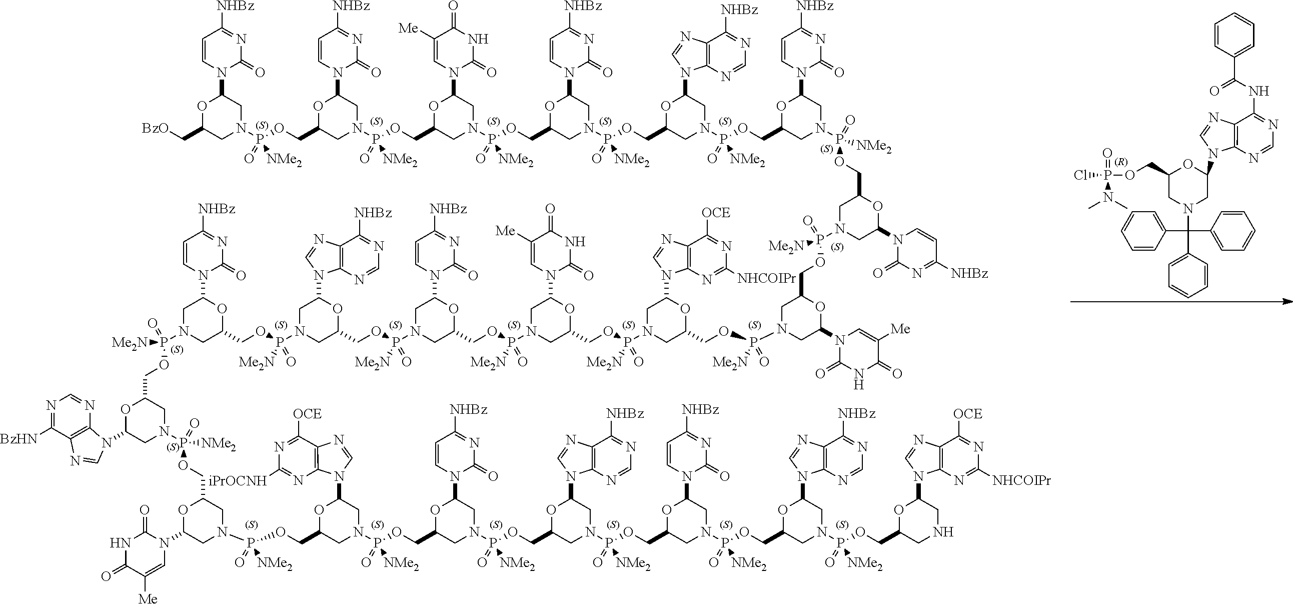

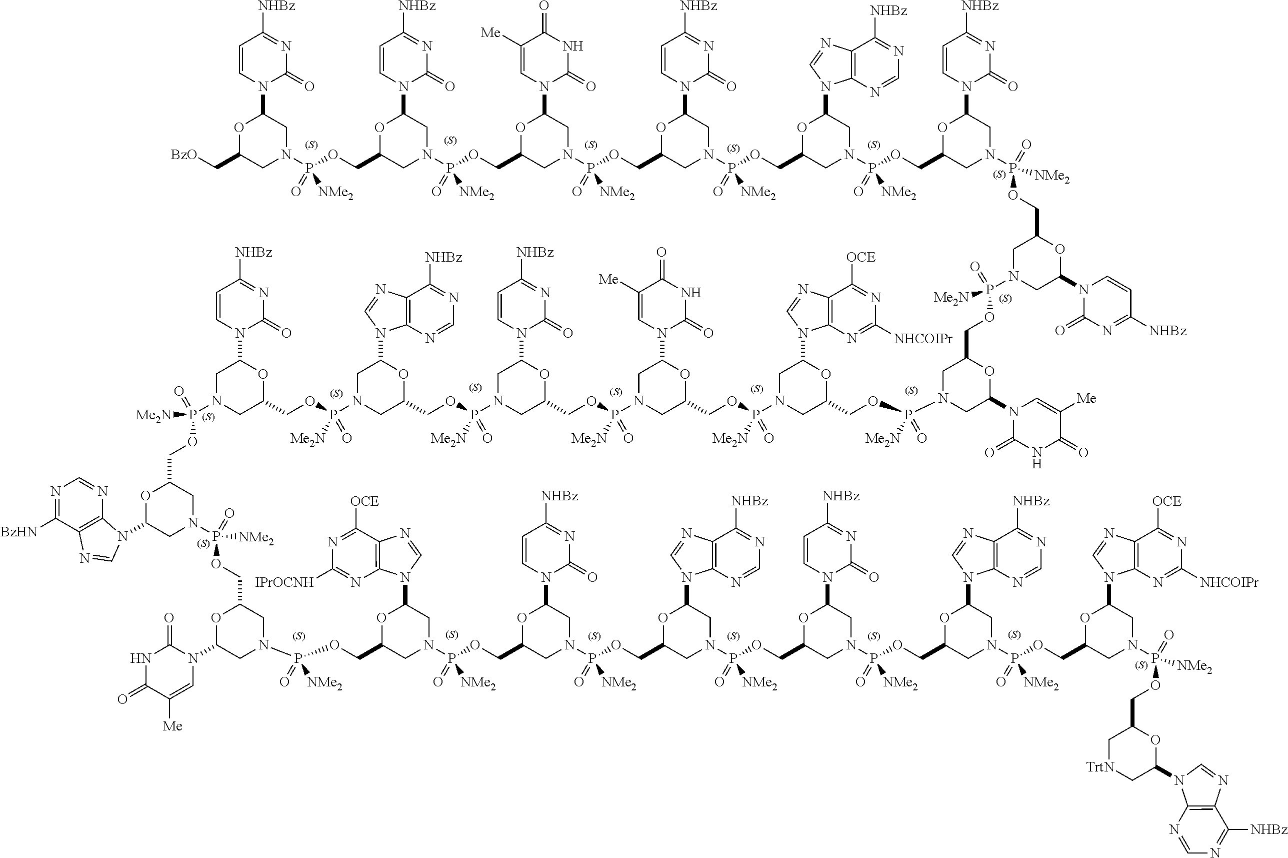

Using General Procedure A: reaction of 21-mer (753 mg) with ((2S,6R)-6-(6-benzamido-9H-purin-9-yl)-4-tritylmorpholin-2-yl)methyl (R)-dimethylphosphoramidochloridate (137 mg) afforded 22-mer (806 mg).

Deprotection of 22-mer:

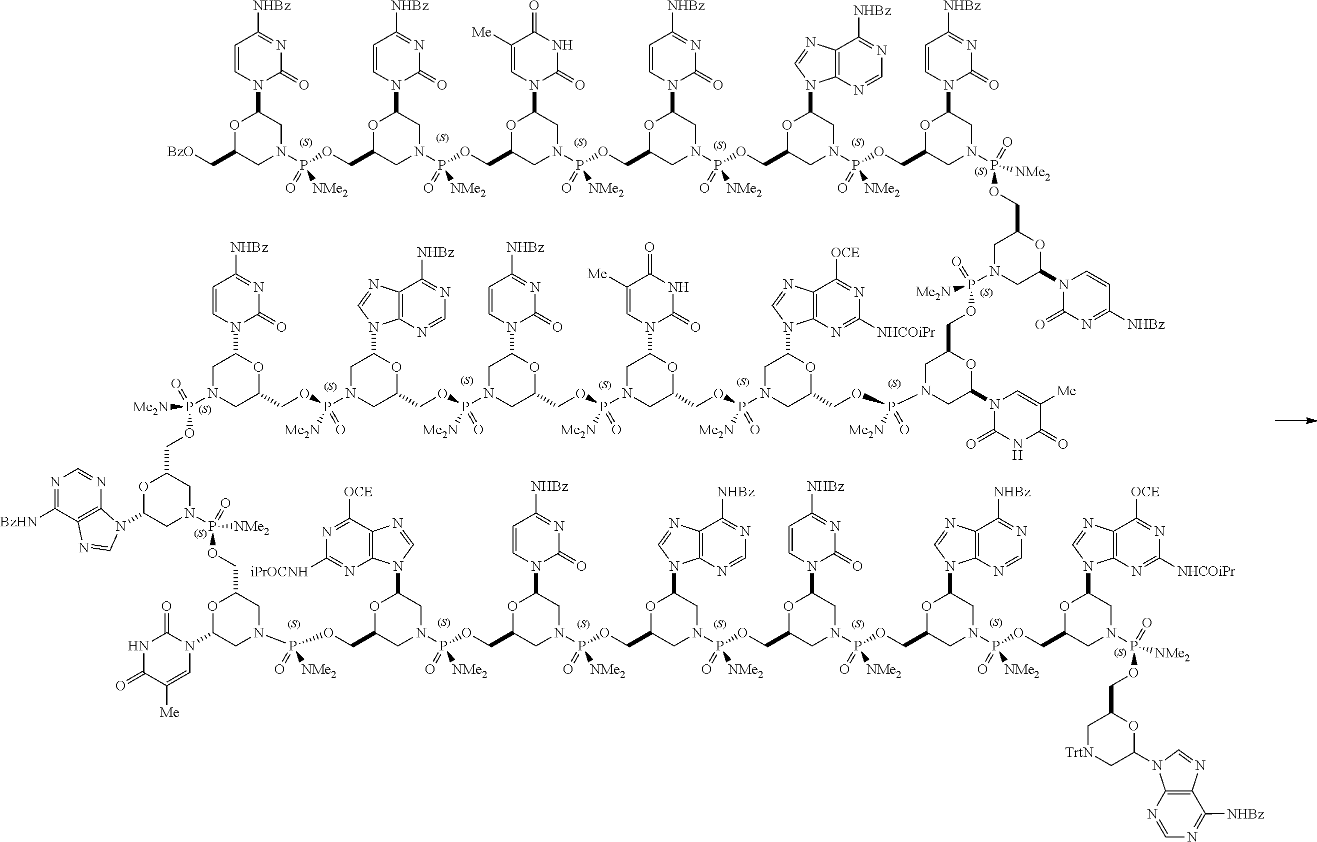

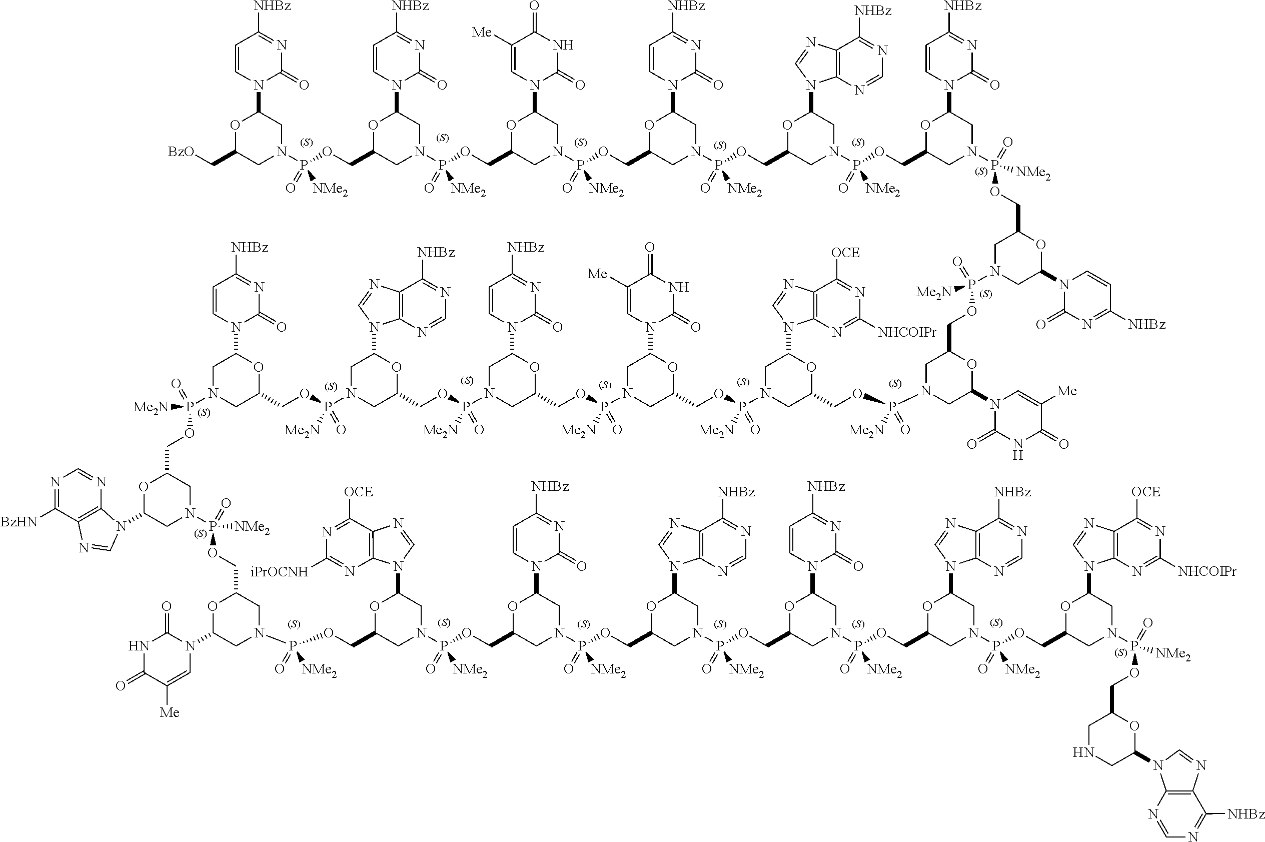

Using General Procedure B: reaction of trityl protected 22-mer (806 mg) afforded free base 22-mer (785 mg).

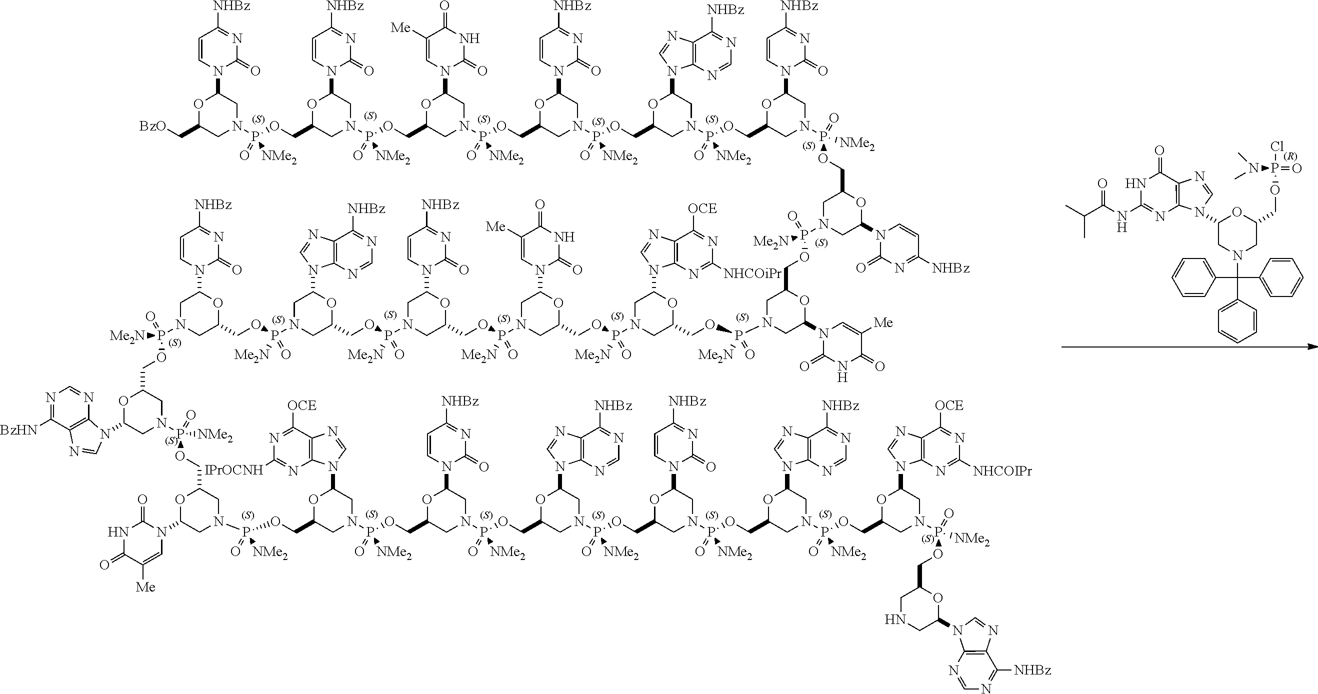

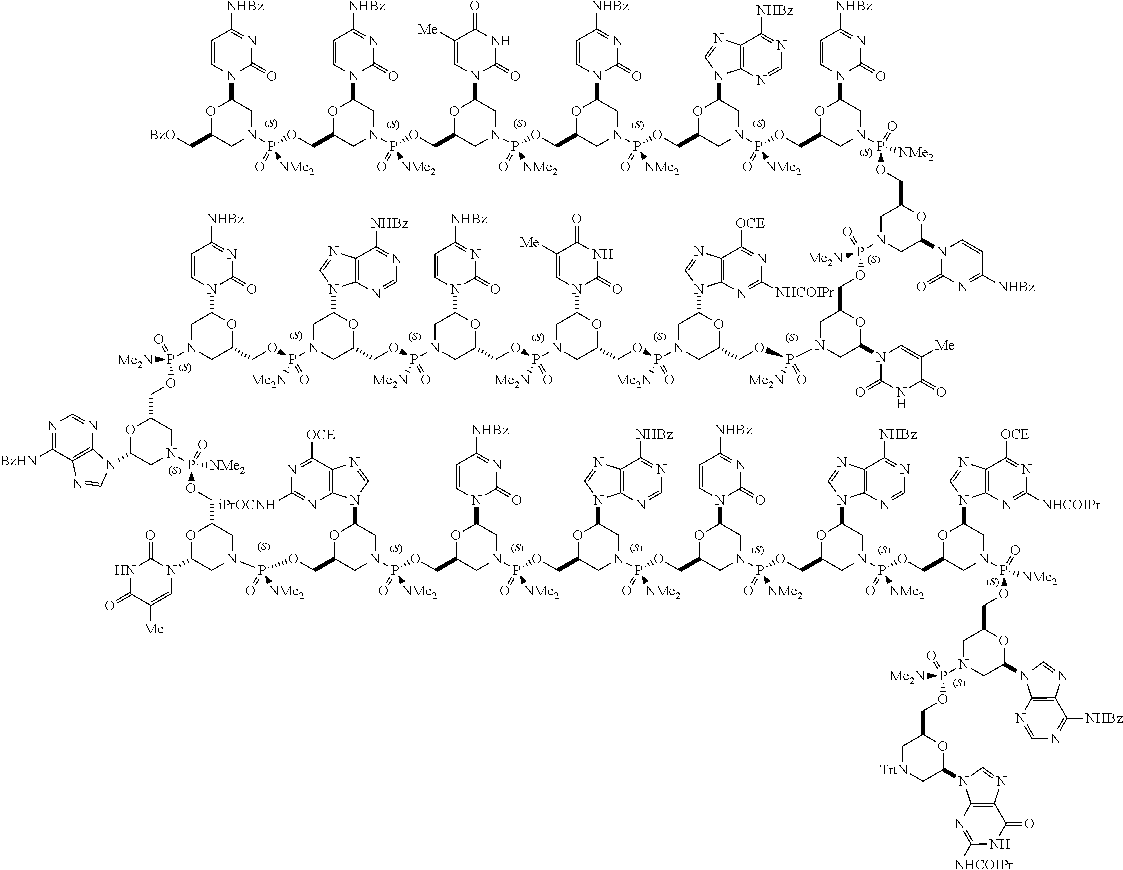

Using General Procedure A: reaction of 22-mer (780 mg) with ((2S,6R)-6-(2-isobutyramido-6-oxo-1,6-dihydro-9H-purin-9-yl)-4-tritylmorpholin-2-yl)methyl (R)-dimethyl phosphoramidochloridate (161 mg) afforded 23-mer (837 mg).

Deprotection of 23-mer:

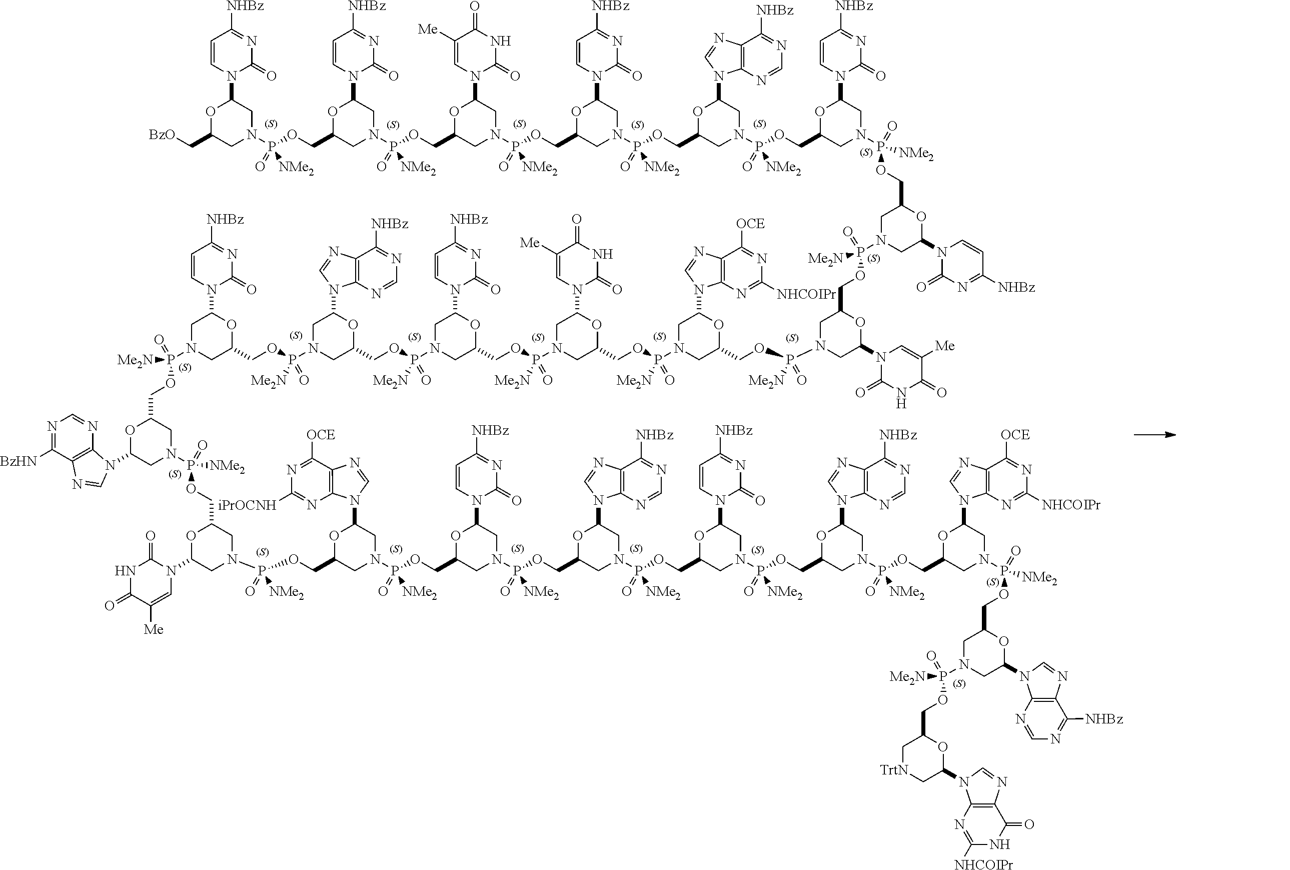

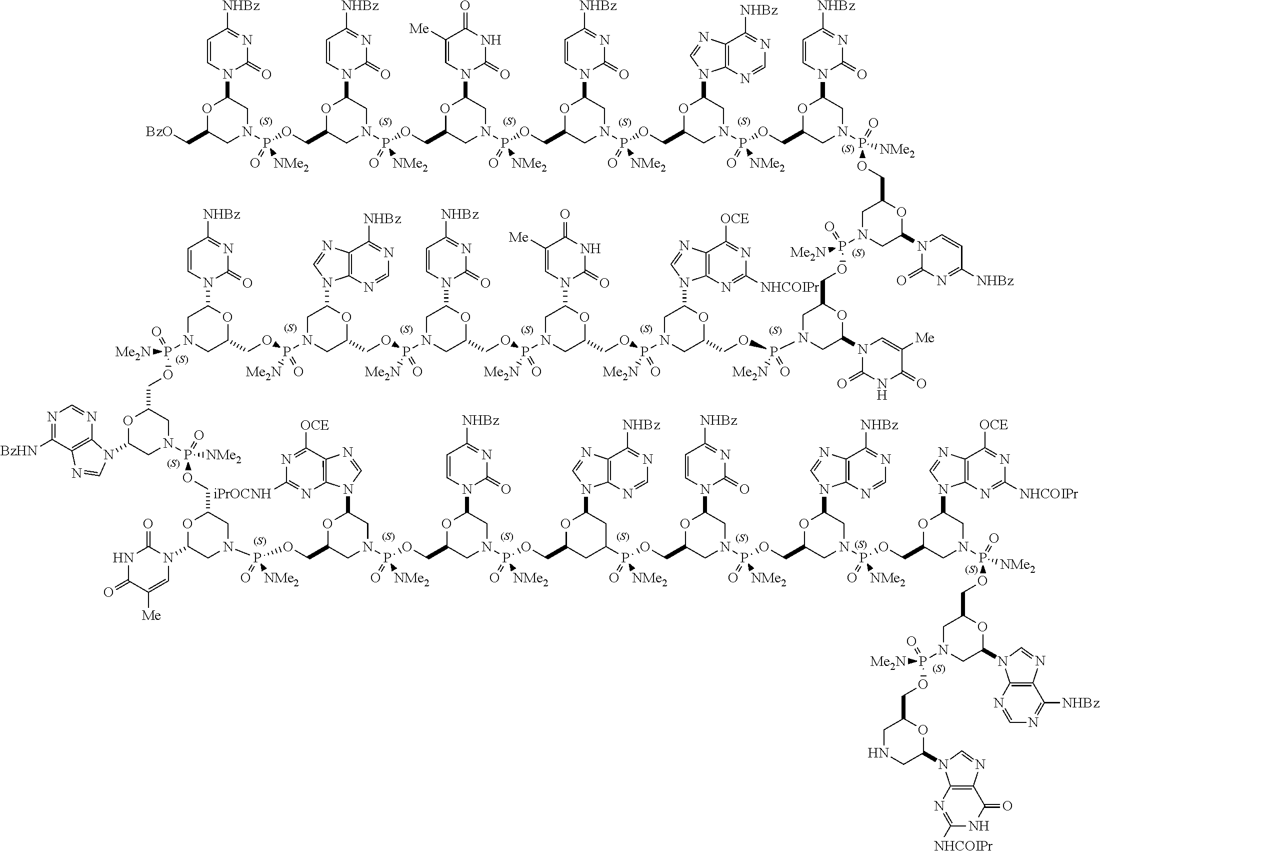

Using General Procedure B: reaction of trityl protected 23-mer (837 mg) afforded free base 23-mer (830 mg).

Coupling to 24-mer:

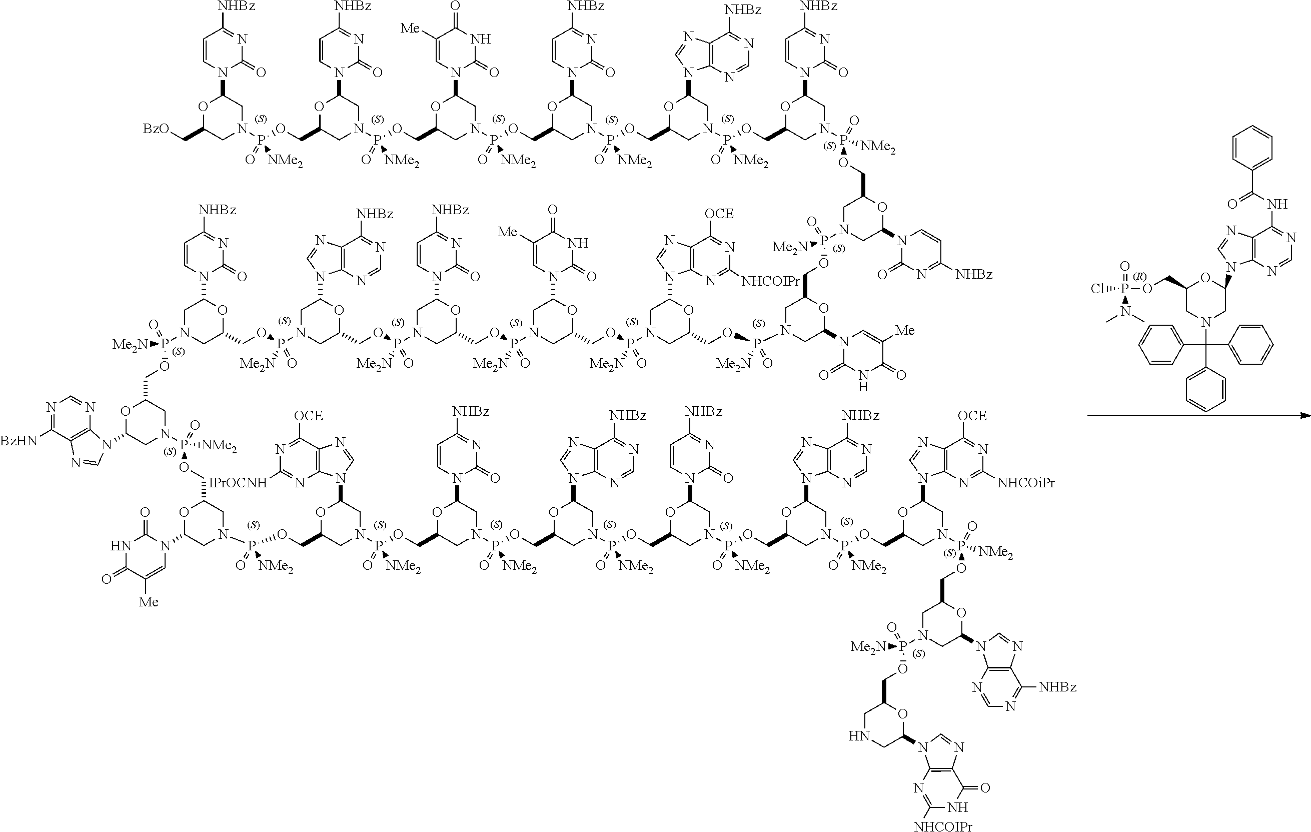

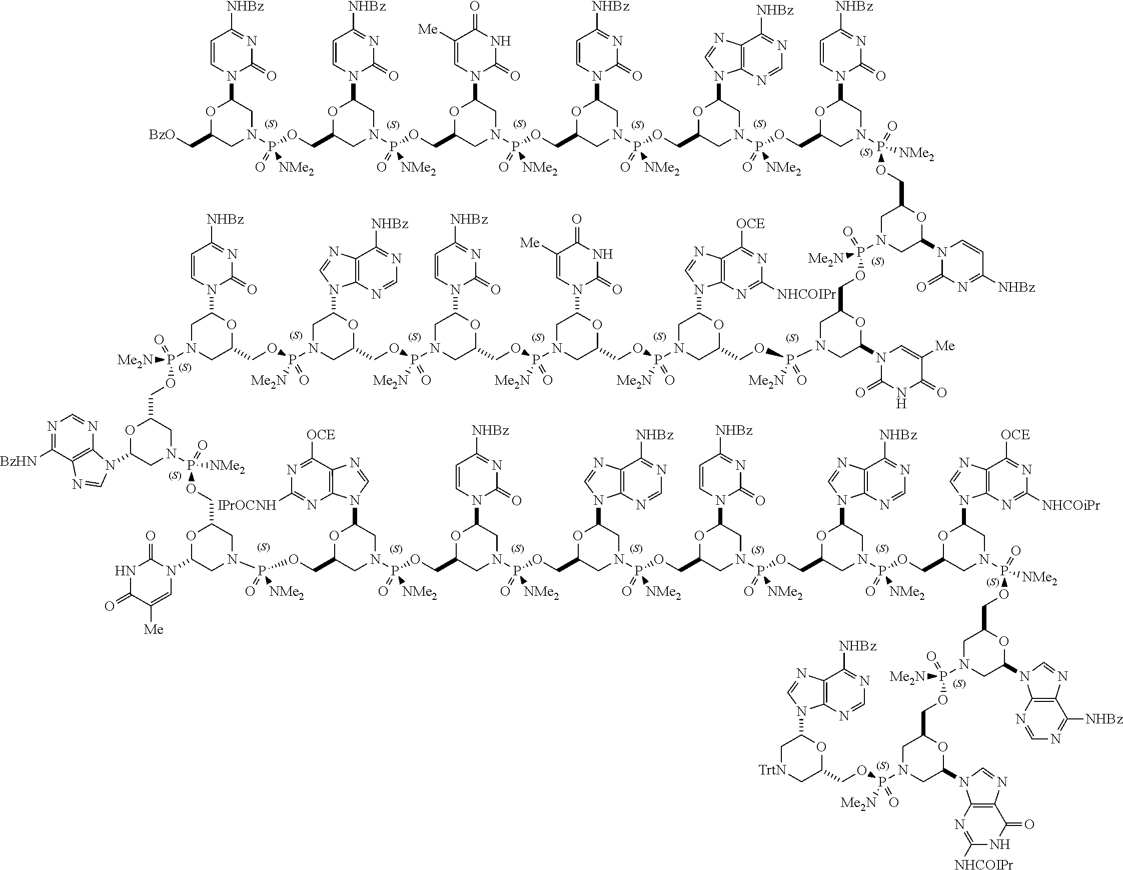

Using General Procedure A: reaction of 23-mer (830 mg) with ((2S,6R)-6-(6-benzamido-9H-purin-9-yl)-4-tritylmorpholin-2-yl)methyl (R)-dimethylphosphoramidochloridate (177 mg) afforded 24-mer (800 mg).

Deprotection of 24-mer:

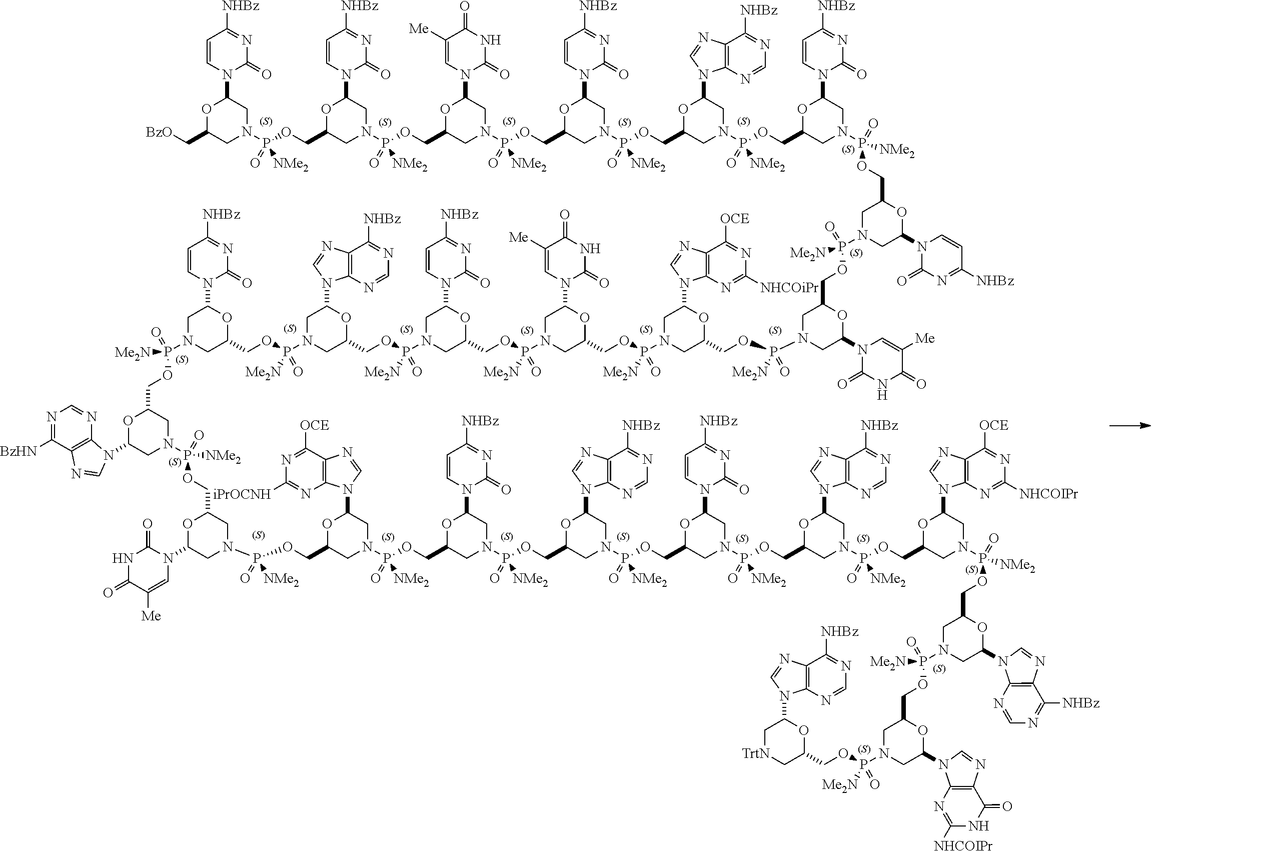

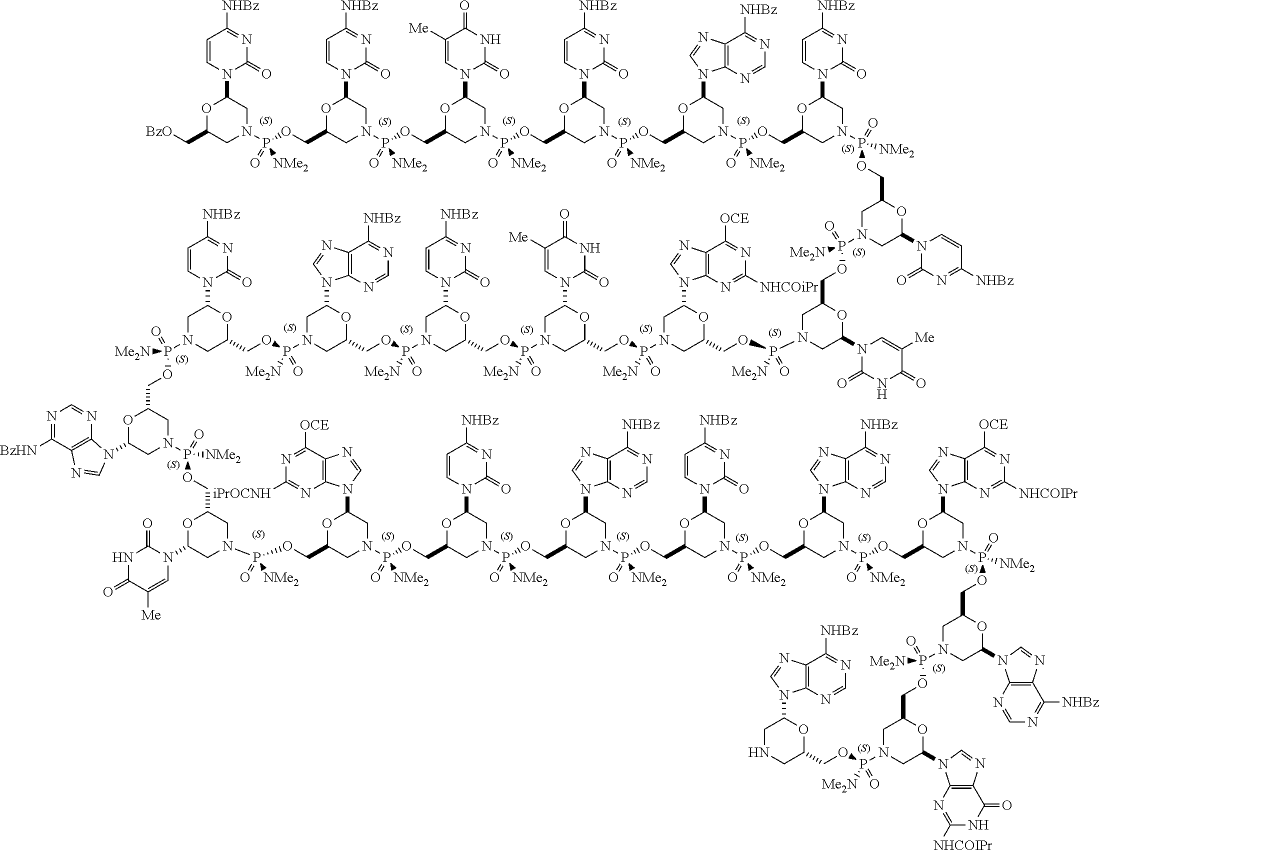

Using General Procedure B: reaction of trityl protected 24-mer (800 mg) afforded free base 24-mer (793 mg).

Coupling to 25-mer:

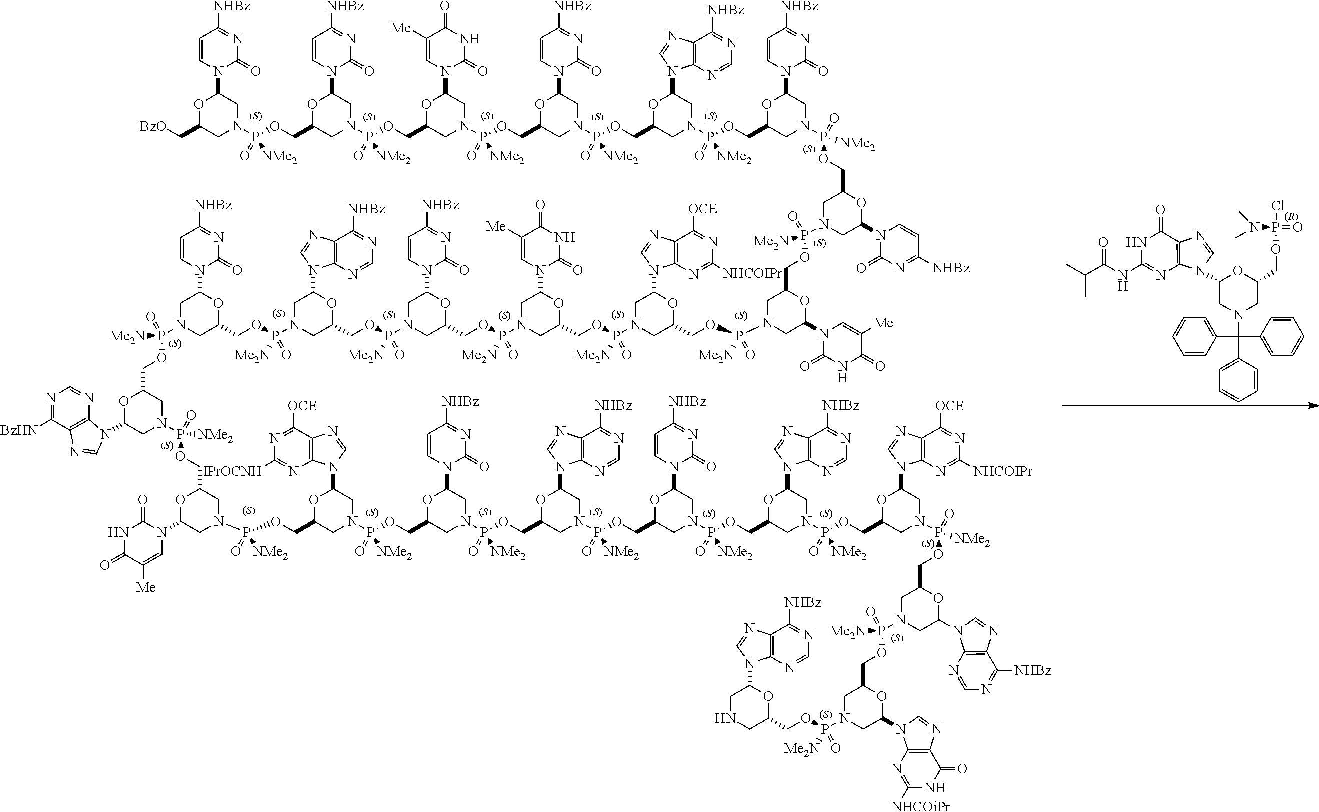

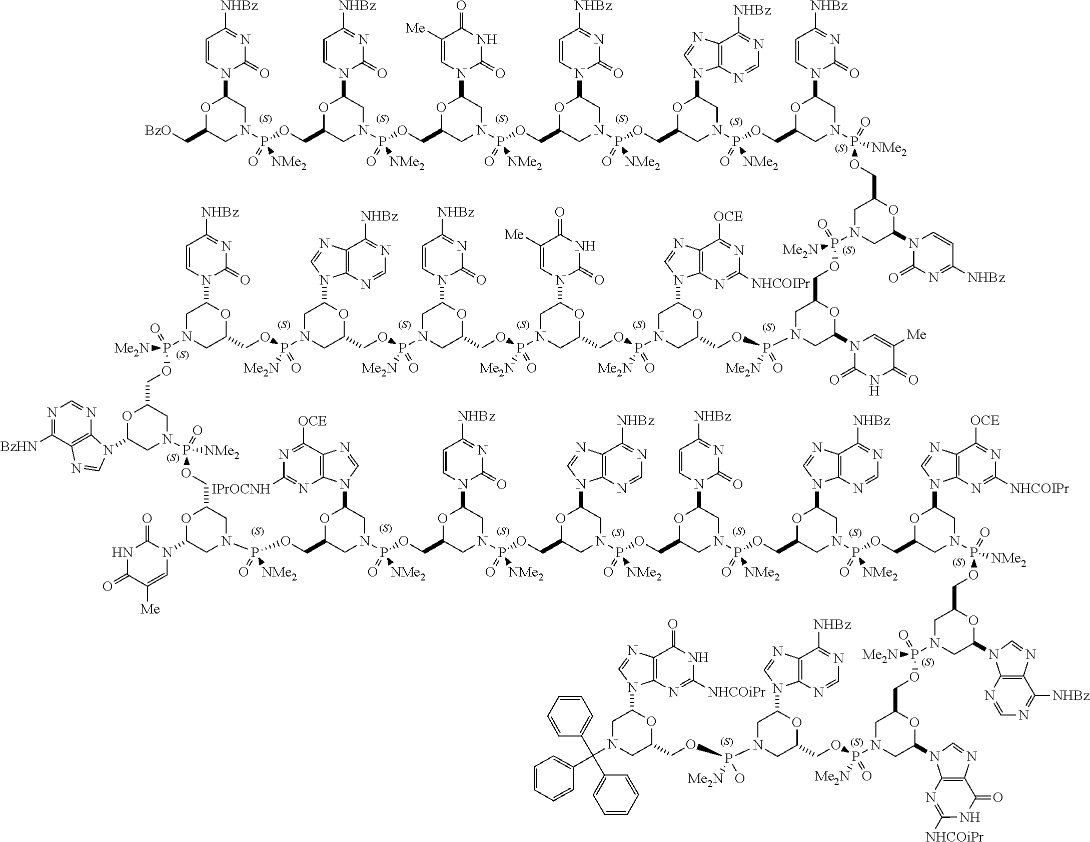

Using General Procedure A: reaction of 24-mer (793 mg) with ((2S,6R)-6-(2-isobutyramido-6-oxo-1,6-dihydro-9H-purin-9-yl)-4-tritylmorpholin-2-yl)methyl (R)-dimethylphosphoramidochloridate (150 mg) afforded 25-mer (818 mg). MS (ESI) m/z: Calcd for C₄₅₆H₁₇₁ N₁₄₇O₁₁₈P₂₄: [M+7H/7]+1535.53; Found: 1535.56.

### Base Deprotection of 25-Mer:

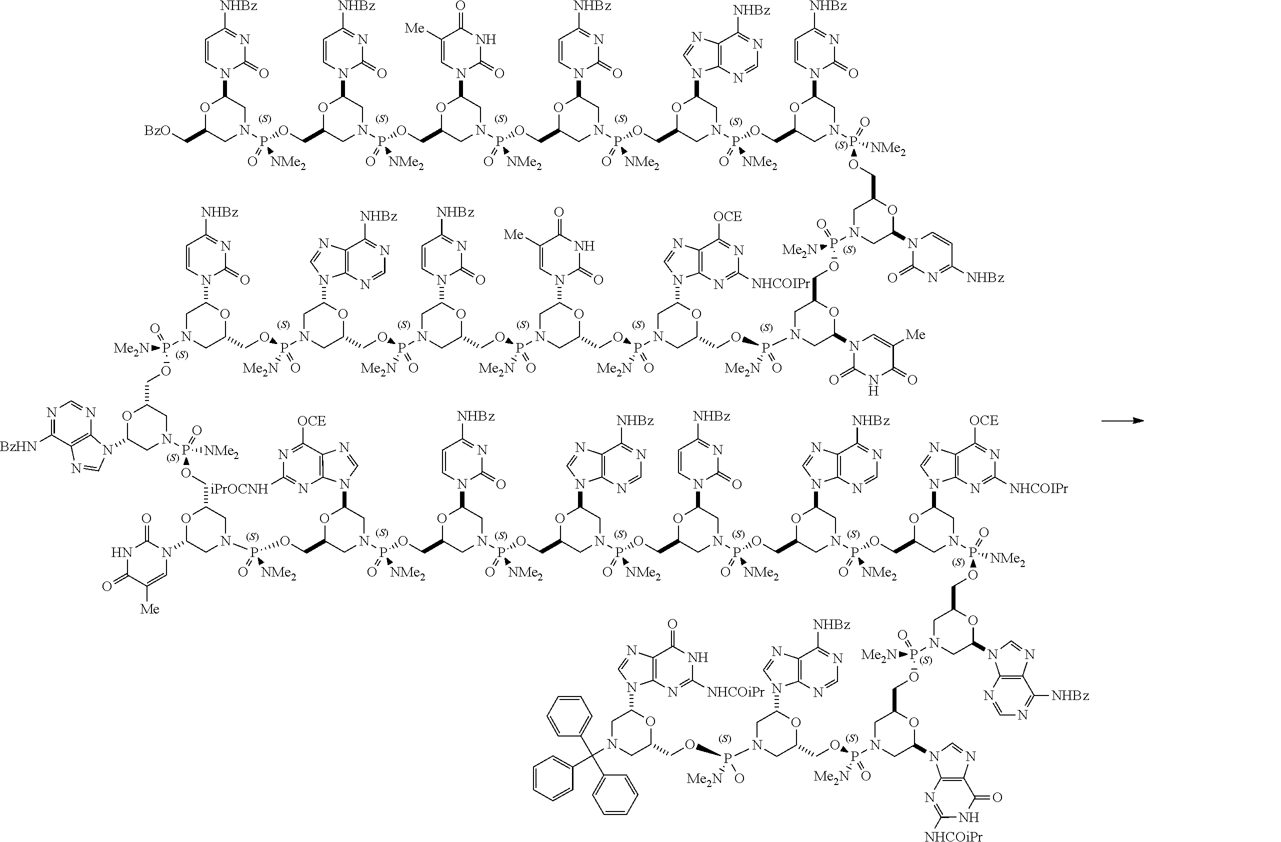

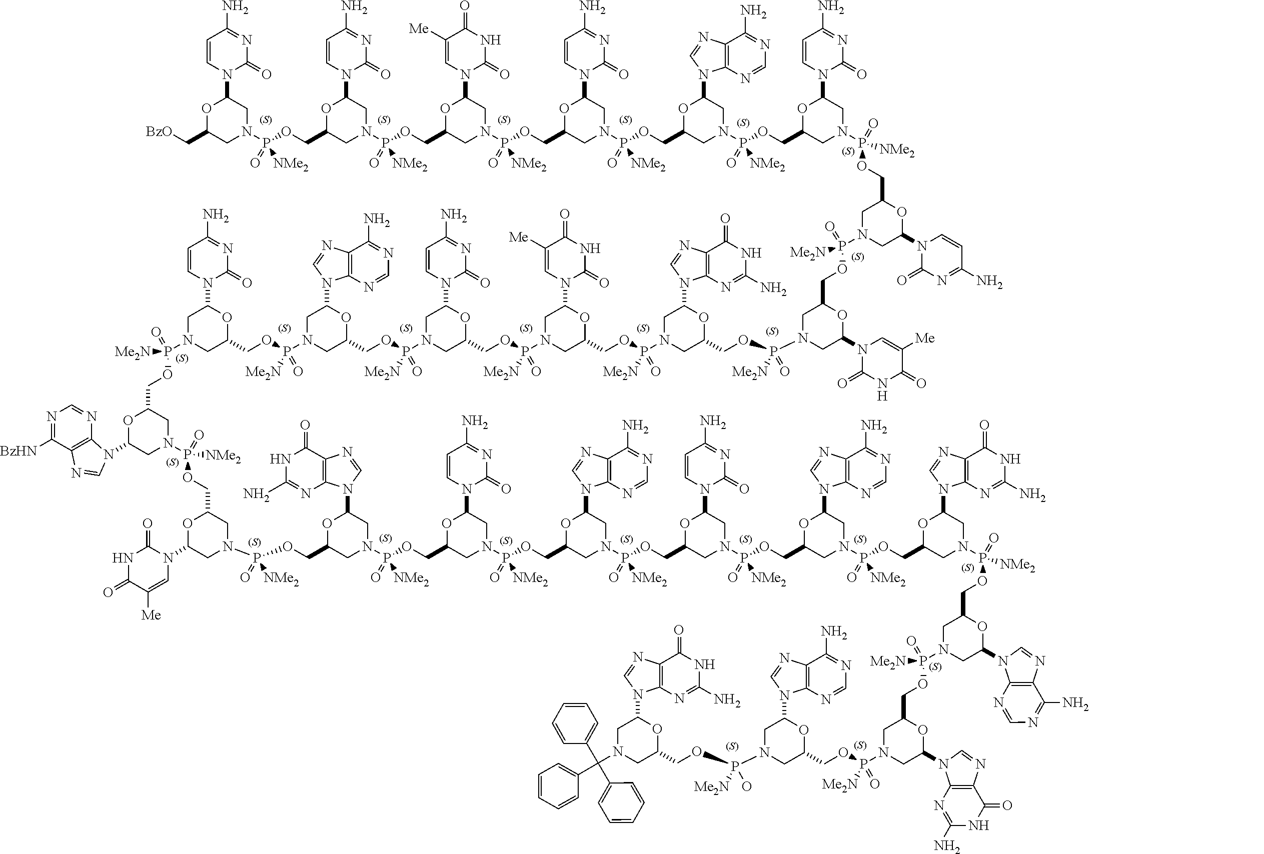

plastic fritted funnel and rinsed with ca 5-10 mL of water for a total adjusted volume of 25 mL. The resulting solution was used for purification by reverse phase HPLC using the conditions below. The desired peak fractions were evaporated to afford a total of 148 mg deprotected Trityl-on 25-mer a white solid. MS (ESI) m/z: Calcd for C₃₀₈N₁₄₄O₉P₂₄: [M+1H] 8462.97; Found: 8463.00 (deconvoluted HRMS spectrum).

Purification conditions for trityl—protected PMO:

| Column: | Waters Xbridge prep C18 5 um OBD | | | |
|---|---|---|---|---|
| | 19 × 100 mm (Part Number: 186002978) | | | |
| Instrument: | Waters Prep HPLC | | | |
| Mobile phase A: | 10 mM NH4HCO3, pH 10 (with NH4OH) | | | |
| Mobile phase B: | 15% of Mobile Phase A and 85% acetonitrile | | | |
| Column Temperature: | Unregulated | | | |
| Gradient: | | | | |
| | TIME (min) | A % | B % | |
| | 0 | 80 | 20 | Initial |
| | 12 | 65 | 35 | Elution Gradient |
| | 13 | 0 | 100 | |
| | 14 | 0 | 100 | |
| | 15 | 80 | 20 | Reset Conditions |

| | |
|---|---|
| Flow Rate (mL/min): | 40 |
| Wavelength: | DAD + 260 nm + timed collection |

### Trityl Deprotection of 25-Mer:

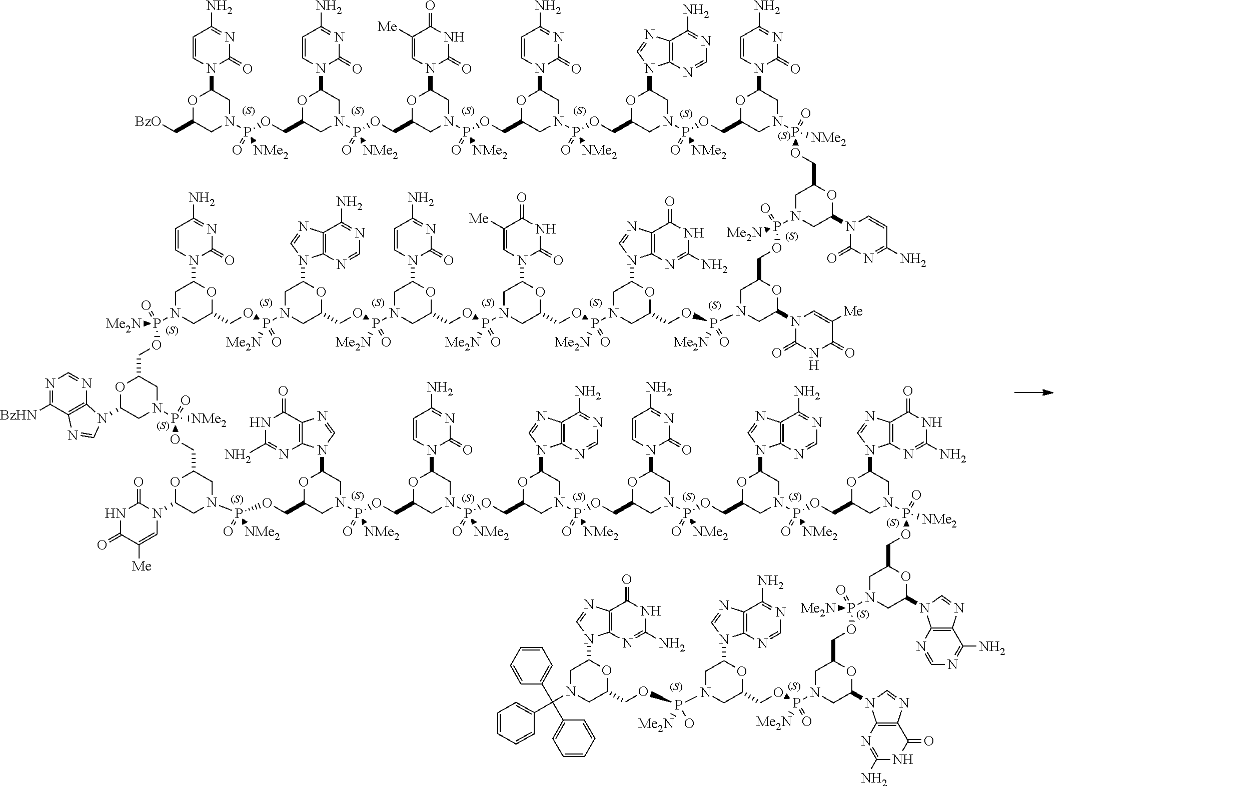

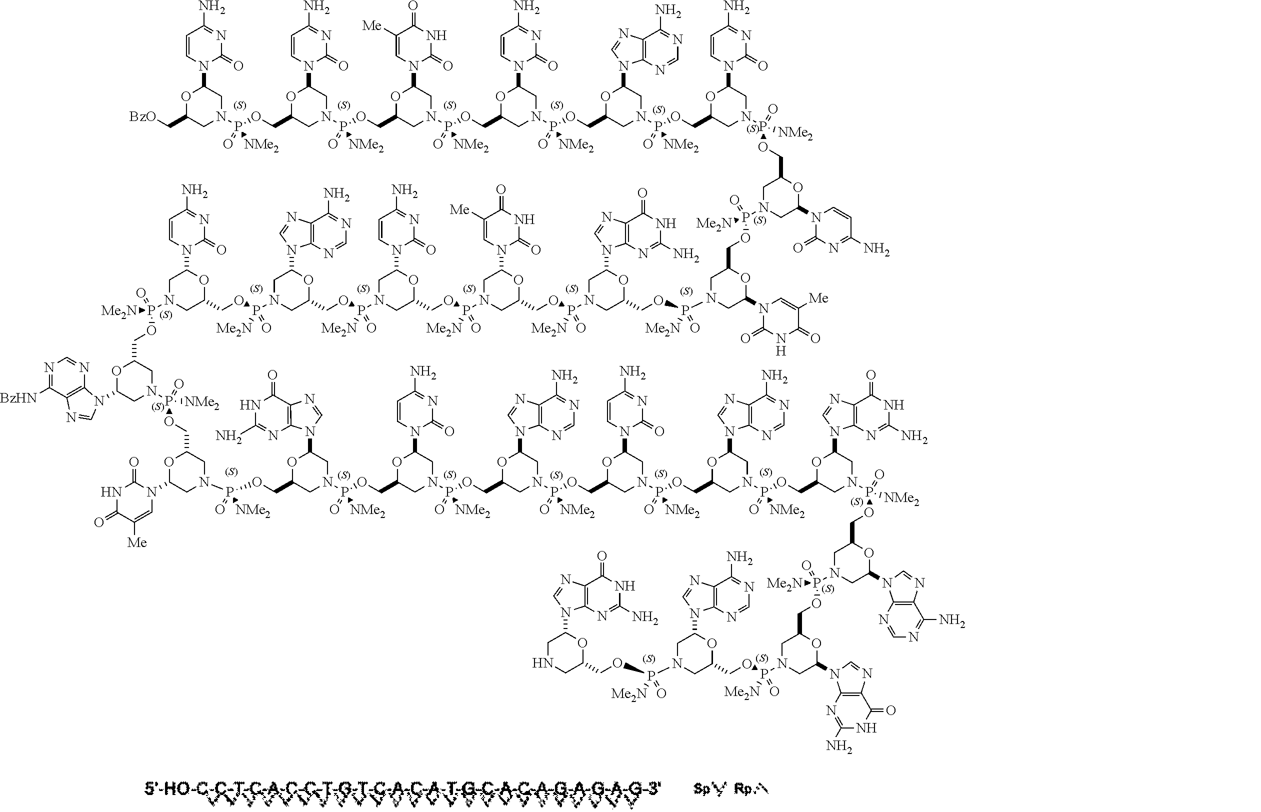

To a vial with trityl protected 25-mer (3.7 mg) was added 0.1 M phosphoric acid (250 μL). The vial was agitated at rt for 4h when the reaction was deemed completed (two consecutive checks by UPLC MS shows starting material peak was converted to a earlier eluting peak). Added 0.1M ammonium hydroxide (250 μL) and filtered through a syringe filter of 0.2 μM. The filter was rinsed with 0.4 mL of water and collected into a vial. The sample was purified by reverse phase HPLC using the method in the below table. The desired fractions were combined and evaporated under vacuum, then lyophilized to afford the desired product 1.2 mg of 25-mer PMO (PMO-302). MS (ESI) m/z: Calcd for C₂₈₉H₄₅₀N₁₄₄O₉₆P₂₄: [M+1H]⁺8220.86; Found: 8220.87 (deconvoluted HRMS spectrum).

Purification conditions for fully-deprotected PMO:

| Column: | Waters Xbridge prep C18 5 um OBD | | | |
|---|---|---|---|---|
| | 19 × 100 mm (Part Number: 186002978) | | | |
| Instrument: | Waters Prep HPLC | | | |
| Mobile phase A: | Water + 0.1% Ammonium Hydroxide | | | |
| Mobile phase B: | Acetonitrile + 0.1% Ammonium Hydroxide | | | |
| Column Temperature: | Unregulated | | | |
| Gradient: | | | | |
| | TIME (min) | A % | B % | |
| | 0 | 95 | 5 | Initial |
| | 1.1 | 95 | 5 | Elution Gradient |
| | 11.1 | 80 | 20 | |
| | 12.1 | 5 | 95 | |
| | 13.1 | 5 | 95 | |
| | 13.2 | 95 | 5 | Reset Conditions |
| | 15 | 95 | 5 | |

| | |
|---|---|
| Flow Rate (mL/min): | 40 |
| Wavelength: | DAD + 260 nm + timed collection |

### Example 4: Evaluation of Splice Modulation Properties of CD33 Exon-2 Targeting Oligonucleotides

Different technologies can be used to assess the activity/properties of CD33 targeting oligonucleotides using various human, mouse, and non-human primate cell lines.

### In Vitro Assay Methods:

U-188 MG (human glioblastoma cell lines) and human iCell Microglia cells were used for screening of CD33 Exon-2 skipping ASOs (CD33 Oligonucleotides). U-118 MG cell lines were purchased from ATCC. iCell Microglia cells were purchased from Fujifilm (Cellular Dynamics). Both cellular models were cultured and maintained using appropriate media suggested in the vendor protocols. Screening was performed in 96 WP formats, seeding about 20,000 cells per well and treating with specified concentrations of modified ASOs using Endo-Porter or Lipofectamine reagents. Cells were further incubated at 37° C. in a cell culture incubator for 48 hours before isolating the total RNA. Various experiments were carried out in biological duplicates. Total RNA was isolated and converted to cDNA as per vendor protocol, then Taqman gene expression assays were used to quantify Exon-2 skipped and un-skipped CD33 mRNA transcripts. Human house-keeping genes such as HPRT1 or GAPDH1 expressions were used to normalize the target transcript expressions.

### Evaluation of PMO-ASO Sequences

PMO ASOs were tested for their efficacy in inducing skipping of Exon-2 in a CD33 gene transcript in U-118 MG glioblastoma cells in vitro. PMO ASOs were designed to cover CD33 Exon-2 and its surrounding introns (SEQ ID NO: 1) in 25 nucleotide sections that moved down SEQ ID NO:1 5′ to 3′ five nucleotides at a time. Oligonucleotides were tested using two concentrations (0.5 μM and 0.167 μM) and delivered using Endo-Porter reagents. Cells harvested and RNA isolated at 48 hours of post treatment. Total RNA was converted to cDNA as per vendor protocol and Taqman gene expression assays were used to quantify Exon-2 skipped and un-skipped CD33 mRNA transcripts. Human house-keeping gene HPRT1 expression was used as a loading control for each experiment. Each oligonucleotide was tested twice at each concentration.

The skipping efficiency of the oligonucleotides was calculated using the following formula.

$$Skipping \% = \frac{( Skipped Value )}{( Skipped ) * ( Un - skipped )} \times 100$$

Skipping efficiency is represented on a scale of 0 to 100, wherein 100 represents 100% skipping of CD33 Exon-2 skipping. Each experiment used a negative control oligonucleotide, NTC (Non-Targeting Control), which does not target CD33.

TABLE 3

| | | SEQ. | | | |
|---|---|---|---|---|---|
| | | ID | Conc. | Exon-2 | |
| ASO NO: | Sequence (5′ to 3′) | NO: | (μM) | Skipping % | |
| PBS | Not Applicable | PBS | 0 | 15.69 | 22.01 |
| PBS | Not Applicable | PBS | 0 | 18.13 | 13.26 |
| PMO-016 | CAGTACCAGGGTCCCATCCCAGCCC | 16 | 0.5 | 20.58 | 25.01 |
| PMO-016 | CAGTACCAGGGTCCCATCCCAGCCC | 16 | 0.167 | 17.69 | 20.34 |
| PMO-017 | CCAGGGTCCCATCCCAGCCCTGCCC | 17 | 0.5 | 17.00 | 21.53 |
| PMO-017 | CCAGGGTCCCATCCCAGCCCTGCCC | 17 | 0.167 | 19.12 | 19.13 |
| PMO-018 | GTCCCATCCCAGCCCTGCCCTGCAG | 18 | 0.5 | 19.50 | 19.62 |
| PMO-018 | GTCCCATCCCAGCCCTGCCCTGCAG | 18 | 0.167 | 18.34 | 20.68 |
| PMO-019 | ATCCCAGCCCTGCCCTGCAGTACCC | 19 | 0.5 | 18.31 | 17.77 |
| PMO-019 | ATCCCAGCCCTGCCCTGCAGTACCC | 19 | 0.167 | 17.82 | 18.09 |
| PMO-020 | AGCCCTGCCCTGCAGTACCCATGAA | 20 | 0.5 | ND | 17.85 |
| PMO-020 | AGCCCTGCCCTGCAGTACCCATGAA | 20 | 0.167 | 19.28 | 17.35 |
| PMO-021 | TGCCCTGCAGTACCCATGAACTTCC | 21 | 0.5 | 17.12 | 14.33 |
| PMO-021 | TGCCCTGCAGTACCCATGAACTTCC | 21 | 0.167 | 19.94 | 15.79 |
| PMO-022 | TGCAGTACCCATGAACTTCCCTTGC | 22 | 0.5 | 10.76 | 9.32 |
| PMO-022 | TGCAGTACCCATGAACTTCCCTTGC | 22 | 0.167 | 17.90 | 17.11 |
| PMO-023 | TACCCATGAACTTCCCTTGCGGCCA | 23 | 0.5 | ND | 10.73 |
| PMO-023 | TACCCATGAACTTCCCTTGCGGCCA | 23 | 0.167 | 16.04 | 12.77 |
| PMO-024 | ATGAACTTCCCTTGCGGCCACTTCT | 24 | 0.5 | 14.47 | 12.34 |
| PMO-024 | ATGAACTTCCCTTGCGGCCACTTCT | 24 | 0.167 | 16.55 | 16.86 |
| PMO-025 | CTTCCCTTGCGGCCACTTCTGAAGC | 25 | 0.5 | 11.82 | 11.09 |
| PMO-025 | CTTCCCTTGCGGCCACTTCTGAAGC | 25 | 0.167 | 16.10 | 16.30 |
| PMO-026 | CTTGCGGCCACTTCTGAAGCCTGTG | 26 | 0.5 | 11.01 | 10.46 |
| PMO-026 | CTTGCGGCCACTTCTGAAGCCTGTG | 26 | 0.167 | 12.54 | 14.08 |
| PMO-027 | GGCCACTTCTGAAGCCTGTGCCTCA | 27 | 0.5 | 23.17 | 24.10 |
| PMO-027 | GGCCACTTCTGAAGCCTGTGCCTCA | 27 | 0.167 | 17.64 | 18.26 |
| PMO-028 | CTTCTGAAGCCTGTGCCTCACCTGT | 28 | 0.5 | 29.39 | 27.55 |
| PMO-028 | CTTCTGAAGCCTGTGCCTCACCTGT | 28 | 0.167 | 19.87 | 17.91 |
| PMO-029 | GAAGCCTGTGCCTCACCTGTCACAT | 29 | 0.5 | 28.57 | 24.78 |
| PMO-029 | GAAGCCTGTGCCTCACCTGTCACAT | 29 | 0.167 | 20.96 | 20.73 |
| PMO-030 | CTGTGCCTCACCTGTCACATGCACA | 30 | 0.5 | 25.85 | 27.90 |
| PMO-030 | CTGTGCCTCACCTGTCACATGCACA | 30 | 0.167 | 19.42 | 22.08 |
| PMO-002 | CCTCACCTGTCACATGCACAGAGAG | 2 | 0.5 | 38.65 | 37.45 |
| PMO-002 | CCTCACCTGTCACATGCACAGAGAG | 2 | 0.167 | 26.85 | 26.74 |
| PMO-003 | CCTGTCACATGCACAGAGAGCTGGG | 3 | 0.5 | 34.57 | 48.48 |
| PMO-003 | CCTGTCACATGCACAGAGAGCTGGG | 3 | 0.167 | 22.33 | 29.52 |
| PMO-031 | CACATGCACAGAGAGCTGGGGAGAT | 31 | 0.5 | 18.84 | 23.01 |
| PMO-031 | CACATGCACAGAGAGCTGGGGAGAT | 31 | 0.167 | 17.14 | 15.10 |
| PMO-032 | GCACAGAGAGCTGGGGAGATTTGTA | 32 | 0.5 | 17.35 | 16.34 |
| PMO-032 | GCACAGAGAGCTGGGGAGATTTGTA | 32 | 0.167 | 17.78 | 14.79 |
| PMO-033 | GAGAGCTGGGGAGATTTGTAACTGT | 33 | 0.5 | 23.37 | 18.65 |
| PMO-033 | GAGAGCTGGGGAGATTTGTAACTGT | 33 | 0.167 | 22.58 | 21.78 |
| PMO-034 | CTGGGGAGATTTGTAACTGTATTTG | 34 | 0.5 | ND | 18.38 |
| PMO-034 | CTGGGGAGATTTGTAACTGTATTTG | 34 | 0.167 | 17.67 | 16.19 |
| PMO-035 | GAGATTTGTAACTGTATTTGGTACT | 35 | 0.5 | 27.36 | 26.10 |
| PMO-035 | GAGATTTGTAACTGTATTTGGTACT | 35 | 0.167 | ND | 23.64 |
| PMO-036 | TTGTAACTGTATTTGGTACTTCCTC | 36 | 0.5 | 40.31 | 31.62 |
| PMO-036 | TTGTAACTGTATTTGGTACTTCCTC | 36 | 0.167 | 25.77 | 26.98 |
| PMO-037 | ACTGTATTTGGTACTTCCTCTCTCC | 37 | 0.5 | 39.08 | 44.31 |
| PMO-037 | ACTGTATTTGGTACTTCCTCTCTCC | 37 | 0.167 | 32.28 | 32.79 |
| PMO-004 | ATTTGGTACTTCCTCTCTCCATCCG | 4 | 0.5 | 38.03 | 43.18 |
| PMO-004 | ATTTGGTACTTCCTCTCTCCATCCG | 4 | 0.167 | 35.68 | 27.95 |
| PMO-038 | GTACTTCCTCTCTCCATCCGAAAGA | 38 | 0.5 | 32.51 | 37.10 |
| PMO-038 | GTACTTCCTCTCTCCATCCGAAAGA | 38 | 0.167 | 25.18 | 23.40 |
| PMO-039 | TCCTCTCTCCATCCGAAAGAAGTAT | 39 | 0.5 | 43.59 | 35.11 |
| PMO-039 | TCCTCTCTCCATCCGAAAGAAGTAT | 39 | 0.167 | 28.26 | 31.00 |
| PMO-005 | TCTCCATCCGAAAGAAGTATGAACC | 5 | 0.5 | 35.76 | 37.72 |
| PMO-005 | TCTCCATCCGAAAGAAGTATGAACC | 5 | 0.167 | 28.69 | 28.28 |
| PMO-040 | ATCCGAAAGAAGTATGAACCATTAT | 40 | 0.5 | 35.90 | 29.79 |
| PMO-040 | ATCCGAAAGAAGTATGAACCATTAT | 40 | 0.167 | 26.41 | 24.05 |
| PMO-041 | AAAGAAGTATGAACCATTATCCCTC | 41 | 0.5 | 26.90 | 27.85 |
| PMO-041 | AAAGAAGTATGAACCATTATCCCTC | 41 | 0.167 | 25.80 | 27.27 |
| PMO-042 | AGTATGAACCATTATCCCTCCTCCT | 42 | 0.5 | 21.24 | 19.46 |
| PMO-042 | AGTATGAACCATTATCCCTCCTCCT | 42 | 0.167 | 19.56 | 21.66 |
| PMO-043 | GAACCATTATCCCTCCTCCTGGCGT | 43 | 0.5 | 30.13 | 27.10 |
| PMO-043 | GAACCATTATCCCTCCTCCTGGCGT | 43 | 0.167 | 24.70 | 25.35 |
| PMO-044 | ATTATCCCTCCTCCTGGCGTCTACG | 44 | 0.5 | 26.86 | 24.63 |
| PMO-044 | ATTATCCCTCCTCCTGGCGTCTACG | 44 | 0.167 | 20.68 | 26.29 |
| PMO-045 | CCCTCCTCCTGGCGTCTACGATGCT | 45 | 0.5 | 23.56 | 22.61 |
| PMO-045 | CCCTCCTCCTGGCGTCTACGATGCT | 45 | 0.167 | 19.23 | 23.34 |
| PMO-046 | CTCCTGGCGTCTACGATGCTCAGGG | 46 | 0.5 | 24.16 | 28.53 |
| PMO-046 | CTCCTGGCGTCTACGATGCTCAGGG | 46 | 0.167 | 26.02 | 25.22 |
| PMO-047 | GGCGTCTACGATGCTCAGGGAGCAG | 47 | 0.5 | 23.39 | 26.54 |
| PMO-047 | GGCGTCTACGATGCTCAGGGAGCAG | 47 | 0.167 | 24.89 | 23.35 |
| PMO-048 | CTACGATGCTCAGGGAGCAGTTGTT | 48 | 0.5 | 23.09 | 26.70 |
| PMO-048 | CTACGATGCTCAGGGAGCAGTTGTT | 48 | 0.167 | 24.86 | 20.24 |
| PMO-049 | ATGCTCAGGGAGCAGTTGTTCCTAC | 49 | 0.5 | 23.21 | 23.31 |
| PMO-049 | ATGCTCAGGGAGCAGTTGTTCCTAC | 49 | 0.167 | 20.58 | ND |
| PMO-050 | CAGGGAGCAGTTGTTCCTACTGGGA | 50 | 0.5 | 26.92 | 21.31 |
| PMO-050 | CAGGGAGCAGTTGTTCCTACTGGGA | 50 | 0.167 | 17.69 | 20.22 |
| PMO-051 | AGCAGTTGTTCCTACTGGGATCCCC | 51 | 0.5 | 20.55 | 21.10 |
| PMO-051 | AGCAGTTGTTCCTACTGGGATCCCC | 51 | 0.167 | 21.30 | 18.81 |
| PMO-052 | TTGTTCCTACTGGGATCCCCAAGGA | 52 | 0.5 | 14.29 | 17.26 |
| PMO-052 | TTGTTCCTACTGGGATCCCCAAGGA | 52 | 0.167 | 26.38 | 19.68 |
| PMO-053 | CCTACTGGGATCCCCAAGGAGGCGG | 53 | 0.5 | 18.37 | 15.87 |
| PMO-053 | CCTACTGGGATCCCCAAGGAGGCGG | 53 | 0.167 | 13.70 | 21.48 |
| PMO-054 | TGGGATCCCCAAGGAGGCGGAATCT | 54 | 0.5 | 18.17 | 26.18 |
| PMO-054 | TGGGATCCCCAAGGAGGCGGAATCT | 54 | 0.167 | 19.79 | 19.06 |
| PMO-055 | TCCCCAAGGAGGCGGAATCTGCCCT | 55 | 0.5 | 19.77 | 18.45 |
| PMO-055 | TCCCCAAGGAGGCGGAATCTGCCCT | 55 | 0.167 | 21.96 | 13.22 |
| PMO-056 | AAGGAGGCGGAATCTGCCCTGAGTC | 56 | 0.5 | 18.42 | 28.31 |
| PMO-056 | AAGGAGGCGGAATCTGCCCTGAGTC | 56 | 0.167 | 20.39 | 18.38 |
| PMO-057 | GGCGGAATCTGCCCTGAGTCTCCTC | 57 | 0.5 | 16.83 | 14.78 |
| PMO-057 | GGCGGAATCTGCCCTGAGTCTCCTC | 57 | 0.167 | 21.10 | 19.33 |
| PMO-058 | AATCTGCCCTGAGTCTCCTCCTGTA | 58 | 0.5 | 14.23 | 13.92 |
| PMO-058 | AATCTGCCCTGAGTCTCCTCCTGTA | 58 | 0.167 | 15.61 | 20.25 |
| PMO-059 | GCCCTGAGTCTCCTCCTGTACTTCT | 59 | 0.5 | 15.60 | 17.24 |
| PMO-059 | GCCCTGAGTCTCCTCCTGTACTTCT | 59 | 0.167 | 16.31 | 17.25 |
| PMO-060 | GAGTCTCCTCCTGTACTTCTTGATC | 60 | 0.5 | 17.48 | 11.05 |
| PMO-060 | GAGTCTCCTCCTGTACTTCTTGATC | 60 | 0.167 | 17.37 | 17.20 |
| PMO-061 | TCCTCCTGTACTTCTTGATCTAGCT | 61 | 0.5 | 16.82 | 19.32 |
| PMO-061 | TCCTCCTGTACTTCTTGATCTAGCT | 61 | 0.167 | 16.57 | 16.78 |
| PMO-062 | CTGTACTTCTTGATCTAGCTTGTTT | 62 | 0.5 | 12.61 | 15.76 |
| PMO-062 | CTGTACTTCTTGATCTAGCTTGTTT | 62 | 0.167 | 15.85 | 16.60 |
| PMO-063 | CTTCTTGATCTAGCTTGTTTGTGGC | 63 | 0.5 | 7.95 | 7.13 |
| PMO-063 | CTTCTTGATCTAGCTTGTTTGTGGC | 63 | 0.167 | 14.81 | 16.45 |
| PMO-064 | TGATCTAGCTTGTTTGTGGCCACTG | 64 | 0.5 | 11.67 | 15.59 |
| PMO-064 | TGATCTAGCTTGTTTGTGGCCACTG | 64 | 0.167 | 15.34 | 16.81 |
| PMO-065 | TAGCTTGTTTGTGGCCACTGGAGAG | 65 | 0.5 | 9.86 | 12.58 |
| PMO-065 | TAGCTTGTTTGTGGCCACTGGAGAG | 65 | 0.167 | 14.97 | 14.60 |
| PMO-066 | TGTTTGTGGCCACTGGAGAGTCCCT | 66 | 0.5 | 16.61 | 13.93 |
| PMO-066 | TGTTTGTGGCCACTGGAGAGTCCCT | 66 | 0.167 | 15.52 | 14.44 |
| PMO-067 | GTGGCCACTGGAGAGTCCCTGGATA | 67 | 0.5 | 14.40 | 12.62 |
| PMO-067 | GTGGCCACTGGAGAGTCCCTGGATA | 67 | 0.167 | 14.38 | 16.49 |
| PMO-068 | CACTGGAGAGTCCCTGGATATAATG | 68 | 0.5 | 16.74 | 19.00 |
| PMO-068 | CACTGGAGAGTCCCTGGATATAATG | 68 | 0.167 | 16.44 | 16.67 |
| PMO-069 | GAGAGTCCCTGGATATAATGGCTCC | 69 | 0.5 | 17.04 | 17.70 |
| PMO-069 | GAGAGTCCCTGGATATAATGGCTCC | 69 | 0.167 | 19.01 | 19.95 |
| PMO-070 | TCCCTGGATATAATGGCTCCTTCCC | 70 | 0.5 | 17.32 | 16.87 |
| PMO-070 | TCCCTGGATATAATGGCTCCTTCCC | 70 | 0.167 | 16.41 | 14.39 |
| PMO-071 | GGATATAATGGCTCCTTCCCGGAAC | 71 | 0.5 | 15.59 | 18.00 |
| PMO-071 | GGATATAATGGCTCCTTCCCGGAAC | 71 | 0.167 | 18.67 | 28.86 |
| PMO-072 | TAATGGCTCCTTCCCGGAACCAGTA | 72 | 0.5 | 17.61 | 18.80 |
| PMO-072 | TAATGGCTCCTTCCCGGAACCAGTA | 72 | 0.167 | 16.00 | 22.11 |
| PMO-073 | GCTCCTTCCCGGAACCAGTAACCAT | 73 | 0.5 | 15.92 | 16.69 |
| PMO-073 | GCTCCTTCCCGGAACCAGTAACCAT | 73 | 0.167 | 15.96 | 15.51 |
| PMO-074 | TTCCCGGAACCAGTAACCATGAACT | 74 | 0.5 | 14.79 | 19.48 |
| PMO-074 | TTCCCGGAACCAGTAACCATGAACT | 74 | 0.167 | 16.78 | 15.98 |
| PMO-075 | GGAACCAGTAACCATGAACTGGGGA | 75 | 0.5 | 15.96 | 14.76 |
| PMO-075 | GGAACCAGTAACCATGAACTGGGGA | 75 | 0.167 | 20.20 | 20.33 |
| PMO-076 | CAGTAACCATGAACTGGGGAGTTCT | 76 | 0.5 | 15.12 | 15.51 |
| PMO-076 | CAGTAACCATGAACTGGGGAGTTCT | 76 | 0.167 | 15.78 | 17.18 |
| PMO-077 | ACCATGAACTGGGGAGTTCTTGTCG | 77 | 0.5 | 10.79 | 15.88 |
| PMO-077 | ACCATGAACTGGGGAGTTCTTGTCG | 77 | 0.167 | 17.29 | 4.46 |
| PMO-078 | GAACTGGGGAGTTCTTGTCGTAGTA | 78 | 0.5 | 18.50 | 11.38 |
| PMO-078 | GAACTGGGGAGTTCTTGTCGTAGTA | 78 | 0.167 | 13.09 | 16.04 |
| PMO-079 | GGGGAGTTCTTGTCGTAGTAGGGTA | 79 | 0.5 | 11.62 | 12.30 |
| PMO-079 | GGGGAGTTCTTGTCGTAGTAGGGTA | 79 | 0.167 | 15.89 | 15.01 |
| PMO-080 | GTTCTTGTCGTAGTAGGGTATGGGA | 80 | 0.5 | 16.44 | 16.32 |
| PMO-080 | GTTCTTGTCGTAGTAGGGTATGGGA | 80 | 0.167 | 19.96 | 20.31 |
| PMO-081 | TGTCGTAGTAGGGTATGGGATGGAA | 81 | 0.5 | 20.16 | 15.18 |
| PMO-081 | TGTCGTAGTAGGGTATGGGATGGAA | 81 | 0.167 | 17.13 | 16.78 |
| PMO-082 | TAGTAGGGTATGGGATGGAAGAAAG | 82 | 0.5 | 37.51 | 32.78 |
| PMO-082 | TAGTAGGGTATGGGATGGAAGAAAG | 82 | 0.167 | 23.94 | 24.65 |
| PMO-083 | GGGTATGGGATGGAAGAAAGTGCAG | 83 | 0.5 | 29.55 | 31.59 |
| PMO-083 | GGGTATGGGATGGAAGAAAGTGCAG | 83 | 0.167 | 26.54 | 25.26 |
| PMO-006 | TGGGATGGAAGAAAGTGCAGGGCAC | 6 | 0.5 | 33.94 | 28.96 |
| PMO-006 | TGGGATGGAAGAAAGTGCAGGGCAC | 6 | 0.167 | 22.10 | 21.87 |
| PMO-084 | TGGAAGAAAGTGCAGGGCACGAGGA | 84 | 0.5 | 21.23 | 22.41 |
| PMO-084 | TGGAAGAAAGTGCAGGGCACGAGGA | 84 | 0.167 | 20.11 | 20.47 |
| PMO-085 | GAAAGTGCAGGGCACGAGGACGCAC | 85 | 0.5 | 19.04 | 18.64 |
| PMO-085 | GAAAGTGCAGGGCACGAGGACGCAC | 85 | 0.167 | 18.98 | 17.74 |
| PMO-086 | TGCAGGGCACGAGGACGCACAAACC | 86 | 0.5 | 17.81 | 16.75 |
| PMO-086 | TGCAGGGCACGAGGACGCACAAACC | 86 | 0.167 | 18.70 | 17.72 |
| PMO-087 | GGCACGAGGACGCACAAACCCTCCT | 87 | 0.5 | 15.65 | 17.94 |
| PMO-087 | GGCACGAGGACGCACAAACCCTCCT | 87 | 0.167 | 17.82 | 17.12 |
| PMO-088 | GAGGACGCACAAACCCTCCTGTACC | 88 | 0.5 | 22.94 | 18.52 |
| PMO-088 | GAGGACGCACAAACCCTCCTGTACC | 88 | 0.167 | 19.69 | 16.19 |
| PMO-089 | CGCACAAACCCTCCTGTACCGTCAC | 89 | 0.5 | 19.29 | 26.13 |
| PMO-089 | CGCACAAACCCTCCTGTACCGTCAC | 89 | 0.167 | 23.21 | 24.50 |
| PMO-090 | AAACCCTCCTGTACCGTCACTGACT | 90 | 0.5 | 22.94 | 23.01 |
| PMO-090 | AAACCCTCCTGTACCGTCACTGACT | 90 | 0.167 | 19.34 | 21.74 |
| PMO-091 | CTCCTGTACCGTCACTGACTCCTGC | 91 | 0.5 | 18.54 | 21.34 |
| PMO-091 | CTCCTGTACCGTCACTGACTCCTGC | 91 | 0.167 | 22.92 | 21.23 |
| PMO-092 | GTACCGTCACTGACTCCTGCACTTG | 92 | 0.5 | 27.95 | 25.22 |
| PMO-092 | GTACCGTCACTGACTCCTGCACTTG | 92 | 0.167 | 20.83 | 21.67 |
| PMO-093 | GTCACTGACTCCTGCACTTGCAGCC | 93 | 0.5 | 30.17 | 27.61 |
| PMO-093 | GTCACTGACTCCTGCACTTGCAGCC | 93 | 0.167 | 16.83 | 20.18 |
| PMO-094 | TGACTCCTGCACTTGCAGCCAGAAA | 94 | 0.5 | 22.59 | 24.97 |
| PMO-094 | TGACTCCTGCACTTGCAGCCAGAAA | 94 | 0.167 | 19.41 | 19.40 |
| PMO-095 | CCTGCACTTGCAGCCAGAAATTTGG | 95 | 0.5 | 24.04 | 24.47 |
| PMO-095 | CCTGCACTTGCAGCCAGAAATTTGG | 95 | 0.167 | 20.75 | 25.75 |
| PMO-096 | ACTTGCAGCCAGAAATTTGGATCCA | 96 | 0.5 | 27.13 | 31.68 |
| PMO-096 | ACTTGCAGCCAGAAATTTGGATCCA | 96 | 0.167 | 20.64 | 23.17 |
| PMO-007 | CAGCCAGAAATTTGGATCCATAGCC | 7 | 0.5 | 33.54 | 24.73 |
| PMO-007 | CAGCCAGAAATTTGGATCCATAGCC | 7 | 0.167 | 22.72 | 20.38 |
| PMO-097 | AGAAATTTGGATCCATAGCCAGGGC | 97 | 0.5 | 27.54 | 30.55 |
| PMO-097 | AGAAATTTGGATCCATAGCCAGGGC | 97 | 0.167 | 24.97 | 26.23 |
| PMO-098 | TTTGGATCCATAGCCAGGGCCCCTG | 98 | 0.5 | 24.69 | 19.34 |
| PMO-098 | TTTGGATCCATAGCCAGGGCCCCTG | 98 | 0.167 | 20.23 | 13.18 |
| PMO-099 | ATCCATAGCCAGGGCCCCTGTGGGG | 99 | 0.5 | 21.37 | 16.72 |
| PMO-099 | ATCCATAGCCAGGGCCCCTGTGGGG | 99 | 0.167 | 17.63 | 17.91 |
| PMO-100 | TAGCCAGGGCCCCTGTGGGGAAACG | 100 | 0.5 | 18.47 | 16.44 |
| PMO-100 | TAGCCAGGGCCCCTGTGGGGAAACG | 100 | 0.167 | 17.48 | 14.77 |
| PMO-101 | AGGGCCCCTGTGGGGAAACGAGGGT | 101 | 0.5 | 23.22 | 20.19 |
| PMO-101 | AGGGCCCCTGTGGGGAAACGAGGGT | 101 | 0.167 | 18.70 | 15.95 |
| PMO-008 | CCCTGTGGGGAAACGAGGGTCAGCT | 8 | 0.5 | 25.81 | 22.09 |
| PMO-008 | CCCTGTGGGGAAACGAGGGTCAGCT | 8 | 0.167 | 18.18 | 15.45 |
| PMO-102 | TGGGGAAACGAGGGTCAGCTCGGCC | 102 | 0.5 | 25.88 | 26.13 |
| PMO-102 | TGGGGAAACGAGGGTCAGCTCGGCC | 102 | 0.167 | 21.60 | 16.73 |
| PMO-103 | AAACGAGGGTCAGCTCGGCCCAGCC | 103 | 0.5 | 29.05 | 21.15 |
| PMO-103 | AAACGAGGGTCAGCTCGGCCCAGCC | 103 | 0.167 | 19.36 | 14.31 |
| PMO-104 | AGGGTCAGCTCGGCCCAGCCCGACA | 104 | 0.5 | 21.67 | 17.87 |
| PMO-104 | AGGGTCAGCTCGGCCCAGCCCGACA | 104 | 0.167 | 15.37 | 14.73 |
| PMO-105 | CAGCTCGGCCCAGCCCGACAACCCC | 105 | 0.5 | 14.61 | 16.34 |
| PMO-105 | CAGCTCGGCCCAGCCCGACAACCCC | 105 | 0.167 | 16.89 | 14.93 |
| PMO-106 | CGGCCCAGCCCGACAACCCCTCTCC | 106 | 0.5 | 17.13 | 14.47 |
| PMO-106 | CGGCCCAGCCCGACAACCCCTCTCC | 106 | 0.167 | 15.72 | 14.80 |
| PMO-107 | CAGCCCGACAACCCCTCTCCCCACA | 107 | 0.5 | 16.70 | 13.34 |
| PMO-107 | CAGCCCGACAACCCCTCTCCCCACA | 107 | 0.167 | 15.72 | 12.60 |
| PMO-108 | CGACAACCCCTCTCCCCACAGCCAC | 108 | 0.5 | 14.21 | 12.88 |
| PMO-108 | CGACAACCCCTCTCCCCACAGCCAC | 108 | 0.167 | 16.03 | 12.42 |
| PMO-109 | ACCCCTCTCCCCACAGCCACTCACC | 109 | 0.5 | 18.03 | 11.73 |
| PMO-109 | ACCCCTCTCCCCACAGCCACTCACC | 109 | 0.167 | 14.53 | 11.90 |
| PMO-110 | TCTCCCCACAGCCACTCACCTGCCC | 110 | 0.5 | 14.10 | 12.30 |
| PMO-110 | TCTCCCCACAGCCACTCACCTGCCC | 110 | 0.167 | 15.90 | 12.20 |

### Evaluation of MOE-ASO Sequences

MOE ASOs were tested for their efficacy in inducing skipping of Exon-2 in a CD33 gene transcript in U-118 MG glioblastoma cells in vitro. MOE ASOs were designed to cover CD33 Exon-2 and its surrounding introns (SEQ ID NO: 1) in 20 nucleotide sections that moved down SEQ ID NO:15′ to 3′ five nucleotides at a time. Oligonucleotides were tested using different concentrations (10 nM and 3.3 nM) and delivered using the Lipofectamine protocol. Cells were harvested and RNA was isolated at 48 hours of post treatment. Total RNA was converted to cDNA as per vendor protocol and Taqman gene expression assays were used to quantify Exon-2 skipped and un-skipped CD33 mRNA transcripts. Human house-keeping gene HPRT1 expression was used as a loading control for each experiment. Each oligonucleotide was tested twice at each concentration.

The skipping efficiency of the oligonucleotides was calculated using the following formula.

$$Skipping \% = \frac{( Skipped Value )}{( Skipped ) * ( Un - skipped )} \times 100$$

Skipping efficiency is represented on a scale of 0 to 100, wherein 100 represents 100% skipping of CD33 Exon-2 skipping. Each experiment used a negative control oligonucleotide, NTC (Non-Targeting Control), which does not target CD33. Results are reported in Table 4 below.

TABLE 4

| | | SEQ. | | Exon-2 | |
|---|---|---|---|---|---|
| | | ID | Conc. | Skipping | |
| ASO NO: | Sequence (5′ to 3′) | NO: | (nM) | % | |
| PBS | Not Applicable | PBS | 0 | 18.47 | 13.36 |
| PBS | Not Applicable | PBS | 0 | 17.74 | 12.61 |
| MOE-111 | CAGTACCAGGGTCCCATCCC | 111 | 10 | 40.31 | 24.93 |
| MOE-111 | CAGTACCAGGGTCCCATCCC | 111 | 3.3 | 29.81 | 24.10 |
| MOE-112 | CCAGGGTCCCATCCCAGCCC | 112 | 10 | 31.01 | 22.56 |
| MOE-112 | CCAGGGTCCCATCCCAGCCC | 112 | 3.3 | 30.80 | 21.53 |
| MOE-113 | GTCCCATCCCAGCCCTGCCC | 113 | 10 | 22.45 | 9.58 |
| MOE-113 | GTCCCATCCCAGCCCTGCCC | 113 | 3.3 | 22.13 | 11.13 |
| MOE-114 | ATCCCAGCCCTGCCCTGCAG | 114 | 10 | 14.91 | 8.40 |
| MOE-114 | ATCCCAGCCCTGCCCTGCAG | 114 | 3.3 | 17.69 | 11.98 |
| MOE-115 | AGCCCTGCCCTGCAGTACCC | 115 | 10 | 18.87 | 12.24 |
| MOE-115 | AGCCCTGCCCTGCAGTACCC | 115 | 3.3 | 18.83 | 13.28 |
| MOE-116 | TGCCCTGCAGTACCCATGAA | 116 | 10 | ND | 6.70 |
| MOE-116 | TGCCCTGCAGTACCCATGAA | 116 | 3.3 | 9.33 | 5.16 |
| MOE-117 | TGCAGTACCCATGAACTTCC | 117 | 10 | 6.59 | 4.56 |
| MOE-117 | TGCAGTACCCATGAACTTCC | 117 | 3.3 | 6.01 | 3.33 |
| MOE-118 | TACCCATGAACTTCCCTTGC | 118 | 10 | 4.61 | 2.80 |
| MOE-118 | TACCCATGAACTTCCCTTGC | 118 | 3.3 | 3.41 | 2.33 |
| MOE-119 | ATGAACTTCCCTTGCGGCCA | 119 | 10 | 13.83 | 6.59 |
| MOE-119 | ATGAACTTCCCTTGCGGCCA | 119 | 3.3 | 10.67 | 7.25 |
| MOE-120 | CTTCCCTTGCGGCCACTTCT | 120 | 10 | 11.64 | 6.49 |
| MOE-120 | CTTCCCTTGCGGCCACTTCT | 120 | 3.3 | 12.08 | 7.49 |
| MOE-121 | CTTGCGGCCACTTCTGAAGC | 121 | 10 | 26.01 | 16.70 |
| MOE-121 | CTTGCGGCCACTTCTGAAGC | 121 | 3.3 | 17.39 | 11.23 |
| MOE-122 | GGCCACTTCTGAAGCCTGTG | 122 | 10 | 3.21 | 2.84 |
| MOE-122 | GGCCACTTCTGAAGCCTGTG | 122 | 3.3 | 4.52 | 3.12 |
| MOE-123 | CTTCTGAAGCCTGTGCCTCA | 123 | 10 | 63.47 | 55.19 |
| MOE-123 | CTTCTGAAGCCTGTGCCTCA | 123 | 3.3 | 59.19 | 46.69 |
| MOE-124 | GAAGCCTGTGCCTCACCTGT | 124 | 10 | ND | 49.44 |
| MOE-124 | GAAGCCTGTGCCTCACCTGT | 124 | 3.3 | 60.09 | 43.86 |
| MOE-125 | CTGTGCCTCACCTGTCACAT | 125 | 10 | 54.24 | 41.03 |
| MOE-125 | CTGTGCCTCACCTGTCACAT | 125 | 3.3 | 37.81 | 26.33 |
| MOE-126 | CCTCACCTGTCACATGCACA | 126 | 10 | 74.18 | 59.06 |
| MOE-126 | CCTCACCTGTCACATGCACA | 126 | 3.3 | 69.41 | 60.05 |
| MOE-127 | CCTGTCACATGCACAGAGAG | 127 | 10 | 71.43 | 49.59 |
| MOE-127 | CCTGTCACATGCACAGAGAG | 127 | 3.3 | 44.56 | 37.84 |
| MOE-009 | CACATGCACAGAGAGCTGGG | 9 | 10 | 87.63 | 71.68 |
| MOE-009 | CACATGCACAGAGAGCTGGG | 9 | 3.3 | 78.49 | 63.41 |
| MOE-128 | GCACAGAGAGCTGGGGAGAT | 128 | 10 | 87.43 | 77.61 |
| MOE-128 | GCACAGAGAGCTGGGGAGAT | 128 | 3.3 | 82.29 | 73.73 |
| MOE-010 | GAGAGCTGGGGAGATTTGTA | 10 | 10 | 85.60 | 72.24 |
| MOE-010 | GAGAGCTGGGGAGATTTGTA | 10 | 3.3 | 81.85 | 69.90 |
| MOE-129 | CTGGGGAGATTTGTAACTGT | 129 | 10 | 74.19 | 63.26 |
| MOE-129 | CTGGGGAGATTTGTAACTGT | 129 | 3.3 | 65.32 | 56.15 |
| MOE-130 | GAGATTTGTAACTGTATTTG | 130 | 10 | 62.12 | 44.93 |
| MOE-130 | GAGATTTGTAACTGTATTTG | 130 | 3.3 | 60.10 | 38.70 |
| MOE-131 | TTGTAACTGTATTTGGTACT | 131 | 10 | 66.00 | 67.32 |
| MOE-131 | TTGTAACTGTATTTGGTACT | 131 | 3.3 | 61.73 | 68.50 |
| MOE-132 | ACTGTATTTGGTACTTCCTC | 132 | 10 | 70.65 | 73.37 |
| MOE-132 | ACTGTATTTGGTACTTCCTC | 132 | 3.3 | 68.40 | 66.93 |
| MOE-133 | ATTTGGTACTTCCTCTCTCC | 133 | 10 | 75.12 | 75.05 |
| MOE-133 | ATTTGGTACTTCCTCTCTCC | 133 | 3.3 | 67.22 | 67.63 |
| MOE-134 | GTACTTCCTCTCTCCATCCG | 134 | 10 | 67.31 | 68.05 |
| MOE-134 | GTACTTCCTCTCTCCATCCG | 134 | 3.3 | 58.11 | 63.24 |
| MOE-135 | TCCTCTCTCCATCCGAAAGA | 135 | 10 | 72.52 | 78.37 |
| MOE-135 | TCCTCTCTCCATCCGAAAGA | 135 | 3.3 | 65.56 | 71.32 |
| MOE-011 | TCTCCATCCGAAAGAAGTAT | 11 | 10 | 77.95 | 79.80 |
| MOE-011 | TCTCCATCCGAAAGAAGTAT | 11 | 3.3 | 71.38 | 73.17 |
| MOE-012 | ATCCGAAAGAAGTATGAACC | 12 | 10 | 79.43 | 81.71 |
| MOE-012 | ATCCGAAAGAAGTATGAACC | 12 | 3.3 | 71.98 | 73.17 |
| MOE-136 | AAAGAAGTATGAACCATTAT | 136 | 10 | 65.32 | 64.65 |
| MOE-136 | AAAGAAGTATGAACCATTAT | 136 | 3.3 | 63.72 | 64.55 |
| MOE-137 | AGTATGAACCATTATCCCTC | 137 | 10 | 55.07 | 55.88 |
| MOE-137 | AGTATGAACCATTATCCCTC | 137 | 3.3 | 60.39 | 60.78 |
| MOE-138 | GAACCATTATCCCTCCTCCT | 138 | 10 | 45.84 | 49.89 |
| MOE-138 | GAACCATTATCCCTCCTCCT | 138 | 3.3 | 42.18 | 41.02 |
| MOE-139 | ATTATCCCTCCTCCTGGCGT | 139 | 10 | 46.81 | 47.81 |
| MOE-139 | ATTATCCCTCCTCCTGGCGT | 139 | 3.3 | 38.49 | 42.29 |
| MOE-140 | CCCTCCTCCTGGCGTCTACG | 140 | 10 | 48.67 | 51.53 |
| MOE-140 | CCCTCCTCCTGGCGTCTACG | 140 | 3.3 | 45.10 | 45.81 |
| MOE-141 | CTCCTGGCGTCTACGATGCT | 141 | 10 | 53.96 | 58.13 |
| MOE-141 | CTCCTGGCGTCTACGATGCT | 141 | 3.3 | 48.52 | 53.06 |
| MOE-142 | GGCGTCTACGATGCTCAGGG | 142 | 10 | 57.10 | 63.60 |
| MOE-142 | GGCGTCTACGATGCTCAGGG | 142 | 3.3 | 44.22 | 49.17 |
| MOE-143 | CTACGATGCTCAGGGAGCAG | 143 | 10 | 16.30 | 19.21 |
| MOE-143 | CTACGATGCTCAGGGAGCAG | 143 | 3.3 | 17.28 | 18.92 |
| MOE-013 | ATGCTCAGGGAGCAGTTGTT | 13 | 10 | 69.53 | 67.97 |
| MOE-013 | ATGCTCAGGGAGCAGTTGTT | 13 | 3.3 | 60.30 | 62.96 |
| MOE-144 | CAGGGAGCAGTTGTTCCTAC | 144 | 10 | 32.12 | 30.95 |
| MOE-144 | CAGGGAGCAGTTGTTCCTAC | 144 | 3.3 | 29.78 | 30.24 |
| MOE-145 | AGCAGTTGTTCCTACTGGGA | 145 | 10 | 54.95 | 57.38 |
| MOE-145 | AGCAGTTGTTCCTACTGGGA | 145 | 3.3 | 48.94 | 51.28 |
| MOE-146 | TTGTTCCTACTGGGATCCCC | 146 | 10 | 30.91 | 34.58 |
| MOE-146 | TTGTTCCTACTGGGATCCCC | 146 | 3.3 | 27.88 | 30.50 |
| MOE-147 | CCTACTGGGATCCCCAAGGA | 147 | 10 | 23.32 | 22.90 |
| MOE-147 | CCTACTGGGATCCCCAAGGA | 147 | 3.3 | 20.41 | 23.17 |
| MOE-148 | TGGGATCCCCAAGGAGGCGG | 148 | 10 | 28.55 | 30.05 |
| MOE-148 | TGGGATCCCCAAGGAGGCGG | 148 | 3.3 | 23.57 | 25.39 |
| MOE-149 | TCCCCAAGGAGGCGGAATCT | 149 | 10 | 35.42 | 39.05 |
| MOE-149 | TCCCCAAGGAGGCGGAATCT | 149 | 3.3 | 32.06 | 31.12 |
| MOE-150 | AAGGAGGCGGAATCTGCCCT | 150 | 10 | 26.82 | 27.76 |
| MOE-150 | AAGGAGGCGGAATCTGCCCT | 150 | 3.3 | 23.00 | 26.50 |
| MOE-151 | GGCGGAATCTGCCCTGAGTC | 151 | 10 | 24.81 | 30.28 |
| MOE-151 | GGCGGAATCTGCCCTGAGTC | 151 | 3.3 | 21.85 | 21.58 |
| MOE-152 | AATCTGCCCTGAGTCTCCTC | 152 | 10 | 20.14 | 20.99 |
| MOE-152 | AATCTGCCCTGAGTCTCCTC | 152 | 3.3 | 21.41 | 22.14 |
| MOE-153 | GCCCTGAGTCTCCTCCTGTA | 153 | 10 | 39.75 | 41.02 |
| MOE-153 | GCCCTGAGTCTCCTCCTGTA | 153 | 3.3 | 24.02 | 26.60 |
| MOE-014 | GAGTCTCCTCCTGTACTTCT | 14 | 10 | 75.96 | 78.90 |
| MOE-014 | GAGTCTCCTCCTGTACTTCT | 14 | 3.3 | 49.08 | 53.79 |
| MOE-154 | TCCTCCTGTACTTCTTGATC | 154 | 10 | 60.29 | 62.38 |
| MOE-154 | TCCTCCTGTACTTCTTGATC | 154 | 3.3 | 33.27 | 34.20 |
| MOE-155 | CTGTACTTCTTGATCTAGCT | 155 | 10 | 37.05 | 32.79 |
| MOE-155 | CTGTACTTCTTGATCTAGCT | 155 | 3.3 | 22.07 | 21.02 |
| MOE-156 | CTTCTTGATCTAGCTTGTTT | 156 | 10 | 28.56 | 24.36 |
| MOE-156 | CTTCTTGATCTAGCTTGTTT | 156 | 3.3 | 21.49 | 19.64 |
| MOE-157 | TGATCTAGCTTGTTTGTGGC | 157 | 10 | 23.31 | 20.74 |
| MOE-157 | TGATCTAGCTTGTTTGTGGC | 157 | 3.3 | 19.90 | 22.50 |
| MOE-158 | TAGCTTGTTTGTGGCCACTG | 158 | 10 | 23.59 | 24.40 |
| MOE-158 | TAGCTTGTTTGTGGCCACTG | 158 | 3.3 | 20.35 | 24.34 |
| MOE-159 | TGTTTGTGGCCACTGGAGAG | 159 | 10 | 23.61 | 27.17 |
| MOE-159 | TGTTTGTGGCCACTGGAGAG | 159 | 3.3 | 20.37 | 24.19 |
| MOE-160 | GTGGCCACTGGAGAGTCCCT | 160 | 10 | 25.04 | 26.30 |
| MOE-160 | GTGGCCACTGGAGAGTCCCT | 160 | 3.3 | 27.86 | 21.83 |
| MOE-161 | CACTGGAGAGTCCCTGGATA | 161 | 10 | 21.19 | 19.37 |
| MOE-161 | CACTGGAGAGTCCCTGGATA | 161 | 3.3 | 21.64 | 21.42 |
| MOE-162 | GAGAGTCCCTGGATATAATG | 162 | 10 | 30.10 | 30.87 |
| MOE-162 | GAGAGTCCCTGGATATAATG | 162 | 3.3 | 27.48 | 25.80 |
| MOE-163 | TCCCTGGATATAATGGCTCC | 163 | 10 | 16.89 | 19.97 |
| MOE-163 | TCCCTGGATATAATGGCTCC | 163 | 3.3 | 20.87 | 20.85 |
| MOE-164 | GGATATAATGGCTCCTTCCC | 164 | 10 | 20.18 | 18.88 |
| MOE-164 | GGATATAATGGCTCCTTCCC | 164 | 3.3 | 19.10 | 26.05 |
| MOE-165 | TAATGGCTCCTTCCCGGAAC | 165 | 10 | 15.35 | 15.55 |
| MOE-165 | TAATGGCTCCTTCCCGGAAC | 165 | 3.3 | 18.20 | 23.35 |
| MOE-166 | GCTCCTTCCCGGAACCAGTA | 166 | 10 | 24.43 | 23.98 |
| MOE-166 | GCTCCTTCCCGGAACCAGTA | 166 | 3.3 | 23.04 | 28.04 |
| MOE-167 | TTCCCGGAACCAGTAACCAT | 167 | 10 | 17.30 | 18.36 |
| MOE-167 | TTCCCGGAACCAGTAACCAT | 167 | 3.3 | 19.68 | 20.56 |
| MOE-168 | GGAACCAGTAACCATGAACT | 168 | 10 | 28.07 | 33.82 |
| MOE-168 | GGAACCAGTAACCATGAACT | 168 | 3.3 | 29.39 | 27.36 |
| MOE-169 | CAGTAACCATGAACTGGGGA | 169 | 10 | 25.12 | 35.14 |
| MOE-169 | CAGTAACCATGAACTGGGGA | 169 | 3.3 | 18.07 | 16.65 |
| MOE-170 | ACCATGAACTGGGGAGTTCT | 170 | 10 | 36.13 | 40.54 |
| MOE-170 | ACCATGAACTGGGGAGTTCT | 170 | 3.3 | 24.50 | 27.23 |
| MOE-171 | GAACTGGGGAGTTCTTGTCG | 171 | 10 | 36.74 | 40.62 |
| MOE-171 | GAACTGGGGAGTTCTTGTCG | 171 | 3.3 | 25.99 | 25.29 |
| MOE-172 | GGGGAGTTCTTGTCGTAGTA | 172 | 10 | 33.33 | 34.65 |
| MOE-172 | GGGGAGTTCTTGTCGTAGTA | 172 | 3.3 | 28.76 | 29.44 |
| MOE-173 | GTTCTTGTCGTAGTAGGGTA | 173 | 10 | 28.88 | 28.86 |
| MOE-173 | GTTCTTGTCGTAGTAGGGTA | 173 | 3.3 | 23.21 | 25.74 |
| MOE-174 | TGTCGTAGTAGGGTATGGGA | 174 | 10 | 25.89 | 27.21 |
| MOE-174 | TGTCGTAGTAGGGTATGGGA | 174 | 3.3 | 22.43 | 22.39 |
| MOE-175 | TAGTAGGGTATGGGATGGAA | 175 | 10 | 25.20 | 23.10 |
| MOE-175 | TAGTAGGGTATGGGATGGAA | 175 | 3.3 | 22.85 | 24.57 |
| MOE-176 | GGGTATGGGATGGAAGAAAG | 176 | 10 | 22.14 | 20.68 |
| MOE-176 | GGGTATGGGATGGAAGAAAG | 176 | 3.3 | 28.49 | 23.76 |
| MOE-177 | TGGGATGGAAGAAAGTGCAG | 177 | 10 | 31.21 | 31.46 |
| MOE-177 | TGGGATGGAAGAAAGTGCAG | 177 | 3.3 | 23.98 | 27.84 |
| MOE-178 | TGGAAGAAAGTGCAGGGCAC | 178 | 10 | 27.29 | 28.99 |
| MOE-178 | TGGAAGAAAGTGCAGGGCAC | 178 | 3.3 | 19.99 | 27.48 |
| MOE-179 | GAAAGTGCAGGGCACGAGGA | 179 | 10 | 12.36 | 18.07 |
| MOE-179 | GAAAGTGCAGGGCACGAGGA | 179 | 3.3 | 18.80 | 18.04 |
| MOE-180 | TGCAGGGCACGAGGACGCAC | 180 | 10 | 26.85 | 31.33 |
| MOE-180 | TGCAGGGCACGAGGACGCAC | 180 | 3.3 | 25.68 | 24.68 |
| MOE-181 | GGCACGAGGACGCACAAACC | 181 | 10 | 26.06 | 25.11 |
| MOE-181 | GGCACGAGGACGCACAAACC | 181 | 3.3 | 30.02 | 30.36 |
| MOE-182 | GAGGACGCACAAACCCTCCT | 182 | 10 | 34.00 | 32.47 |
| MOE-182 | GAGGACGCACAAACCCTCCT | 182 | 3.3 | 33.30 | 32.57 |
| MOE-015 | CGCACAAACCCTCCTGTACC | 15 | 10 | 65.75 | 69.90 |
| MOE-015 | CGCACAAACCCTCCTGTACC | 15 | 3.3 | 54.43 | 56.86 |
| MOE-183 | AAACCCTCCTGTACCGTCAC | 183 | 10 | 72.72 | 72.60 |
| MOE-183 | AAACCCTCCTGTACCGTCAC | 183 | 3.3 | 56.83 | 58.76 |
| MOE-184 | CTCCTGTACCGTCACTGACT | 184 | 10 | 74.45 | 77.20 |
| MOE-184 | CTCCTGTACCGTCACTGACT | 184 | 3.3 | 47.20 | 51.71 |
| MOE-185 | GTACCGTCACTGACTCCTGC | 185 | 10 | 55.41 | 57.23 |
| MOE-185 | GTACCGTCACTGACTCCTGC | 185 | 3.3 | 42.08 | 40.45 |
| MOE-186 | GTCACTGACTCCTGCACTTG | 186 | 10 | 41.60 | 47.16 |
| MOE-186 | GTCACTGACTCCTGCACTTG | 186 | 3.3 | 32.79 | 39.43 |
| MOE-187 | TGACTCCTGCACTTGCAGCC | 187 | 10 | 57.89 | 63.61 |
| MOE-187 | TGACTCCTGCACTTGCAGCC | 187 | 3.3 | 41.11 | 42.17 |
| MOE-188 | CCTGCACTTGCAGCCAGAAA | 188 | 10 | 54.71 | 57.79 |
| MOE-188 | CCTGCACTTGCAGCCAGAAA | 188 | 3.3 | 36.56 | 37.48 |
| MOE-189 | ACTTGCAGCCAGAAATTTGG | 189 | 10 | 24.18 | 28.93 |
| MOE-189 | ACTTGCAGCCAGAAATTTGG | 189 | 3.3 | 20.14 | 21.92 |
| MOE-190 | CAGCCAGAAATTTGGATCCA | 190 | 10 | 69.64 | 73.25 |
| MOE-190 | CAGCCAGAAATTTGGATCCA | 190 | 3.3 | 59.35 | 66.00 |
| MOE-191 | AGAAATTTGGATCCATAGCC | 191 | 10 | 50.39 | 58.23 |
| MOE-191 | AGAAATTTGGATCCATAGCC | 191 | 3.3 | 42.00 | 44.93 |
| MOE-192 | TTTGGATCCATAGCCAGGGC | 192 | 10 | 24.03 | 23.58 |
| MOE-192 | TTTGGATCCATAGCCAGGGC | 192 | 3.3 | 21.98 | 22.59 |
| MOE-193 | ATCCATAGCCAGGGCCCCTG | 193 | 10 | 38.04 | 40.05 |
| MOE-193 | ATCCATAGCCAGGGCCCCTG | 193 | 3.3 | 26.12 | 26.68 |
| MOE-194 | TAGCCAGGGCCCCTGTGGGG | 194 | 10 | 25.24 | 25.25 |
| MOE-194 | TAGCCAGGGCCCCTGTGGGG | 194 | 3.3 | 23.36 | 23.26 |
| MOE-195 | AGGGCCCCTGTGGGGAAACG | 195 | 10 | 23.50 | 19.99 |
| MOE-195 | AGGGCCCCTGTGGGGAAACG | 195 | 3.3 | 20.64 | 16.35 |
| MOE-196 | CCCTGTGGGGAAACGAGGGT | 196 | 10 | 50.10 | 51.30 |
| MOE-196 | CCCTGTGGGGAAACGAGGGT | 196 | 3.3 | 30.51 | 29.87 |
| MOE-197 | TGGGGAAACGAGGGTCAGCT | 197 | 10 | 48.33 | 45.21 |
| MOE-197 | TGGGGAAACGAGGGTCAGCT | 197 | 3.3 | 35.56 | 29.45 |
| MOE-198 | AAACGAGGGTCAGCTCGGCC | 198 | 10 | 47.94 | 49.11 |
| MOE-198 | AAACGAGGGTCAGCTCGGCC | 198 | 3.3 | 31.84 | 29.60 |
| MOE-199 | AGGGTCAGCTCGGCCCAGCC | 199 | 10 | 32.57 | 37.93 |
| MOE-199 | AGGGTCAGCTCGGCCCAGCC | 199 | 3.3 | 22.79 | 23.61 |
| MOE-200 | CAGCTCGGCCCAGCCCGACA | 200 | 10 | 19.08 | 19.10 |
| MOE-200 | CAGCTCGGCCCAGCCCGACA | 200 | 3.3 | 18.51 | 20.80 |
| MOE-201 | CGGCCCAGCCCGACAACCCC | 201 | 10 | 13.05 | 11.72 |
| MOE-201 | CGGCCCAGCCCGACAACCCC | 201 | 3.3 | 15.20 | 16.19 |
| MOE-202 | CAGCCCGACAACCCCTCTCC | 202 | 10 | 15.84 | 15.39 |
| MOE-202 | CAGCCCGACAACCCCTCTCC | 202 | 3.3 | 16.45 | 14.82 |
| MOE-203 | CGACAACCCCTCTCCCCACA | 203 | 10 | 21.09 | 19.86 |
| MOE-203 | CGACAACCCCTCTCCCCACA | 203 | 3.3 | 20.06 | 16.31 |
| MOE-204 | ACCCCTCTCCCCACAGCCAC | 204 | 10 | 18.35 | 17.90 |
| MOE-204 | ACCCCTCTCCCCACAGCCAC | 204 | 3.3 | 17.42 | 21.23 |
| MOE-205 | TCTCCCCACAGCCACTCACC | 205 | 10 | 16.97 | 17.30 |
| MOE-205 | TCTCCCCACAGCCACTCACC | 205 | 3.3 | 17.67 | 18.40 |
| MOE-206 | CCACAGCCACTCACCTGCCC | 206 | 10 | 26.79 | 27.65 |
| MOE-206 | CCACAGCCACTCACCTGCCC | 206 | 3.3 | 25.58 | 24.45 |

Identification of CD33 Regions with Increased Exon-2 Skipping Activity Using ASOs

PMO and MOE ASOs were designed to cover CD33 Exon-2 and its surrounding introns (SEQ ID NO: 1) in 20-25 nucleotide sections that moved down SEQ ID NO:15′ to 3′ five nucleotides at a time. Exon-2 skipping activity was generated in Tables 3 and 4 for these PMO and MOE ASOs, respectively. Regions that exhibited increased Exon-2 skipping activity were identified where two or more consecutive PMO or MOE ASOs that are complementary to a section of SEQ ID NO:1 showed increased Exon-2 skipping activity. Those regions were:

a. Region 1:
(SEQ ID NO: 213)
(5′-TCTCCCCAGCTCTCTGTGCATGTGACAGGTGAGGCACA-3′)
(see, e.g., PMO-002 and PMO-003)
b. Region 2:
(SEQ ID NO: 214)
(5′-TAATGGTTCATACTTCTTTCGGATGGAGAGAGGAAGTACCAAATAC
AGTTACAAATCT-3′)(see, e.g., PMO-036, PMO-037, PMO-
004, PMO-038, PMO-039, and PMO-005)
c. Region 3:
(SEQ ID NO: 215)
(5′-CCTCGTGCCCTGCACTTTCTTCCATCCCATACCCTACTACGAC-
3′)(see, e.g., PMO-082, PMO-083, and PMO-006)
d. Region 4:
(SEQ ID NO: 216)
(5′-AGGGGCCCTGGCTATGGATCCAAATTTCTGGCTGCAAGTGCAG-
3′)(see, e.g., PMO-096, PMO-007, and PMO-097)
e. Region 5:
(SEQ ID NO: 217)
(5′-ACAGTTACAAATCTCCCCAGCTCTCTGTGCATGTGACAGGTGAGG-
3′)(see, e.g., MOE-009, MOE-128, and MOE-010)
f. Region 6:
(SEQ ID NO: 218)
(5′-GGTTCATACTTCTTTCGGATGGAGAGAGGAAGTACCAAAT-3′)
(see, e.g., MOE-135, MOE-011, and MOE-012)
g. Region 7:
(SEQ ID NO: 219)
(5′-GCAGGAGTCAGTGACGGTACAGGAGGGTTTGTGCG-3′)(see,
e.g., MOE-015, MOE-183, and MOE-184)
h. Region 8:
(SEQ ID NO: 220)
(5′-GGCCGAGCTGACCCTCGTTTCCCCACAGGGGCCC-3′)(see,
e.g., MOE-196 and MOE-197).

### Evaluation of PMO-ASO Sequences at Multiple Concentrations

Oligonucleotides were tested for their efficacy in inducing skipping of Exon-2 in a CD33 gene transcript in U-118 MG glioblastoma cells in vitro. Oligonucleotides were tested using different concentrations (0.156, 0.313, 0.625, 1.25, 2.5, 5.0, 10.0 and 20.0 μM) and delivered using the Endo-Porter protocol. Cells were harvested and RNA was isolated at 48 hours of post treatment.

The skipping efficiency of the oligonucleotides was calculated using the following formula.

$$Skipping \% = \frac{( Skipped Value )}{( Skipped ) * ( Un - skipped )} \times 100$$

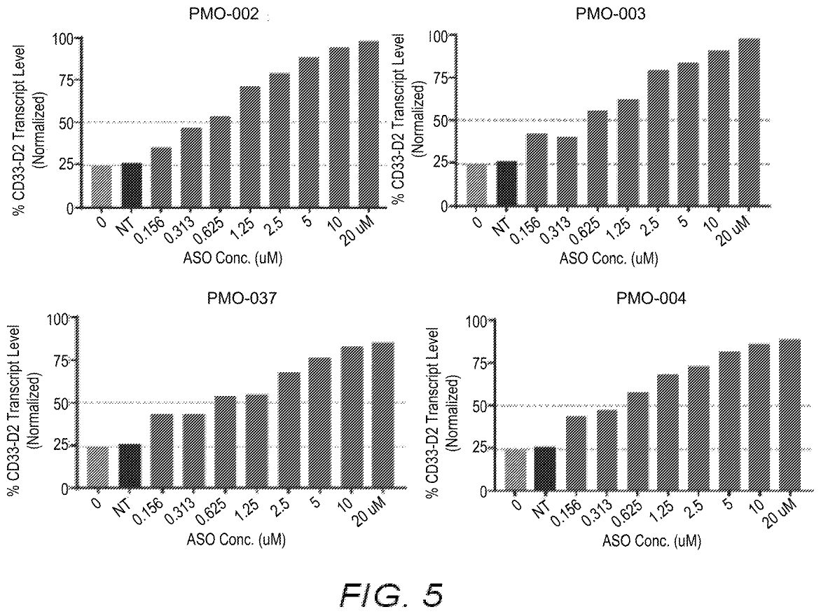

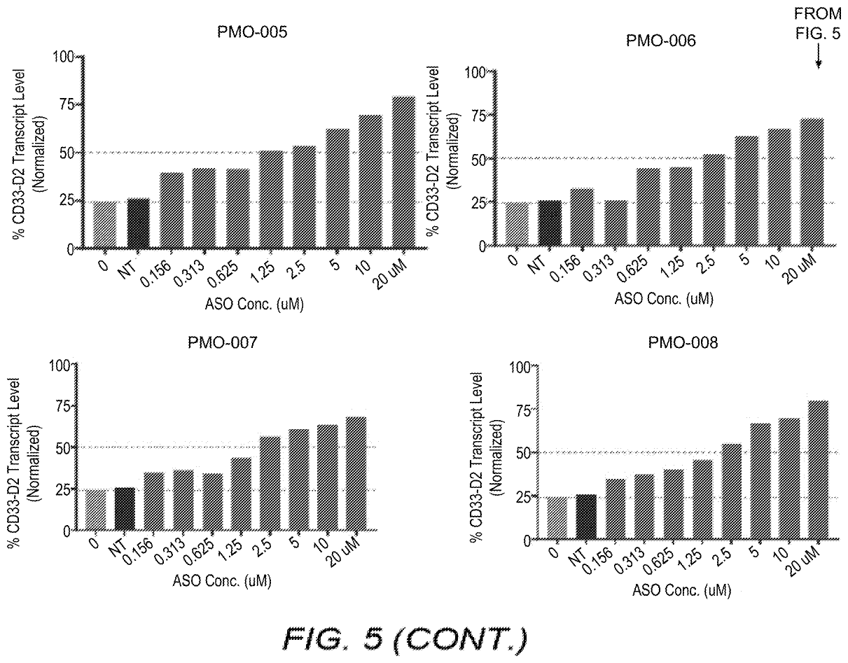

Skipping efficiency is represented on a scale of 0 to 100, wherein 100 represents 100% skipping of CD33 Exon-2 skipping. Each experiment used a negative control oligonucleotide, NTC (Non-Targeting Control), which does not target CD33. Skipping efficiency (% CD33-D2 Transcript Level (Normalized)) are shown in FIG. 5.

### Evaluation of MOE-ASO Sequences at Multiple Concentrations

Oligonucleotides were tested for their efficacy in inducing skipping of Exon-2 in a CD33 gene transcript in U-118 MG glioblastoma cells in vitro. Oligonucleotides were tested using different concentrations (0.082, 0.205, 0.512, 1.28, 3.2, 8.0, 20.0, and 50.0 nM) and delivered using the Lipofectamine protocol. Cells were harvested and RNA was isolated at 48 hours of post treatment.

The skipping efficiency of the oligonucleotides was calculated using the following formula.

$$Skipping \% = \frac{( Skipped Value )}{( Skipped ) * ( Un - skipped )} \times 100$$

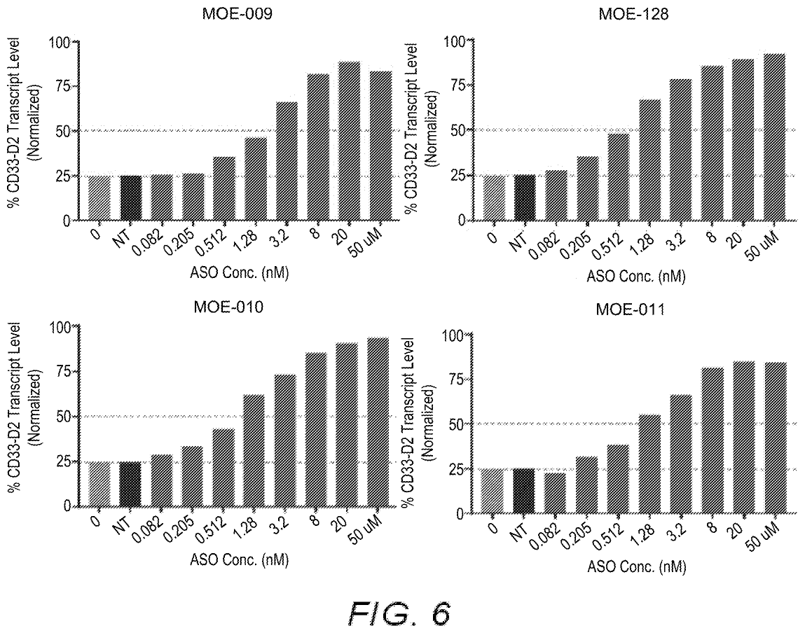

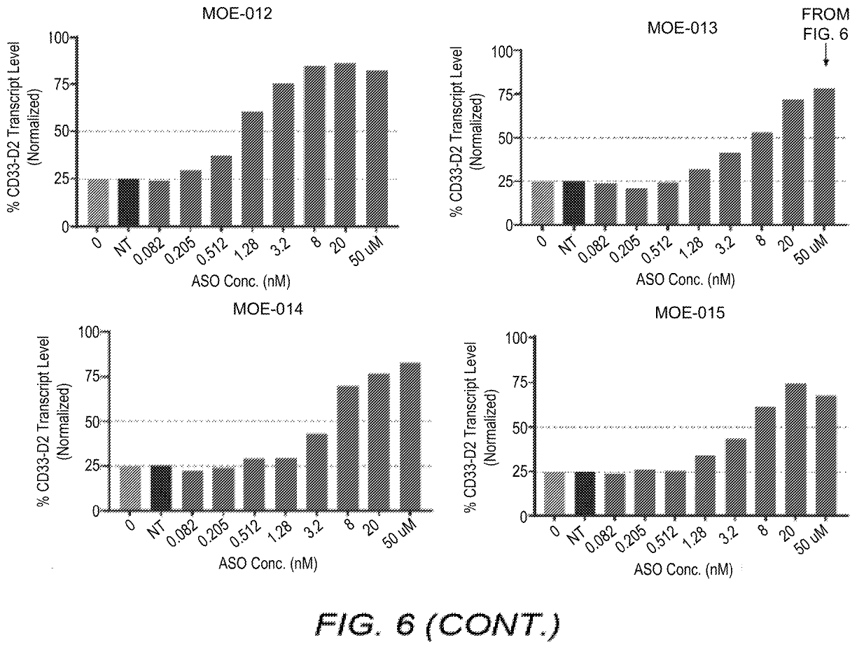

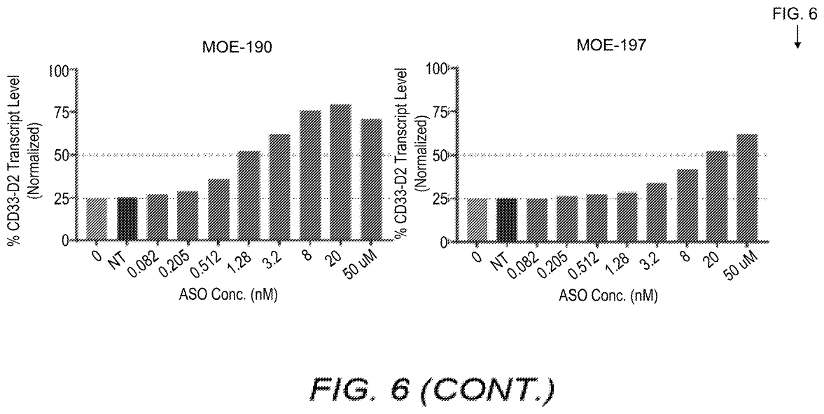

Skipping efficiency is represented on a scale of 0 to 100, wherein 100 represents 100% skipping of CD33 Exon-2 skipping. Each experiment used a negative control oligonucleotide, NT (Non-Targeting Control), which does not target CD33. Skipping efficiency (% CD33-D2 Transcript Level (Normalized)) are shown in FIG. 6.

### Example 5: Evaluation of In Vivo Activity of PMO-002 (SEQ ID NO:2) and MOE-012 (SEQ ID NO:12)

Different technologies can be used to assess the activity/properties of CD33 targeting oligonucleotides using various human, mouse, and non-human primate cell lines.

### In Vivo Assay Methods:

Humanized CD33 mouse models were used to study CD33 Exon-2 skipping ASOs. CRISPR/Cas9 mediated gene editing was used to replace murine CD33 with human genomic CD33, including the signal peptide. Murine 3′ and 5′ untranslated regions were retained. For in vivo experiments, mixed gender cohorts of human CD33 mouse lines on a C57BL/6 background were used, mice were 12-24 weeks old at the time of dosing.

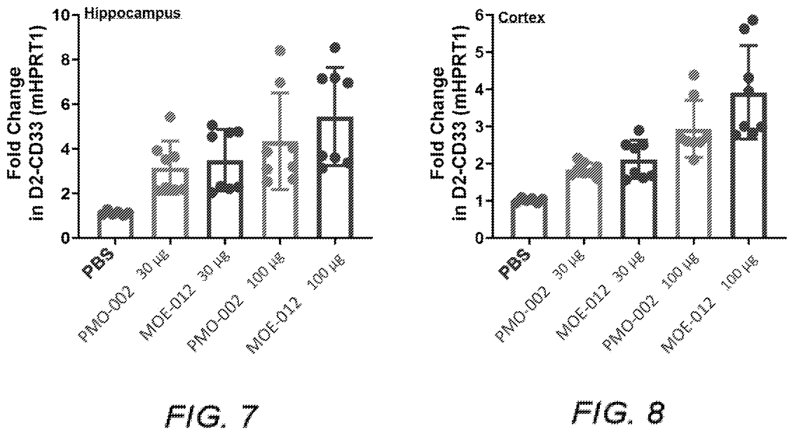

PMO-002 (SEQ ID NO:2) and MOE-012 (SEQ ID NO:12) were administered via intracerebroventricular injection at 30 μg or 100 μg into the right lateral ventricle in a 3 μL bolus on day 1. Mice were necropsied 1 week after the injection. At necropsy, mice were transcardially perfused with PBS under avertin anesthesia. Brains were rapidly removed from the skull and the cortex and hippocampus were dissected from the injected hemisphere for exon skipping evaluation. For RNA isolation, frozen tissue was added with 9× volume of Trizol and homogenized for 3 minutes. 500 μL of the Trizol lysate was transferred to a 1 mL deep well plate. 100 μL of chloroform was added to each sample, shaken vigorously, and centrifuged at 4000×g for 5 minutes. The supernatant (250 μL) was transferred to the binding plate from SV96 total RNA extraction kit (Promega) and RNA was extracted per the same protocol. Total RNA was isolated and converted to cDNA per SV96 protocol (Promega), then Taqman gene expression assays were used to quantify Exon-2 skipped CD33 mRNA transcripts. Mouse house-keeping genes such as HPRT1 or GAPDH1 expressions were used to normalize the target transcript expressions. The fold change of Exon-2 skipped CD33 mRNA in murine hippocampus is displayed in FIG. 7 (n=4, technical duplicates shown in figure). The fold change of Exon-2 skipped CD33 mRNA in murine cortex is displayed in FIG. 8 (n=4, technical duplicates shown in figure). In both the hippocampus and cortex, each ASO increased the amount of Exon-2 skipped CD33 mRNA in vivo for both doses relative to PBS control.

### Example 6: Additional Exemplary PMO-ASOs

PMO oligonucleotides were designed for screening. The designed oligonucleotides were made by GeneTools LLC (website:www.gene-tools.com) by solid-phase method. Table 5 below lists synthesized PMO oligonucleotides with their deconvoluted MS data. These PMO oligonucleotides are complementary to a section of SEQ ID NO:1 showing increased Exon-2 skipping activity. In particular, PMO-221 through PMO 240, PMO-324, PMO-424, PMO-402 and PMO-502 are complementary to Region 1; and PMO-241 through PMO-244 are complementary to Region 2. All PMO oligonucleotides listed in Table 5 below contain a phosphorodiamidate-attached sarcosine (Sar) linker at the 5′ end. All PMO oligonucleotides listed in Table 5 below were synthesized with unmodified cytosine PMO nucleotide. All PMO oligonucleotides listed in Table 5 below have stereorandom internucleotide linkages, and thus are called stereorandom PMO oligonucleotides. The structure of PMO-224 is as follows:

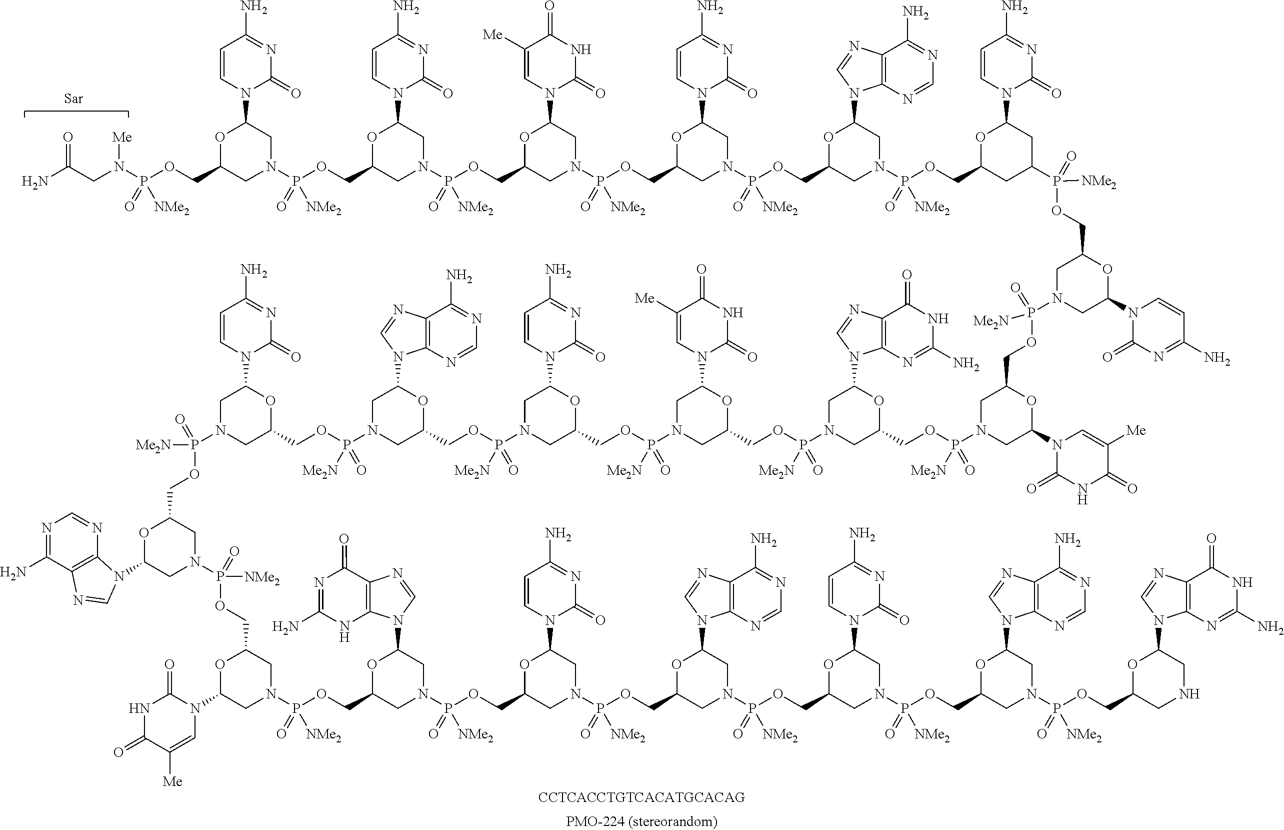

TABLE 5

| | | | MW | |
|---|---|---|---|---|
| | | | (with | |
| | | SEQ. ID | Sarcosine | MS |
| ASO # | Seq. | NO: | linker) | observed |
| PMO-221 | 5′ -CCTCACCTGTCACATGCACAGAG- | 221 | 7703.47 | 7709.4 |
| | 3′ | | | |
| PMO-222 | 5′ -TCACCTGTCACATGCACAGAGAG- | 222 | 7767.52 | 7773.6 |
| | 3′ | | | |
| PMO-223 | 5′ -CTCACCTGTCACATGCACAGAGA- | 223 | 7727.49 | 7733.6 |
| | 3′ | | | |
| PMO-224 | 5′ -CCTCACCTGTCACATGCACAG-3′ | 224 | 7008.88 | 7013.9 |
| PMO-225 | 5′ -ACCTGTCACATGCACAGAGAG-3′ | 225 | 7121.98 | 7127.1 |
| PMO-226 | 5′ -TCACCTGTCACATGCACAGAG-3′ | 226 | 7072.93 | 7077.9 |
| PMO-227 | 5′ - | 227 | 8413.07 | 8418.5 |
| | TCACCTGTCACATGCACAGAGAGCT- | | | |
| | 3′ | | | |
| PMO-228 | 5′ - | 228 | 8315.97 | 8320.0 |
| | CCTGTGCCTCACCTGTCACATGCAC- | | | |
| | 3′ | | | |
| PMO-229 | 5′ - | 229 | 8389.04 | 8393.6 |
| | GTGCCTCACCTGTCACATGCACAGA- | | | |
| | 3′ | | | |
| PMO-230 | 5′ - | 230 | 8389.04 | 8393.7 |
| | TGCCTCACCTGTCACATGCACAGAG- | | | |
| | 3′ | | | |
| PMO-231 | 5′ - | 231 | 8398.06 | 8386.1 |
| | CTCACCTGTCACATGCACAGAGAGC- | | | |
| | 3′ | | | |
| PMO-232 | 5′ - | 232 | 8438.08 | 8441.8 |
| | CACCTGTCACATGCACAGAGAGCTG- | | | |
| | 3′ | | | |
| PMO-233 | 5′ - | 233 | 8478.11 | 8483.7 |
| | ACCTGTCACATGCACAGAGAGCTGG- | | | |
| | 3′ | | | |
| PMO-234 | 5′ - | 234 | 8534.14 | 8539.6 |
| | CTGTCACATGCACAGAGAGCTGGGG- | | | |
| | 3′ | | | |
| PMO-235 | 5′ -CCTGTCACATGCACAGAGAGCTG- | 235 | 7783.52 | 7789.6 |
| | 3′ | | | |
| PMO-236 | 5′ -TGTCACATGCACAGAGAGCTGGG- | 236 | 7863.58 | 7868.6 |
| | 3′ | | | |
| PMO-237 | 5′ -CTGTCACATGCACAGAGAGCTGG- | 237 | 7823.55 | 7828.0 |
| | 3′ | | | |
| PMO-238 | 5′ -TGTCACATGCACAGAGAGCTGG-3′ | 238 | 7508.28 | 7513.6 |
| PMO-239 | 5′ -TCACATGCACAGAGAGCTGGG-3′ | 239 | 7178 | 7183.4 |
| PMO-240 | 5′ -TGTCACATGCACAGAGAGCTG-3′ | 240 | 7152.99 | 7157.2 |
| PMO-241 | 5′ - | 241 | 8326.97 | 8332.8 |
| | CTGTATTTGGTACTTCCTCTCTCCA-3′ | | | |
| PMO-242 | 5′ - | 242 | 8341.98 | 8347.2 |
| | TGTATTTGGTACTTCCTCTCTCCAT-3′ | | | |
| PMO-243 | 5′ - | 243 | 8326.97 | 8332.8 |
| | GTATTTGGTACTTCCTCTCTCCATC-3′ | | | |
| PMO-244 | 5′ - | 244 | 8286.94 | 8292.3 |
| | TATTTGGTACTTCCTCTCTCCATCC-3′ | | | |

### Example 7: Evaluation of in vivo activity of PMO-002. PMO-003. PMO-224. PMO-232. PMO-233. PMO-237. and PMO-238

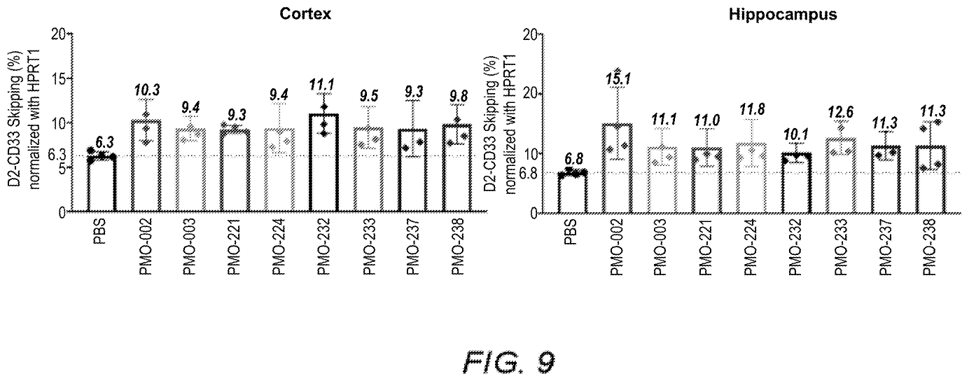

To examine the in vivo effect of five PMO sequences in Tables 1 and 5, a study in hCD33 mice was performed with an CV administered 30 μg dose in a manner identical to Example 5 with the exception of the injection volume being 2.5 μL. Skipping effect was assessed after 7-days. The data represented as Exon-2 CD33 skipping % is shown in FIG. 9.

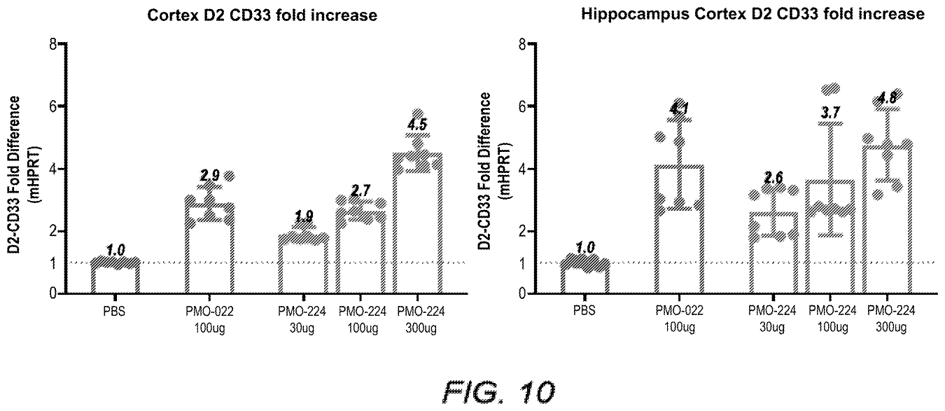

The skipping effect of PMO-224 was assessed in a separate in vivo study with 30 μg, 100 μg and 300 μg doses and injection volume of 10 μL. PMO-002 was also assessed with a 100 μg dose. The data represented as fold-change relative to PBS control is shown in FIG. 10.

### Example 8: Synthesis of PMO Oligonucleotides with Stereopure Internucleotide Linkages and 5′-Sarcosine Linkers

TABLE 6
Stereopure PMO oligonucleotides

| ASO name | Sequence |
|---|---|
| PMO-324 (all Rp | 5′ -CCTCACCTGTCACATGCACAG-3′ |
| internucleotide | |
| linkages) | |
| PMO-424 (all Sp | 5′-CCTCACCTGTCACATGCACAG-3′ |
| internucleotide | |
| linkages) | |
| PMO-402 (all Rp | 5′-CCTCACCTGTCACATGCACAGAGAG-3′ |
| internucleotide | |
| linkages) | |
| PMO-502 (all Sp | 5′-CCTCACCTGTCACATGCACAGAGAG-3′ |
| internucleotide | |
| linkages) | |

### Solution Phase Synthesis of Stereopure PMO Oligonucleotides:

Solution phase synthesis of 5′-sarcosine capped stereopure oligonucleotides in Table 6 was conducted using similar methods to those methods described in Example 3 (using general Procedures A and B) with the exception of Step 1 which started with coupling sarcosine benzyl ester to a stereopure cytosine dimethylphosphoramidochloridate. Briefly, the synthesis includes iterative steps of deprotection/free basing/coupling as depicted here for all Sp internucleotide linkages):

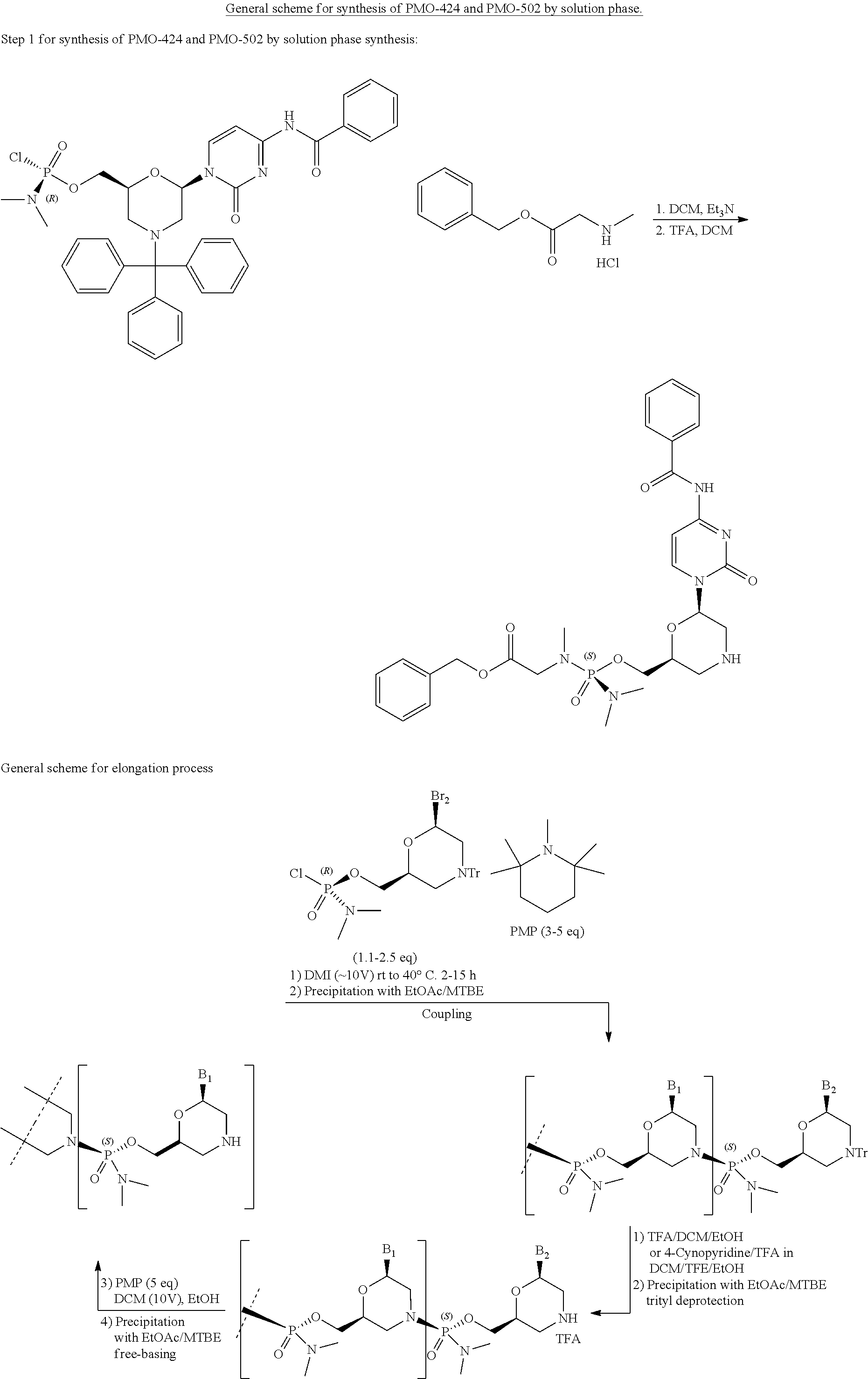

Briefly, the synthesis includes iterative steps of deprotection/free basing/coupling as depicted here for all Rp internucleotide linkages):

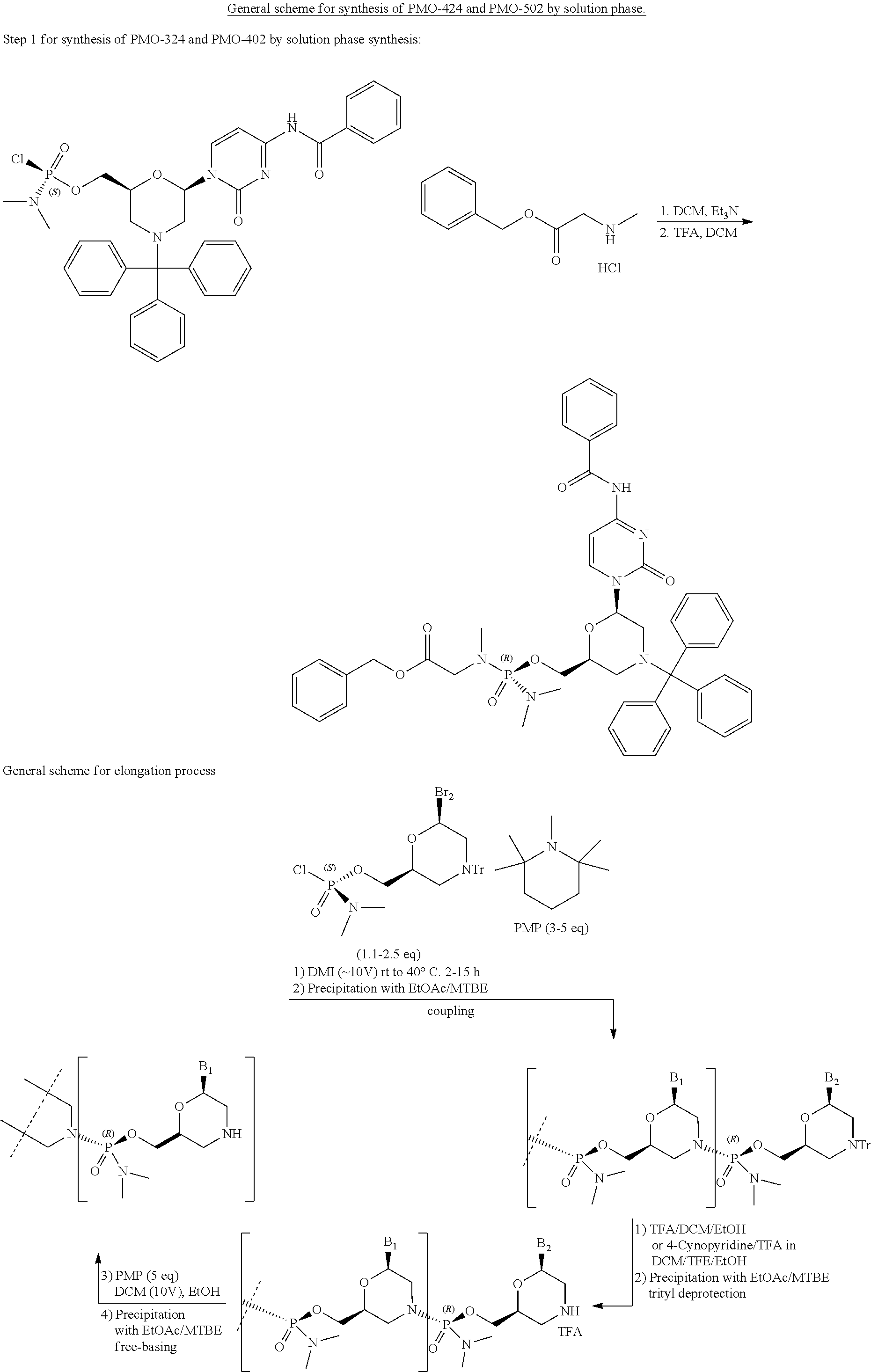

At the conclusion of each individual step, precipitation of the oligonucleotide was achieved by addition of non-polar solvent such as MTBE and/or EtOAc. In elongation steps up to sixmer, purification of the 3′-N-trityl protected oligonucleotide was conducted by silica gel chromatography using DCM/MeOH as eluent.

Upon reaching the desired oligonucleotide length (21-mer for PMO-324, PMO-424, and 25-mer for PMO-402 and PMO-502), the 3′-N-trityl protected sequence was subjected to base deprotection as follows.

Base deprotection for solution phase synthesis:

The 3′-N-trityl protected PMO oligonucleotide residue from the final coupling step (1 wt.) was dissolved in MeOH (8 vols) and then 7N NH₃ in MeOH (20 vols) was added. The reaction mixture was heated to 50-55° C. for at least 48 hours. The solution was filtered to remove any solids, and rinsed with 1:1 MeOH/7N NH₃ in MeOH. Purification by preparative-scale chromatography using a reverse phase gradient as outlined in Table 7 afforded the 3′-N-trityl protected PMO after solvent evaporation.

TABLE 7
Analytical and purification conditions for stereopure
3′-N-trityl protected PMO oligonucleotides
Analytical

| | |
|---|---|
| Column | XBridge, Premier BEH C18 300 Å 2.5 μm 150 × 2.1 mm |
| | (Part number: 186009894) |
| Flow rate | 0.8 mL/min |
| Column | room temperature |
| temperature | |
| Buffer A | 10 mM ammonium bicarbonate in water |
| Buffer B | 10 mM ammonium bicarbonate/MeOH/MeCN (10/10/80) |
| Gradient | 5 to 99% B (6 min) |

Preparative

| | |
|---|---|
| Column | Phenomenex Luna C18 5 μm, 300 × 4.6 mm |
| | (Part number: 00H-4252-E0) |
| Flow rate | 1.5 mL/min |
| Column | 40° C. |
| temperature | |
| Buffer A | Water + 0.1% Et₃N |
| Buffer B | MeCN + 0.1% Et₃N |
| Gradient | 5 to 99% B (23 min) |

Final trityl deprotection: To the base-deprotected PMO oligonucleotide from HPLC purification was added 0.1 N phosphoric acid (at least 20 equivalents) and the reaction was monitored by HPLC. Upon completion of trityl deprotection assessed by two consecutive HPLC runs, the reaction mixture was basified by addition of ammonium hydroxide (at least 40 equivalents). The solution was filtered and the final PMO oligonucleotide was purified by HPLC under the conditions in Table 8.

TABLE 8
Analytical and purification conditions for fully
deprotected stereopure PMO oligonucleotides
Analytical

| | |
|---|---|
| Column | XBridge, Premier BEH C18 300 Å 2.5 μm 150 × 2.1 mm |
| | (Part number: 186009894) |
| Flow rate | 0.8 mL/min |
| Column | room temperature |
| temperature | |
| Buffer A | 10 mM ammonium bicarbonate in water |
| Buffer B | 10 mM ammonium bicarbonate/MeOH/MeCN (10/10/80) |
| Gradient | 5 to 99% B (6 min) |

Preparative

| | |
|---|---|
| Column | Waters, XBridge Prep C18 OBD 5 μm, 19 × 100 mm |
| | (Part number: 186002978) |
| Flow rate | 25.5 mL/min |
| Column | room temperature |
| temperature | |
| Buffer A | 10 mM ammonium bicarbonate in water |
| Buffer B | 10 mM ammonium bicarbonate/MeOH/MeCN |
| | (10/10/80) |
| Gradient | 5 to 28% B (28 min) |

### Example 9: Analytical Data for Stereopure PMO Oligonucleotides

### The Melting Temperature (Tm) of PMO Oligonucleotides:

### Tm Measurement Device: Shimadzu UV-2700 UV-Vis Spectrophotometer

ASO samples were prepared by dissolving ˜0.6-0.8 mg of solid to ˜3.2 μg/mL using nuclease free water. Reverse complementary RNA (obtained from IDT Technologies Inc.) was dissolved to 400 μM in nuclease free water. 10 μL aliquots of each stock solutions were diluted to 1 mL using nuclease free water to determine their concentrations by UV-Vis Spectrophotomer. Test Samples (500 μL) were prepared containing 4.0 μM PMO with 4.0 μM reverse complimentary RNA in buffer (100 mM NaCl, 10 mM Na Phosphate pH 7.0 with 0.1 mM EDTA). Test samples were incubated in a 1 mL cuvette and heated from 15° C. to 105° C. at 0.5° C./min. UV absorbance increase due to strand melting was monitored at 260 nm. Prior to the experiment, the samples were melted and reannealed by heating from 25° C. to 95° C. at 5° C./min and cooling to starting temperatures to ensure complete annealing. Shimadzu Tm Analysis software was used to calculate the Tm (curve inflection point: 50% melting) using the derivative function.

### Analytical Data for Stereopure PMO Oligonucleotides.

### PMO-424:

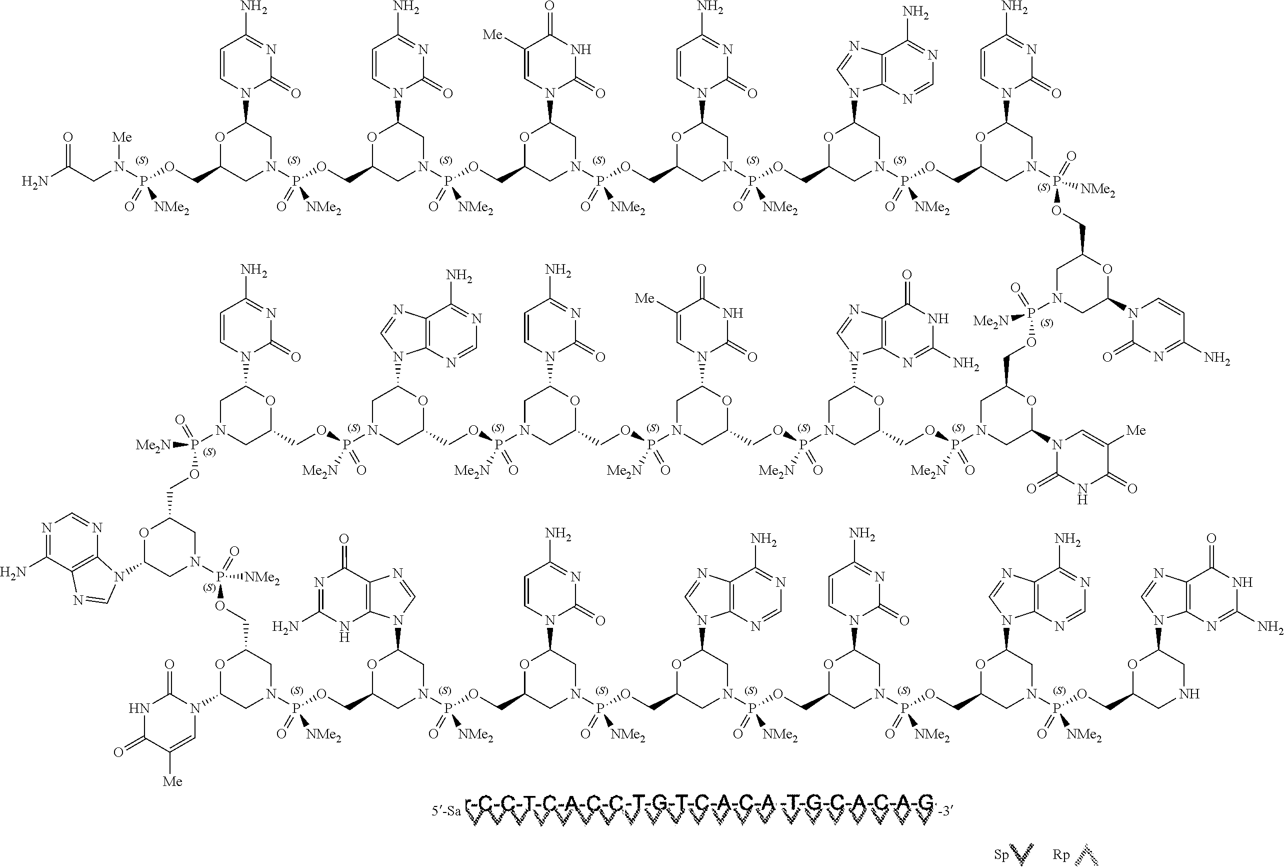

P³¹ NMR (D₂O, 162 MHz) δ 21.5, 18.7, 18.6, 18.5, 18.4, 18.3, 18.3, 18.1, 18.0, 17.9. ESI-TOF-MS Calcd.: 7009.02 for C₂₄₆H₃₉₀N₁₁₉O₈₄P₂₁; Found: 7008.51.

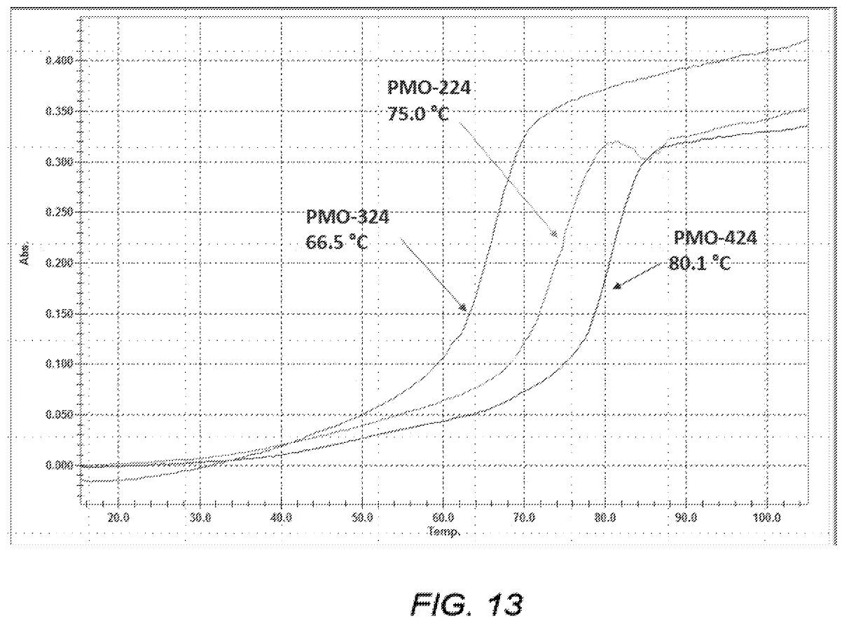

Tm=80.1° C. (Tm of stereorandom=75.0° C.). See FIG. 13. See FIG. 11 for HPLC and HRMS data.
PMO-324:

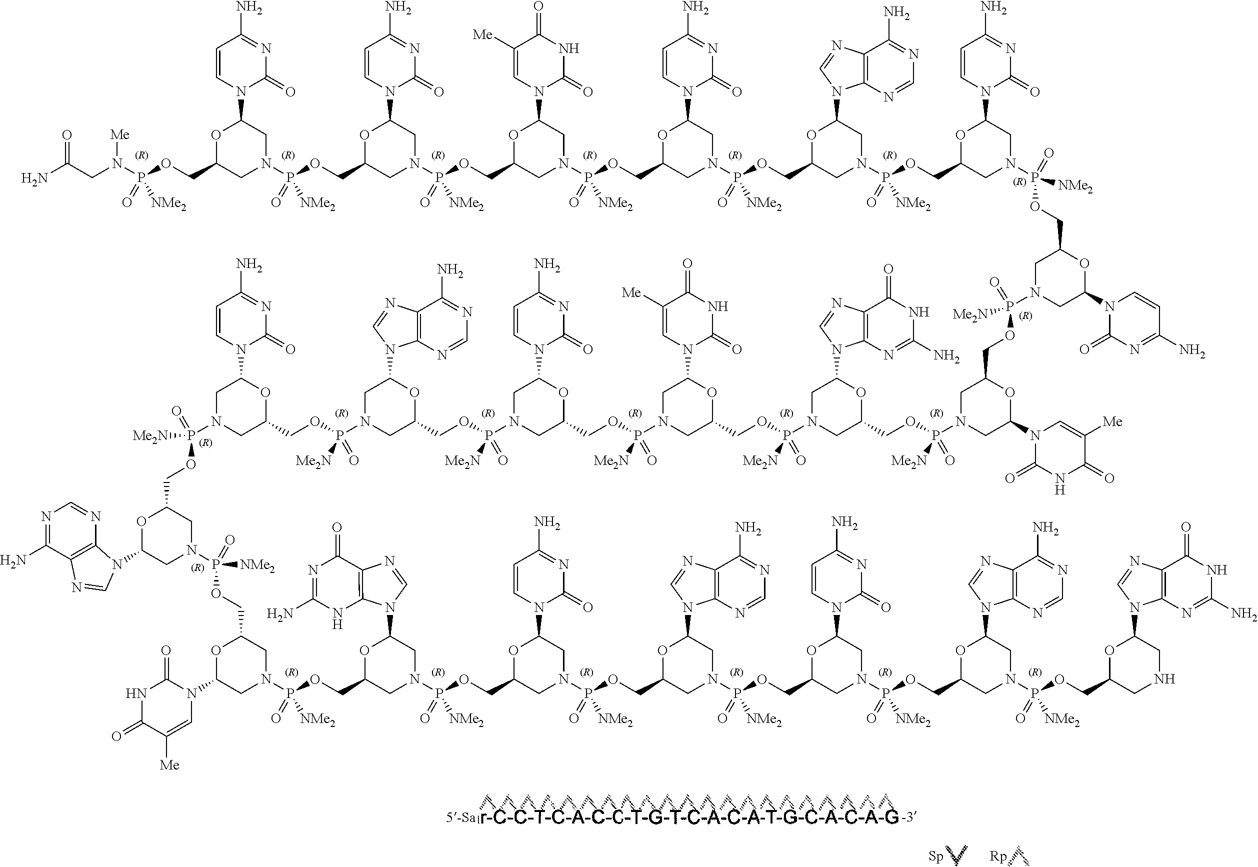

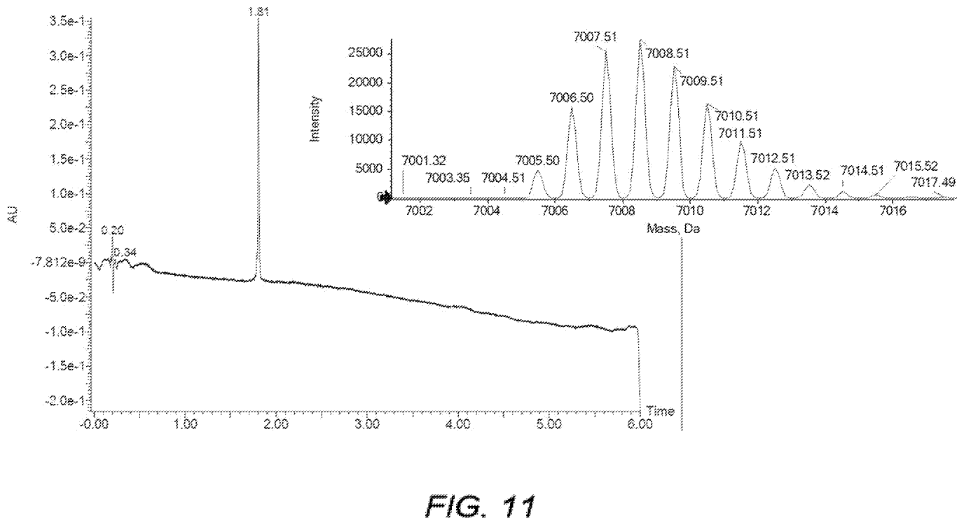

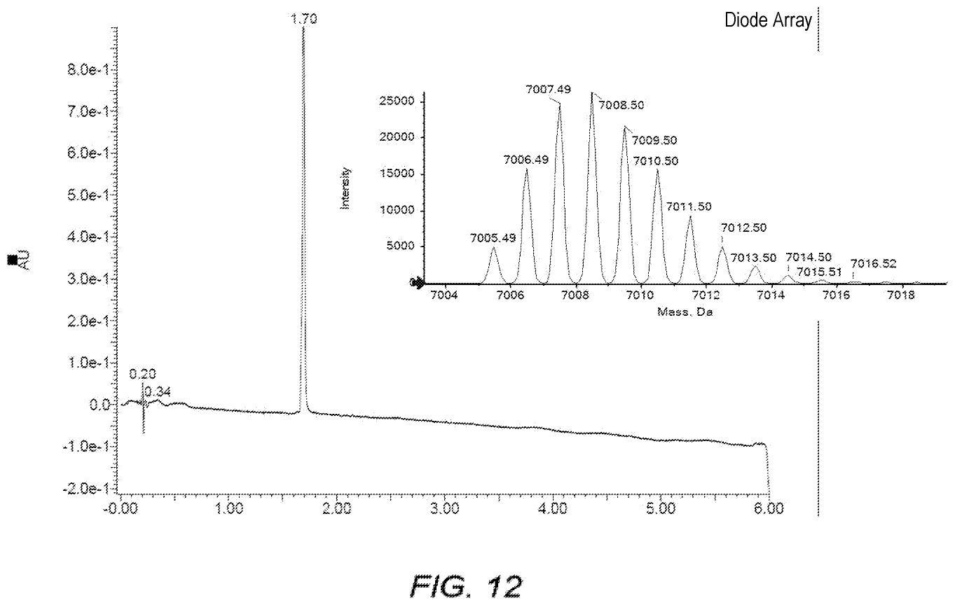

ESI-TOF-MS Calcd.: 7009.02 for C₂₄₆H₃₉₀N₁₁₉O₈₄P₂₁;
Found: 7008.50.
Tm=66.5° C. (Tm of stereorandom=75.0° C.). See FIG. 13.
See FIG. 12 for HPLC and HRMS data.

PMO-502:

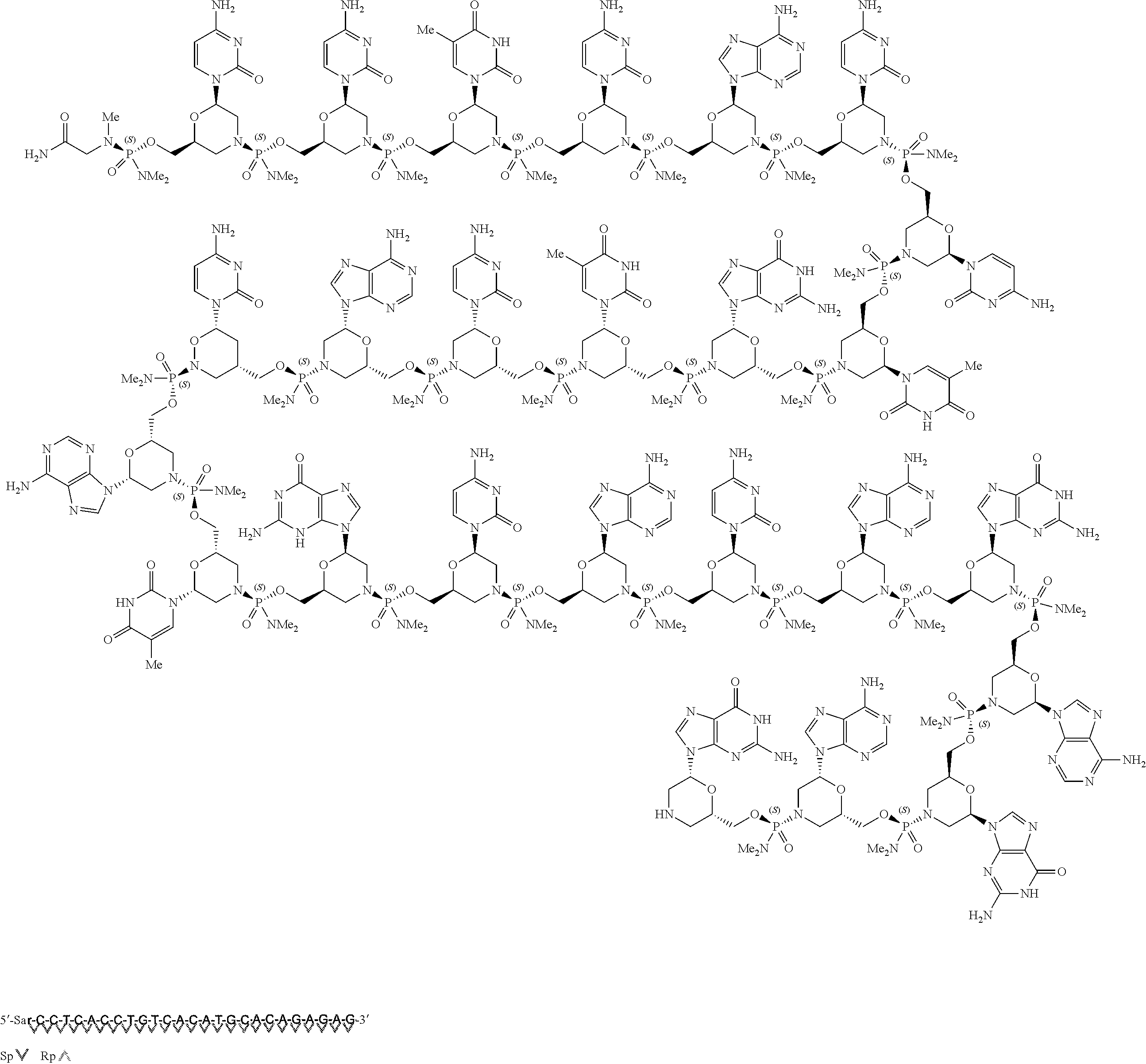

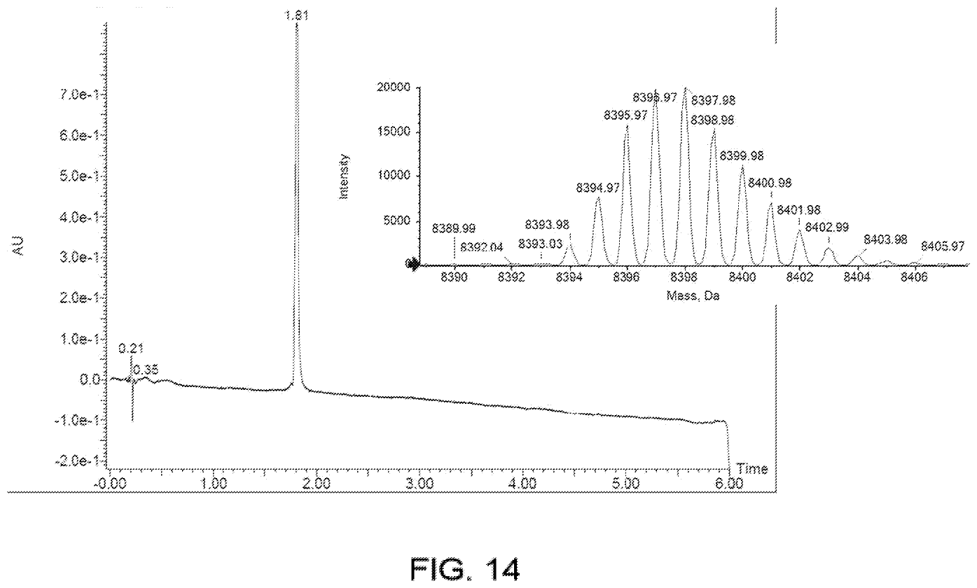

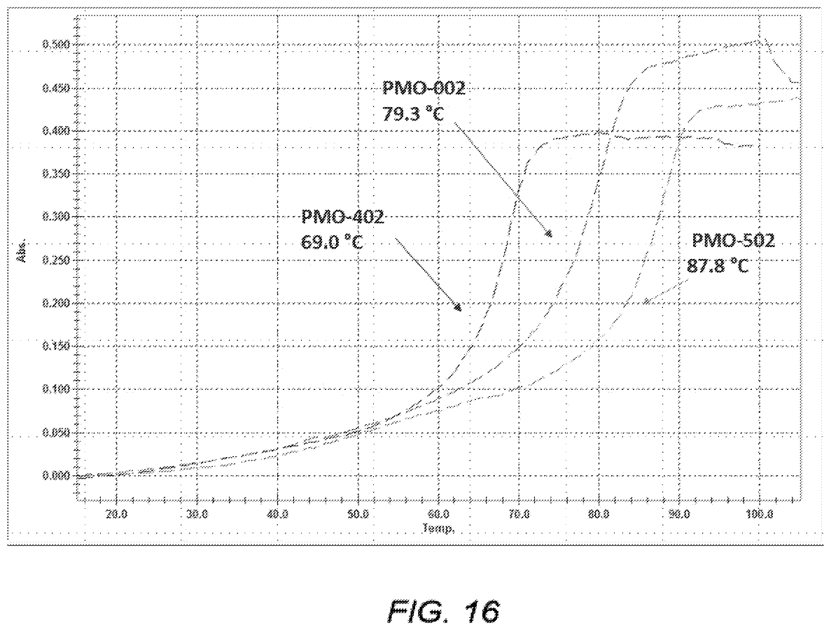

ESI-TOF-MS Calcd.: 8398.20 for C₂₉₄H₄₆₂N₁₄₇O₉₃P₂₅
Found: 8397.98.
Tm=87.8° C. (Tm of stereorandom=79.3° C.). See FIG. 16.
See FIG. 14 for HPLC and HRMS data.

PMO-402:

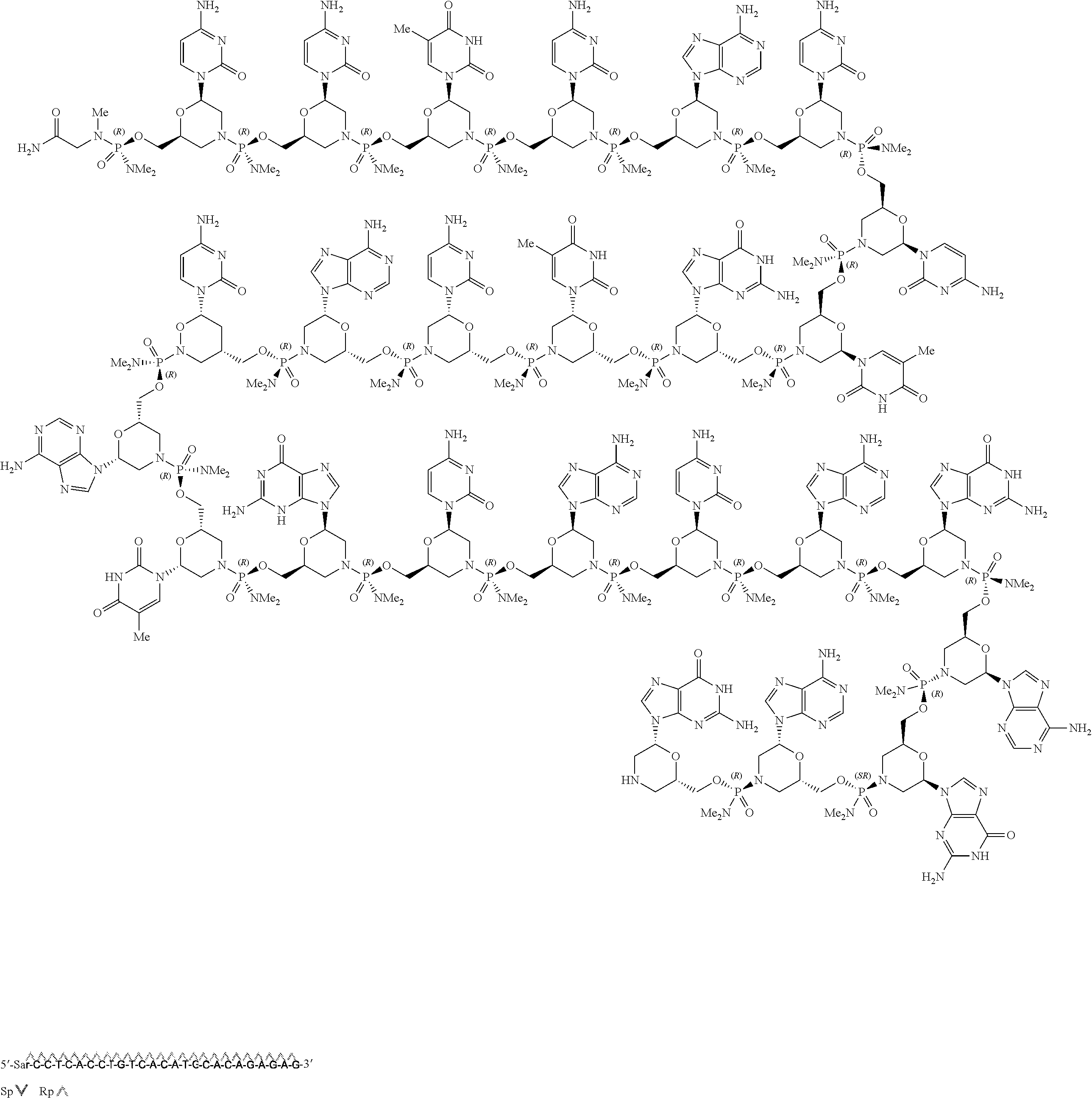

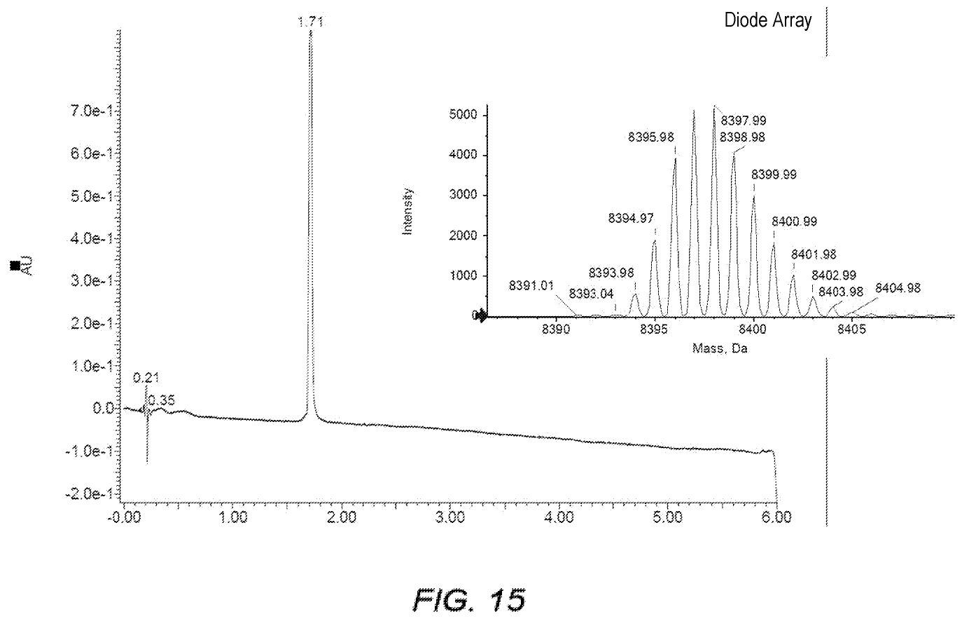

ESI-TOF-MS Calcd.: 8398.20 for C₂₉₄H₄₆₂N₁₄₇O₉₈P₂₅
Found: 8397.99.
Tm=69.0° C. (Tm of stereorandom=79.3° C.). See FIG. 16.
See FIG. 15 for HPLC and HRMS data.

### Example 10: Solid phase Synthesis of stereopure PMOs using peptide synthesizer

Deprotection of Fmoc on Sar-Wang resin:

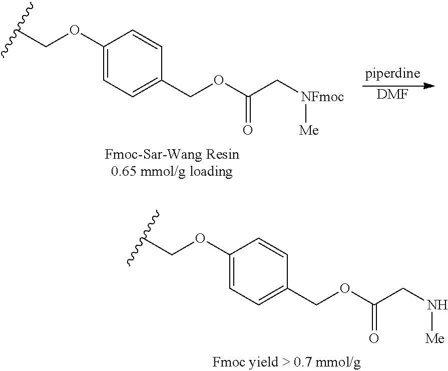

Fmoc-SAR-Wang resin (purchased from Aapptec, RWG103, Lot #9953380, 0.65 mmol/g, 110-200 mesh) (1 g, 650 mmol) was treated with DMF (8 mL), allowed resin to swell for 2 h and drained DMF. The resin was treated with 20% piperidine in DMF (6 mL), shaked for 3 minutes, removed solvent, and dried for 1 minute under N₂ gas (repeated the same sequence for 4 times). Finally, the resin was washed with DMF (5 mL×5 times), washed with CH₂Cl₂ (5 mL×5 times), and dried under vacuum using N₂ gas for overnight to give 0.8 g of resin.

Calculation of resin loading: To the collected piperidine solution was added 20% piperidine in DMF to make final volume of 40 mL. Now, 0.1 mL of solution was diluted 100 times with DMF and measured UV absorbance at 301 nm of the Fmoc group per gram. The loading amount of the resin was >700 μmol/g.

### Conditions for UV Measurement

Solvent: 20% piperidine in DMF
Wave length: 301 nm
ε=7800

### General Procedure for Solid-Phase Synthesis of PMOs:

(a) Synthetic Flow for PMOs with all-Sp Internucleotide Linkages

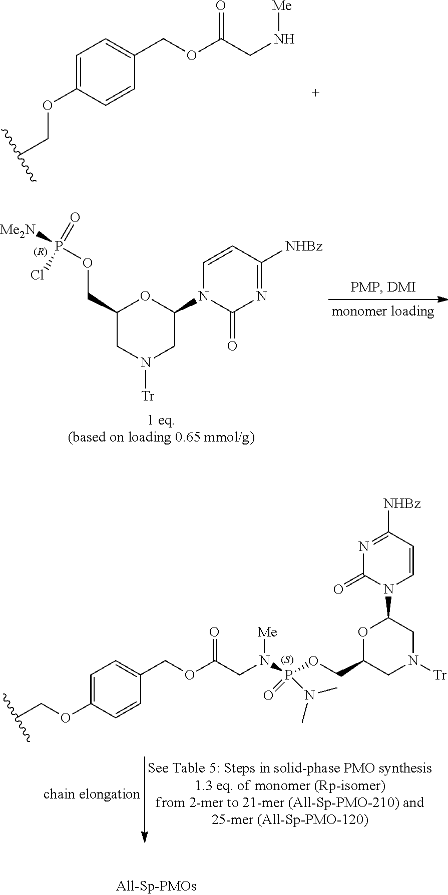

(b) Synthetic Flow for PMOs with all-Rp Internucleotide Linkages

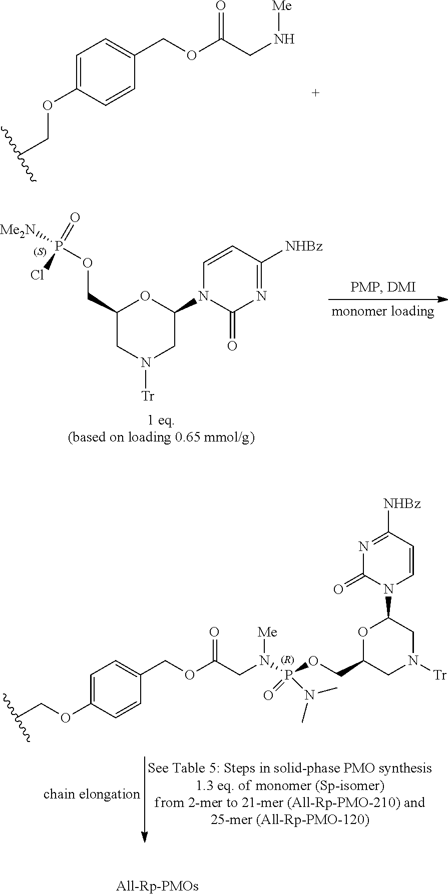

Fmoc deprotected resin (1.10 g, loading: 0.650 mmol/g) was transferred into the peptide synthesizer reaction vessel, washed with CH₂Cl₂ (20 mL×5 times), washed with acetonitrile (20 mL×5 times), and dried. Stereopure cytosine dimethylphosphoramidochloridate (1 eq.) was added to flask as a solid. Then, 1,2,2,6,6-pentamethylpiperidine (PMP, 10.0 eq.) and anhydrous 1,3-dimethyl-2-imidazolidinone (DMI, 5.0 mL) were added to vessel and shaked at room temperature for 20 hours. LCMS of the reaction aliquot showed no monomer in the solution (indicates all monomer was loaded on resin). Then, steps 5-9 in Table 9 were performed.

TABLE 9
Steps in solid-phase PMO synthesis

| Step | Reaction | Reagent | |
|---|---|---|---|
| 1 | Detritylation | 2% 3-cyanopyridine-TFA, 0.9% EtOH, 20% | 7 cycles |
| | | TFE/DCM (80 mL) then CH₂Cl₂ (15 mL × 2) | |
| 2 | Neutralization | 10% Hunig's base in NMP | 30 ml × |
| | | | 4 times |
| 3 | Wash | 1,3-dimethyl-2-imidazolidinone (30 mL × 2 times) | |
| | | MeCN (25 mL × 10 times), | |
| | | CH₂Cl₂ (25 mL × 15 times) | |
| 4 | Coupling | Monomer (1.3 eq), DMI (0.05M), PMP (10 eq.) | 20 h |
| 5 | Wash | MeCN (25 mL × 10 times), | |
| | | CH₂Cl₂ (25 mL × 10 times) | |
| 6 | Capping | 2,6-Lutidine (30% in MeCN, 6 mL) Ac₂O (20% in | 3 times |
| | | MeCN, 6 mL) | |
| 7 | Wash | CH₂Cl₂ (25 mL × 10 times) | |
| 8 | Ac₂O removal | 10% Morpholine in NMP | 40 ml × |
| | | | 4 times |
| 9 | Wash | CH₂Cl₂ (30 ml × 10 times) | |

### Preparation of Detritylation Solution:

To a solution of 4-cyanopyridine (10.1 g; 1.055 eq) in dichloromethane (790 mL) is added trifluoroacetic acid (10.5 g; 1.0 eq), followed by 2,2,2-trifluoroethanol (198 mL) and ethanol (10 mL), and the solution is stirred for 3 hours.

After the first monomer loading on resin, the synthetic cycle (as shown in Table 9) was started. The synthesis had a series of iterative steps including deprotection/neutralization/coupling/capping. The required monomer (purities of monomers were characterized by HPLC-Mass before use) was added in each cycle to obtain the titled nucleotide sequence.

In each synthetic cycle, after coupling reaction (step 4, Table 9) a bit of resin was subjected to cleavage conditions (0.1 mL of 7N NH₃/MeOH, at 55° C., 4 h), and recorded RP HPLC-Mass for coupling efficiency (RP HPLC-Mass showed two peaks for methyl ester and amide in ˜2:1 ratio. For complete conversion of methyl ester to amide, the cleavage reaction was left overnight stirring at 55° C.). The cleavage protocol was iterated from 2-mer to 21-mer for PMO-324 and PMO-424 and to 25-mer for PMO-402 and PMO-502. The RP HPLC-Mass was recorded using conditions in Table 10.

TABLE 10
Analytical conditions for reaction monitoring in PMO synthesis.
Analytical

| | |
|---|---|
| Column | Acquity UPLC BEH C18 1.7 μm 50 × 2.1 mm |
| | (Part number: 186002350) |
| Flow rate | 0.8 mL/min |
| Column | 60° C. |
| temperature | |
| Buffer A | 10 mM ammonium bicarbonate in water |
| Buffer B | 10 mM ammonium bicarbonate/MeOH/MeCN (10/10/80) |
| Gradient | 5 to 99% B (6 min) |

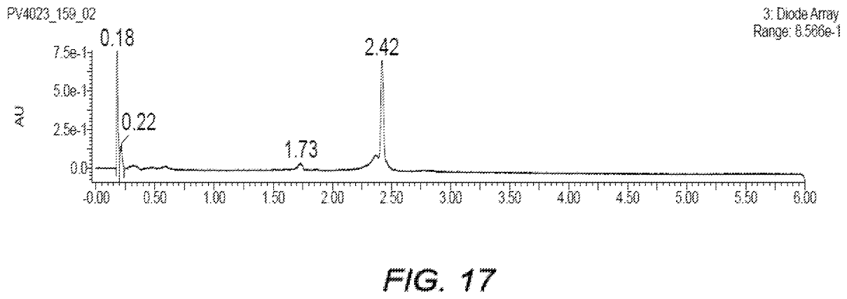

For example, FIG. 17 shows the UV chromatogram of trityl-protected 21-mer (all-Sp-Sar-CCTCACCTGTCACATGCACAG-Tr) after cleavage from resin.

Cleavage from the Resin and Base Deprotection:

After completion of desired oligonucleotide length, the synthesized PMO-loaded resin was dried, transferred to centrifugal bottle, and charged with 7N NH₃/MeOH (˜0.5 mL/1 μmol). The mixture was stirred at 50-55° C. for 60 hours. The reaction was cooled to room temperature, filtered the solids, and washed with methanol. The resulting filtrate was concentrated under reduced pressure to approximate final volume of −20 mL, then, filtered any solids over 0.4 micron membrane filter. The filtrate was concentrated to dryness and weighed. The obtained crude residue was dissolved with 60 mL of solvent mixture of aq. 50 mM Et₃NHOAc (used cell culture water)/MeCN (1/1) with Et₃N (0.1%). The filtrate was purified by reversed phase HPLC conditions as shown in Table 11.

TABLE 11
Analytical and purification conditions for
sterepure 3′-N-trityl protected PMOs.
Analytical

| | |
|---|---|
| Column | XBridge, Premier BEH C18 300 Å 2.5 μm 150 × 2.1 mm |
| | (Part number: 186009894) |
| Flow rate | 0.8 mL/min |
| Column | 60° C. |
| temperature | |
| Solution A | 10 mM ammonium bicarbonate in water |
| Solution B | 10 mM ammonium bicarbonate/MeOH/MeCN |
| | (10/10/80) |
| Gradient | 5 to 99% B (6 min) |

Preparative

| | |
|---|---|
| Column | Waters, XBridge Prep C18 OBD 5 μm, 19 × 100 mm |
| | (Part number: 186002978) |
| Flow rate | 25.5 mL/min |
| Column | room temperature |
| temperature | |
| Solution A | 10 mM ammonium bicarbonate in water |
| Solution B | 10 mM ammonium bicarbonate/MeOH/MeCN (10/10/80) |
| Gradient | 5 to 38% B (28 min) |

### Final Detritylation:

To a flask containing the recovered 3′-N-Tr-PMO (1 eq.) was added freshly prepared 0.1 M aq. phosphoric acid (20 eq.) and the mixture was stirred at room temperature for 2 hours (a white turbid solution was formed within 10 minutes). Reaction completion was checked by two consecutive LCMS runs (shows staring material peak was converted to an earlier eluting peak. HPLC sample was prepared in water only). The reaction was basified by the addition of 28% ammonium hydroxide (40 eq.), stirred for 30 min, filtered the solids through membrane filter (0.45 μm), and washed with water. The resulting filtrate was purified by reverse phase HPLC (Table 12).

TABLE 12
Analytical and purification conditions
for fully deprotected stereopure PMOs
Analytical

| | |
|---|---|
| Column | XBridge, Premier BEH C18 300 Å 2.5 μm 150 × 2.1 mm |
| | (Part number: 186009894) |
| Flow rate | 0.8 mL/min |
| Column | 60° C. |
| temperature | |
| Solution A | 10 mM ammonium bicarbonate in water |
| Solution B | 10 mM ammonium bicarbonate/MeOH/MeCN (10/10/80) |
| Gradient | 5 to 99% B (6 min) |

Preparative

| | |
|---|---|
| Column | Waters, XBridge Prep C18 OBD 5 μm, 19 × 100 mm |
| | (Part number: 186002978) |
| Flow rate | 25.5 mL/min |
| Column | room temperature |
| temperature | |
| Solution A | 10 mM ammonium bicarbonate in water |
| Solution B | 10 mM ammonium bicarbonate/MeOH/MeCN |
| | (10/10/80) |
| Gradient | 5 to 28% B (28 min) |

Each fraction was analyzed (on HPLC) and the product containing fractions were dried using Genevac. The final product was dissolved in endotoxin-free water, the solution was filtered through Amicon 3K filter to remove any inorganic salt impurites. The aqueous solution obtained was freeze-dried to give the title compound as a white cotton-like solid.

### Analytical Data for Stereopure PMOs Prepared by Solid-Phase Synthesis:

### PMO-424:

P³¹ NMR (D₂O, 162 MHz) δ 21.5, 18.7, 18.6, 18.5, 18.4, 18.3, 18.3, 18.1, 18.0, 17.9.
LRMS: Calcd. m/z for [M+5H]⁵⁺ ion of C₂₄₆H₃₉₀N₁₁₉O₈₄P₂₁ (m/z=7009.02): 1402.80;
found: 1402.62

### PMO-324:

LRMS: Calcd. m/z for [M+5H]⁵⁺ ion of C₂₄₆H₃₉₀N₁₁₉O₈₄P₂₁ (m/z=7009.02): 1402.80;
found: 1403.4

### PMO-402:

LRMS: Calcd. m/z for [M+6H]⁶⁺ ion of C₂₉₄H₄₆₂N₁₄₇O₉P₂₅ (m/z=8396.92): 1400.66;
found: 1401.2

### Example 11: Evaluation of In Vivo Activity of PMO-402, PMO-502, PMO-324, and PMO-424

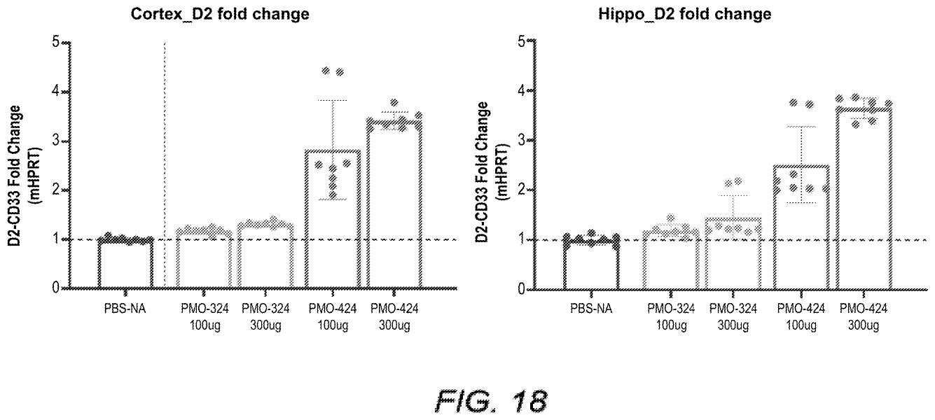

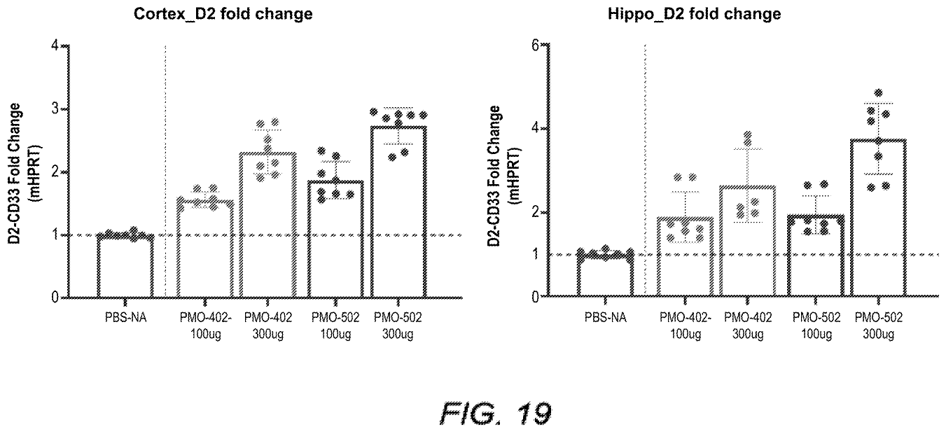

To examine the in vivo effect of PMO-402, PMO-502, PMO-324, and PMO-424 prepared in Example 8, a study in hCD33 mice was performed with 100 μg and 300 μg doses, administered by ICV. The study was performed in a manner identical to Example 5 with the exception of the administration volume of 10 μL. Skipping effect was assessed after 7-days. The data represented as fold-change relative to PBS control is shown in FIGS. 18 and 19.

### Example 12: Additional Exemplary MOE-ASOs

Phosphorothioate oligonucleotides were designed for screening. All oligonucleotides listed in Table 13 below contain ribonucleotides with phosphorothioate backbone except when noted (e.g. solid line (−)=phosphodiester (PO) bond). All oligonucleotides listed in Table 13 below were synthesized with 5-methylcytosine ribonucleotide. All oligonucleotides listed in Table 13 below have stereorandom phosphorothioate internucleotide linkages, and thus are called stereorandom oligonucleotides. All oligonucleotides listed in Table 13 are complementary to Region 6: (SEQ ID NO:218).

TABLE 13
ASOs targeting CD33

| | | | MW | |
|---|---|---|---|---|
| | | | (free | |
| | | SEQ | form | |
| | | ID | calcu- | MS |
| ASO# | Sequence (5′ to 3′) | No: | lated) | observed |
| MOE-245 | CTCCATCCGAAAGAAGTATG | 245 | 7966.9 | 7967.1 |
| MOE-246 | TCCATCCGAAAGAAGTATGA | 246 | 7976.9 | 7976.9 |
| MOE-247 | CCATCCGAAAGAAGTATGAA | 247 | 7985.9 | 7986.9 |
| MOE-248 | CATCCGAAAGAAGTATGAAC | 248 | 7985.9 | 7986.4 |
| MOE-249 | TCCGAAAGAAGTATGAACCA | 249 | 7985.9 | 7986.6 |
| MOE-250 | CCGAAAGAAGTATGAACCAT | 250 | 7985.9 | 7986.2 |
| MOE-251 | ATCCGAAAGAAGTATGAA | 251 | 7199.2 | 7199.9 |
| MOE-252 | CCGAAAGAAGTATGAACC | 252 | 7188.1 | 7188.3 |
| MOE-253 | TCCGAAAGAAGTATGAAC | 253 | 7189.2 | 7188.2 |
| MOE-254 | CCATCCGAAAGAAGTATG | 254 | 7179.2 | 7179.1 |
| MOE-255 | TCCATCCGAAAGAAGTAT | 255 | 7154.2 | 7154.2 |
| MOE-256 | GAAAGAAGTATGAACCAT | 256 | 7199.2 | 7197.9 |
| MOE-257 | ATC—CGAAAGAAGTATGA—ACC | 12 | 7953.8 | 7953.9 |
| MOE-258 | ATCC—GAAAGAAGTATG—AACC | 12 | 7953.8 | 7953.3 |
| MOE-259 | ATCCG—AAAGAAGTAT—GAACC | 12 | 7953.8 | 7953.4 |
| MOE-260 | ATCCG—AAAGAAGTA—TGAACC | 12 | 7953.8 | 7953.4 |
| MOE-261 | ATCC—GAAAGA—AGTATG—AACC | 12 | 7937.7 | 7937.3 |
| MOE-262 | ATCC—gAAAGAAGTATGAACC | 12 | 7923.8 | 7924.3 |
| MOE-263 | ATCC—gAAAGAAGTATG—aACC | 12 | 7861.7 | 7861.9 |
| MOE-264 | ATCC—gAAAGAaGTATG—aACC | 12 | 7815.6 | 7815.8 |
| MOE-265 | CCGA—aAGAAGTATGAACC | 252 | 7126.1 | 7126.5 |
| MOE-266 | CCGA—aAGAAGTATG—aACC | 252 | 7064.0 | 7064.1 |
| MOE-267 | CCGA—aAGAAGtATG—aACC | 252 | 7017.9 | 7018.0 |
| MOE-268 | CCG—AAAGAAGTATGA—ACC | 252 | 7156.1 | 7156.7 |
| MOE-269 | CCGA—AAGAAGTATG—AACC | 252 | 7156.1 | 7156.6 |
| MOE-270 | CCGAA—AGAA—GTATG—AACC | 252 | 7140.0 | 7140.7 |
| MOE-271 | CCGAA—AGAAGTAT—GAACC | 252 | 7156.1 | 7156.7 |
| MOE-272 | CCG—A—AAGAAGTATGAACC | 252 | 7156.1 | 7156.7 |
| MOE-273 | CCG—AA—AGAAGTATGAACC | 252 | 7156.1 | 7156.8 |
| MOE-274 | CCGAAAGAAGTATG—A—ACC | 252 | 7156.1 | 7156.8 |
| MOE-275 | mAmTfCfCfGfAfAfAfGfAfAf | 12 | 6814.1 | 6814.7 |
| | GfTfAfTfGfAfAmCmC | | | |
| MOE-276 | fAfTfCfCfGmAmAmAmGmAmAm | 12 | 6862.3 | 6862.9 |
| | GmTmAfTfGfAfAfCfC | | | |

X (A, T, C, G) = 2′-MOE ribonucleotide, C = 5-Methyl cytosine, lower case letter = LNA (locked nucleic acid), (—) = PO bond, fx = 2′-fluoro ribonucleotide, mX = 2′-OMe ribonucleotide

All oligonucleotides listed in Table 14 below contain a 2′-O-MOE modified ribonucleotides and a hydroxyl group at the 5′ end. Oligonucleotides in Table 14 contain stereopure phosphorothioate internucleotide linkages, and thus are called stereopure MOE oligonucleotides. All oligonucleotides listed in Table 14 are complementary to Region 6: (SEQ ID NO:218).

TABLE 14
Stereopure ASOs targeting CD33

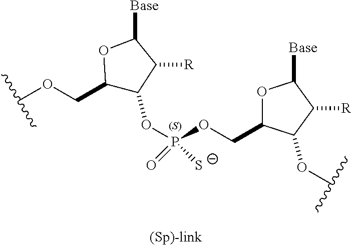

[symbol]

= Sp

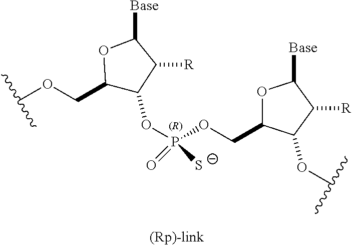

[symbol]

= Rp

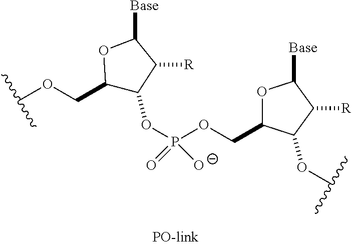

[symbol]

= PO

| ASO# | Sequence (5′ to 3′) | SEQ ID No: |
|---|---|---|
| MOE-277 | 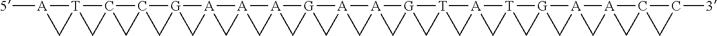 | 12 |
| MOE-278 | 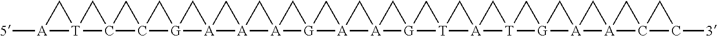 | 12 |
| MOE-279 | 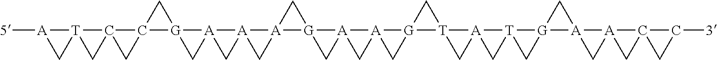 | 12 |
| MOE-280 | 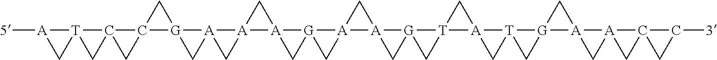 | 12 |
| MOE-281 | 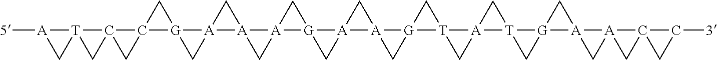 | 12 |
| MOE-282 | 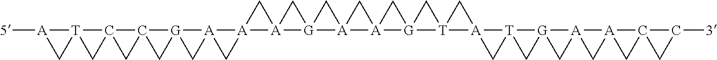 | 12 |
| MOE-283 | 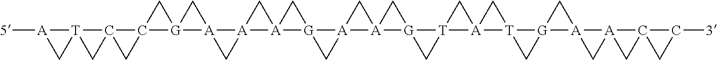 | 12 |
| MOE-284 | 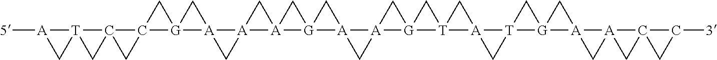 | 12 |
| MOE-285 | 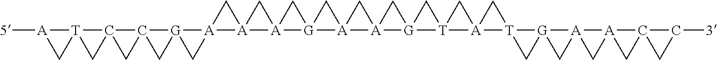 | 12 |
| MOE-286 | 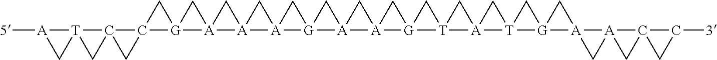 | 12 |
| MOE-287 | 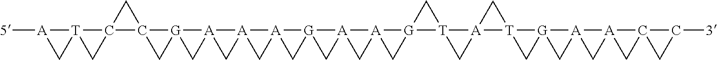 | 12 |
| MOE-288 | 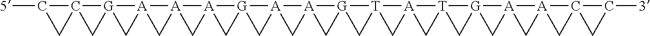 | 252 |
| MOE-289 | 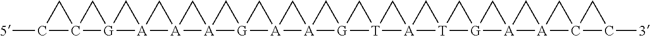 | 252 |
| MOE-290 | 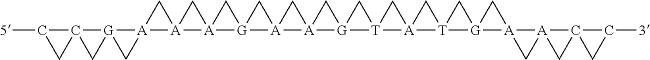 | 252 |
| MOE-291 | 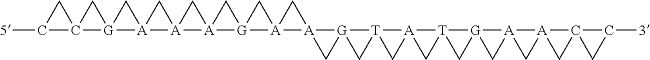 | 252 |
| MOE-292 | 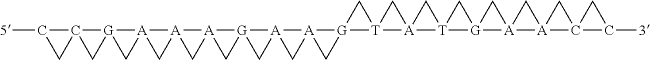 | 252 |
| MOE-293 | 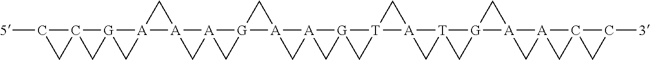 | 252 |
| MOE-294 | 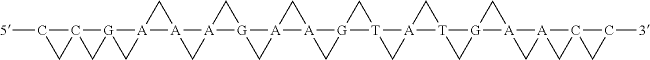 | 252 |
| MOE-295 | 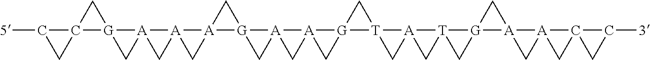 | 252 |
| MOE-296 | 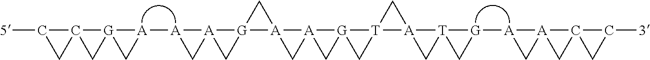 | 252 |
| MOE-297 | 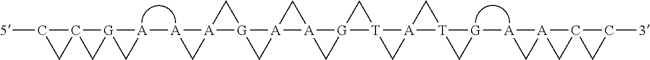 | 252 |
| MOE-298 | 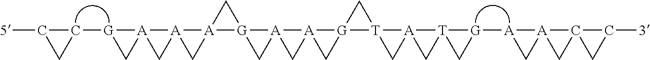 | 252 |
| MOE-299 | 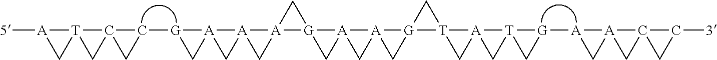 | 12 |
| MOE-300 | 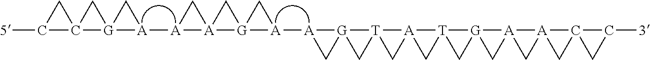 | 252 |
| MOE-301 | 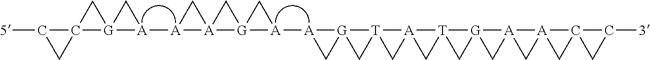 | 252 |
| MOE-303 | 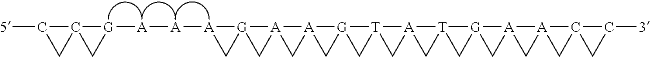 | 252 |
| MOE-304 | 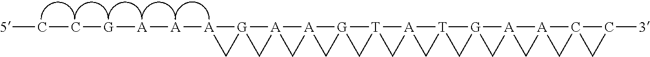 | 252 |
| MOE-305 | 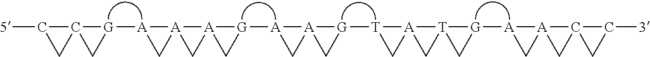 | 252 |
| MOE-306 | 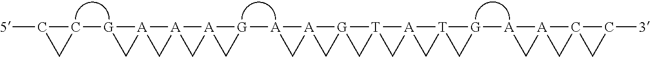 | 252 |
| MOE-307 | 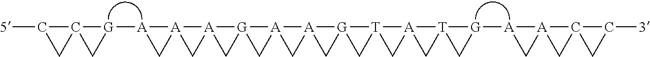 | 252 |
| MOE-308 | 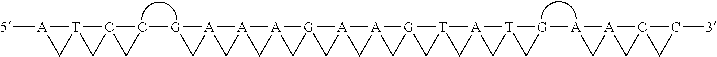 | 12 |
| MOE-309 | 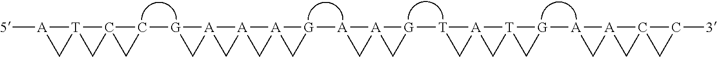 | 12 |
| MOE-310 | 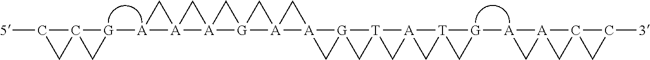 | 252 |
| MOE-311 | 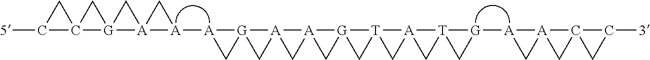 | 252 |

### Example 13: Preparation of Stereopure 2′-MOE Phosphorothiolate Oligonucleotides Protected 2′-O-MOE-3′-OH Monomers

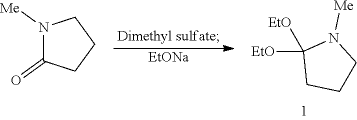 [SMILES: "EtONa" ; CN1CCCC1=O ; CCOC1(OCC)CCCN1C]

(1) 2,2-diethoxy-1-methylpyrrolidine: A mixture of NMP (100 mL, 1039.008 mmol) and dimethyl sulfate (99 mL, 1039.008 mmol) was stirred and heated to 80° C. (sand bath) overnight, then allowed to cool to rt. After cooling, the homogeneous liquid was washed with ether (2×100 mL) and the residual solvent was removed in vacuo. The obtained residue was dissolved in CH₂Cl₂ (400 mL), dried over anhydrous MgSO₄, filtered, washed with CH₂Cl₂ (100 mL) and concentrated under reduced pressure to give 5-methoxy-1-methyl-3,4-dihydro-2H-pyrrol-1-ium as a brown color viscous liquid (solidified at −20° C. storage); ¹H NMR (400 MHz, CDCl₃) δ 4.35-4.40 (m, 3H), 3.98-4.05 (m, 2H), 3.69-3.73 (m, 3H), 3.31-3.38 (m, 2H), 3.19-3.22 (m, 3H), 2.37-2.48 (m, 2H).

The crude product (obtained above) was added to a solution of sodium ethanolate (370 g, 1142.909 mmol, 21% sodium ethoxide in ethanol) at 50 to 55° C. over 1 hour by cannula or dropping funnel under N₂ atmosphere. After stirring at the same temperature for 3 hours, the reaction was cooled to room temperature. The precipitated white solid was filtered, washed with ethanol (50 mL) and the filtrate was concentrated (maintain water-bath temperature ˜30° C.). Fractional distillation of crude residue under house vacuum at 55-65° C. gave 2,2-diethoxy-1-methylpyrrolidine (115 g, 66% yield) as a pale yellow or colorless liquid. The pure product was stored at −20° C.; ¹H NMR (400 MHz, CDCl₃) δ 3.44-3.60 (m, 4H), 2.83-2.91 (m, 3H), 2.33-2.40 (m, 4H), 1.90-1.98 (m, 2H), 1.72-1.87 (m, 2H), 1.15-1.22 (m, 6H).

### General Procedure 1: Pya (N-methylpyrrolidine) protection of 2′-O-MOE G, A, and mC

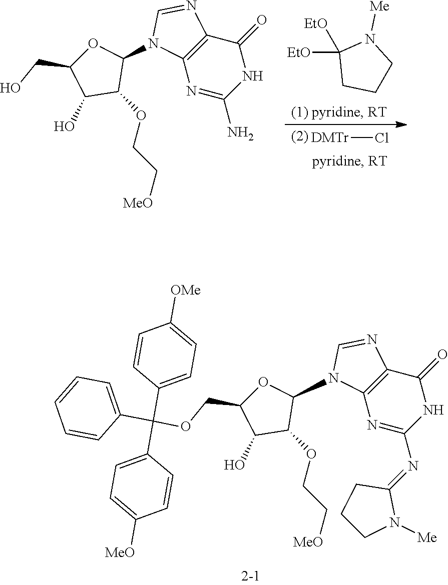

(2-1) 9-((2R,3R,4R,5R)-5-((bis(4-methoxyphenyl)(phenyl)methoxy)methyl)-4-hydroxy-3-(2-methoxyethoxy)tetrahydrofuran-2-yl)-2-(1-methylpyrrolidin-2-ylidene)amino)-1,9-dihydro-6H-purin-6-one:

2-amino-9-((2R,3R,4R,5R)-4-hydroxy-5-(hydroxymethyl)-3-(2-methoxyethoxy)tetrahydrofuran-2-yl)-1,9-dihydro-6H-purin-6-one (13.8 g, 40.431 mmol) was chased under vacuum with anhydrous pyridine (100 mL) for two times. To the concentrated residue was added anhydrous pyridine (114 mL, 1418.073 mmol) followed by 2,2-diethoxy-1-methylpyrrolidine (14.01 g, 80.862 mmol) slowly at room temperature. The reaction was stirred at room temperature overnight, changing from a white turbid solution to a brown clear solution. Water (0.1 mL/6 mmol) was added, and the mixture was concentrated under vacuum, then chased with pyridine and MeCN 3 times. To the resulting residue were added pyridine (105 mL, 1298.197 mmol) and 1-[chloro-(4-methoxyphenyl)-phenylmethyl]-4-methoxybenzene (15.62 g, 46.108 mmol) at room temperature. After stirring at rt overnight, the reaction mixture was worked up with saturated NaHCO₃ (150 mL) and EtOAc (300 mL×2), and the residue was purified by a silica-gel column chromatography (100 g Star Silica, EtOAc/Hept 30 to 100% then EtOAc/MeOH 0 to 30%) to give 2-1 as a foamy solid in 77% yield; ¹H NMR (400 MHz, CDCl3) δ 9.28-9.36 (m, 1H), 7.67-7.72 (m, 1H), 7.32-7.39 (m, 2H), 7.16-7.28 (m, 6H), 7.08-7.16 (m, 1H), 6.69-6.78 (m, 4H), 5.92-5.96 (m, 1H), 4.29-4.37 (m, 2H), 4.11-4.17 (m, 1H), 3.74-3.82 (m, 1H), 3.68-3.73 (m, 7H), 3.55-3.63 (m, 1H), 3.45-3.52 (m, 1H), 3.34-3.41 (m, 3H), 3.25-3.33 (m, 5H), 3.01-3.09 (m, 2H), 2.92-2.96 (m, 3H), 1.90-2.00 (m, 2H); MS (ESI, m/z) calculated for [C₃₉H₄₄N₅O₈+H⁺] 725.33 found 725.4.

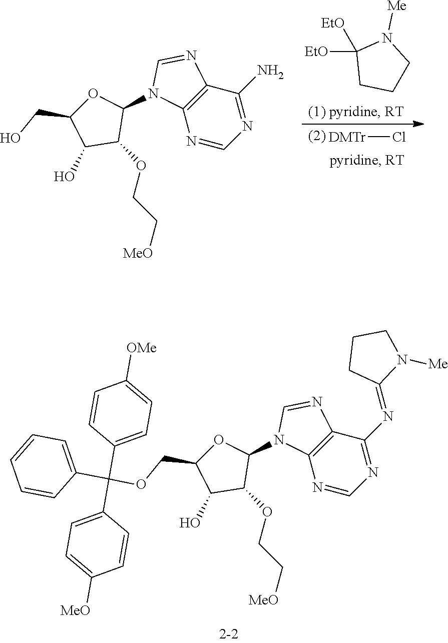

(2-2) (2R,3R,4R,5R)-2-((bis(4-methoxyphenyl)(phenyl)methoxy)methyl)-4-(2-methoxyethoxy)-5-(6-1-methylpyrrolidin-2-ylidene)amino)-9H-purin-9-yl)tetrahydrofuran-3-ol:

Prepared according to general procedure 1, foamy solid, 89% yield; ¹H NMR (400 MHz, DMSO-d₆) δ 8.36 (d, J=8.0 Hz, 2H), 7.40-7.31 (m, 2H), 7.29-7.16 (m, 7H), 6.87-6.77 (m, 4H), 6.07 (d, J=4.8 Hz, 1H), 5.18 (d, J=6.0 Hz, 1H), 4.69 (t, J=5.2 Hz, 1H), 4.44 (q, J=5.2 Hz, 1H), 4.11-4.05 (m, 1H), 3.76-3.71 (m, 7H), 3.62 (dt, J=11.2, 4.8 Hz, 1H), 3.49 (t, J=7.2 Hz, 2H), 3.42 (t, J=4.8 Hz, 2H), 3.23 (d, J=4.8 Hz, 2H), 3.14 (s, 3H), 3.04 (s, 3H), 2.85 (t, J=8.0 Hz, 2H), 2.02-1.93 (m, 2H); MS (ESI, m/z) calculated for [C₃₉H₄₄N₆O₇+H⁺] 709.33 found 709.20.

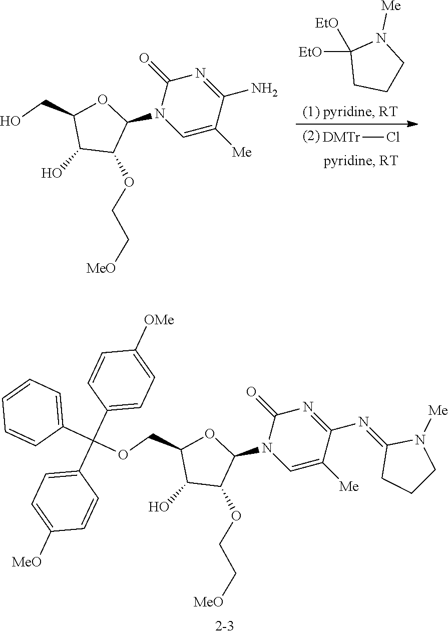

(2-3) 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-4-(1-methylpyrrolidin-2-ylidene)amino)pyrimidin-2(1H)-one:

Prepared according to general procedure 1, 87% yield; foamy solid; ¹H NMR (400 MHz, CDCl₃) δ 7.77-7.81 (m, 1H), 7.46-7.51 (m, 2H), 7.34-7.41 (m, 4H), 7.27-7.33 (m, 2H), 7.20-7.27 (m, 1H), 6.82-6.88 (m, 4H), 5.99-6.04 (m, 1H), 4.34-4.43 (m, 1H), 4.25-4.33 (m, 1H), 4.08-4.15 (m, 1H), 3.99-4.05 (m, 1H), 3.90-3.99 (m, 1H), 3.74-3.83 (m, 6H), 3.54-3.64 (m, 3H), 3.42-3.50 (m, 3H), 3.42 (s, 3H), 3.29-3.34 (m, 1H), 3.07-3.29 (m, 2H), 3.03-3.07 (m, 3H), 2.00-2.11 (m, 2H), 1.53-1.58 (m, 3H); MS (ESI, m/z) calculated for [C₃₉H₄₄N₆O₈+H⁺] 699.33 found 699.25.

Pivaloylmethyl (POM) protection of T:

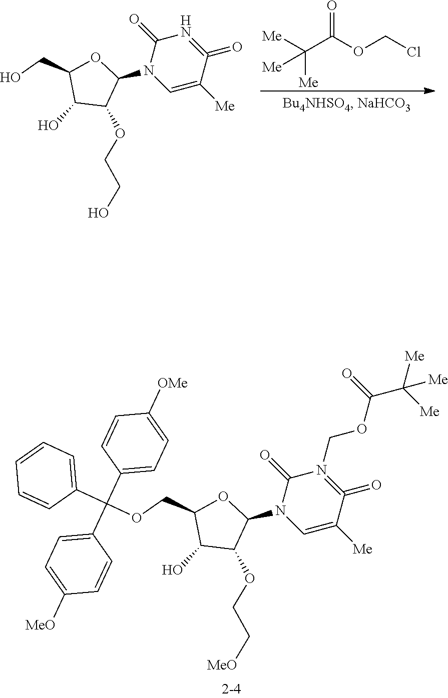

(2-4) (3-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-2,6-dioxo-3,6-dihydropyrimidin-1(2H)-yl)methyl pivalate:

Step 1: To 1-((2R,3R,4R,5R)-4-hydroxy-5-(hydroxymethyl)-3-(2-methoxyethoxy)tetrahydrofuran-2-yl)-5-methylpyrimidine-2,4(1H,3H)-dione (14.2 g, 44.893 mmol) in pyridine (99 mL, 1228.96 mmol) was added 1-[chloro-(4-methoxyphenyl)-phenylmethyl]-4-methoxybenzene (18.25 g, 53.871 mmol) at room temperature. Upon completion, as monitored by UPLC-MS, saturated NaHCO₃ (80 mL) was added to the mixture, extracted with EtOAc (200 mL×2), and purified by a silica-gel column chromatography (100 g, Star silica, EtOAc/Hept 10 to 100%) to give 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methylpyrimidine-2,4(1H,3H)-dione (25 g, 40.408 mmol) in 90% yield.

¹H NMR (400 MHz, DMSO-d₆) δ 11.37 (s, 1H), 7.49 (s, 1H), 7.39 (d, J=7.6 Hz, 2H), 7.35-7.21 (m, 8H), 6.90 (d, J=8.8 Hz, 4H), 5.85 (d, J=4.8 Hz, 1H), 5.12 (d, J=6.0 Hz, 1H), 4.23 (q, J=5.2 Hz, 1H), 4.09 (t, J=4.8 Hz, 1H), 4.02-3.95 (m, 1H), 3.79-3.67 (m, 8H), 3.48 (t, J=4.7 Hz, 2H), 3.26-3.20 (m, 5H), 1.40 (s, 3H).

Step 2: To an aqueous solution of Na₂CO₃ (242 mL, 121.225 mmol) were added 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methylpyrimidine-2,4(1H,3H)-dione (25 g, 40.408 mmol) in DCM (250 mL, 3885.69 mmol), Tetrabutylammoniumhydrogensulfate (5.49 g, 16.163 mmol), and chloromethyl pivalate (7.30 g, 48.49 mmol) at room temperature. The reaction mixture was stirred at room temperature for 16 h. Some starting material remained unreacted by UPLC-Mass analysis, thus, added 700 mg of chloromethyl pivalate at room temperature. After stirring at rt for another 2 days, the mixture was worked up with saturated NaHCO₃ (50 mL) and extracted with EtOAc (100 mL×3), and purified by a column chromatography (100 g snap, EtOAc/Hept 10 to 60%) to give (3-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-2,6-dioxo-3,6-dihydropyrimidin-1(2H)-yl)methyl pivalate (23 g, 31.4 mmol, 78% yield) along with recovered starting material (3.25 g).

¹H NMR (400 MHz, DMSO-d₆) δ 7.62 (s, 1H), 7.40 (d, J=7.6 Hz, 2H), 7.36-7.20 (m, 7H), 6.90 (d, J=8.8 Hz, 4H), 5.89 (d, J=4.8 Hz, 1H), 5.84-5.73 (m, 2H), 5.17 (d, J=6.0 Hz, 1H), 4.26 (q, J=5.6 Hz, 1H), 4.12 (t, J=4.8 Hz, 1H), 4.02-3.98 (m, 1H), 3.78-3.70 (m, 8H), 3.51-3.40 (m, 2H), 3.28-3.20 (m, 5H), 1.44 (s, 3H), 1.10 (s, 9H); MS (ESI, m/z) calculated for [C₄₀H₄₈N₂O₁₁+Na+] 755.32 found 755.1.

### 2′-O-MOE-3′-PSI Activated Monomers

General Procedure 2¹: PSI activation

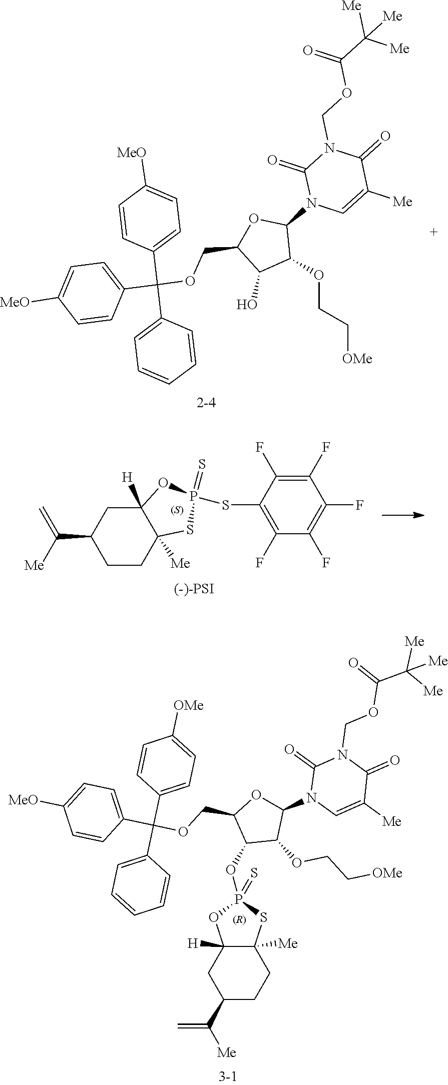

(3-1) (3-((2R,3R,4R,5R)-5-((bis(4-methoxyphenyl)(phenyl)methoxy)methyl)-3-(2-methoxyethoxy)-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-5-methyl-2,6-dioxo-3,6-dihydropyrimidin-1(2H)-yl)methyl pivalate:

(2S,3aS,6R,7aS)-3a-methyl-2-((perfluorophenyl)thio)-6-(prop-1-en-2-yl)hexahydrobenzo[d][1,3,2]oxathiaphosphole 2-sulfide (3.70 g, 8.29 mmol) ((−)-PSI reagent) and (3-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-2,6-dioxo-3,6-dihydropyrimidin-1(2H)-yl)methyl pivalate (4.50 g, 6.141 mmol) were dissolved in THF (20.47 mL, 6.141 mmol) and acetonitrile (20.47 mL, 6.141 mmol), and the solution was cooled in ice bath. To the mixture was added DBU (1.203 mL, 7.983 mmol) and it was stirred at 0° C. until the reaction was completed (0.5-2 h) as monitored by UPLC-MS. The reaction mixture was diluted by EtOAc was washed with saturated NaH₂PO₄ (aq.) solution, then saturated NaHCO₃ (aq.), dried over Na₂SO₄, and purified by a silica gel chromatography (50 g Star, Hept: EtOAc gradient to 70% to give 3-1 as a white solid (5.3 g, 88% yield).

¹H NMR (400 MHz, CD₃CN) δ ppm 7.46-7.54 (3H, m), 7.33-7.39 (6H, m), 7.26-7.32 (1H, m), 6.91 (4H, d, J=8.75 Hz), 5.97 (1H, d, J=6.38 Hz), 5.86-5.93 (2H, m), 5.45-5.52 (1H, m), 5.02 (1H, s), 4.93 (1H, s), 4.45-4.54 (2H, m), 4.26 (1H, d, J=2.88 Hz), 3.77-3.84 (8H, m), 3.47-3.62 (2H, m), 3.42 (1H, dd, J=11.01, 2.88 Hz), 3.29-3.33 (1H, m), 3.28 (3H, s), 2.64 (1H, br s), 2.25-2.32 (1H, m), 2.12-2.14 (3H, m), 2.07 (1H, br dd, J=13.70, 4.44 Hz), 1.99-1.99 (1H, m), 1.81-1.95 (2H, m), 1.80 (3H, s), 1.69 (3H, s), 1.44 (3H, s), 1.18 (9H, s); ³¹P NMR (162 MHz, CD₃CN) δ ppm 101.69; MS (ESI, m/z) calculated for [C₅₀H₆₃N₂O₁₂PS₂+Na⁺] 1001.35 found 1001.4.

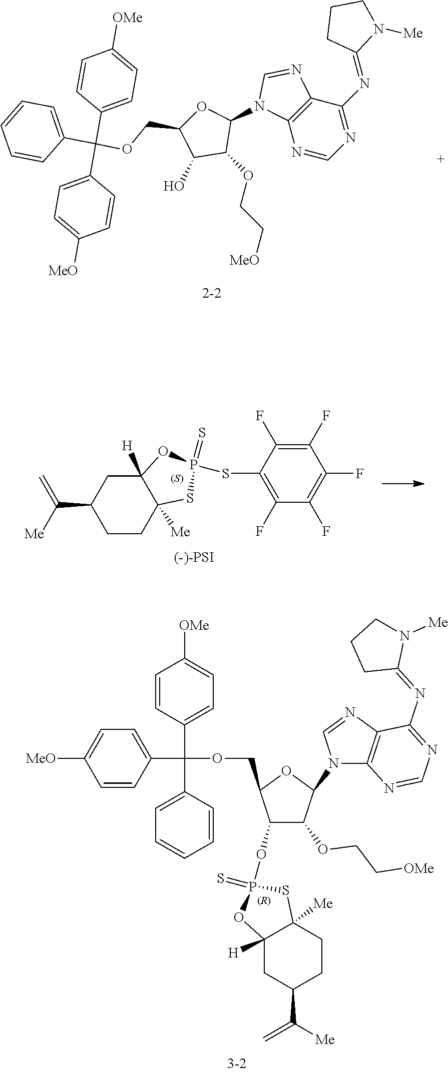

(3-2) (2R,3aS,6R,7aS)-2-(((2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-yl)oxy)-3a-methyl-6-(prop-1-en-2-yl)hexahydrobenzo[d][1,3,2]oxathiaphosphole 2-sulfide:

Prepared according to general procedure 2 with (−)-PSI reagent, 78% yield; foamy solid; ¹H NMR (400 MHz, CDC₃) δ 8.40-8.46 (m, 1H), 8.00-8.03 (m, 1H), 7.36-7.41 (m, 2H), 7.24-7.30 (m, 4H), 7.17-7.22 (m, 2H), 7.08-7.16 (m, 1H), 6.69-6.78 (m, 4H), 6.05 (d, J=7.5 Hz, 1H), 5.45-5.59 (m, 1H), 5.04 (dd, J=7.5, 4.7 Hz, 1H), 4.95 (s, 1H), 4.78-4.93 (m, 1H), 4.50 (dt, J=12.6, 3.3 Hz, 1H), 4.27-4.33 (m, 1H), 3.59-3.79 (m, 10H), 3.31-3.46 (m, 5H), 3.10-3.15 (m, 3H), 3.06-3.10 (m, 3H), 2.83-2.97 (m, 2H), 2.47-2.54 (m, 1H), 2.16-2.24 (m, 1H), 2.03-2.13 (m, 1H), 1.94-2.03 (m, 2H), 1.74-1.94 (m, 4H), 1.66-1.69 (m, 3H), 1.60-1.65 (m, 3H); 13C NMR (101 MHz, CDC₃) δ 166.9, 160.9, 158.6, 158.5, 152.8, 151.6, 144.8, 144.5, 140.1, 135.6, 135.6, 130.2, 130.1, 128.2, 128.0, 126.9, 126.6, 113.3, 112.2, 86.8, 85.4, 85.4, 83.6, 83.5, 80.1, 80.0, 77.3, 76.9, 72.3, 70.6, 68.0, 65.7, 63.0, 58.9, 55.2, 51.6, 38.9, 33.7, 33.7, 32.0, 30.1, 27.8, 27.6, 25.6, 23.5, 22.7, 21.8, 19.7; ³¹P NMR (162 MHz, CDCl₃) δ 101.34; MS (ESI, m/z) calculated for [C₄₉H₅₉N₆O₈PS₂+H⁺] 955.36 found 956.3.

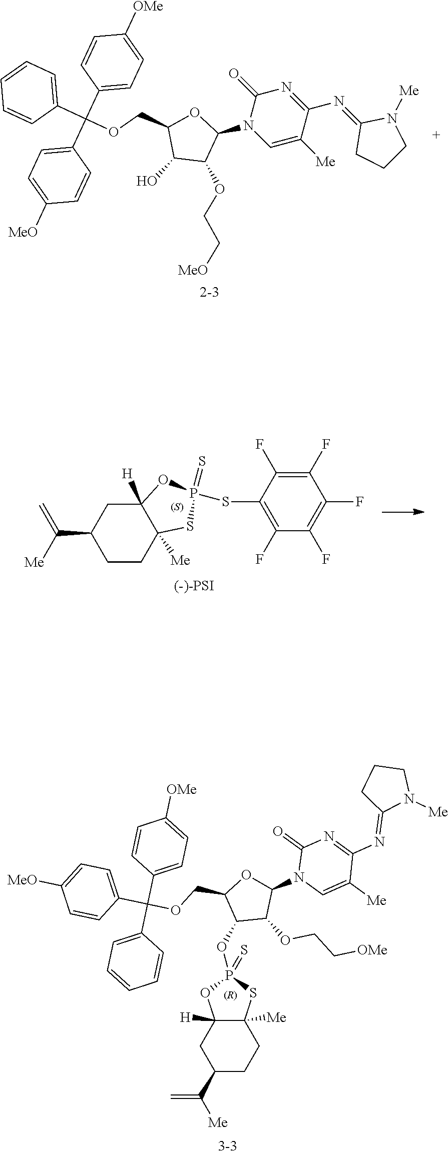

(3-3) 1-((2R,3R,4R,5R)-5-((bis(4-methoxyphenyl)(phenyl)methoxy)methyl)-3-(2-methoxyethoxy)-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-5-methyl-4-((1-methylpyrrolidin-2-ylidene)amino)pyrimidin-2(1H)-one:

Prepared according to general procedure 2 with (−)-PSI reagent, foamy solid, 70% yield; ¹H NMR (400 MHz, CD₃CN) δ ppm 7.59 (1H, s), 7.50 (2H, d, J=7.38 Hz), 7.32-7.41 (6H, m), 7.25-7.31 (1H, m), 6.90 (4H, d, J=8.63 Hz), 5.99 (1H, d, J=5.00 Hz), 5.44 (1H, dt, J=12.98, 5.02 Hz), 5.02 (1H, s), 4.93 (1H, s), 4.47 (1H, dt, J=12.73, 3.20 Hz), 4.34 (1H, t, J=5.07 Hz), 4.22-4.28 (1H, m), 3.86-3.94 (1H, m), 3.79 (6H, s), 3.44-3.61 (4H, m), 3.33-3.41 (3H, m), 3.30 (3H, s), 3.05-3.09 (1H, m), 3.04 (3H, s), 2.76 (1H, s), 2.64 (1H, br s), 2.20-2.30 (2H, m), 2.01-2.09 (3H, m), 1.99-1.99 (2H, m), 1.81-1.92 (1H, m), 1.80 (3H, s), 1.68 (3H, s), 1.55 (3H, s); ³¹P NMR (162 MHz, CD₃CN) δ ppm 101.51; MS (ESI, m/z) calculated for [C₄₉H₆₁N₄O₉PS₂+H⁺] 945.36 found 945.4.

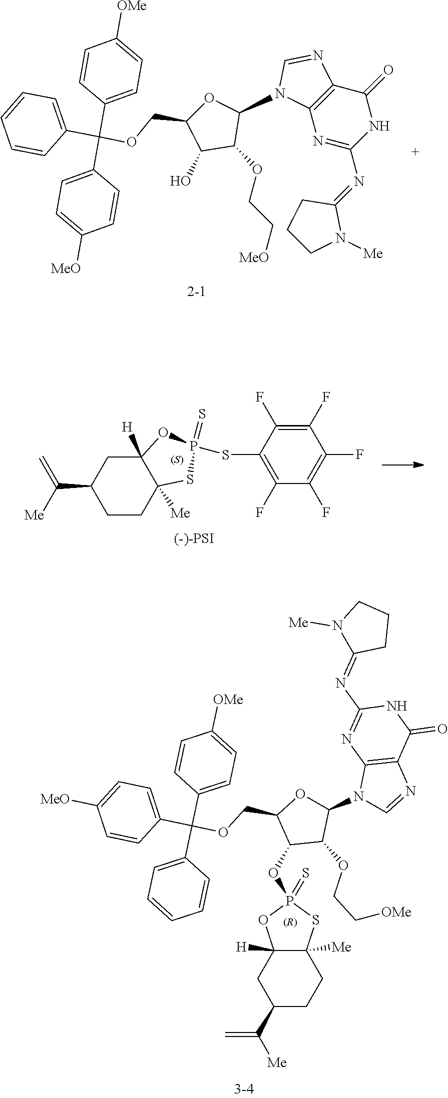

(3-4) 9-((2R,3R,4R,5R)-5-((bis(4-methoxyphenyl)(phenyl)methoxy)methyl)-3-(2-methoxyethoxy)-4-(((2R,3aS,6R,7aS)-3a-methyl-6-(prop-1-en-2-yl)-2-sulfidohexahydrobenzo[d][1,3,2]oxathiaphosphol-2-yl)oxy)tetrahydrofuran-2-yl)-2-((˜1-methylpyrrolidin-2-ylidene)amino)-1,9-dihydro-6H-purin-6-one:

Prepared according to general procedure 2 with (−)-PSI reagent, foamy solid, 80% yield; ¹H NMR (400 MHz, CD₃CN) δ ppm 9.18 (1H, br s), 7.74 (1H, s), 7.43 (2H, d, J=7.38 Hz), 7.22-7.33 (7H, m), 6.85 (4H, dd, J=9.01, 2.63 Hz), 5.89 (1H, d, J=5.50 Hz), 5.48 (1H, dt, J=13.54, 4.80 Hz), 4.98 (1H, s), 4.92 (1H, s), 4.85 (1H, t, J=5.38 Hz), 4.50 (1H, dt, J=12.69, 3.22 Hz), 4.23 (1H, q, J=4.09 Hz), 3.79 (6H, s), 3.67-3.77 (2H, m), 3.43-3.51 (4H, m), 3.32 (2H, qd, J=10.94, 4.06 Hz), 3.21 (3H, s), 3.03 (3H, s), 2.99-3.02 (1H, m), 2.63 (1H, br s), 2.27 (1H, br d, J=13.13 Hz), 2.12-2.15 (1H, m), 2.01-2.06 (1H, m), 1.99-1.99 (4H, m), 1.79-1.93 (2H, m), 1.77 (3H, s), 1.68 (3H, s); ³¹P NMR (162 MHz, CD₃CN) δ ppm 101.36; MS (ESI, m/z) calculated for [C₄₉H₅₉N₆O₉PS₂+H⁺] 971.35 found 971.4.

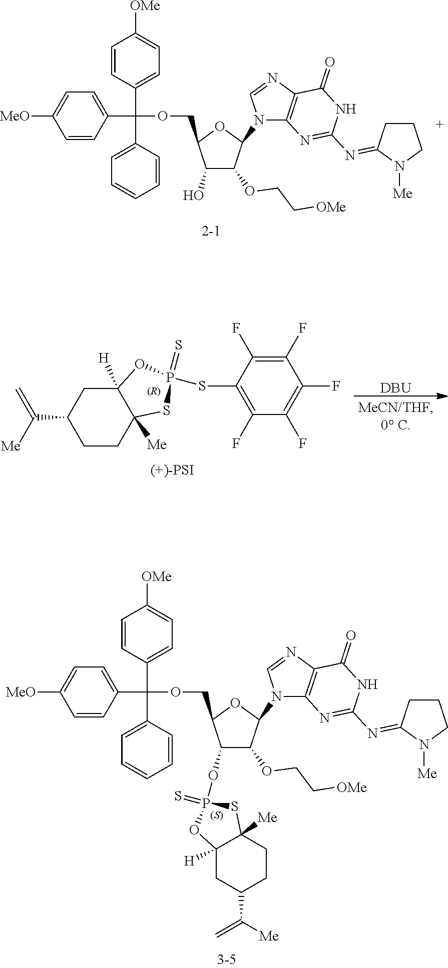

(3-5) 9-((2R,3R,4R,5R)-5-((bis(4-methoxyphenyl)(phenyl)methoxy)methyl)-3-(2-methoxyethoxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-2-((1-methylpyrrolidin-2-ylidene)amino)-1,9-dihydro-6H-purin-6-one:

Prepared according to general procedure 2 with (+)-PSI reagent, white foamy solid; ¹H NMR (400 MHz, CD₃CN, 296 K) δ (ppm)=9.56 (br s, 1H), 7.74 (s, 1H), 7.44 (d, J=7.5 Hz, 2H), 7.34-7.28 (m, 6H), 7.28-7.21 (m, 1H), 6.86 (dd, J=2.4, 8.9 Hz, 4H), 5.89 (d, J=6.4 Hz, 1H), 5.44-5.36 (m, 1H), 4.99 (s, 1H), 4.89 (s, 1H), 4.78 (t, J=5.8 Hz, 1H), 4.45 (td, J=3.0, 12.7 Hz, 1H), 4.27 (q, J=3.9 Hz, 1H), 3.78 (s, 6H), 3.75-3.70 (m, 1H), 3.67-3.57 (m, 1H), 3.47-3.40 (m, 2H), 3.39-3.32 (m, 4H), 3.12 (s, 3H), 3.09-2.92 (m, 5H), 2.63 (br s, 1H), 2.30-2.15 (m, 2H), 2.04 (br dd, J=4.0, 12.9 Hz, 1H), 2.00-1.90 (m, 4H), 1.82 (br s, 1H), 1.79-1.75 (m, 3H), 1.68 (s, 3H); ¹³C NMR (101 MHz, CD₃CN, 298 K) δ (ppm)=170.8, 160.1, 159.3, 158.3, 152.1, 147.2, 146.2, 137.9, 136.9, 136.9, 131.5, 131.4, 129.4, 129.3, 128.3, 114.5, 112.4, 87.9, 87.6, 87.3, 83.5, 83.4, 81.8, 78.2, 78.1, 73.1, 72.3, 66.4, 64.4, 59.4, 56.3, 52.4, 40.2, 34.9, 34.8, 32.5, 32.4, 28.7, 28.6, 24.2, 23.2, 22.3, 20.8; ³¹P NMR (162 MHz, CD₃CN) δ 101.9; MS (ESI, m/z) calculated for [C₄₉H₅₉N₆O₉PS₂+H⁺] 971.35 found 971.1.

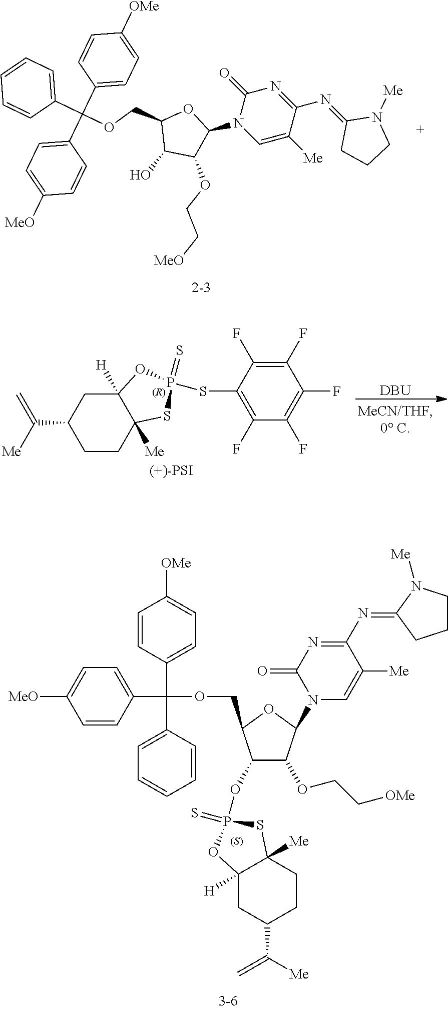

(3-6) 1-((2R,3R,4R,5R)-5-((bis(4-methoxyphenyl)(phenyl)methoxy)methyl)-3-(2-methoxyethoxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-5-methyl-4-(((E)-1-methylpyrrolidin-2-ylidene)amino)pyrimidin-2(1H)-one: Prepared according to general procedure 2 with (+)-PSI reagent, white foamy solid; ¹H NMR (400 MHz, CD₃CN, 296 K) δ (ppm)=7.57 (s, 1H), 7.50 (d, J=7.5 Hz, 2H), 7.40-7.31 (m, 6H), 7.31-7.23 (m, 1H), 6.90 (d, J=8.9 Hz, 4H), 6.05 (d, J=5.8 Hz, 1H), 5.49-5.40 (m, 1H), 4.99 (s, 1H), 4.88 (s, 1H), 4.43 (td, J=3.1, 12.6 Hz, 1H), 4.34 (t, J=5.4 Hz, 1H), 4.26 (br d, J=3.4 Hz, 1H), 3.85-3.72 (m, 8H), 3.54-3.45 (m, 4H), 3.38 (d, J=2.8 Hz, 2H), 3.26 (s, 3H), 3.11-3.05 (m, 2H), 3.03 (s, 4H), 2.62 (br s, 1H), 2.22 (br d, J=12.3 Hz, 1H), 2.13-2.01 (m, 3H), 2.01-1.92 (m, 2H), 1.92-1.79 (m, 2H), 1.76 (s, 3H), 1.68 (s, 3H), 1.56 (s, 3H); ¹³C NMR (101 MHz, CD₃CN, 298 K) δ (ppm)=172.6, 170.0, 160.2, 147.2, 146.1, 138.4, 137.0, 136.8, 131.5, 131.5, 129.5, 129.4, 128.4, 114.6, 112.5, 88.7, 88.2, 87.8, 82.9, 82.9, 82.3, 82.2, 77.7, 77.6, 73.3, 71.8, 66.7, 63.9, 59.5, 56.3, 52.5, 40.2, 34.9, 34.8, 32.4, 31.8, 28.7, 28.6, 24.2, 23.2, 22.3, 20.8; ³¹P NMR (162 MHz, CD₃CN) δ 101.8; MS (ESI, m/z) calculated for [C₄₉H₆₁N₄O₉PS₂+H⁺1 945.36 found 946.5.

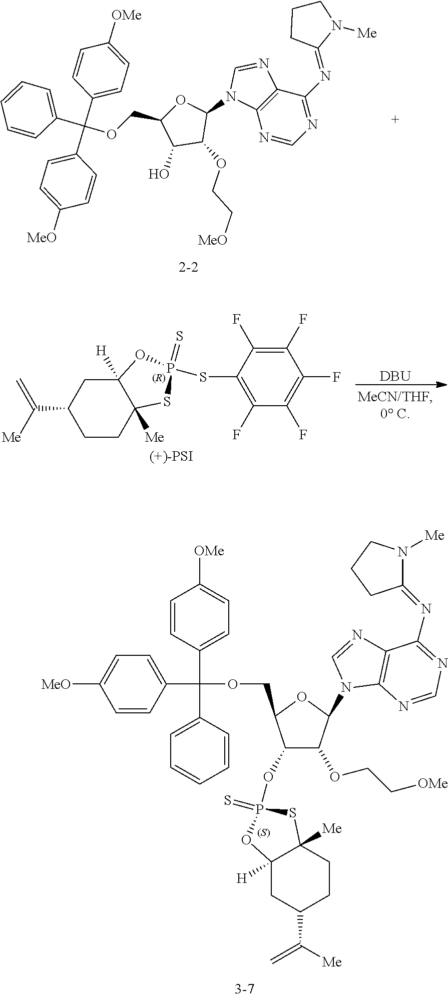

(3-7) (2S,3aR,6S,7aR)-2-(((2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-yl)oxy)-3a-methyl-6-(prop-1-en-2-yl)hexahydrobenzo[d][1,3,2]oxathiaphosphole 2-sulfide:

Prepared according to general procedure 2 with (+)-PSI reagent, white foamy solid; 1H NMR (400 MHz, CD₃CN, 296 K) δ (ppm)=8.35 (s, 1H), 8.06 (s, 1H), 7.47 (d, J=7.4 Hz, 2H), 7.34 (dd, J=1.6, 8.8 Hz, 4H), 7.30 (s, 2H), 7.26-7.19 (m, 1H), 6.85 (d, J=8.9 Hz, 4H), 6.01 (d, J=6.6 Hz, 1H), 5.60-5.53 (m, 1H), 5.10 (t, J=5.7 Hz, 1H), 4.99 (s, 1H), 4.91 (s, 1H), 4.49 (td, J=3.0, 12.6 Hz, 1H), 4.41-4.32 (m, 1H), 3.78 (s, 6H), 3.77-3.71 (m, 1H), 3.67 (br t, J=6.4 Hz, 2H), 3.65-3.57 (m, 1H), 3.52 (t, J=7.1 Hz, 2H), 3.45 (br d, J=4.5 Hz, 1H), 3.40-3.34 (m, 3H), 3.09 (s, 3H), 3.07 (s, 3H), 2.93 (t, J=7.9 Hz, 2H), 2.64 (br s, 1H), 2.27 (br d, J=13.4 Hz, 1H), 2.18-2.06 (m, 1H), 2.06-2.01 (m, 2H), 2.00-1.86 (m, 1H), 1.78 (s, 3H), 1.70 (s, 3H); ¹³C NMR (101 MHz, CD₃CN, 297 K) δ (ppm)=168.5, 162.1, 160.1, 153.5, 152.6, 147.2, 146.3, 142.2, 137.0, 131.5, 131.5, 129.4, 129.2, 129.2, 128.3, 128.0, 114.5, 112.5, 87.8, 87.7, 87.6, 83.9, 83.8, 80.8, 80.8, 78.4, 78.4, 72.9, 72.1, 68.7, 66.6, 64.2, 59.3, 56.3, 52.5, 40.2, 34.9, 34.8, 32.4, 31.3, 28.7, 28.6, 26.6, 24.2, 23.2, 22.3, 20.8; ³¹P NMR (162 MHz, CD₃CN) δ 101.7; MS (ESI, m/z) calculated for [C₄₉H₅₉N₆O₈PS₂+H⁺] 955.36 found 956.6.

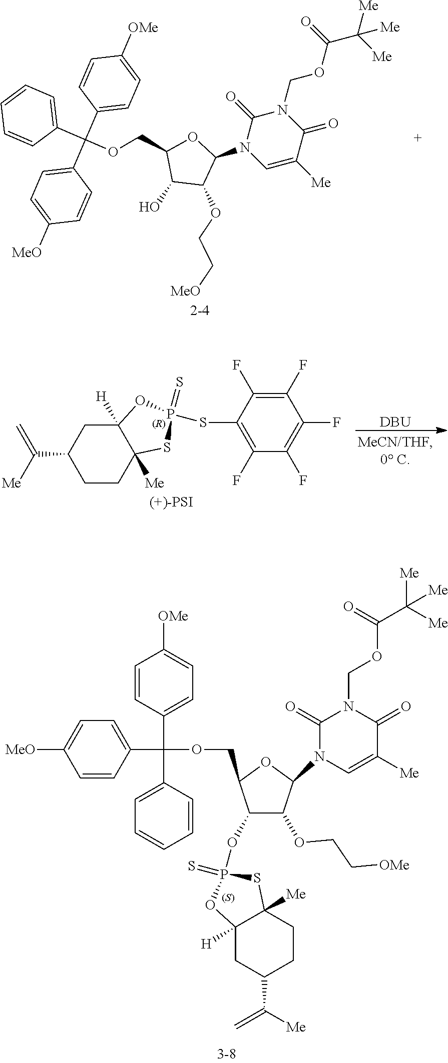

(3-8) (3-((2R,3R,4R,5R)-5-((bis(4-methoxyphenyl)(phenyl)methoxy)methyl)-3-(2-methoxyethoxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-5-methyl-2,6-dioxo-3,6-dihydropyrimidin-1(2H)-yl)methyl pivalate:

Prepared according to general procedure 2 with (+)-PSI reagent, foamy solid; ¹H NMR (400 MHz, CD₃CN) δ ppm 7.53 (1H, s), 7.48 (2H, d, J=7.63 Hz), 7.33-7.39 (6H, m), 7.26-7.32 (1H, m), 6.92 (4H, d, J=8.76 Hz), 6.01 (1H, d, J=6.88 Hz), 5.86-5.92 (2H, m), 5.45 (1H, ddd, J=11.60, 4.78, 2.75 Hz), 5.01 (1H, s), 4.90 (1H, s), 4.44-4.49 (2H, m), 4.29 (1H, br d, J=2.63 Hz), 3.80 (6H, s), 3.76-3.79 (1H, m), 3.32-3.54 (4H, m), 3.23 (3H, s), 2.60-2.66 (1H, m), 2.24 (2H, br d, J=12.76 Hz), 2.07 (1H, br dd, J=13.01, 3.75 Hz), 1.99-2.01 (2H, m), 1.80-1.92 (2H, m), 1.78 (3H, s), 1.68 (3H, s), 1.46 (3H, s), 1.18 (9H, s); ³¹P NMR (162 MHz, CD₃CN) δ ppm 102.18; MS (ESI, m/z) calculated for [C₅₀H₆₃N₂O₁₂PS₂+Na+] 1001.35 found 1001.1.

General Procedure 3: PO-PSI monomer from PS-PSI monomer

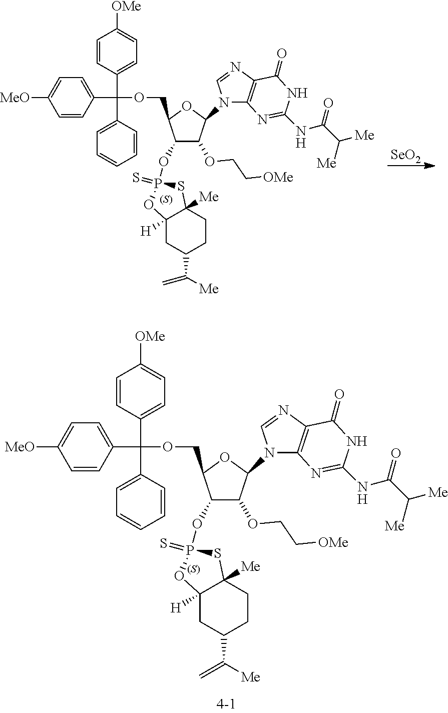

(4-1)N-(9-((2R,3R,4R,5R)-5-((bis(4-methoxyphenyl)(phenyl)methoxy)methyl)-3-(2-methoxyethoxy)-4-(((2S,3aR,6S,7aR)-3a-methyl-2-oxido-6-(prop-1-en-2-yl)hexahydrobenzo[d][1,3,2]oxathiaphosphol-2-yl)oxy)tetrahydrofuran-2-yl)-6-oxo-6,9-dihydro-1H-purin-2-yl)isobutyramide:

To N-(9-((2R,3R,4R,5R)-5-((bis(4-methoxyphenyl)(phenyl)methoxy)methyl)-3-(2-methoxyethoxy)-4-(((2S,3aR,6S,7aR)-3a-methyl-6-(prop-1-en-2-yl)-2-sulfidohexahydrobenzo[d][1,3,2]oxathiaphosphol-2-yl)oxy)tetrahydrofuran-2-yl)-6-oxo-6,9-dihydro-1H-purin-2-yl)isobutyramide (1 g, 1.042 mmol) in MeCN (15.00 mL, 15 vol) was added SeO₂ (1.0 eq., 0.116 g, 1.042 mmol) in ice-bath. Additional SeO₂ (0.116 g, 1.042 mmol) was added at 0° C. until the reaction was completed at 0° C. Total 3 equivalents of SeO₂ were used. Upon completion as monitored by UPLC-MS, the mixture was filtered over celite and dry SiO₂ (EtOAc/THF). The filtrate was washed with saturated NaHCO₃ (10 mL), dried over Na₂SO₄, filtered (dry SiO₂) and concentrated. The residue was purified by a silica-gel column chromatography (50 g, Hept/EtOAc, 20 to 100 then EtOAc/THF 0 to 100%) to give the 4-1 (0.55 g, 56% yield). MS (ESI, m/z) calculated for [C₄₈H₅₈N₅O₁₁PS—H⁺] 942.36 found 942.53.

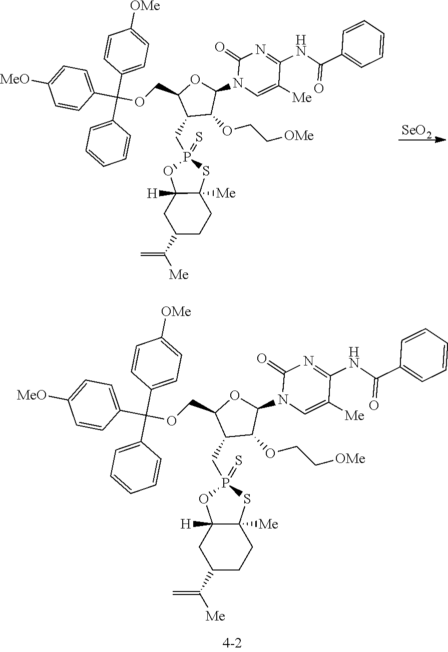

(4-2)N-(1-((2R,3R,4R,5R)-5-((bis(4-methoxyphenyl)(phenyl)methoxy)methyl)-3-(2-methoxyethoxy)-4-(((2R,3aS,6R,7aS)-3a-methyl-2-oxido-6-(prop-1-en-2-yl)hexahydrobenzo[d][1,3,2]oxathiaphosphol-2-yl)oxy)tetrahydrofuran-2-yl)-5-methyl-2-oxo-1,2-dihydropyrimidin-4-yl)benzamide:

Prepared according to general procedure 3, white foamy solid; 59% yield; ³¹P NMR (162 MHz, acetonitrile-d₃) δ 40.36; MS (ESI, m/z) calculated for [C₅₁H₅₈N₃O₁₁PS+H⁺] 952.35 found 952.35.

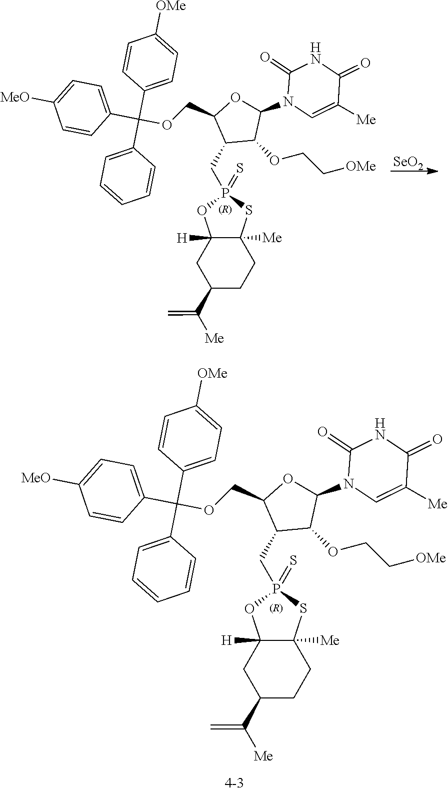

(4-3) 1-((2R,3R,4R,5R)-5-((bis(4-methoxyphenyl)(phenyl)methoxy)methyl)-3-(2-methoxyethoxy)-4-(((2R,3aS,6R,7aS)-3a-methyl-2-oxido-6-(prop-1-en-2-yl)hexahydrobenzo[d][1,3,2]oxathiaphosphol-2-yl)oxy)tetrahydrofuran-2-yl)-5-methylpyrimidine-2,4(1H,3H)-dione:

Prepared according to general procedure 3, white foamy solid; 46% yield; ³¹P NMR (162 MHz, acetonitrile-d₃) δ 40.42; MS (ESI, m/z) calculated for [C₄₄H₅₃N₂O₁₁PS+Na⁺] 871.30 found 871.28.

PO-PSI reagent from cyclohexyl epoxide:

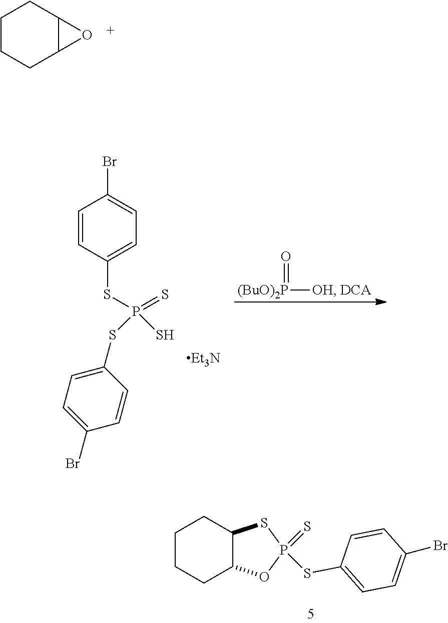

(5) rac-2-((4-bromophenyl)thio)hexahydrobenzo[d][1,3,2]oxathiaphosphole 2-sulfide:

A solution of triethylamine bis(4-bromophenyl) phosphorotetrathioate (50.0 g, 87.2 mmol) and cyclohexene oxide (13.2 mL, 131 mmol) in chloroform (175 mL) was treated with dibutyl phosphate (16.2 mL, 87.2 mmol) and dichloroacetic acid (10.8 mL, 131 mmol). After stirring at room temperature for 15 hours, the mixture was concentrated in vacuo. The residue was diluted with water (125 mL) and n-heptane (125 mL), cooled with an ice bath, and stirred at 0° C. for 2 hours. The resulting precipitate was filtered, and washed subsequently with water (100 mL) and n-heptane (125 mL). The filter cake was dissolved in CH₂Cl₂ (200 mL) and the aqueous layer was removed. The organic layer was concentrated in vacuo to ca. 50 mL and treated with n-heptane (75 mL). The mixture was stirred at room temperature for 20 min and concentrated in vacuo to ca. 50 mL. The resulting precipitate was filtered, washed with n-heptane (20 mL), and dried over N₂ purge for 2 hours to give the title compound (30.1 g, 91%).

¹H NMR (400 MHz, CDC₃, 296 K) (a 1:2 mixture of diastereomers) δ (ppm)=7.58-7.51 (m, 8H), 7.47-7.41 (m, 4H), 4.04 (dt, J=3.9, 10.7 Hz, 1H), 3.65-3.56 (m, 4H), 2.27-2.12 (m, 6H), 1.89 (m, 3H), 1.81 (m, 3H), 1.75-1.58 (m, 3H), 1.49-1.25 (m, 8H), 1.23-1.16 (m, 1H), 1.07-0.86 (m, 1H); ³¹P NMR (162 MHz, CDC₃, 296 K) δ (ppm)=107.01 (s, 1P), 103.23 (s, 2P); MS (ESI) m/z: [M+H]⁺ calcd for C₁₂H₁₅BrOPS₃ 380.91; Found 380.84.

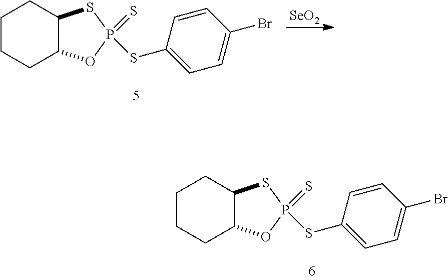

### (6) rac-2-((4-bromophenyl)thio)hexahydrobenzo[d][1,3,2]oxathiaphosphole 2-oxide

A solution of (3aR,7aR)-2-((4-bromophenyl)thio)hexahydrobenzo[d][1,3,2]oxathiaphosphole 2-sulfide (10.0 g, 26.2 mmol) in CH₂Cl₂ (170 mL) was treated with SeO₂ (2.91 g, 26.2 mmol) and stirred at room temperature for 2 hours. Additional SeO₂ (2.91 g, 26.2 mmol) was added and stirring was continued at rt for additional 19 hours. The reaction mixture was filtered through a dry silica gel pad and rinsed with CH₂Cl₂. The filtrate was washed with 10% NaH₂PO₄ (70.0 mL), dried over MgSO₄ and concentrated in vacuo. The residue was treated with n-heptane (46 mL) and the resulting slurry was stirred at room temperature for 20 minutes. The precipitate was filtered, washed with n-heptane (20 mL) and dried over N₂ purge to give the title compound (6.18 g, 64.5%).

¹H NMR (400 MHz, CDC₃, 296 K) (ca. 1:2 mixture of two diastereomers) δ (ppm)=7.58-7.48 (m, 12H), 4.10 (dt, J=4.1, 10.8 Hz, 1H), 3.60 (dt, J=3.6, 10.8 Hz, 2H), 3.37 (dt, J=3.9, 10.8 Hz, 2H), 2.43-2.36 (m, 1H), 2.25-2.07 (m, 5H), 1.98-1.83 (m, 4H), 1.83-1.73 (m, 3H), 1.63-1.48 (m, 3H), 1.46-1.23 (m, 8H), 1.11-0.99 (m, 1H); ³¹P NMR (162 MHz, CDC₃, 297 K) δ (ppm)=62.54 (s, 1P), 56.98 (s, 2P); MS (ESI) m/z: [M+H]⁺ calcd for C₁₂H₁₅BrO₂PS₂ 364.94; Found 364.97.

General Procedure 4: PO-PSI monomer

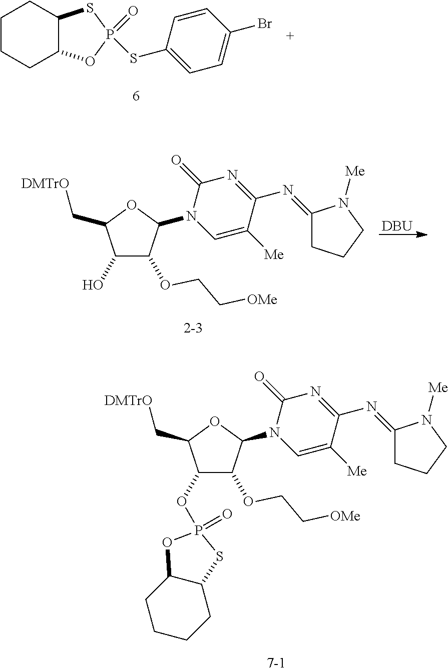

(7-1) 1-((2R,3R,4R,5R)-5-((bis(4-methoxyphenyl)(phenyl)methoxy)methyl)-3-(2-methoxyethoxy)-4-(((3aR,7aR)-2-oxidohexahydrobenzo[d][1,3,2]oxathiaphosphol-2-yl)oxy)tetrahydrofuran-2-yl)-5-methyl-4-(((E)-1-methylpyrrolidin-2-ylidene)amino)pyrimidin-2(1H)-one:

1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-4-(((E)-1-methylpyrrolidin-2-ylidene)amino)pyrimidin-2(1H)-one (4.30 g, 6.15 mmol) and (3aR,7aR)-2-((4-bromophenyl)thio)hexahydrobenzo[d][1,3,2]oxathiaphosphole 2-oxide (3.15 g, 8.62 mmol) was azeotroped three times with acetonitrile (43 mL). The residue was dissolved in acetonitrile (43 mL), cooled to 0° C., and treated with DBU (1.6 mL, 8.3 mmol). The mixture was stirred at 0° C. for 2 hours, quenched with saturated NaH₂PO₄ (40 mL), and diluted with ethyl acetate (50 mL). The organic layer was separated, and the aqueous layer was extracted twice with ethyl acetate (50 mL). The organic layers were combined, washed with sat. NaHCO₃ (20 mL), dried over MgSO₄, and concentrated in vacuo. The residue was purified by silica gel column chromatography (ethyl acetate in n-heptane=17% to 100% and then THF in ethyl acetate=0% to 100%) to give the title compound (3.07 g, 57.1%) as a foaming solid.

MS (ESI) m/z: [M+H]⁺ calcd for C₄₅H₅₆N₄O₁₀PS 875.3; Found 875.1.

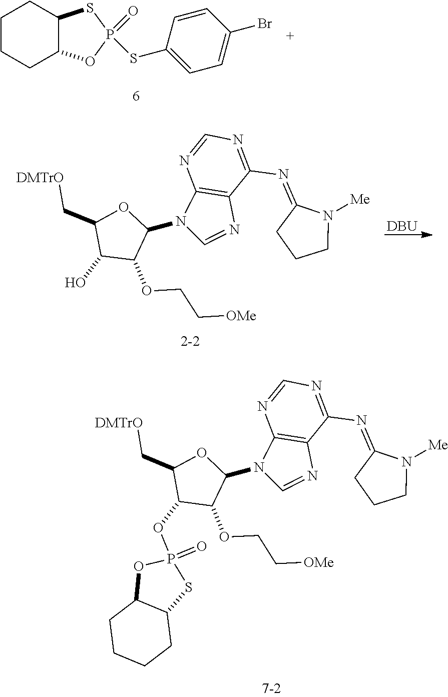

(7-2) (3aR,7aR)-2-(((2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-yl)oxy)hexahydrobenzo[d][1,3,2]oxathiaphosphole 2-oxide:

(2R,3R,4R,5R)-2-((bis(4-methoxyphenyl)(phenyl)methoxy)methyl)-4-(2-methoxyethoxy)-5-(6-(((E)-1-methylpyrrolidin-2-ylidene)amino)-9H-purin-9-yl)tetrahydrofuran-3-ol (2.70 g, 3.81 mmol) and (3aR,7aR)-2-((4-bromophenyl)thio)hexahydrobenzo[d][1,3,2]oxathiaphosphole 2-oxide (1.95 g, 5.33 mmol) was azeotroped three times with acetonitrile (25.4 mL) in the rotary evaporator. The residue was dissolved in acetonitrile (25.4 mL), cooled to 0° C., and treated with DBU (0.78 mL, 5.1 mmol). The mixture was stirred at 0° C. for 2 hours, quenched with saturated NaH₂PO₄ (30 mL), and diluted with ethyl acetate (30 mL). The organic layer was separated, and the aqueous layer was extracted twice with ethyl acetate (30 mL). The organic layers were combined, washed with saturated NaHCO₃ (20 mL), dried over MgSO₄, and concentrated in vacuo. The residue was purified by column chromatography (ethyl acetate in n-heptane=17% to 100%, and then THF in ethyl acetate=0% to 100%) to give the title compound (2.10 g, 62.3%) as a foamy solid.

MS (ESI) m/z: [M+H]⁺ calcd for C₄₅H₅₄N₆O₉PS 884.33 Found 884.45.

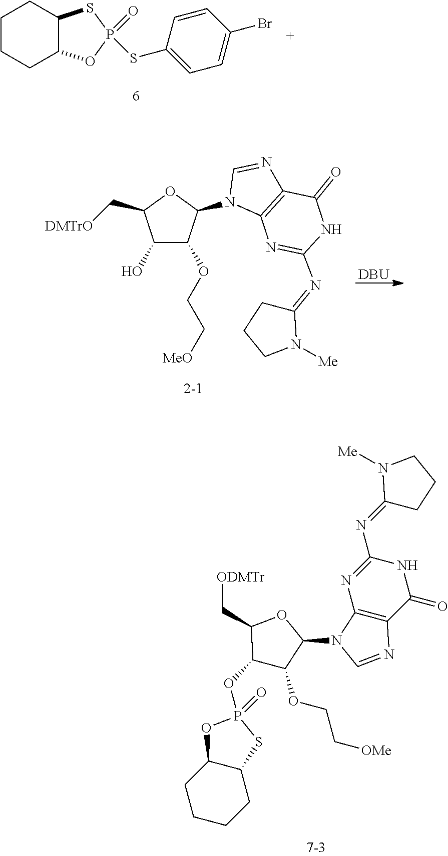

(7-3) 9-((2R,3R,4R,5R)-5-((bis(4-methoxyphenyl)(phenyl)methoxy)methyl)-3-(2-methoxyethoxy)-4-(((3aR,7aR)-2-oxidohexahydrobenzo[d][1,3,2]oxathiaphosphol-2-yl)oxy)tetrahydrofuran-2-yl)-2-((1-methylpyrrolidin-2-ylidene)amino)-1,9-dihydro-6H-purin-6-one:

Prepared according to general procedure 4, a white foamy solid; 80% yield; MS (ESI, m/z) calculated for [C₄₅H₅₃N₆O₁₀PS+H⁺] 901.33 found 901.1.

### General Procedure 5: Synthesis of Monomer Succinates

To the protected nucleoside (1.0 eq.) and succinic anhydride (1.5 eq.) were added DCM (8 vol) and Et₃N (3.0 eq.) at room temperature. The mixture was stirred overnight at room temperature. To the mixture was added phosphate buffer (pH 7, 6 vol) and extracted with DCM (8 vol) 3 times. Then the organic layers were concentrated and purified by a column chromatography (Heptane/EtOAc, 10 to 100%).

### (5-Methyl-C-MOE Succinate):

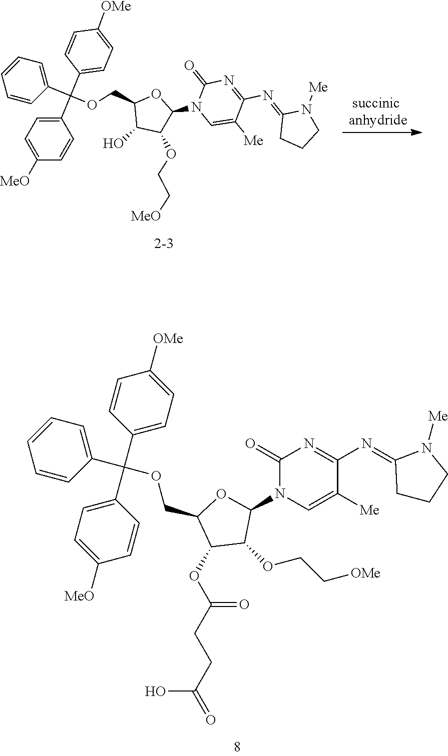 [SMILES: COCCO[C@@H]1[C@H](O)[C@@H](COC(c2ccccc2)(c2ccc(OC)cc2)c2ccc(OC)cc2)O[C@H]1n1cc(C)c(/N=C2\CCCN2C)nc1=O ; COCCO[C@@H]1[C@H](OC(=O)CCC(=O)O)[C@@H](COC(c2ccccc2)(c2ccc(OC)cc2)c2ccc(OC)cc2)O[C@H]1n1cc(C)c(/N=C2\CCCN2C)nc1=O]

(8) 4-(((2R,3R,4R,5R)-2-((bis(4-methoxyphenyl)(phenyl)methoxy)methyl)-4-(2-methoxyethoxy)-5-(5-methyl-4-(((E)-1-methylpyrrolidin-2-ylidene)amino)-2-oxopyrimidin-1(2H)-yl)tetrahydrofuran-3-yl)oxy)-4-oxobutanoic acid:

To 1-((2R,3R,4R,5R)-5-((bis(4-methoxyphenyl)(phenyl)methoxy)methyl)-4-hydroxy-3-(2-methoxyethoxy)tetrahydrofuran-2-yl)-5-methyl-4-(((E)-1-methylpyrrolidin-2-ylidene)amino)pyrimidin-2(1H)-one (5 g, 7.155 mmol) and succinic anhydride (1.074 g, 10.732 mmol) in DCM (40.0 mL, 621.71 mmol) was added Et₃N (2.99 mL, 21.465 mmol) at room temperature. The mixture was stirred overnight at room temperature. To the mixture was added phosphate buffer (pH 7, 30 mL) and extracted with DCM (50 mL×3). Then the organic layers were concentrated and purified by a column chromatography (Hept/EtOAc, 10 to 100%) to give 4-(((2R,3R,4R,5R)-2-((bis(4-methoxyphenyl)(phenyl)methoxy)methyl)-4-(2-methoxyethoxy)-5-(5-methyl-4-(((E)-1-methylpyrrolidin-2-ylidene)amino)-2-oxopyrimidin-1(2H)-yl)tetrahydrofuran-3-yl)oxy)-4-oxobutanoic acid (4.92 g, 6.16 mmol, 86% yield).

¹H NMR (400 MHz, CD₃CN) δ ppm 7.59 (1H, s), 7.47 (2H, d, J=7.50 Hz), 7.31-7.39 (6H, m), 7.24-7.31 (1H, m), 6.91 (4H, d, J=8.63 Hz), 6.04 (1H, d, J=5.25 Hz), 5.35 (1H, t, J=5.13 Hz), 4.35 (1H, t, J=5.32 Hz), 4.14-4.25 (1H, m), 3.79 (6H, s), 3.74-3.78 (1H, m), 3.66 (1H, dt, J=11.44, 4.28 Hz), 3.44-3.52 (4H, m), 3.33-3.40 (2H, m), 3.26 (3H, s), 3.05-3.11 (2H, m), 3.04 (3H, s), 2.50-2.65 (4H, m), 2.00-2.08 (2H, m), 1.99 (1H, s), 1.61 (3H, s); MS (ESI, m/z) Calculated for [C₄₃H₅₀N₄O₁₁+H⁺] 799.35; Found 799.9.

### General Procedure 6: Solid Phase Synthesis of Stereo-Controlled PS MOE ASO

A general procedure for automated solid-phase synthesis of stereo-controlled PS-oligonucleotides was modified from the reported procedures in Knouse et al., “Unlocking P(V): Reagents for chiral phosphorothioate synthesis,” Science 2018, 361 (6408), 1234-1238; and Huang et al., “A P(V) platform for oligonucleotide synthesis,” Science 2021, 373 (6560), 1265-1270.

### Automated Solid-Phase Oligonucleotide Synthesis:

### Part 1. Loading to Resin: Preparation of 1mer

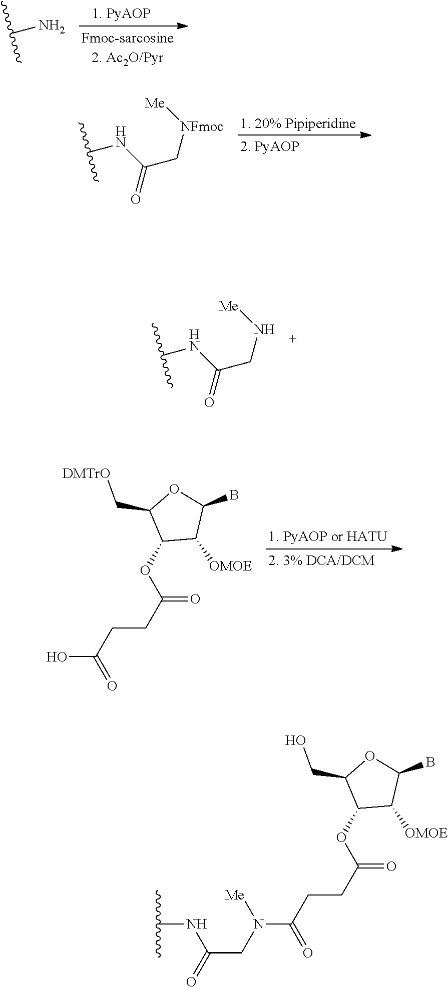

TentaGel S-NH₂ (AC354610050, ACROS Organics, loading 0.2 to 0.3 mmol/g) (4 g, ˜ 1 mmol) was placed in a 50 mL solid phase reaction flask and washed with DMF (10 mL×3), DCM (10 mL×3) and DMF (10 mL×3). To the resin were added N-(((9H-fluoren-9-yl)methoxy)carbonyl)-N-methylglycine (3.11 g, 10.00 mmol) in DMF (5.00 mL) and ((3H-[1,2,3]triazolo[4,5-b]pyridin-3-yl)oxy)tri(pyrrolidin-1-yl)phosphonium hexafluorophosphate(V) (5.21 g, 10.00 mmol) in DMF (5 mL) followed by N-4-methylmorpholine (2199 mL, 20.00 mmol) at room temperature. It was shaken at 400 rpms. After 24 hours, the liquid was drained and the resin was rinsed with DMF (10 mL×3), DCM (10 mL×3) and DMF (10 mL×3). To the resin was added premixed pyridine (4.85 mL, 60.00 mmol) and Ac₂O (0.944 mL, 10.00 mmol) at room temperature. After 3 minutes, the solution was drained and premixed pyridine (4.85 mL, 60.00 mmol) and Ac₂O (0.944 mL, 10.00 mmol) was added at room temperature. After 3 minutes, the liquid was drained and the resin was washed with DMF (10 mL×3), DCM (10 mL×3) and DMF (10 mL×3).

Then, the resin was treated with 30 mL of 20% piperidine in DMF and the solution was collected after 3 minutes. This process was repeated 5 times and the resin was washed with DMF (10 mL×3), DCM (10 mL×3) and DMF (10 mL×3). A solution of 20% piperidine in DMF was added to the collected solution to make 300 mL in a volumetric flask. An aliquot of this solution was diluted 10-fold with 20% piperidine in DMF and the UV absorbance of the piperidine-fulvene adduct was measured (λ=301 nm, ε=7800 M³¹¹ cm³¹¹, λ=2.41) to give 230 μmol/g as an estimated loading.

The resin was washed with DMF (10 mL×3), DCM (10 mL×3) and DMF (10 mL×3). To the resin was added 4-(((2R,3R,4R,5R)-2-((bis(4-methoxyphenyl)(phenyl)methoxy)methyl)-4-(2-methoxyethoxy)-5-(5-methyl-4-(((E)-1-methylpyrrolidin-2-ylidene)amino)-2-oxopyrimidin-1(2H)-yl)tetrahydrofuran-3-yl)oxy)-4-oxobutanoic acid (1.5 eq., 1.198 g, 1.5 mmol)) in DMF (5 mL) followed by ((3H-[1,2,3]Triazolo[4,5-b]pyridin-3-yl)oxy)tri(pyrrolidin-1-yl)phosphonium hexafluorophosphate(V) (1.7 eq., 0.886 g, 1.7 mmol) in DMF (5 mL) and N-4-Methylmorpholine (2 eq. 0.258 g, 2 mmol). The mixture was shaken at room temperature for 3 days and washed with DMF (10 mL×3), DCM (10 mL×3) and DMF (10 mL×3).

The resin was washed with DMF (10 mL×3), DCM (10 mL×3). It was treated for 2 minutes with 3% dichloroacetic acid (DCA) in DCM (20 mL) followed by DCM (20 mL) washing to remove the DMTr group. The process was repeated (>5 times) until no color was observed. Then the resin was washed with DCM (10 mL×3), DMF (10 mL×3) and MeCN (10 mL×3).

The combined deprotection solutions were diluted with 3% DCA in DCM. The UV absorbance of the DMTr cation was measured (λ=410 nm, ε=30,400 M³¹¹ cm³¹¹) to quantify the loading (0.2 mmol/g).

Part 2. Automated synthesis on K & A H-8-SE Oligo Synthesizer

The prepared 5′-O-DMTr-nucleotide-loaded TentaGel-SAR (20 μmol, 200 μmol/g) was packed in an empty 6 mL syringe column (Biocomma Limited, Cat #RSSC-6) and washed with MeCN. The stereopure oligonucleotides were synthesized on K &A H-8-SE Oligo Synthesizer following the cycles shown in Table 15 using stereopure PSI monomers and PO-PSI monomers. As shown in the schemes below: Sp phosphorothioate linkage was obtained using Rp-PSI-monomers that were prepared from (−)-PSI reagent; Rp phosphorothioate linkage was obtained using Sp-PSI-monomers that were synthesized from (+)-PSI, and PO internucleotide linkages were obtained using PO-PSI monomers¹.

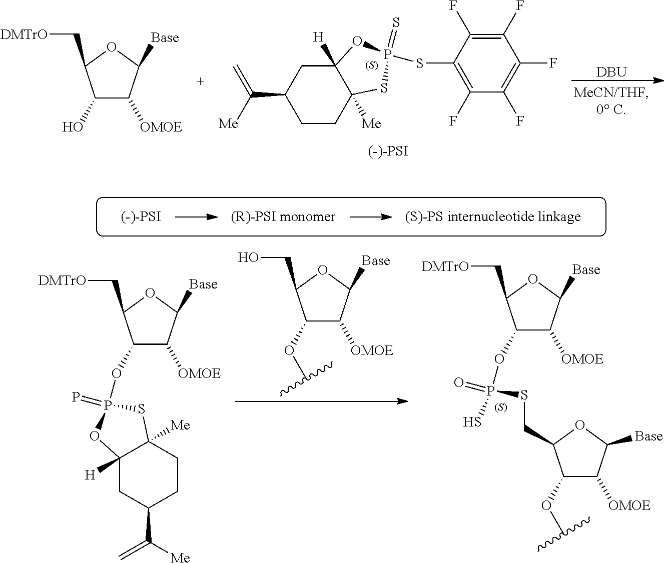

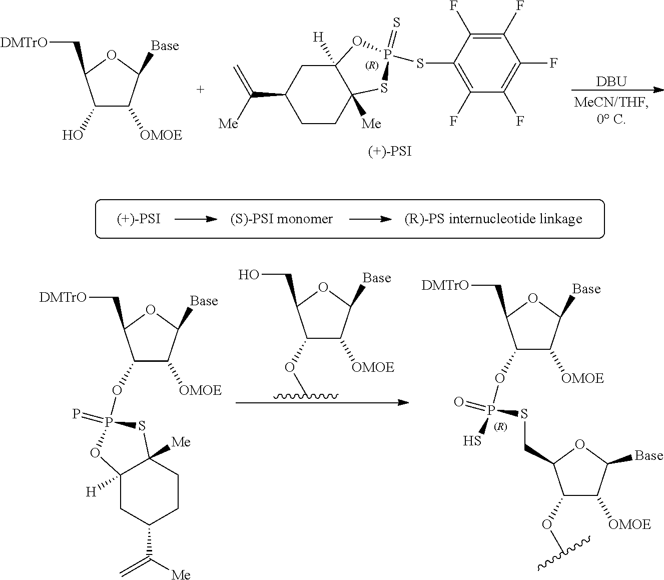

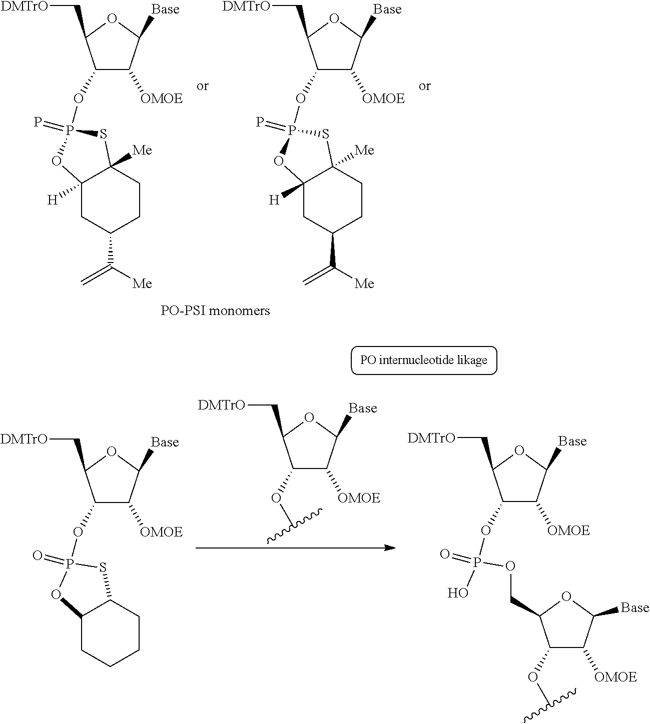

Monomers in the synthesis of Sp, Rp phosphorothioate and PO (phosphodiester) internucleotide linkages.

TABLE 15
Protocol for automated solid phase synthesis of MOE PS oligonucleotides

| | Step | Comment | Reagent/solvent | Time | | cycles |
|---|---|---|---|---|---|---|
| 1 | Deblock | PhMe wash | 3% DCA in PhMe | 1 | min | 5 |
| | | between cycles | | | | |
| 2 | Wash | | MeCN | 3 | min | 5 |
| 3 | Base Wash* | | 2,6-lutidine/DBU/MeCN (2/1/20) | 3 | min | 2 |
| 4 | Wash | | MeCN | 3 | min | 5 |
| 5 | Coupling | | Monomer (>10 eq., 0.1N), 2,6-lutidine | 16 | hr | 1 |
| | | | (>50 eq.), DBU (13 eq.) (1N/0.3N)** | | | |
| 6 | Wash | | MeCN | 3 | min | 5 |
| 7 | Capping | solvent (MeCN) | 20% AC₂O, 30% 2,6-lutidine, 20% | 1 | min | 2 |
| | | | N—Me-imidazole (1/1/1) | | | |
| 8 | Wash | | MeCN | 3 | min | 5 |

*Base wash solution: MeCN/2,6-lutidine/DBU = 20/2/1 (v/v/v)
**Coupling: monomer (0.2 mmol) in MeCN (2 mL, 0.1M) and base solution [2,6-lutidine 1.1 mL (1M), DBU 0.5 mL (0.3M), MeCN 10 mL] (0.9 mL) (>10 eq. DBU and >40 eq. 2,6-utidine) was transferred to the column. It was shaken for 16 hours, it was drained and washed, and conversion was analyzed by RP HPLC-Mass after cleavage from a bit of resin (28% NH₄OH/EtOH/NH₄OAc (9/2/1, v/v/w), 65° C., 4 hours). >95% conversion was achieved while lower conversions were observed either without 2,6-lutidine or shorter reaction time: no 2,6-lutidine, DBU (15 eq.): ~50% conversion or Monomer (10 eq.), 2,6-lutidine (50 eq.), DBU (15 eq.), 8 h: ~80% conversion.

Analytical HPLC Method 1-RP HPLC-Mass: Column: Acquity UPLC BEH C18 1.7 μm 2.1×50 mm (Part Number: 186002350); Solvents: Buffer A (10 mM ammonium bicarbonate in water), Buffer B (100 mM ammonium bicarbonate/MeOH/MeCN=10/10/80); temperature: 60° C.; Flow rate: 0.8 mL/min; Gradient: 5˜ 99% B gradient (6 min).

Part 3. Cleavage from Resin and Deprotection:

After completion of the last cycle (DMTr-On), the resin in cleavage solution (28% NH₄OH/NH₄OAc/EtOH (10/1/1, ˜1 mL/1 μmol) was heated at 65° C. for 2 days in a closed bottle. It was cooled to room temperature, filtered, and then concentrated. The failed sequences were removed and DMTr group was deprotected by the below C18 cartridge protocol. The collected fractions were concentrated and purified by an Ion-Pairing Reverse-Phase (IR-RP) HPLC.

### C18 Column Protocol:

Sep-Pak cartridge [Waters, Sep-Pak Vac 35 cc (10 g) C18 Cartridge] was equilibrated with MeOH (2 CV), MeCN (2 CV) followed by 2 N Et₃NHOAc (2 column volumes (CV)). The crude sample in 0.1 N Et₃NHOAc was loaded on a cartridge. The cartridge was washed with 2 N NaCl/MeCN (5/1, v/v) to elute truncated sequences, and 3% TFA in water (150 mL), then water (50 mL). The crude DMTr-off PS-oligonucleotide was eluted with 50 mL of acetonitrile-water (1:1, v/v) containing 0.5% of 28% NH₄OH. The solution containing crude DMTr-off oligonucleotide was dried under vacuum. The weight was measured by Nanodrop (RNA-40) and ³¹P NMR was taken. It was analyzed by RP-HPLC, IEX-HPLC and UPLC/MS.

Analytical HPLC Method 2-lon-pairing RP HPLC-Mass: Column: XBridge Premier BEH C18 (2.5 μm, 150×2.1 mm); Temperature: 60° C.; Flow rate: 1 mL/minute; Detection wavelength: 260 nm; Solvents: buffer A: 100 mM HFIP/8.6 mM Et₃N (H₂O), buffer B:100% MeOH; Gradient: 5% to 30% B gradient (15 minutes).

Analytical HPLC Method 3-lon-pairing RP HPLC-Mass: Column: XBridge Premier BEH C18 (300 Å, 2.5 μm, 150×2.1 mm); Temperature: 60° C., Flow rate: 0.5 mL/minute; Detection wavelength: 260 nm; Solvents: Buffer A: 100 mM n-C₆H₁₃NH₃OAc (H₂O/MeCN 9/1) Buffer B: 100 mM C₆H₁₃NH₃OAc (H₂O/MeCN 1/1); Gradient: 80% to 100% B gradient (15 minutes).

Analytical HPLC Method 4-lon-pairing RP HPLC-Mass: Column: XBridge Premier BEH C18 (300 Å, 2.5 μm, 150×2.1 mm); Temperature: 60° C., Flow rate: 0.5 mL/min. Detection wavelength: 260 nm; Solvents: buffer A: 10 mM n-Hexylamine/50 mM HFIP in water, buffer B: MeCN; Gradient: 23˜ 28% Buffer B gradient (15 minutes).

### Part 4. HPLC Purification and Desalting:

The crude material after SepPak treatment was purified by a ion-pairing RP HPLC by the following methods using sterile water (WFI from Baxter, VWR cat. 68000-955).

Preparative HPLC Method 1: Column: XBridge Prep C18 OBD Prep (10 μm, 19×250 mm); Flow rate: 30 mL/minute. Detection wavelength: 260 nm; Solvents: buffer A: 8.6 mM TEA/100 mM HFIP in water, Buffer B: MeOH; Gradient: 10-37% Buffer B gradient (30 minute).

Preparative HPLC Method 2: Column: Xbridge BEH C18 (10 μm, 10×250 mm); Flow rate: 14 mL/minute. Detection wavelength: 260 nm; Solvents: buffer A: 100 mM C₆H₁₃NH₃OAc (H₂O/MeCN 9/1), Buffer B: 100 mM C₆H₁₃NH₃OAc (H₂O/MeCN 1/1); Gradient: 50% to 75% gradient (26 min)

Preparative HPLC Method 3: Column: XBridge C18 OBD Prep (300 Å, 5 μm, 19×250 mm); Flow rate: 30 mL/minute; Detection wavelength: 260 nm; Solvents: buffer A: 10 mM HA/50 mM HFIP in water, Buffer B: MeCN; Gradient: 23˜ 28% Buffer B gradient (30 minutes).

The fractions containing the desired compound were concentrated and dissolved with 0.2 N NaCl in EtOH/water (1/4). The resulting solution was desalted by membrane filtration by using a 3000MW cut-off (3K centrifugal membrane tube, Amicon Ultra-15, Ultracel-3K (3400 rpm, 45 minutes) (cat.UFC900396 from Sigma-Aldrich) or Macrosep Devices (cat. MAP003C₃₈) from PALL, 3400 rpm, 40 minutes, 15 mL WFI×3). The final desalted solution was filtered (0.2 micron sterile syringe filter). The absorbance of the diluted solution was measured at 260 nm on a Nanodrop UV-Vis spectrophotometer to give a yield (7˜ 15% yield) and endotoxin level was confirmed to be less than 0.06 EU/mg by a kinetic chromogenic LAL method (Charles River, Endosafe©nexgen-PTS).

Part 5. Tm Measurement with Reverse Complementary RNA and NMR

Tm measurement device: Shimadzu UV-2700 UV-Vis Spectrophotometer

Protocol 1: ASO samples were prepared at a concentration of 400 μM using deionized water. IDT's reverse complementary RNA (rcRNA) was dissolved to 400 μM using UltraPure Distilled water. 10 μL aliquots of each stock solutions were diluted to 1 mL using ultra pure distilled water and their actual concentrations were measured by UV-Vis Spectrophotomer. Test samples (500 μL) were prepared containing 4.0 μM ASO with 4.0 μM rcRNA in buffer (100 mM NaCl, 10 mM Na phosphate pH 7.0 with 0.1 mM EDTA). Test samples were incubated in a 1 mL cuvette and heated from 15° C. to 105° C. at 0.5° C./minute. UV absorbance increase due to strand melting was monitored at 260 nm. Prior to the experiment, the samples were melted and reannealed by heating from 25° C. to 95° C. at 5° C./minute and cooling to starting temperatures to ensure complete annealing. Shimadzu Tm Analysis software was used to calculate the Tm (curve inflection point: 50% melting) using the derivative function.

Protocol 2: ASO samples were prepared at a concentration of 200 μM using PBS and then followed the same procedure as protocol 1 with adjusted amount.

³¹P NMR (162 MHz)³ was taken in stock phosphate buffer (100 mM, pD=7.4) that was prepared with 135.5 mg of K₂DPO₄ and 31.2 mg of KD₂PO₄ in 10 mL D₂O after C18 purification and deprotection of DMTr. See Evstigneev et al., “Hexamer oligonucleotide topology and assembly under solution phase NMR and theoretical modeling scrutiny,” Biopolymers 2010, 93 (12), 1023-1038.

### Exemplary Compounds

All nucleotides are 2′-MOE unless specified and “C” represent 5′-Methyl cytosine. A. Compound MOE-277: 20mer, all Sp

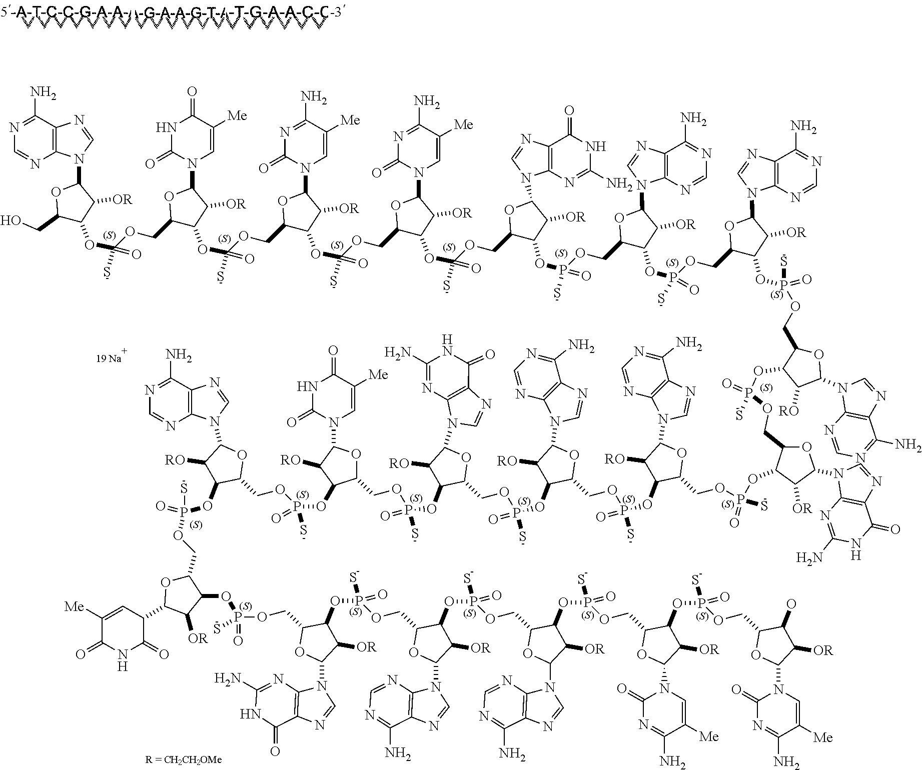

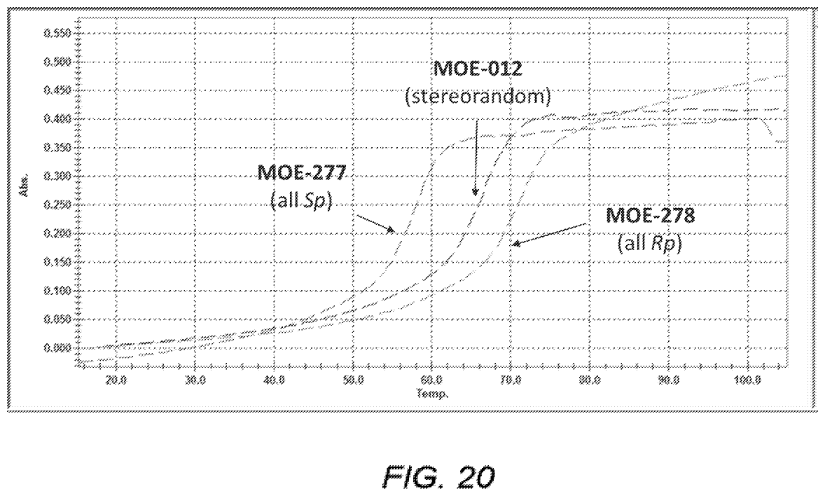

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ion using the most abundant natural isotopes, was detected by low resolution mass spectrometry at m/z 1995.05; Tm=57.8° C. (Tm of stereorandom=66.5° C.) by Protocol 1. The Tm of MOE-277 is shown in FIG. 20.

B. Compound MOE-278: 20mer, all Rp

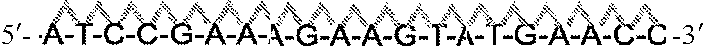

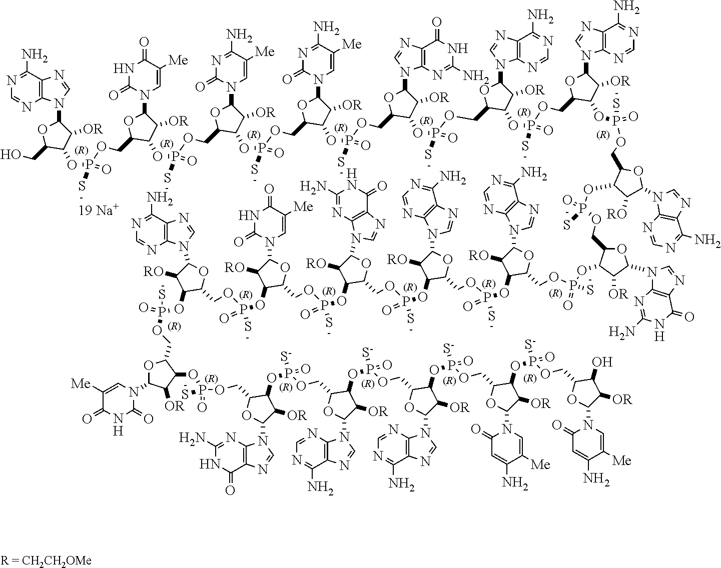

Purified by Preparative HPLC Method 2: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.492; Tm=71.5° C. (Tm of stereorandom=66.5° C.) by Protocol 1. The Tm of MOE-278 is shown in FIG. 20.

C. Compound MOE-279: 20mer, 4Rp

5′- [symbol] [symbol] -3′

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.25. Tm=61.4° C. (Tm of stereorandom=66.5° C.) by Protocol 1.

D. Compound MOE-280: 20mer, 5Rp

5′- [symbol] [symbol] -3′

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1994.88. Tm=62.7° C. (Tm of stereorandom=66.5° C.) by Protocol 1.

E. Compound MOE-281: 20mer, 7Rp

5′ [symbol] [symbol] -3′

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.06. Tm=62.3° C. (Tm of stereorandom=66.5° C.) by Protocol 1.

F. Compound MOE-282: 20mer, 7Rp

5′- [symbol] [symbol] [symbol] -3′

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1994.81. Tm=63.5° C. (Tm of stereorandom=66.5° C.) by Protocol 1.

### G. Compound MOE-283: 20mer 9R

5′- [symbol] [symbol] -3′

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.43. Tm=64.8° C. (Tm of stereorandom=66.5° C.) by Protocol 1.

H. Compound MOE-284: 20mer, 10Rp

5′ [symbol] [symbol] -3′

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1994.96; Tm=66.2° C. (Tm of stereorandom=66.5° C.) by Protocol 1.

I. Compound MOE-285: 20mer, 9Rp

5′- [symbol] [symbol] [symbol] -3′

Purified by Preparative HPLC Method 1: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.50; Tm=63.4° C. (Tm of stereorandom=66.5° C.) by Protocol 1.

J. Compound MOE-286: 20mer, 13Rp

5′- [symbol] [symbol] -3′

Purified by Preparative HPLC Method 2: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1994.55.

K. Compound MOE-287: 20mer, 3Rp

5′ [symbol] [symbol] -3′

Purified by Preparative HPLC Method 2: C₂₆₀H₃₇₂N₈₃O₁₃₃P₁₉S₁₉ Mw=7985.47 with a theoretical value of m/z 1995.36 as the [M-4H]⁴⁻ ion using the most abundant natural isotopes, was detected by low resolution mass spectrometry at m/z 1995.08; Tm=59.7° C. (Tm of stereorandom=66.5° C.) by Protocol 1.

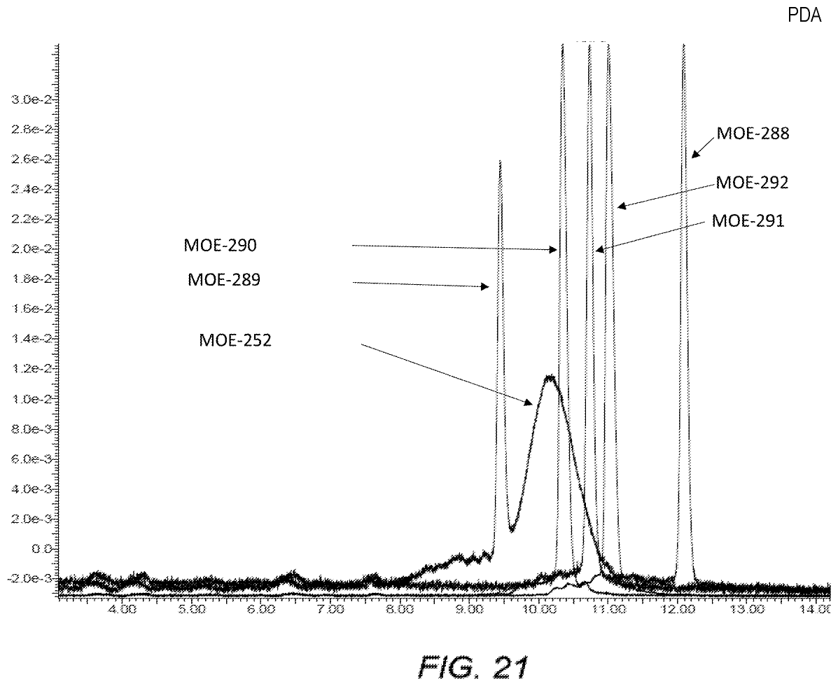

FIG. 20 shows the Tms of MOE-012, MOE-277, and MOE-278. FIG. 21 shows the Tms of MOE-277 to MOE-287.

L. Compound MOE-288: 18mer, all Sp

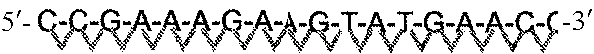

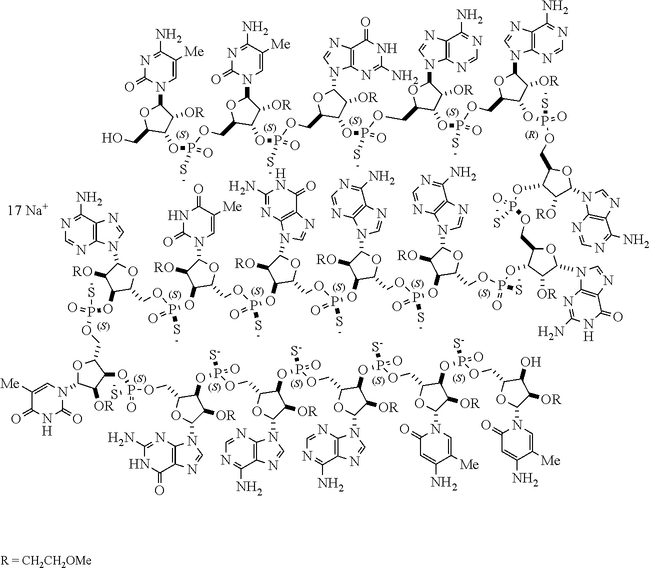

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.45; Tm=58.4° C. (Tm of stereorandom=65.9° C.) by Protocol 2.

³¹P NMR (162 MHz) δ ppm 56.09, 55.82, 55.78, 55.56, 55.52, 55.32, 55.21, 55.15, 55.06

M. Compound MOE-289: 18mer, all Rp

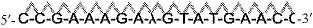

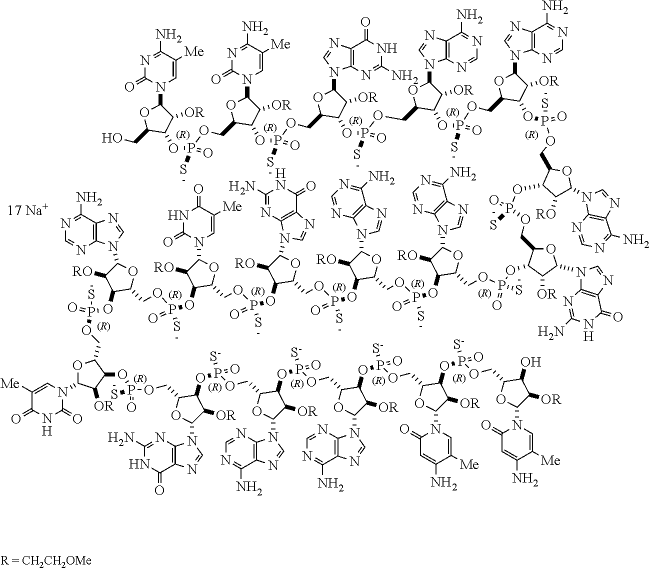

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.38; Tm=70.4° C. (Tm of stereorandom=65.9° C.) by Protocol 2.

³¹P NMR (162 MHz) δ ppm 56.09, 55.82, 55.78, 55.56, 55.52, 55.32, 55.21, 55.15, 55.06

N. Compound MOE-290: 18mer, 11Rp

5′-

[symbol]

[symbol]

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.57; Tm=66.6° C. (Tm of stereorandom=65.9° C.) by Protocol 2.

³¹P NMR (162 MHz) δ ppm 58.53, 58.22, 58.08, 57.82, 57.60, 57.40, 57.12, 55.67, 55.54, 55.30, 55.12

O. Compound MOE-291: 18mer, 8Rp

5′-

[symbol]

[symbol]

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.31; Tm=62.5° C. (Tm of stereorandom=65.9° C.) by Protocol 2.

³¹P NMR (162 MHz) δ ppm 57.07, 56.83, 56.71, 56.54, 56.31, 55.16, 54.81, 54.33, 54.24, 54.12, 54.23

P. Compound MOE-292: 18mer, 8Rp

5′

[symbol]

[symbol]

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1794.95; Tm=62.6° C. (Tm of stereorandom=65.9° C.) by Protocol 2.

³¹P NMR (162 MHz) δ ppm 60.01, 59.40, 59.36, 58.87, 58.50, 58.14, 57.67, 57.37, 57.15, 56.66, 56.48, 55.83, 55.55, 55.26

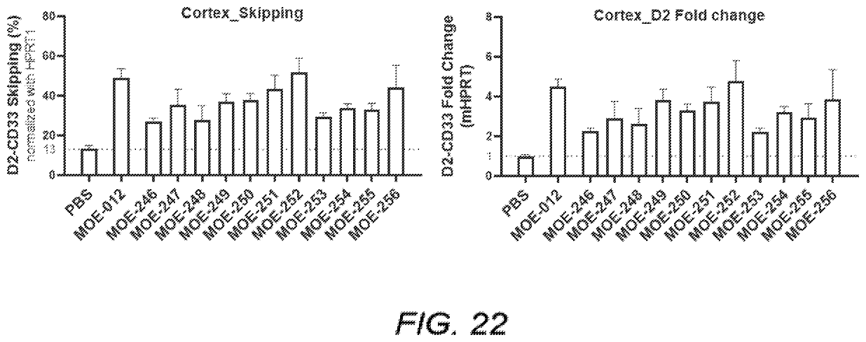

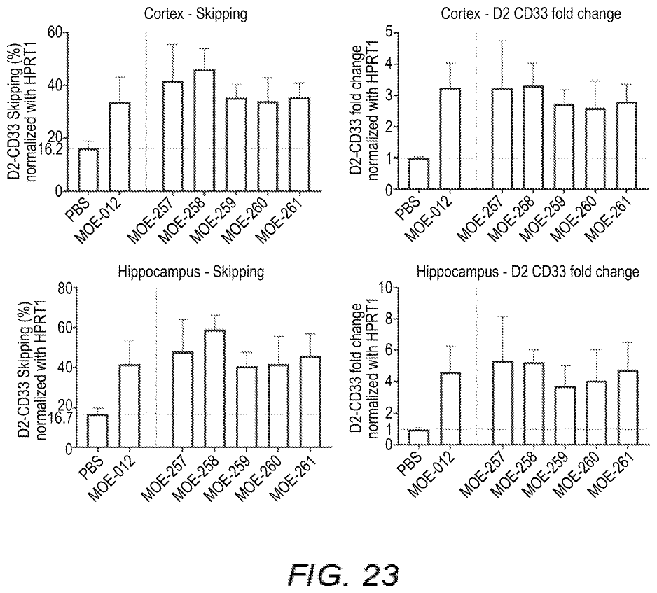

FIG. 22 shows the TMs of MOE-288 to MOE-292. FIG. 23 shows an example of overlay HPLC chromatogram (MOE-252 and MOE-288 to MOE-292 by Analytical HPLC Method 4.

Q. Compound MOE-293: 18mer, 4Rp

5′-

[symbol]

[symbol]

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.95; Tm=59.5° C. (Tm of stereorandom=65.9° C.) by Protocol 2.

³¹P NMR (162 MHz) δ ppm 56.79, 56.19, 55.09, 54.93, 54.85, 54.67, 54.53

R. Compound MOE-294: 18mer, 6Rp

5′-

[symbol]

[symbol]

Purified by Preparative HPLC Method 3: C₂₃₄H₃₃₅N₇₆O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.54; Tm=59.7° C. (Tm of stereorandom=65.9° C.) by Protocol 2.

³¹P NMR (162 MHz) δ ppm 57.84, 57.43, 57.17, 56.92, 56.80, 55.98, 55.86, 55.62, 55.58, 55.46, 55.27, 55.11, 55.06, 55.00

S. Compound MOE-295: 18mer, 4Rp

5′-

[symbol]

[symbol]

Purified by Preparative HPLC Method 3: C₂₃₄H₃₃₅N_{7M}O₁₁₉P₁₇S₁₇ Mw=7186.34 with a theoretical value of m/z 1795.59 as the [M-4H]⁴⁻ ion using the most abundant natural isotopes, was detected by low resolution mass spectrometry at m/z 1795.82; Tm=60.6° C. (Tm of stereorandom=65.9° C.) by Protocol 2.

³¹P NMR (162 MHz) δ ppm 57.71, 57.25, 57.06, 56.11, 55.79, 55.68, 55.48, 55.35, 55.21, 55.11

T. Compound MOE-296: 18mer, 2Rp/2PO

5′ [symbol] [symbol] -3′

Purified by Preparative HPLC Method 3: C₂₃₄H₃₃₅N₇₈O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.56; Tm=61.3° C. (Tm of stereorandom=65.9° C.) by Protocol 2.

³¹P NMR (162 MHz) δ ppm 58.26, 58.20, 57.84, 57.65, 57.49, 57.40, 57.16, 56.97, 0.33

U. Compound MOE-297: 18mer, 4Rp/2PO

5′- [symbol] [symbol] -3′

Purified by Preparative HPLC Method 3: C₂₃₄H₃₃₅N₇₈O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.35; Tm=62.7° C. (Tm of stereorandom=65.9° C.) by Protocol 2.

³¹P NMR (162 MHz) δ ppm 57.24, 57.04, 56.42, 56.36, 55.83, 55.69, 55.56, 55.36, 55.17, 55.07, 54.64,−1.03,−1.13

V. Compound MOE-298: 18mer, 2Rp/2PO

5′- [symbol] [symbol] -3′

Purified by Preparative HPLC Method 3: C₂₃₄H₃₃₅N₇₈O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.40; Tm=61.6° C. (Tm of stereorandom=65.9° C.) by Protocol 2.

³¹P NMR (162 MHz) δ ppm 57.20, 57.08, 56.77, 56.55, 56.17, 56.10,−0.72

W. Compound MOE-299: 20mer, 2Rp/2PO

5′- [symbol] [symbol] -3′

Purified by Preparative HPLC Method 3: C₂₈₀H₃₇₂N₈₃O₁₃₅P₁₉S₁₇ Mw=7950.51 with a theoretical value of m/z 1986.62 as the [M-4H]⁴⁻ ion using the most abundant natural isotopes, was detected by low resolution mass spectrometry at m/z 1987.01; Tm=61.5° C. (Tm of stereorandom=69.6° C.) by Protocol 1.

³¹P NMR (162 MHz) δ ppm 57.48, 57.22, 56.14, 55.90, 55.65, 55.77, 55.38, 55.30, 55.25, 55.21, 55.06, 54.94,−0.95,−0.99

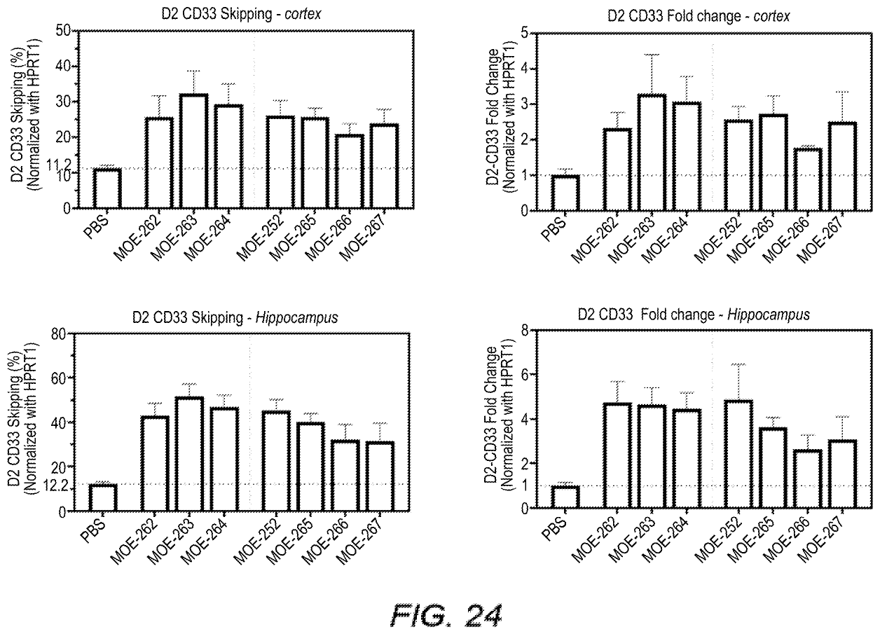

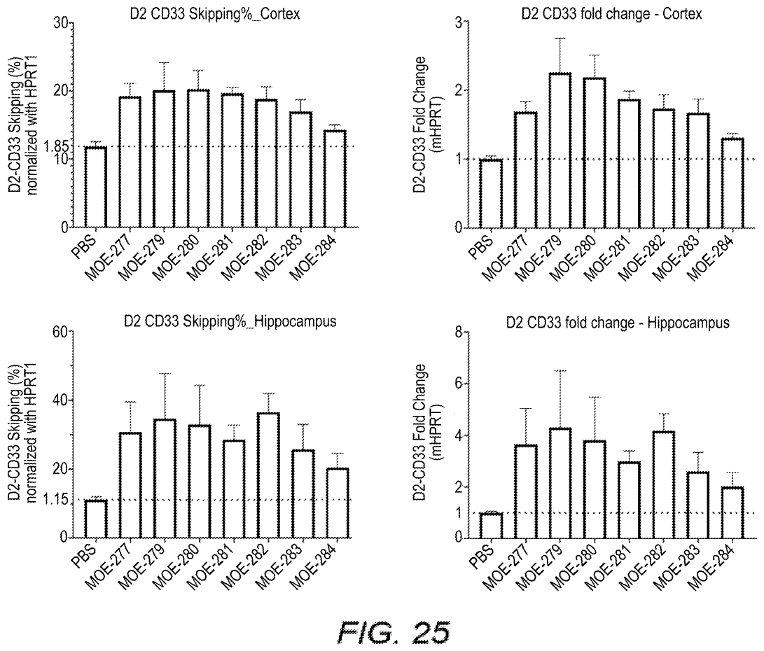

FIG. 24 shows the TMs of MOE-252 and MOE-293 to MOE-298. FIG. 25 shows the TMs of MOE-029 and MOE-299.−

X. Compound MOE-300: 18mer, 6Rp/2PO

5′ [symbol] [symbol] -3′

Purified by Preparative HPLC Method 3: C₂₃₄H₃₃₅N_{7M}O₁₂₁P₁₇S₁ₛ Mw=7154.38 with a theoretical value of m/z 1787.59 as the [M-4H]⁴⁻ ion using the most abundant natural isotopes, was detected by low resolution mass spectrometry at m/z 1787.49; Tm=63.2° C. (Tm of stereorandom=65.9° C.) by Protocol 2.

³¹P NMR (162 MHz) δ ppm 58.39, 58.07, 57.85, 57.69, 56.36, 56.06, 55.78, 55.70, 55.57, 55.38, 55.33, 55.29,−0.97

Y. Compound MOE-301: 18mer, 5Rp/2PO

5′- [symbol] [symbol] -3′

Purified by Preparative HPLC Method 3: C₂₃₄H₃₃₅N_{7M}O₁₂₁P₁₇S₁ₛ Mw=7154.38 with a theoretical value of m/z 1787.59 as the [M-4H]⁴⁻ ion using the most abundant natural isotopes, was detected by low resolution mass spectrometry at m/z 1787.66; Tm=62.2° C. (Tm of stereorandom=65.9° C.) by Protocol 2.

³¹P NMR (162 MHz) δ ppm 58.57, 58.02, 57.81, 57.65, 56.31, 56.02, 55.73, 55.64, 55.52, 55.33, 55.27, 55.25, 55.11,−1.00

Z. Compound MOE-303: 18mer, 3PO

5′- [symbol] [symbol] -3′

Purified by Preparative HPLC Method 3: C₂₃₄H₃₃₅N₇₆O₁₂₂P₁₇S₁₄ Mw=7137.40 with a theoretical value of m/z 1783.25 as the [M-4H]⁴⁻ ion using the most abundant natural isotopes, was detected by low resolution mass spectrometry at m/z 1782.76; Tm=59.5° C. (Tm of stereorandom=65.9° C.) by Protocol 2.

³¹P NMR (162 MHz) δ ppm 56.36, 56.11, 55.93, 55.84, 55.69, 55.64, 55.53, 55.38, −0.88, −0.97

AA. Compound MOE-304: 18mer, 5PO

5′- [symbol] [symbol] -3′

Purified by Preparative HPLC Method 3: C₂₃₄H₃₃₅N₇₆O₁₂₄P₁₇S₁₂ Mw=7106.45 with a theoretical value of m/z 1775.61 as the [M-4H]⁴⁻ ion using the most abundant natural isotopes, was detected by low resolution mass spectrometry at m/z 1775.83; Tm=61.6° C. (Tm of stereorandom=65.9° C.) by Protocol 2.

³¹P NMR (162 MHz, Solvent) δ ppm 56.29, 55.75, 55.71, 55.65, 55.53, 55.42,−0.62,−0.79,−0.89,−0.98

BB. Compound MOE-305: 18mer, 4PO

5′- [symbol] [symbol] -3′

Purified by Preparative HPLC Method 3: C₂₃₄H₃₃₅N₇₆O₁₂₃P₁₇S₁₃ Mw=7122.43 with a theoretical value of m/z 1779.50 as the [M-4H]⁴⁻ ion using the most abundant natural isotopes, was detected by low resolution mass spectrometry at m/z 1779.42; Tm=61.4° C. (Tm of stereorandom=65.9° C.) by Protocol 2.

³¹P NMR (162 MHz) δ ppm 55.54, 55.49, 55.72, 55.26, 55.14, 55.11, 54.98,−1.02,−1.06, −1.14,−1.47

CC. Compound MOE-306: 18mer, 3PO

5′- [symbol] [symbol] -3′

Purified by Preparative HPLC Method 3: C₂₃₄H₃₃₅N₇₆O₁₂₂P₁₇S₁₄ Mw=7137.40 with a theoretical value of m/z 1783.25 as the [M-4H]⁴⁻ ion using the most abundant natural isotopes, was detected by low resolution mass spectrometry at m/z 1783.54; Tm=60.3° C. (Tm of stereorandom=65.9° C.) by Protocol 2.

³¹P NMR (162 MHz) δ ppm 56.18, 55.88, 55.53, 55.37, 55.30, 55.64, 55.16, 55.07, −0.84, −0.90, −0.95

DD. Compound MOE-307: 18mer, 2PO

5′- [symbol] [symbol] -3′

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1788.19; Tm=59.0° C. (Tm of stereorandom=65.9° C.) by Protocol 2.

³¹P NMR (162 MHz) δ ppm 56.03, 55.84, 55.70, 55.56, 55.45, 55.27, 55.24, 55.11, 54.95,−1.10,−1.20

EE. Compound MOE-308: 20mer, 2PO

5′- [symbol] [symbol] -3′

Purified by Preparative HPLC Method 3: C₂₆₀H₃₇₂N₈₃O₁₃₅P₁₉S₁₇ Mw=7950.51 with a theoretical value of m/z 1986.62 as the [M-4H]⁴⁻ ion using the most abundant natural isotopes, was detected by low resolution mass spectrometry at m/z 1986.92; Tm=58.5° C. (Tm of stereorandom=69.6° C.) by Protocol 1.

³¹P NMR (162 MHz) δ ppm 55.91, 55.76, 55.52, 55.20, 55.10, 54.99, 54.89,−0.99,−1.05, -1.09

### FF. Compound MOE-309: 20mer 4PO

5′- [symbol] [symbol] -3′

Purified by Preparative HPLC Method 3: C₂₆₀H₃₇₂N₈₃O₁₃₇P₁₉S₁₅ Mw=7919.50 with a theoretical value of m/z 1978.87 as the [M-4H]⁴⁻ ion using the most abundant natural isotopes, was detected by low resolution mass spectrometry at m/z 1978.66; Tm=62.8° C. (Tm of stereorandom=69.6° C.) by Protocol 1.

³¹P NMR (162 MHz) δ ppm 55.83, 55.65, 55.50, 55.41, 55.19, 55.02, 55.11, 54.77,−1.04, -1.11,−1.15,−1.55

GG. Compound MOE-310: 18mer, 5R/2PO

5′- [symbol] [symbol] -3′

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.64; Tm=63.4° C. (Tm of stereorandom=65.9° C.) by Protocol 2.

³¹P NMR (162 MHz) δ ppm 58.22, 57.54, 56.08, 55.88, 55.41, 55.26, 55.17, 55.10, 55.04, 54.95,−1.02

### HH. Compound MOE-311: 18mer 4R/2PO

5′- [symbol] [symbol] -3′

Purified by Preparative HPLC Method 3: C₂₃₄H₃₃₅N₇₆O₁₂₁P₁₇S₁₅ Mw=7154.38 with a theoretical value of m/z 1787.59 as the [M-4H]⁴⁻ ion using the most abundant natural isotopes, was detected by low resolution mass spectrometry at m/z 1787.34; Tm=61.0° C. (Tm of stereorandom=65.9° C.) by Protocol 2.

³¹P NMR (162 MHz) δ ppm 58.21, 57.66, 56.05, 55.89, 55.57, 55.48, 55.38, 55.28, 55.26, 55.05, 54.96,−1.03,−1.25

### Example 14: In Vitro Assay to Assess Skipping Efficiency of Phosphorothioate (PS) Oligonucleotides in Mouse BMDMs

Freshly isolated mouse BMDM cells were cultured and maintained using appropriate media (Dulbecco's Modified Eagle's Medium containing 10% fetal bovine serum) plus recombinant murine CSF. The Assay was performed in 96 well plate format, seeding about 30,000 cells per well and treating with the ASO at a concentration of 1 μM, 3 μM, and 10 μM without addition of lipofectamine. Cells were incubated at 37° C. in a cell culture incubator for 48 hours before isolating the total RNA. Total RNA was isolated and converted to cDNA per vendor protocol, then Taqman gene expression assays were used to quantify Exon-2: skipped CD33 (Forward primer: CGCTGCTGCTACTGCTG (SEQ ID NO:207); Reverse Primer: TTCTAGAGTGCCAGGGATGA (SEQ ID NO:208); and probe: TGTGGGCAGACTTGACCCACAG (SEQ ID NO:209)) and un-skipped CD33 (Forward primer: GGATGGAGAGAGGAAGTA (SEQ ID NO:210); Reverse Primer: GTGCCAGGGATGAGGATTT (SEQ ID NO:211); and probe: TGCATGTGACAGACTTGACCCACA (SEQ ID NO:212)) mRNA transcripts. Mouse house-keeping gene HPRT1 (Assay ID: Hs02800695_m1; ThermoFisher Scientific) expressions was used to normalize the target transcript expressions. Non-targeting (NTC) MOE sequence CCTTCCCTGAAGGTTCCTCC (SEQ ID NO: 257) was used (Mullick et al. (2011) J. Lipid Res. 52, 885). The in vitro skipping data for select ASOs listed in Tables 13 and 14 is shown in Tables 16, 17, 18, 19, 20 and 21.

TABLE 16
In vitro % CD33 Exon-2 skipping data for select ASOs in
mouse BMDMs. Skipping for non-targeting control ASO (NTC)
is shown as control for the individual experiment.

| ASO | % D2 Skipping | % D2 Skipping | % D2 Skipping |
|---|---|---|---|
| number | at 10 μM | at 3 μM | at 1 μM |
| NTC | 14.53 | 14.22 | 16.67 |
| MOE-245 | 49.82 | 50.36 | 43.74 |
| MOE-246 | 38.21 | 27.62 | 27.53 |
| MOE-247 | 49.35 | 30.45 | 26.93 |
| MOE-248 | 39.88 | 23.61 | 23.61 |
| MOE-249 | 50.10 | 32.40 | 28.12 |
| MOE-250 | 50.03 | 36.50 | 31.20 |
| MOE-251 | 51.65 | 31.55 | 28.51 |
| MOE-252 | 47.31 | 31.88 | 28.61 |
| MOE-253 | 37.21 | 24.99 | 21.68 |
| MOE-254 | 48.19 | 31.22 | 27.38 |
| MOE-255 | 35.16 | 26.89 | 27.46 |

TABLE 17
In vitro % CD33 Exon-2 skipping data for select ASOs in
mouse BMDMs. Skipping for non-targeting control ASO (NTC)
is shown as control for the individual experiment.

| ASO | % D2 Skipping | % D2 Skipping | % D2 Skipping |
|---|---|---|---|
| number | at 10 μM | at 3 μM | at 1 μM |
| NTC | 12.29 | 12.87 | 13.10 |
| MOE-256 | 66.49 | 49.30 | 40.29 |
| MOE-257 | 68.67 | 40.30 | 35.85 |
| MOE-258 | 72.87 | 45.15 | 39.69 |
| MOE-259 | 69.53 | 42.52 | 35.47 |
| MOE-260 | 68.57 | 43.35 | 35.01 |
| MOE-261 | 65.92 | 38.36 | 32.69 |

TABLE 18
In vitro % CD33 Exon-2 skipping data for select ASOs in
mouse BMDMs. Skipping for non-targeting control ASO (NTC)
is shown as control for the individual experiment.

| ASO | % D2 Skipping | % D2 Skipping | % D2 Skipping |
|---|---|---|---|
| number | at 10 μM | at 3 μM | at 1 μM |
| NTC | 14.36 | 14.07 | 15.28 |
| MOE-262 | 68.80 | 41.56 | 34.94 |
| MOE-263 | 61.75 | 42.95 | 39.23 |
| MOE-264 | 64.99 | 42.68 | 38.54 |
| MOE-265 | 66.12 | 40.89 | 35.98 |
| MOE-266 | 68.07 | 42.00 | 38.88 |
| MOE-267 | 63.18 | 42.88 | 37.10 |

TABLE 19
In vitro % CD33 Exon-2 skipping data for select ASOs in
mouse BMDMs. Skipping for non-targeting control ASO (NTC)
is shown as control for the individual experiment.

| ASO | % D2 Skipping | % D2 Skipping | % D2 Skipping |
|---|---|---|---|
| number | at 10 μM | at 3 μM | at 1 μM |
| NTC | 13.90 | 13.27 | 16.43 |
| MOE-277 | 64.10 | 37.96 | 29.41 |
| MOE-278 | 34.60 | 21.94 | 21.37 |
| MOE-279 | 71.80 | 45.73 | 38.89 |
| MOE-280 | 62.39 | 39.16 | 31.20 |
| MOE-281 | 62.76 | 43.98 | 36.66 |
| MOE-282 | 63.53 | 35.93 | 49.15 |
| MOE-283 | 52.25 | 41.27 | 38.59 |
| MOE-284 | 44.54 | 34.82 | 30.51 |
| MOE-285 | 47.44 | 37.69 | 34.09 |
| MOE-286 | 32.65 | 25.61 | 26.96 |
| MOE-287 | 48.50 | 39.66 | 38.68 |

TABLE 20
In vitro % CD33 Exon-2 skipping data for select
ASOs in mouse BMDMs. Skipping for blank PBS is
shown as control for the individual experiment.

| ASO | % D2 Skipping | % D2 Skipping | % D2 Skipping |
|---|---|---|---|
| number | at 10 μM | at 3 μM | at 1 μM |
| PBS | 13.97 | 13.97 | 13.97 |
| MOE-268 | 68.05 | 47.09 | 34.43 |
| MOE-269 | 67.10 | 44.97 | 32.63 |
| MOE-270 | 61.13 | 43.03 | 30.94 |
| MOE-271 | 68.60 | 48.46 | 36.29 |
| MOE-272 | 74.17 | 54.61 | 40.17 |
| MOE-273 | 73.23 | 50.45 | 39.60 |
| MOE-274 | 69.18 | 46.22 | 37.73 |
| MOE-275 | 44.96 | 32.26 | 20.87 |
| MOE-276 | 70.31 | 58.36 | 41.69 |

TABLE 21
In vitro % CD33 Exon-2 skipping data for select ASOs in
mouse BMDMs. Skipping for non-targeting control ASO (NTC)
is shown as control for the individual experiment.

| ASO | % D2 Skipping | % D2 Skipping | % D2 Skipping |
|---|---|---|---|
| number | at 10 μM | at 3 μM | at 1 μM |
| NTC | 10.16 | 8.49 | 8.63 |
| MOE-288 | 78.23 | 60.11 | 36.87 |
| MOE-289 | 50.20 | 32.50 | 24.91 |
| MOE-290 | 68.08 | 43.32 | 35.52 |
| MOE-291 | 73.67 | 52.99 | 37.79 |
| MOE-292 | 71.01 | 55.63 | 40.32 |

Select ASO sequences were tested for their efficacy in inducing skipping of Exon-2 in a CD33 gene transcript in U-118 MG glioblastoma cells in vitro. The experimental details are identical to those used in Example 4 (Evaluation of splice modulation properties of CD33 Exon-2 targeting oligonucleotides/In vitro assay methods/Evaluation of MOE-ASO Sequences). In this experiment, MOE-ASOs were evaluated at 3.33 nM, 10 nM and 30 nM concentrations. The data is shown in Table 22.

TABLE 22
In vitro % D2 skipping data for select ASOs in
U118-MG cells Skipping for non-targeting control
ASO (NTC) and water are shown as control.

| | % D2 skipping | % D2 skipping | % D2 skipping |
|---|---|---|---|
| ASO | at 30 nM | at 10 nM | at 3.33 nM |
| MOE-252 | 41.82 | 31.99 | 16.92 |
| MOE-277 | 37.63 | 31.54 | 23.93 |
| MOE-278 | 52.14 | 37.51 | 19.02 |
| MOE-279 | 50.02 | 37.48 | 31.68 |
| MOE-280 | 56.77 | 40.14 | 25.78 |
| MOE-281 | 48.42 | 54.49 | 31.16 |
| MOE-282 | 36.11 | 36.55 | 20.57 |
| MOE-283 | 48.42 | 43.26 | 27.82 |
| MOE-284 | 51.64 | 38.60 | 24.11 |
| MOE-285 | 48.53 | 35.77 | 25.33 |
| MOE-275 | 74.89 | 63.06 | 44.09 |
| MOE-288 | 35.20 | 28.44 | 27.02 |
| MOE-289 | 45.72 | 28.84 | 18.90 |
| MOE-290 | 46.64 | 35.73 | 23.58 |
| MOE-291 | 52.54 | 42.30 | 30.26 |
| MOE-292 | 51.13 | 36.48 | 24.71 |
| MOE-293 | 47.16 | 39.80 | 26.46 |
| MOE-294 | 61.46 | 42.57 | 30.78 |
| MOE-295 | 54.12 | 38.56 | 27.56 |
| MOE-296 | 49.18 | 31.38 | 23.25 |
| MOE-297 | 59.64 | 43.28 | 26.22 |
| MOE-298 | 57.83 | 39.06 | 20.83 |
| MOE-299 | 55.34 | 47.80 | 38.21 |
| NTC | 14.52 | 15.33 | 20.26 |

| | |
|---|---|
| Water | 17.84 |

### Example 15: In Vivo Assay Methods

Humanized 0033 mouse models were used to study CD33 Exon-2 skipping ASOs. CRISPR/Cas9 mediated gene editing was used to replace murine CD33 with human genomic CD33, including the signal peptide. Murine 3′ and 5′ untranslated regions were retained. For in vivo experiments, mixed gender cohorts of human 0033 mouse lines on a C57BL/6 background were used, mice were 12-24 weeks old at the time of dosing.

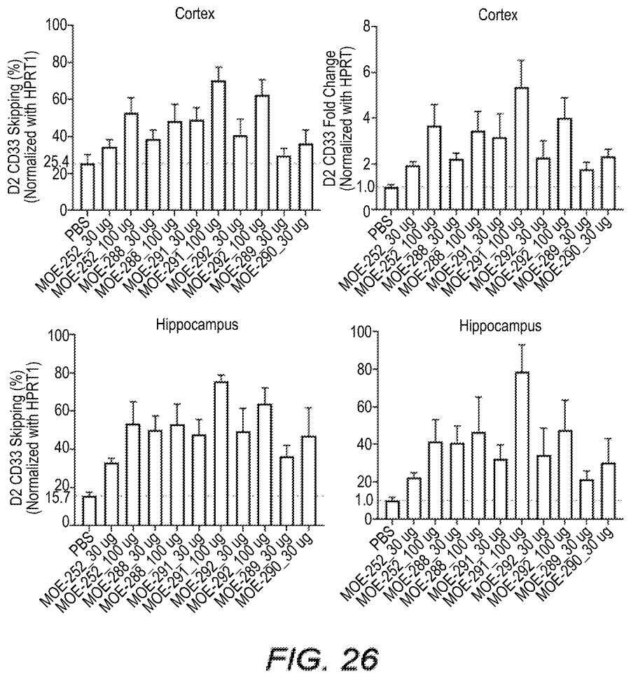

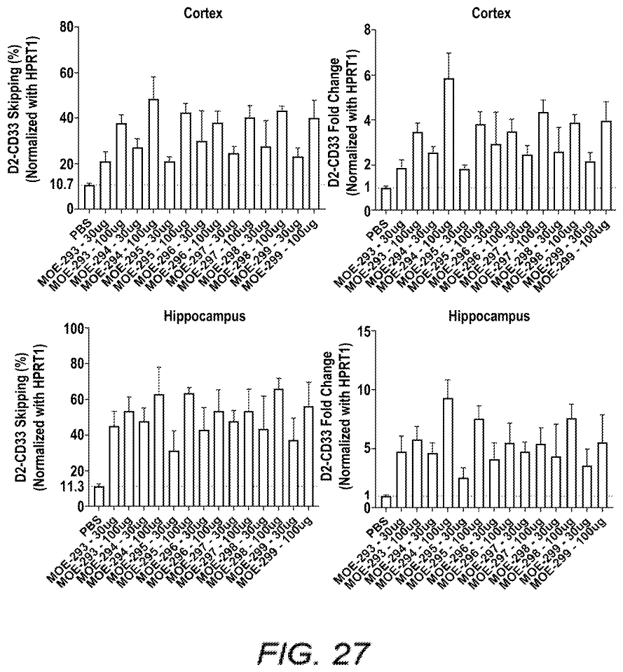

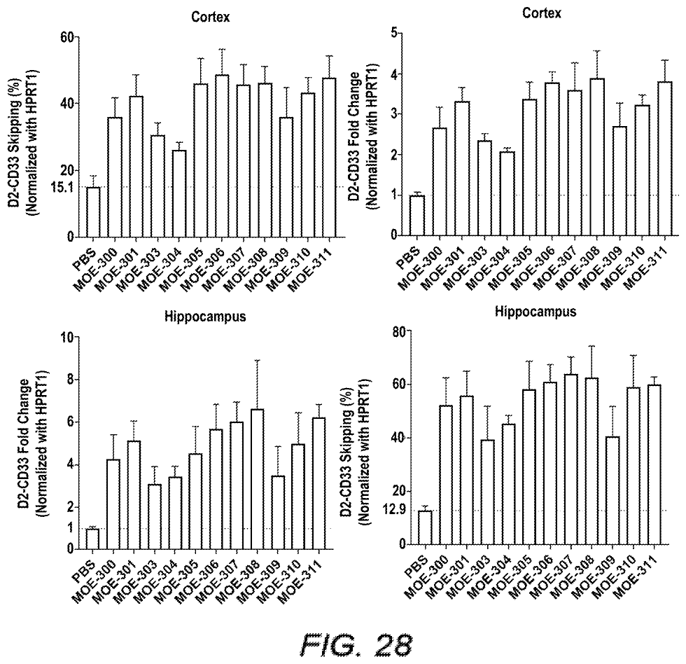

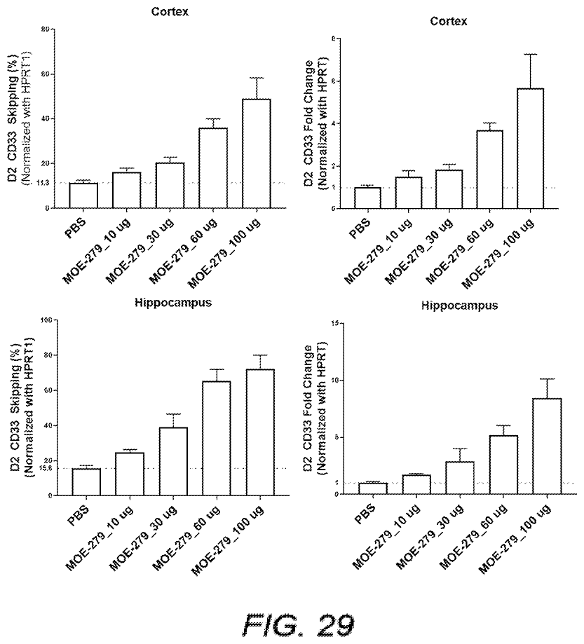

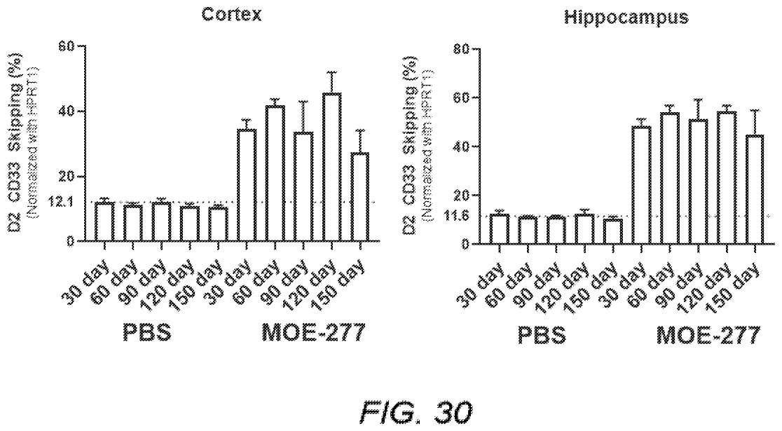

ASOs were administered via intracerebroventricular injection at the appropriate dose into the right lateral ventricle in a 10 μL bolus on day 1. Mice were necropsied 14 days after the injection, unless noted otherwise. At necropsy, mice were transcardially perfused with PBS under avertin anesthesia. Brains were rapidly removed from the skull, and the cortex and hippocampus were dissected from the injected hemisphere for exon skipping evaluation. For RNA isolation, frozen tissue was added with 9× volume of Trizol and homogenized for 3 minutes. 500 μL of the Trizol lysate was transferred to a 1 mL deep well plate. 100 μL of chloroform was added to each sample, shaken vigorously, and centrifuged at 4000×g for 5 minutes. The supernatant (250 μL) was transferred to the binding plate from SV96 total RNA extraction kit (Promega) and RNA was extracted per the same protocol. Total RNA was isolated and converted to cDNA per SV96 protocol (Promega), then Taqman gene expression assays were used to quantify Exon-2 skipped CD33 mRNA transcripts. Mouse house-keeping gene HPRT1 expression was used to normalize the target transcript expressions. The data can be expressed as fold change of Exon-2 skipped CD33 mRNA as compared with PBS treated group. Alternatively, the data can be expressed as the amount (%) of Exon-2 skipped CD33 mRNA in vivo relative to PBS control. The in vivo skipping data for select sequences listed in Tables 13 and 14 is shown in FIGS. 22-28. In vivo dose response for MOE-279 is shown in FIG. 29. Duration of effect of MOE-277 after a single ICV dose of 100 μg is shown in FIG. 30.

### Example 16: Hybridization ELISA for Determining Concentration of ASOs in Brain Tissues

Concentrations of ASO was quantified in mouse cortex and hippocampus using a hybridization-based immunoassay method (HELISA). Two single-stranded DNA oligonucleotides with complementary sequences to MOE-277 were designed as Detection probe: TCTTTCGGAT/3′-Bio (TCTTTCGGAT (SEQ ID NO:258)); and Capture probe: 5′-DigN/GGTTCATACT (GGTTCATACT (SEQ ID NO: 259))(Integrated DNA Technologies, Coralville, IA).

Tissues were lysed in TRIzol, 1:10 (Thermo Fisher Scientific, Waltham, MA), and were diluted in hybridization buffer (1:100, 1M NaCl in TE-Buffer and 0.1% Tween20). MOE-277 was spiked in diluted tissue homogenate to prepare standard curves and quality control (QC) samples. 35 μL of diluted samples, standards and QCs were transferred to a 96-well PCR plate. 35 μl of detection probe solution (100 nM in hybridization buffer), was added to the PCR plate containing standards and samples. Sample and detection probe were hybridized on a thermal cycler under the following conditions: 95° C. for 10 minutes, 37° C. for 60 minutes, and a final hold at 4° C.

MSD Gold 96-well Streptavidin SECTOR plate (Meso Scale Diagnostics, LLC., Rockville, MD) was blocked with 150 μL of Casein in TBS blocker (Thermo Fisher Scientific, Waltham, MA) at room temperature for 1.5 hours. After washing, 25 μL of capture probe (200 nM in hybridization buffer), was added to the MSD plate and incubated at 37° C., 300 rpm for 1 hour. After the wash step, 25 μL of samples, standards and QCs were transferred to an MSD plate in duplicate, and were incubated at 37° C. for 1 hour on a shaking platform (300 rpm). The plate was then washed 3 times and incubated for 1 hour with 50 μL of 1 μg/mL ruthenium labeled anti-digoxygenin antibody in Casein in TBS Blocking Buffer and 0.05% Tween20.

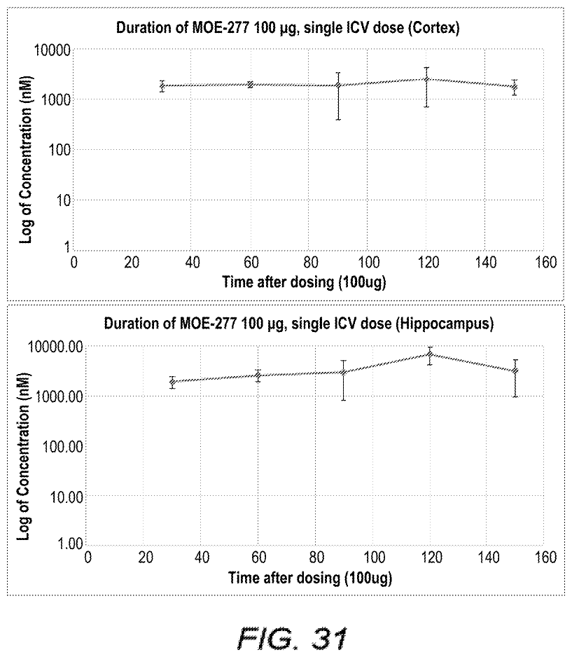

After the final wash, 150 μL of 2× MSD Read Buffer T (Meso Scale Diagnostics, LLC., Rockville, MD) was added and the plate was read on an MSD Sector S 600 instrument (Meso Scale Diagnostics, LLC., Rockville, MD). A nonlinear regression analysis was performed to calculate the concentrations of reference compound from the signal intensities via interpolation from a calibration curve using 4-parameter logistic (4PL) model (weighting factor=1/Y2) in Discovery Workbench 4.012.1 (Meso Scale Diagnostics, LLC., Rockville, MD). PK data analysis for duration of MOE-277 is shown in FIG. 31.

Those having ordinary skill in the art will appreciate that the disclosure can be modified in ways not specifically described herein. The disclosure is not to be limited in scope by the specific embodiments described herein, which are for illustrative purposes only. The disclosure includes any modifications and variations, including all functionally equivalent productions, compositions, and methods.

The entire disclosures of all publications cited herein are hereby incorporated by reference. No admission is made that any such publication constitutes prior art or is part of the common general knowledge of those having ordinary skill in the art.

1-188. (canceled)

189. An antisense oligonucleotide of 16-30 nucleotides in length, wherein the antisense oligonucleotide is complementary to a portion of SEQ ID NO:1, and wherein the antisense oligonucleotide has a CD33 Exon-2 skipping efficiency of 30% or greater according to a Standard Exon-Skipping Efficiency Assay for the antisense oligonucleotide.

190. The antisense oligonucleotide of claim 189, wherein the antisense oligonucleotide is complementary to a portion of SEQ ID NO:213, SEQ ID NO:214, SEQ ID NO:215, SEQ ID NO:216, SEQ ID NO:217, SEQ ID NO:218, SEQ ID NO:219, and/or SEQ ID NO:220.

191. The antisense oligonucleotide of claim 190, wherein the antisense oligonucleotide is complementary to a portion of SEQ ID NO:213 or SEQ ID NO:218.

192. The antisense oligonucleotide of claim 189, wherein the antisense oligonucleotide is 18-25 nucleotides in length.

193. The antisense oligonucleotide of claim 189, wherein the antisense oligonucleotide comprises at least one non-natural sugar moiety, at least one non-natural internucleotide linkage, or at least one non-natural sugar moiety and at least one non-natural internucleotide linkage.

194. The antisense oligonucleotide of claim 193, wherein the at least one non-natural sugar moiety comprises 2′-O-MOE, and wherein the antisense oligonucleotide has a CD33 Exon-2 skipping efficiency of 30% or greater according to a Standard Exon-Skipping Efficiency Assay for MOE ASOs.

195. The antisense oligonucleotide of claim 193, wherein the antisense oligonucleotide comprises a PMO, and wherein the antisense oligonucleotide has a CD33 Exon-2 skipping efficiency of 30% or greater according to a Standard Exon-Skipping Efficiency Assay for PMO ASOs.

196. The antisense oligonucleotide of claim 193, wherein the non-natural internucleotide linkages are stereopure.

197. The antisense oligonucleotide of claim 196, wherein the non-natural internucleotide linkages are all Sp.

198. The antisense oligonucleotide of claim 196, wherein the non-natural internucleotide linkages are all Rp.

199. The antisense oligonucleotide of claim 196, wherein the non-natural internucleotide linkages are independently selected from Sp and Rp.

200. The antisense oligonucleotide of claim 193, wherein the non-natural internucleotide linkages are stereorandom.

201. The antisense oligonucleotide of claim 193, wherein the antisense oligonucleotide comprises at least one modified nucleobase.

202. The antisense oligonucleotide of claim 189, wherein the antisense oligonucleotide comprises all or a portion of PMO-002 (SEQ ID NO:2); PMO-003 (SEQ ID NO:3); PMO-036 (SEQ ID NO:36); PMO-037 (SEQ ID NO:37); PMO-004 (SEQ ID NO:4); PMO-038 (SEQ ID NO:38); PMO-039 (SEQ ID NO:39); PMO-005 (SEQ ID NO:5); PMO-082 (SEQ ID NO:82); PMO-083 (SEQ ID NO:83); PMO-006 (SEQ ID NO:6); PMO-096 (SEQ ID NO:96); PMO-007 (SEQ ID NO:7); PMO-097 (SEQ ID NO:97); PMO-008 (SEQ ID NO:8); MOE-009 (SEQ ID NO:9); MOE-128 (SEQ ID NO:128); MOE-010 (SEQ ID NO:10); MOE-132 (SEQ ID NO:132); MOE-135 (SEQ ID NO:135); MOE-011 (SEQ ID NO: 11); MOE-012 (SEQ ID NO:12); MOE-136 (SEQ ID NO:136); MOE-013 (SEQ ID NO:13); MOE-014 (SEQ ID NO:14); MOE-015 (SEQ ID NO:15); MOE-183 (SEQ ID NO:183); MOE-184 (SEQ ID NO:184); MOE-190 (SEQ ID NO:190); MOE-196 (SEQ ID NO:196); MOE-197 (SEQ ID NO:197); PMO-221 (SEQ ID NO:221); PMO-222 (SEQ ID NO:222); PMO-223 (SEQ ID NO:223); PMO-224 (SEQ ID NO:224); PMO-225 (SEQ ID NO:225); PMO-226 (SEQ ID NO:226); PMO-227 (SEQ ID NO:227); PMO-228 (SEQ ID NO:228); PMO-229 (SEQ ID NO:229); PMO-230 (SEQ ID NO:230); PMO-231 (SEQ ID NO:231); PMO-232 (SEQ ID NO:232); PMO-233 (SEQ ID NO:233); PMO-234 (SEQ ID NO:234); PMO-235 (SEQ ID NO:235); PMO-236 (SEQ ID NO:236); PMO-237 (SEQ ID NO:237); PMO-238 (SEQ ID NO:238); PMO-239 (SEQ ID NO:239); PMO-240 (SEQ ID NO:240); PMO-241 (SEQ ID NO:241); PMO-242 (SEQ ID NO:242); PMO-243 (SEQ ID NO:243); PMO-244 (SEQ ID NO:244); PMO-324 (SEQ ID NO:224), Stereopattern: RRRRRRRRRRRRRRRRRRRR; PMO-424 (SEQ ID NO:224), Stereopattern: SSSSSSSSSSSSSSSSSSSS; PMO-402 (SEQ ID NO:002), Stereopattern: RRRRRRRRRRRRRRRRRRRRRRRR; PMO-502 (SEQ ID NO:002), Stereopattern: SSSSSSSSSSSSSSSSSSSSSSSS; MOE-245 (SEQ ID NO:245); MOE-246 (SEQ ID NO:246); MOE-247 (SEQ ID NO:247); MOE-248 (SEQ ID NO:248); MOE-249 (SEQ ID NO:249); MOE-250 (SEQ ID NO:250); MOE-251 (SEQ ID NO:251); MOE-252 (SEQ ID NO:252); MOE-253 (SEQ ID NO:253); MOE-254 (SEQ ID NO:254); MOE-255 (SEQ ID NO:255); MOE-256 (SEQ ID NO:256); MOE-257 (SEQ ID NO:012); MOE-258 (SEQ ID NO:012); MOE-259 (SEQ ID NO:012); MOE-260 (SEQ ID NO:012); MOE-261 (SEQ ID NO:012); MOE-262 (SEQ ID NO:012); MOE-263 (SEQ ID NO:012); MOE-264 (SEQ ID NO:012); MOE-265 (SEQ ID NO:252); MOE-266 (SEQ ID NO:252); MOE-267 (SEQ ID NO:252); MOE-268 (SEQ ID NO:252); MOE-269 (SEQ ID NO:252); MOE-270 (SEQ ID NO:252); MOE-271 (SEQ ID NO:252); MOE-272 (SEQ ID NO:252); MOE-273 (SEQ ID NO:252); MOE-274 (SEQ ID NO:252); MOE-275 (SEQ ID NO:012); MOE-276 (SEQ ID NO:012); MOE-277 (SEQ ID NO:012), Stereopattern: SSSSSSSSSSSSSSSSSSS; MOE-278 (SEQ ID NO:012), Stereopattern: RRRRRRRRRRRRRRRRRRR; MOE-279 (SEQ ID NO:012), Stereopattern: SSSRSSSRSSSRSSSRSSS; MOE-280 (SEQ ID NO:012), Stereopattern: SSSRSSRSSRSSRSSRSSS; MOE-281 (SEQ ID NO:012), Stereopattern: SSSRSRSRSRSRSRSRSSS; MOE-282 (SEQ ID NO:012), Stereopattern: SSSSSSRRRRRRRSSSSSS; MOE-283 (SEQ ID NO:012), Stereopattern: SSSRRSRRSRRSRRSRSSS; MOE-284 (SEQ ID NO:012), Stereopattern: SSSRRSRRRSRRRSRRSSS; MOE-285 (SEQ ID NO:012), Stereopattern: SSSSSRRRRRRRRRSSSSS; MOE-286 (SEQ ID NO:012), Stereopattern: SSSRRRRRRRRRRRRRSS; MOE-287 (SEQ ID NO:012), Stereopattern: SSRSSSSSSSSRSRSSSSS; MOE-288 (SEQ ID NO:252), Stereopattern: SSSSSSSSSSSSSSSSS; MOE-289 (SEQ ID NO:252), Stereopattern: RRRRRRRRRRRRRRRRR; MOE-290 (SEQ ID NO:252, Stereopattern: SSSRRRRRRRRRRRSSS; MOE-291 (SEQ ID NO:252), Stereopattern: RRRRRRRRSSSSSSSSS; MOE-292 (SEQ ID NO:252), Stereopattern: SSSSSSSSSRRRRRRRR; MOE-293 (SEQ ID NO:252), Stereopattern: SSSRSSRSSSRSSRSSS; MOE-294 (SEQ ID NO:252), Stereopattern: SSSRSRSRSRSRSRSSS; MOE-295 (SEQ ID NO:252), Stereopattern: SRSSSRSSSRSSSRSSS; MOE-296 (SEQ ID NO:252), Stereopattern: SSSOSSRSSSRSSOSSS; MOE-297 (SEQ ID NO:252), Stereopattern: SSSOSRSRSRSSSOSSS; MOE-298 (SEQ ID NO:252), Stereopattern: SOSSSRSSSRSSSOSSS; MOE-299 (SEQ ID NO:12), Stereopattern: SSSOSSSRSSSRSSSOSSS; MOE-300 (SEQ ID NO:252), Stereopattern: RRRORRROSSSSSSSSS; MOE-301 (SEQ ID NO:252), Stereopattern: SRRORRROSSSSSSSSS; MOE-303 (SEQ ID NO:252), Stereopattern: SSOOOSSSSSSSSSSSS; MOE-304 (SEQ ID NO:252), Stereopattern: OOOOOSSSSSSSSSSSS; MOE-305 (SEQ ID NO:252), Stereopattern: SSOSSSOSSOSSSOSSS; MOE-306 (SEQ ID NO:252), Stereopattern: SOSSSSOSSSSSSOSSS; MOE-307 (SEQ ID NO:252), Stereopattern: SSOSSSSSSSSSSOSSS; MOE-308 (SEQ ID NO:12), Stereopattern: SSSOSSSSSSSSSSSOSSS; MOE-309 (SEQ ID NO:12), Stereopattern: SSSOSSSSOSSOSSSOSSS; MOE-310 (SEQ ID NO:252), Stereopattern: SSORRRRRSSSSSOSSS; or MOE-311 (SEQ ID NO:252), Stereopattern: RRRRROSSSSSSSOSSS.

203. The antisense oligonucleotide of claim 202, wherein the antisense oligonucleotide comprises all or a portion of PMO-002 (SEQ ID NO:2), PMO-424 (SEQ ID NO:224; Stereopattern: SSSSSSSSSSSSSSSSSSSS), or MOE-307 (SEQ ID NO:252; Stereopattern: SSOSSSSSSSSSSOSSS).

204. A composition comprising the antisense oligonucleotide of claim 189 and a pharmaceutically acceptable carrier or excipient.

205. A method of inducing Exon-2 skipping in the CD33 gene during pre-mRNA splicing, comprising introducing a nucleic acid molecule into a cell, wherein the nucleic acid molecule is the antisense oligonucleotide of claim 189, hybridizes to a target region of the CD33 gene, and induces Exon-2 skipping during pre-mRNA splicing of the CD33 gene, and wherein the Exon-2 skipping efficiency of the antisense oligonucleotide is 30% or greater according to a Standard Exon-Skipping Efficiency Assay for ASOs.

206. The method of claim 205, wherein the antisense oligonucleotide comprises at least one 2′-O-MOE non-natural sugar moiety, or wherein the antisense oligonucleotide comprises a PMO.

207. A method of treating a subject having a neurodegenerative disease comprising administering to said subject a therapeutically effective amount of the antisense oligonucleotide of claim 189.

208. The method of claim 207, wherein the neurodegenerative disease is Alzheimer's Disease.
